(19) United States
(12) Patent Application Publication
(10) Pub. No.: US 2024/0325533 A1
(43) Pub. Date: Oct. 3, 2024

(54) MODIFIED IMMUNE CELLS AND METHODS OF USING THE SAME

(71) Applicant: Beam Therapeutics Inc., Cambridge, MA (US)

(72) Inventors: RYAN MURRAY, Cambridge, MA (US); Michail Sitkovsky, Boston, MA (US); Stephen Hatfield, Boston, MA (US)

(73) Assignee: Beam Therapeutics Inc., Cambridge, MA (US)

(21) Appl. No.: 18/737,787

(22) Filed: Jun. 7, 2024

Related U.S. Application Data
(63) Continuation of application No. PCT/US2022/081241, filed on Dec. 9, 2022.
(60) Provisional application No. 63/378,607, filed on Oct. 6, 2022.
(60) Provisional application No. 63/355,036, filed on Jun. 23, 2022.
(60) Provisional application No. 63/288,462, filed on Dec. 10, 2021.

(51) Int. Cl. A61K 39/00; A61P 35/00; C07K 14/725; C07K 16/28; C12N 9/22; C12N 9/78; C12N 15/11; C12N 15/90

(52) U.S. Cl. CPC A61K 39/4634; A61K 39/4611; A61K 39/4643; A61P 35/00; C07K 14/7051; C07K 16/2863; C12N 9/22; C12N 9/78; C12N 15/11; C12N 15/907; C12Y 305/04004; A61K 2239/26; C12N 2310/20

(57) ABSTRACT

The present invention features modified immune cells (e.g., T- or NK-cells) having increased resistance to hypoxia-adenosinergic immunosuppression. Methods for producing and using the same are also provided.

### CROSS REFERENCE TO RELATED APPLICATIONS

The present application is a U.S. National Stage filed under 35 U.S.C. § 111(a), which is a continuation of and claims priority to International Application No. PCT/US2022/081241, filed Dec. 9, 2022, which claims priority to and benefit of claims priority to U.S. Provisional Applications No. 63/378,607, filed Oct. 6, 2022, 63/355,036, filed Jun. 23, 2022, and 63/288,462, filed Dec. 10, 2021, the entire contents of each of which are hereby incorporated herein by reference in their entirety.

### SEQUENCE LISTING

This application contains a Sequence Listing which has been submitted electronically in XML format and is hereby incorporated by reference in its entirety. The Sequence Listing XML file, created on Jun. 7, 2024, is named 180802-050005US_SL.xml, and is 1,083,967 bytes in size.

### BACKGROUND OF THE INVENTION

Autologous and allogeneic immunotherapies are neoplasia treatment approaches in which immune cells expressing chimeric antigen receptors are administered to a subject. To generate an immune cell that expresses a chimeric antigen receptor (CAR), the immune cell is first collected from the subject (autologous) or a donor separate from the subject receiving treatment (allogeneic) and genetically modified to express the chimeric antigen receptor. The resulting cell expresses the chimeric antigen receptor on its cell surface (e.g., CAR T-cell), and upon administration to the subject, the chimeric antigen receptor binds to the marker expressed by the neoplastic cell. This interaction with the neoplasia marker activates the CAR-T cell, which then kills the neoplastic cell. But for autologous or allogeneic cell therapy to be effective and efficient, significant conditions and cellular responses, such as hypoxia-adenosinergic immunosuppression, must be overcome or avoided. Editing genes involved in these processes can enhance CAR-T cell function and resistance to immunosuppression or inhibition, but current methodologies for making such edits have the potential to induce large, genomic rearrangements in the CAR-T cell, thereby negatively impacting its efficacy. Thus, there is a significant need for techniques to more precisely modify immune cells, especially CAR-T cells. This application is directed to this and other important needs.

### SUMMARY OF THE INVENTION

As described below, the present invention features modified immune cells (e.g., T- or NK-cells) having increased resistance to hypoxia-adenosinergic immunosuppression. Methods for producing and using the same are also provided.

In one aspect, the invention features a method for producing a modified immune cell containing an alteration in a hypoxic and/or adenosinergic pathway. The method involves contacting the cell with (i) a base editor or a polynucleotide encoding the base editor and (ii) one or more guide polynucleotides or a polynucleotide encoding the guide polynucleotides. The base editor contains a programmable DNA binding domain and a deaminase domain. Each of the guide polynucleotides directs the base editor to effect a nucleobase alteration in a gene encoding a polypeptide component of the hypoxic and/or adenosinergic pathway or a regulatory element thereof, thereby producing a modified immune cell.

In another aspect, the invention features a method for producing a modified immune cell. The method involves contacting the cell with (i) a base editor or a polynucleotide encoding the base editor and (ii) one or more guide polynucleotides or a polynucleotide encoding the guide polynucleotides. The base editor contains a programmable DNA binding domain and a deaminase domain. Each of the guide polynucleotides directs the base editor to effect a nucleobase alteration in a gene selected from one or more of A2AR, A2BR, HIF1α, and HIF1α.I3, thereby producing a modified immune cell.

In another aspect, the invention features a method for reducing the expression of a Hypoxia-Inducible Factor 1-alpha (HIF1α) or HIF1α.I3 polypeptide and/or polynucleotide in a cell. The method involves contacting a cell containing a HIF1α or HIF1α.I3 gene with (i) a base editor or a polynucleotide encoding the base editor and (ii) one or more guide polynucleotides or a polynucleotide encoding the guide polynucleotides. The base editor contains a programmable DNA binding domain and a deaminase domain. Each of the guide polynucleotides directs the base editor to effect a nucleobase alteration in a HIF1α, and/or HIF1α.I3 gene that alters a splice acceptor or splice donor site, introduces a stop codon, or otherwise disrupts expression of the gene, thereby reducing expression of a Hypoxia-Inducible Factor 1-alpha (HIF1α) or HIF1α.I3 polypeptide and/or polynucleotide in the cell.

In another aspect, the invention features a method for reducing the expression of an Adenosine A_{2A} Receptor (A2AR) or A_{2B} Receptor (A2BR) polypeptide and/or polynucleotide in a cell. The method involves contacting a cell containing an A2AR or A2BR gene with (i) a base editor or a polynucleotide encoding the base editor and (ii) one or more guide polynucleotides or a polynucleotide encoding the guide polynucleotides. The base editor contains a programmable DNA binding domain and a deaminase domain. Each of the guide polynucleotides directs the base editor to effect a nucleobase alteration in a A2AR or A2BR gene that alters a splice acceptor or splice donor site, introduces a stop codon, or otherwise disrupts expression of the gene, thereby reducing expression of an A2AR or A2BR polypeptide and/or polynucleotide in the cell.

In another aspect, the invention features a base editor system that contains (i) a base editor, or a polynucleotide encoding the same and (ii) a guide polynucleotide or a polynucleotide encoding the guide polynucleotide. The base editor contains a programmable DNA binding domain and a deaminase domain. The guide polynucleotide contains a sequence selected from one or more of: UCACCGGAGCGGGAUGCGGA (SEQ ID NO: 387); CUGCUCACCGGAGCGGGAUG (SEQ ID NO: 388); CACUCCCAGGGCUGCGGGGA (SEQ ID NO: 389); CCACUCCCAGGGCUGCGGGG (SEQ ID NO: 390); GCGACGACAGCUGAAGCAGA (SEQ ID NO: 391); UGGAGAGCCAGCCUCUGCCG (SEQ ID NO: 392); GGAGAGCCAGCCUCUGCCGG (SEQ ID NO: 393); ACAUGAGCCAGAGAGGGGCG (SEQ ID NO: 394); GAGGCAGCAAGAACCUUUCA (SEQ ID NO: 395); UGGCCCACACUCCUGGCGGG (SEQ ID NO: 396); CGUUGGCCCACACUCCUGGC (SEQ ID NO: 397); UCUCCCCAGGUACAAUGGCU (SEQ ID NO: 398); CAGUUGUUCCAACCUAGCAU (SEQ ID NO: 399); GGCCAUGCUGCUGGAGACAC (SEQ ID NO: 400); UCACCUGAGCGGGACACAGA (SEQ ID NO: 401); UUACUGUUCCACCCCAGGAA (SEQ ID NO: 402); UUUAAACAGGUAUAAAAGUU (SEQ ID NO: 403); GCUUCAGCGCACUGAGCUGA (SEQ ID NO: 404); UGCCAAGCAGAUGUCAAGAG (SEQ ID NO: 405); CUUACUAUCAUGAUGAGUUU (SEQ ID NO: 406); CAUAUACCUGAGUAGAAAAU (SEQ ID NO: 407); UCAUAUACCUGAGUAGAAAA (SEQ ID NO: 408); UGUUUACAGUUUGAACUAAC (SEQ ID NO: 409); UCAUUAGGCCUUGUGAAAAA (SEQ ID NO: 410); ACACAGGUAUUGCACUGCAC (SEQ ID NO: 411); UAACAGAAUUACCGAAUUGA (SEQ ID NO: 412); AACAGAAUUACCGAAUUGAU (SEQ ID NO: 413); UUUCAGAACUACAGUUCCUG (SEQ ID NO: 414); AGCUCCCAAUGUCGGAGUUU (SEQ ID NO: 415); GAGCUCCCAAUGUCGGAGUU (SEQ ID NO: 416); UUAAAUGAGCUCCCAAUGUC (SEQ ID NO: 417); UUUAAAUGAGCUCCCAAUGU (SEQ ID NO: 418); and ACCAUACCCAUUUUCUAUUC (SEQ ID NO: 419).

In one aspect, the invention features a cell containing the base editor system of any of the above aspects.

In another aspect, the invention features a pharmaceutical composition containing an effective amount a modified immune cell of any of the above aspects. In an embodiment, the pharmaceutical composition further contains a pharmaceutically acceptable excipient.

In another aspect, the invention features a composition containing a guide polynucleotide and a polynucleotide encoding a fusion protein containing a polynucleotide programmable DNA binding domain and a deaminase domain. The guide polynucleotide contains a nucleic acid sequence that is complementary to a gene selected from one or more of A2AR, A2BR, HIF1α, and HIF1α.I3 genes.

In another aspect, the invention features a kit containing a modified immune cell of any of the above aspects. In an embodiment, the kit further contains written instructions for using the modified immune cell or the pharmaceutical composition of any of the above aspects.

In another aspect, the invention features a modified immune effector cell. The modified immune effector cell expresses a chimeric antigen receptor targeting an antigen associated with a disease or disorder. The modified immune effector cell contains reduced or undetectable expression of the following polypeptides: A2AR, CD3ε, B2M, and CIITA.

In another aspect, the invention features a method of treating cancer in a subject, the method involves administering to the subject an effective amount of a modified immune cell of any of the above aspects. In an embodiment, the cancer is a solid tumor.

In one aspect, the invention features a modified immune cell produced according to the method of any one of the above aspects.

In one aspect, the invention features a modified immune cell containing a nucleobase alteration that reduces or eliminates expression of a polypeptide selected from one or more of A2AR, A2BR, HIF1α, and HIF1α.I3.

In one aspect, the invention features a modified immune effector cell. The modified immune effector cell expresses a chimeric antigen receptor targeting an antigen associated with a disease or disorder. The modified immune effector cell comprises reduced or undetectable expression of the following polypeptides: A2AR, B2M, CD3ε, CIITA, PD1, and TGFbR2.

In any of the above aspects, or embodiments thereof, the nuclease-active nucleic acid programmable DNA binding domain is a Cas12b.

In any of the above aspects, or embodiments thereof, the polypeptide component of the hypoxic and/or adenosinergic pathway is selected from one or more of A2AR, A2BR, HIF1α, and HIF1α.I3.

In any of the above aspects, or embodiments thereof, the method increases resistance to hypoxic-adenosinergic immunosuppression of the modified immune cell. In any of the above aspects, or embodiments thereof, the method increases cytokine production of the modified immune cell relative to an unmodified reference immune cell.

In any of the above aspects, or embodiments thereof, the one or more guide polynucleotides target a site selected from those listed in Table 1A and/or contains a spacer listed in Table 1A or Table 1B.

In any of the above aspects, or embodiments thereof, the deaminase is an adenosine deaminase or a cytidine deaminase. In any of the above aspects, or embodiments thereof, the deaminase domain is an adenosine deaminase domain, and guides 158, 170, and 173 are used to edit an HIF1α target site.

In any of the above aspects, or embodiments thereof, the method reduces or virtually eliminates HIF1α expression. In any of the above aspects, or embodiments thereof, the method increases cytokine production in the cell relative to an unmodified reference immune cell.

In any of the above aspects, or embodiments thereof, the deaminase domain is a cytidine deaminase domain editor, and guides 145 and 155 are used to are used to edit an A2AR target site. In any of the above aspects, or embodiments thereof, the method reduces or virtually eliminates A2AR expression.

In any of the above aspects, or embodiments thereof, the method reduces adenosine signaling, results in lack of upregulation of pCREB in the presence of 2-chloroadenosine, and or protects the cell from adenosine-mediated cytokine production.

In any of the above aspects, or embodiments thereof, the deaminase domain is a cytidine deaminase domain, and guides 222, 223, 225, and 226 are used to edit a A2BR target site. In any of the above aspects, or embodiments thereof, the deaminase domain is an adenosine deaminase domain, and guides 221 and 224 are used to edit an A2BR target site. In any of the above aspects, or embodiments thereof, the deaminase domain is an adenosine deaminase domain and guide 155 is used to edit an A2BR target site.

In any of the above aspects, or embodiments thereof, the cell is a T cell or NK cell. In any of the above aspects, or embodiments thereof, the cell is a chimeric antigen receptor T (CAR-T) cell.

In any of the above aspects, or embodiments thereof, the method results in a reduction in hypoxia/adenosine-mediated suppression of cytotoxic T cell function. In any of the above aspects, or embodiments thereof, the reduction is a 10% or greater reduction. In any of the above aspects, or embodiments thereof, the reduction is a 25% or greater reduction.

In any of the above aspects, or embodiments thereof, the base editor contains a complex containing the deaminase domain, the polynucleotide programmable DNA, and the guide polynucleotide, or the base editor is a fusion protein containing the polynucleotide programmable DNA binding polypeptide fused to the deaminase domain.

In any of the above aspects, or embodiments thereof, the programmable DNA binding domain is Cas9 or Cas12. In any of the above aspects, or embodiments thereof, the programmable DNA binding domain is a Staphylococcus aureus Cas9 (SaCas9), Streptococcus thermophilus 1 Cas9 (St1Cas9), a Streptococcus pyogenes Cas9 (SpCas9), or variants thereof. In any of the above aspects, or embodiments thereof, the programmable DNA binding domain contains a nuclease dead Cas9 (dCas9), a Cas9 nickase (nCas9), or a nuclease active Cas9.

In any of the above aspects, or embodiments thereof, the base editor further contains one or more uracil glycosylase inhibitors (UGIs). In any of the above aspects, or embodiments thereof, the base editor further contains one or more nuclear localization signals (NLS). In embodiments, the NLS is a bipartite NLS.

In any of the above aspects, or embodiments thereof, the cell is obtained from a healthy subject.

In any of the above aspects, or embodiments thereof, the guide polynucleotide directs the base editor to effect a nucleobase alteration that results in a premature stop codon in the gene.

In any of the above aspects, or embodiments thereof, the nucleobase alteration is an A-to-G or C-to-T alteration. In any of the above aspects, or embodiments thereof, the nucleobase alteration is at a splice acceptor site of the gene. In embodiments, the splice acceptor site is a splice acceptor site 5′ of an exon of the gene.

In any of the above aspects, or embodiments thereof, the nucleobase alteration results in less than 15% indels in a genome of the cell. In any of the above aspects, or embodiments thereof, the nucleobase alteration results in less than 5% indels in a genome of the cell. In any of the above aspects, or embodiments thereof, the nucleobase alteration results in less than 2% indels in a genome of the cell.

In any of the above aspects, or embodiments thereof, the cell is a mammalian cell or a human cell.

In any of the above aspects, or embodiments thereof, the deaminase domain contains an adenosine deaminase domain. In any of the above aspects, or embodiments thereof, the adenosine deaminase domain is TadA7.10, a Tad8, or a Tad9. In any of the above aspects, or embodiments thereof, the adenosine deaminase domain contains a TadA deaminase domain. In any of the above aspects, or embodiments thereof, the adenosine deaminase domain is a TadA containing a V28S mutation or a T166R mutation as numbered in the amino acid sequence MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1) or a corresponding mutation thereof. In any of the above aspects, or embodiments thereof, the adenosine deaminase domain contains one or more of the following mutations: Y147T, Y147R, Q154S, Y123H, and Q154R as numbered in the amino acid sequence MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1) or a corresponding mutation thereof. In any of the above aspects, or embodiments thereof, the adenosine deaminase domain contains a combination of mutations selected from one or more of: Y147T Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R as numbered in SEQ ID NO: 2 or corresponding mutations thereof. In any of the above aspects, or embodiments thereof, the adenosine deaminase domain contains a TadA dimer. In any of the above aspects, or embodiments thereof, the adenosine deaminase domain contains an adenosine deaminase monomer.

In any of the above aspects, or embodiments thereof, the modified immune cell has increased resistance to hypoxic-adenosinergic immunosuppression and/or increased cytokine production relative to an unmodified reference immune cell. In any of the above aspects, or embodiments thereof, the modified immune cell is a T cell or an NK cell. In any of the above aspects, or embodiments thereof, the modified immune cell expresses a chimeric antigen receptor (CAR). In any of the above aspects, or embodiments thereof, the immune cell is obtained from a healthy subject.

In any of the above aspects, or embodiments thereof, the subject is a human subject.

In any of the above aspects, or embodiments thereof, the cell contains or further contains a combination of alterations to polypeptides, where the combination of polypeptides is selected from one or more of: a) β2M, TAP1, TAP2, and Tapasin; b) TRAC, CD52, CIITA, HLA-E, HLA-G, PD-L1, PD1, and CD47; c) TRAC, CD52, and CIITA; d) HLA-E, HLA-G, PD-L1, PD1, and CD47; e) one or more of 02M, TAP1, TAP2, and Tapasin, and one or more of HLA-E, HLA-G, PD-L1, PD1, and CD47; f) B2M, CD3ε, and CIITA; g) A2AR, B2M, CD3ε, and CIITA; and h) A2AR, B2M, CD3ε, CIITA, PD1, and TGFbR2.

In any of the above aspects, or embodiments thereof, the cell is a mammalian cell, a human cell, or a motor neuron. In any of the above aspects, or embodiments thereof, the cell is in vivo, ex vivo, or in vitro. In any of the above aspects, or embodiments thereof, the cell is an autologous cell isolated from a subject. In any of the above aspects, or embodiments thereof, the cell is an allogeneic cell.

In any of the above aspects, or embodiments thereof, the guide polynucleotide targets a site selected from those listed in Table 1A and/or contains a spacer listed in Table 1A or 1B.

In any of the above aspects, or embodiments thereof, deaminase domain is a cytidine and/or adenosine deaminase domain.

In any of the above aspects, or embodiments thereof, the polynucleotide encoding the fusion protein contains mRNA.

In any of the above aspects, or embodiments thereof, the method further involves altering the cell to reduce or eliminate expression of one or more polypeptides selected from one or more of B2M, CD3ε, PD1, CIITA, CTLA4, LAG3, TIM3, TGFbR1, and TGFbR2. In any of the above aspects, or embodiments thereof, the method further involves altering the cell to reduce or eliminate expression of each of HLA Class I polypeptides, HLA Class II polypeptides, and A2AR. In any of the above aspects, or embodiments thereof, the method further involves altering the cell to reduce or eliminate expression of the following polypeptides: CD3ε, B2M, and CIITA. In any of the above aspects, or embodiments thereof, the method further involves altering the cell to reduce or eliminate expression of the following polypeptides: A2AR and HIF1α. In any of the above aspects, or embodiments thereof, the method further involves altering the cell to reduce or eliminate expression of one or more polypeptides selected from one or more of CD3ε, CD3δ, CD3γ, B2M, CIITA, TRAC, and TRBC. In any of the above aspects, or embodiments thereof, the method further involves over-expressing Human Leukocyte Antigen-E (HLA-E) or Human Leukocyte Antigen-G (HLA-G) in the cell.

In any of the aspects provided herein, or embodiments thereof, the disease or disorder is a neoplasia.

In any of the above aspects, or embodiments thereof, the guide polynucleotide comprises a scaffold comprising the nucleotide sequence GUUUUAGAGCUAGAAAUAGCAAGUUAAAAUAAGGCUAGUCCGUUAUCAACUUGA AAAAGUGGCACCGAGUCGGUGCUUUU (Cas9 scaffold; SEQ ID NO: 317).

In any of the above aspects, or embodiments thereof, the method involves reducing the expression of the A2AR polypeptide and/or polynucleotide in the cell.

In any of the above aspects, or embodiments thereof, the neoplasia is a solid tumor.

In any of the above aspects, or embodiments thereof, the method involves, or further involves, contacting the cell with one or more guide polynucleotides, or one or more polynucleotides encoding the same, containing a sequence selected from one or more of the following: TSBTx2043 (targeting an A2AR polynucleotide), TSBTx4073 (targeting a CD3ε polynucleotide), TSBTx760 (targetinga B2M polynucleotide), TSBTx763 (targeting a CIITA polynucleotide), and TSBTx025 (targeting a PD1 polynucleotide) (see sequences provided in Tables 1A and 1B). In any of the above aspects, or embodiments thereof, the method involves, or further involves, contacting the cell with one, two, three, four, or five guide polynucleotides, or one or more polynucleotides encoding the same, where the guide polynucleotides are selected from: TSBTx2043, TSBTx4073, TSBTx760, TSBTx763, and TSBTx025. In any of the above aspects, or embodiments thereof, the method involves, or further involves, contacting the cell with one, two, three, or four guide polynucleotides, or one or more polynucleotides encoding the same, where the guide polynucleotides are selected from: TSBTx2043, TSBTx4073, TSBTx763, and TSBTx025. In any of the above aspects, or embodiments thereof, the base editor is ABE8.20.

### Definitions

By “anti-Epidermal Growth Factor Receptor chimeric antigen receptor (anti-EGFR CAR) polypeptide” is meant a CAR that specifically binds an EGFR, wherein such binding activates the CAR-T cell, and having at least about 85% amino acid sequence identity to the following sequence:

(SEQ ID NO: 454)
MALPVTALLLPLALLLHAARPQILLTQSPVILSVSPGERVSFSCRASQSIGTNIHWYQQRTN
GSPRLLIKYASESISGIPSRFSGSGSGTDFTLSINSVESEDIADYYCOONNNWPTTFGAGTKLEL
KGGGGSGGGGSGGGGSQVOLKQSGPGLVQPSQSLSITCTVSGFSLTNYGVHWVROSPG
KGLEWLGVIWSGGNTDYNTPFTSRLSINKDNSKSQVFFKMNSLQSQDTAIYYCARALTY
YDYEFAYWGQGTLVTVSSTTTPAPRPPTPAPTIASQPLSLRPEACRPAAGGAVHTRGL
DFACDIYIWAPLAGTCGVLLLSLVITLYCKRGRKKLLYIFKQPEMRPVQTTQEEDGCSCR

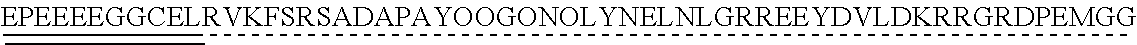

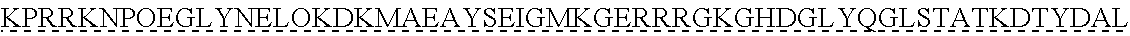

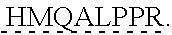

In the above sequence, bold text indicates a signal peptide, italic text indicates a cetuximab VL domain, underlined text indicates a G4S linker, bold italic text indicates a cetuximab VH domain, bold underlined text indicates a CD8a hinge domain, plain text indicates a CD8a transmembrane domain, bold, italic, underlined text indicates a tail CD8 domain, double underlined text indicates a 4-1BB intracellular signaling/costimulatory domain, and text [symbol] indicates a CD3zeta intracellular signaling domain.

By “anti-EGFR chimeric antigen receptor (anti-EGFR CAR) polynucleotide” is meant a nucleic acid molecule encoding an anti-EGFR CAR polypeptide, as well as the introns, exons, and regulatory sequences associated with its expression, or fragments thereof. In embodiments, an anti-EGFR CAR polynucleotide is the genomic sequence, mRNA, or gene associated with and/or required for anti-EGFR CAR expression. Exemplary anti-EGFR CAR nucleotide sequences are provided below.
>EGFR coding sequence

(SEQ ID NO: 455)
ATGGCACTGCCAGTGACAGCTCTCCTGTTGCCACTCGCCCTTCTGCTGCA
TGCTGCAAGGCCTCAAATCCTGCTCACTCAGAGCCCGGTGATTTTGTCCG
TCTCCCCCGGCGAGCGCGTATCATTTTCATGTAGGGCTTCTCAGAGCATC
GGCACCAATATTCACTGGTATCAGCAGCGCACAAATGGCAGCCCAAGACT
GCTCATTAAGTATGCCTCAGAATCAATTTCAGGCATCCCAAGCCGCTTCT
CCGGCTCAGGCTCCGGGACCGACTTTACATTGAGCATTAACTCAGTGGAA
TCTGAAGACATCGCCGATTACTACTGTCAACAGAATAATAACTGGCCGAC
GACGTTTGGCGCCGGAACTAAACTGGAACTGAAGGGGGGAGGGGGCTCTG
GAGGTGGAGGGTCCGGAGGAGGCGGGTCACAAGTGCAGCTGAAGCAATCT
GGACCTGGACTCGTTCAGCCTTCTCAGAGCCTCTCCATCACTTGCACTGT
AAGTGGCTTCTCACTGACCAACTATGGGGTGCACTGGGTGAGACAGTCCC
CCGGAAAGGGGCTGGAATGGCTCGGAGTTATTTGGAGCGGAGGAAATACG
GACTACAACACCCCGTTTACATCCAGACTCTCCATAAATAAGGATAACAG
CAAAAGCCAGGTGTTTTTTAAAATGAACAGCCTGCAGAGCCAAGATACAG
CTATCTATTATTGTGCGCGCGCACTGACATACTATGACTACGAGTTTGCA
TACTGGGGCCAAGGGACCCTTGTCACAGTCTCATCAACCACAACACCTGC
TCCAAGGCCCCCCACACCCGCTCCAACTATAGCCAGCCAACCATTGAGCC
TCAGACCTGAAGCTTGCAGGCCCGCAGCAGGAGGCGCCGTCCATACGCGA
GGCCTGGACTTCGCGTGTGATATTTATATTTGGGCCCCTTTGGCCGGAAC
ATGTGGGGTGTTGCTTCTCTCCCTTGTGATCACTCTGTATTGTAAGCGCG
GGAGAAAGAAGCTCCTGTACATCTTCAAGCAGCCTTTTATGCGACCTGTG
CAAACCACTCAGGAAGAAGATGGGTGTTCATGCCGCTTCCCCGAGGAGGA
AGAAGGAGGGTGTGAACTGAGGGTGAAATTTTCTAGAAGCGCCGATGCTC
CCGCATATCAGCAGGGTCAGAATCAGCTCTACAATGAATTGAATCTCGGC
AGGCGAGAAGAGTACGATGTTCTGGACAAGAGACGGGGCAGGGATCCCGA
GATGGGGGGAAAGCCCCGGAGAAAAAATCCTCAGGAGGGGTTGTACAATG
AGCTGCAGAAGGACAAGATGGCTGAAGCCTATAGCGAGATCGGAATGAAA
GGCGAAAGACGCAGAGGCAAGGGGCATGACGGTCTGTACCAGGGTCTCTC
TACAGCCACCAAGGACACTTATGATGCGTTGCATATGCAAGCCTTGCCAC
CCCGCTAA.

>pBB1245EGFR(NoAAA).CD8.TM.41BB.CD3Z plasmid sequence

TCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGG
TGTCGGGGCTGGCTTAACTATGCGGCATCAGAGCAGATTGTACTGAGAGTGCACCATCATATGC
CAGCCTATGGTGACATTGATTATTGACTAGTTATTAATAGTAATCAATTACGGGGTCATTAGTT
CATAGCCCATATATGGAGTTCCGCGTTACATAACTTACGGTAAATGGCCCGCCTGGCTGACCGC
CCAACGACCCCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCATTATG
CCCAGTACATGACCTTATGGGACTTTCCTACTTGGCAGTACATCTACGTATTAGTCATCGCTAT
TACCATGGTGATGCGGTTTTGGCAGTACATCAATGGGCGTGGATAGCGGTTTGACTCACGGGGA
TTTCCAAGTCTCCACCCCATTGACGTCAATGGGAGTTTGTTTTGGCACCAAAATCAACGGGACT
TTCCAAAATGTCGTAACAACTCCGCCCCATTGACGCAAATGGGCGGTAGGCGTGTACGGTGGGA
GGTCTATATAAGCAGAGCTCGTTTAGTGAACCGGGTCTCTCTGGTTAGACCAGATCTGAGCCTG
GGAGCTCTCTGGCTAACTAGGGAACCCACTGCTTAAGCCTCAATAAAGCTTGCCTTGAGTGCTC
AAAGTAGTGTGTGCCCGTCTGTTGTGTGACTCTGGTAACTAGAGATCCCTCAGACCCTTTTAGT
CAGTGTGGAAAATCTCTAGCAGTGGCGCCCGAACAGGGACTTGAAAGCGAAAGTAAAGCCAGAG
GAGATCTCTCGACGCAGGACTCGGCTTGCTGAAGCGCGCACGGCAAGAGGCGAGGGGCGGCGAC
TGGTGAGTACGCCAAAAATTTTGACTAGCGGAGGCTAGAAGGAGAGAGTAGGGTGCGAGAGCGT
CGGTATTAAGCGGGGGAGAATTAGATAAATGGGAAAAAATTCGGTTAAGGCCAGGGGGAAAGAA
ACAATATAAACTAAAACATATAGTTAGGGCAAGCAGGGAGCTAGAACGATTCGCAGTTAATCCT
GGCCTTTTAGAGACATCAGAAGGCTGTAGACAAATACTGGGACAGCTACAACCATCCCTTCAGA
CAGGATCAGAAGAACTTAGATCATTATATAATACAATAGCAGTCCTCTATTGTGTGCATCAAAG
GATAGATGTAAAAGACACCAAGGAAGCCTTAGATAAGATAGAGGAAGAGCAAAACAAAAGTAAG
AAAAAGGCACAGCAAGCAGCAGCTGACACAGGAAACAACAGCCAGGTCAGCCAAAATTACCCTA
TAGTGCAGAACCTCCAGGGGCAAATGGTACATCAGGCCATATCACCTAGAACTTTAAATTAAGA
CAGCAGTACAAATGGCAGTATTCATCCACAATTTTAAAAGAAAAGGGGGGATTGGGGGGTACAG
TGCAGGGGAAAGAATAGTAGACATAATAGCAACAGACATACAAACTAAAGAATTACAAAAACAA
ATTACAAAAATTCAAAATTTTCGGGTTTATTACAGGGACAGCAGAGATCCAGTTTGGAAAGGAC
CAGCAAAGCTCCTCTGGAAAGGTGAAGGGGCAGTAGTAATACAAGATAATAGTGACATAAAAGT
AGTGCCAAGAAGAAAAGCAAAGATCATCAGGGATTATGGAAAACAGATGGCAGGTGATGATTGT
GTGGCAAGTAGACAGGATGAGGATTAACACATGGAAAAGATTAGTAAAACACCATAGCTCTAGA
GCGATCCCGATCTTCAGACCTGGAGGAGGAGATATGAGGGACAATTGGAGAAGTGAATTATATA
AATATAAAGTAGTAAAAATTGAACCATTAGGAGTAGCACCCACCAAGGCAAAGAGAAGAGTGGT
GCAGAGAGAAAAAAGAGCAGTGGGAATAGGAGCTTTGTTCCTTGGGTTCTTGGGAGCAGCAGGA
AGCACTATGGGCGCAGCGTCAATGACGCTGACGGTACAGGCCAGACAATTATTGTCTGGTATAG
TGCAGCAGCAGAACAATTTGCTGAGGGCTATTGAGGCGCAACAGCATCTGTTGCAACTCACAGT
CTGGGGCATCAAGCAGCTCCAGGCAAGAATCCTGGCTGTGGAAAGATACCTAAAGGATCAACAG
CTCCTGGGGATTTGGGGTTGCTCTGGAAAACTCATTTGCACCACTGCTGTGCCTTGGAATGCTA
GTTGGAGTAATAAATCTCTGGAACAGATTTGGAATCACACGACCTGGATGGAGTGGGACAGAGA
AATTAACAATTACACAAGCTTGGTAGGTTTAAGAATAGTTTTTGCTGTACTTTCTATAGTGAAT
AGAGTTAGGCAGGGATATTCACCATTATCGTTTCAGACCCACCTCCCAACCCCGAGGGGACCCG
ACAGGCCCGAAGGAATAGAAGAAGAAGGTGGAGAGAGAGACAGAGACAGATCCATTCGATTAGT
GAACGGATCCATCTCGACGGAATGAAAGACCCCACCTGTAGGTTTGGCAAGCTAGGATCAAGGT
TAGGAACAGAGAGACAGCAGAATATGGGCCAAACAGGATATCTGTGGTAAGCAGTTCCTGCCCC
GGCTCAGGGCCAAGAACAGTTGGAACAGCAGAATATGGGCCAAACAGGATATCTGTGGTAAGCA
GTTCCTGCCCCGGCTCAGGGCCAAGAACAGATGGTCCCCAGATGCGGTCCCGCCCTCAGCAGTT
TCTAGAGAACCATCAGATGTTTCCAGGGTGCCCCAAGGACCTGAAATGACCCTGTGCCTTATTT
GAACTAACCAATCAGTTCGCTTCTCGCTTCTGTTCGCGCGCTTCTGCTCCCCGAGCTCAATAAA
AGAGCCCACAACCCCTCACTCGGCGCGATTCACCTGACGCGTCTACGCCACCATGGCACTGCCA
GTGACAGCTCTCCTGTTGCCACTCGCCCTTCTGCTGCATGCTGCAAGGCCTCAAATCCTGCTCA
CTCAGAGCCCGGTGATTTTGTCCGTCTCCCCCGGCGAGCGCGTATCATTTTCATGTAGGGCTTC
TCAGAGCATCGGCACCAATATTCACTGGTATCAGCAGCGCACAAATGGCAGCCCAAGACTGCTC
ATTAAGTATGCCTCAGAATCAATTTCAGGCATCCCAAGCCGCTTCTCCGGCTCAGGCTCCGGGA
CCGACTTTACATTGAGCATTAACTCAGTGGAATCTGAAGACATCGCCGATTACTACTGTCAACA
GAATAATAACTGGCCGACGACGTTTGGCGCCGGAACTAAACTGGAACTGAAGGGGGGAGGGGGC
TCTGGAGGTGGAGGGTCCGGAGGAGGCGGGTCACAAGTGCAGCTGAAGCAATCTGGACCTGGAC
TCGTTCAGCCTTCTCAGAGCCTCTCCATCACTTGCACTGTAAGTGGCTTCTCACTGACCAACTA
TGGGGTGCACTGGGTGAGACAGTCCCCCGGAAAGGGGCTGGAATGGCTCGGAGTTATTTGGAGC
GGAGGAAATACGGACTACAACACCCCGTTTACATCCAGACTCTCCATAAATAAGGATAACAGCA
AAAGCCAGGTGTTTTTTAAAATGAACAGCCTGCAGAGCCAAGATACAGCTATCTATTATTGTGC
GCGCGCACTGACATACTATGACTACGAGTTTGCATACTGGGGCCAAGGGACCCTTGTCACAGTC
TCATCAACCACAACACCTGCTCCAAGGCCCCCCACACCCGCTCCAACTATAGCCAGCCAACCAT
TGAGCCTCAGACCTGAAGCTTGCAGGCCCGCAGCAGGAGGCGCCGTCCATACGCGAGGCCTGGA
CTTCGCGTGTGATATTTATATTTGGGCCCCTTTGGCCGGAACATGTGGGGTGTTGCTTCTCTCC
CTTGTGATCACTCTGTATTGTAAGCGCGGGAGAAAGAAGCTCCTGTACATCTTCAAGCAGCCTT
TTATGCGACCTGTGCAAACCACTCAGGAAGAAGATGGGTGTTCATGCCGCTTCCCCGAGGAGGA
AGAAGGAGGGTGTGAACTGAGGGTGAAATTTTCTAGAAGCGCCGATGCTCCCGCATATCAGCAG
GGTCAGAATCAGCTCTACAATGAATTGAATCTCGGCAGGCGAGAAGAGTACGATGTTCTGGACA
AGAGACGGGGCAGGGATCCCGAGATGGGGGGAAAGCCCCGGAGAAAAAATCCTCAGGAGGGGTT
GTACAATGAGCTGCAGAAGGACAAGATGGCTGAAGCCTATAGCGAGATCGGAATGAAAGGCGAA
AGACGCAGAGGCAAGGGGCATGACGGTCTGTACCAGGGTCTCTCTACAGCCACCAAGGACACTT
ATGATGCGTTGCATATGCAAGCCTTGCCACCCCGCTAATGACAGGTACCTTTAAGACCAATGAC
TTACAAGGCAGCTGTAGATCTTAGCCACTTTTTAAAAGAAAAGGGGGGACTGGAAGGGCTAATT
CACTCCCAAAGAAGACAAGATCTGCTTTTTGCCTGTACTGGGTCTCTCTGGTTAGACCAGATCT
GAGCCTGGGAGCTCTCTGGCTAACTAGGGAACCCACTGCTTAAGCCTCAATAAAGCTTGCCTTG
AGTGCTTCAATGTGTGTGTTGGTTTTTTGTGTGTCGAAATTCTAGCGATTCTAGCTTGGCGTAA
TCATGGTCATAGCTGTTTCCTGTGTGAAATTGTTATCCGCTCACAATTCCACACAACATACGAG
CCGGAAGCATAAAGTGTAAAGCCTGGGGTGCCTAATGAGTGAGCTAACTCACATTAATTGCGTT
GCGCTCACTGCCCGCTTTCCAGTCGGGAAACCTGTCGTGCCAGCTGCATTAATGAATCGGCCAA
CGCGCGGGGAGAGGCGGTTTGCGTATTGGGCGCTCTTCCGCTTCCTCGCTCACTGACTCGCTGC
GCTCGGTCGTTCGGCTGCGGCGAGCGGTATCAGCTCACTCAAAGGCGGTAATACGGTTATCCAC
AGAATCAGGGGATAACGCAGGAAAGAACATGTGAGCAAAAGGCCAGCAAAAGGCCAGGAACCGT
AAAAAGGCCGCGTTGCTGGCGTTTTTCCATAGGCTCCGCCCCCCTGACGAGCATCACAAAAATC
GACGCTCAAGTCAGAGGTGGCGAAACCCGACAGGACTATAAAGATACCAGGCGTTTCCCCCTGG
AAGCTCCCTCGTGCGCTCTCCTGTTCCGACCCTGCCGCTTACCGGATACCTGTCCGCCTTTCTC
CCTTCGGGAAGCGTGGCGCTTTCTCATAGCTCACGCTGTAGGTATCTCAGTTCGGTGTAGGTCG
TTCGCTCCAAGCTGGGCTGTGTGCACGAACCCCCCGTTCAGCCCGACCGCTGCGCCTTATCCGG
TAACTATCGTCTTGAGTCCAACCCGGTAAGACACGACTTATCGCCACTGGCAGCAGCCACTGGT
AACAGGATTAGCAGAGCGAGGTATGTAGGCGGTGCTACAGAGTTCTTGAAGTGGTGGCCTAACT
ACGGCTACACTAGAAGAACAGTATTTGGTATCTGCGCTCTGCTGAAGCCAGTTACCTTCGGAAA
AAGAGTTGGTAGCTCTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGATCTTTTCTACGGGGT
CTGACGCTCAGTGGAACGAAAACTCACGTTAAGGGATTTTGGTCATGAGATTATCAAAAAGGAT
CTTCACCTAGATCCTTTTAAATTAAAAATGAAGTTTTAAATCAATCTAAAGTATATATGAGTAA
ACTTGGTCTGACAGTTACCAATGCTTAATCAGTGAGGCACCTATCTCAGCGATCTGTCTATTTC
GTTCATCCATAGTTGCCTGACTCCCCGTCGTGTAGATAACTACGATACGGGAGGGCTTACCATC
TGGCCCCAGTGCTGCAATGATACCGCGAGACCCACGCTCACCGGCTCCAGATTTATCAGCAATA
AACCAGCCAGCCGGAAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGTTTGCGCAACGTTGT
TGCCATTGCTACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGTTAGCTCCTTCG
GTCCTCCGATCGTTGTCAGAAGTAAGTTGGCCGCAGTGTTATCACTCATGGTTATGGCAGCACT
GCATAATTCTCTTACTGTCATGCCATCCGTAAGATGCTTTTCTGTGACTGGTGAGTACTCAACC
AAGTCATTCTGAGAATAGTGTATGCGGCGACCGAGTTGCTCTTGCCCGGCGTCAATACGGGATA
ATACCGCGCCACATAGCAGAACTTTAAAAGTGCTCATCATTGGAAAACGTTCTTCGGGGCGAAA
ACTCTCAAGGATCTTACCGCTGTTGAGATCCAGTTCGATGTAACCCACTCGTGCACCCAACTGA
TCTTCAGCATCTTTTACTTTCACCAGCGTTTCTGGGTGAGCAAAAACAGGAAGGCAAAATGCCG
CAAAAAAGGGAATAAGGGCGACACGGAAATGTTGAATACTCATACTCTTCCTTTTTCAATATTA
TTGAAGCATTTATCAGGGTTATTGTCTCATGAGCGGATACATATTTGAATGTATTTAGAAAAAT
AAACAAATAGGGGTTCCGCGCACATTTCCCCGAAAAGTGCCACCTGGGACTAGCTTTTTGCAAA
AGCCTAGGCCTCCAAAAAAGCCTCCTCACTACTTCTGGAATAGCTCAGAGGCCGAGGCGGCCTC
GGCCTCTGCATAAATAAAAAAAATTAGTCAGCCATGGGGCGGAGAATGGGCGGAACTGGGCGGA
GTTAGGGGCGGGATGGGCGGAGTTAGGGGCGGGACTATGGTTGCTGACTAATTGAGATGAGCTT
GCATGCCGACATTGATTATTGACTAGTCCCTAAGAAACCATTCTTATCATGACATTAACCTATA
AAAATAGGCGTATCACGAGGCCCTTTCGTC.

By “epidermal growth factor receptor (EGFR) polypeptide” is meant an EGFR protein or fragment thereof, having cell signaling activity and having at least about 85% amino acid sequence identity to GenBank Accession No. AAH94761.1. An exemplary EGFR amino acid sequence from Homo Sapiens is provided below (GenBank Accession No. AAH94761.1):

(SEQ ID NO: 457)
MRPSGTAGAALLALLAALCPASRALEEKKVCQGTSNKLTQLGTFEDHFLS
LQRMFNNCEVVLGNLEITYVQRNYDLSFLKTIQEVAGYVLIALNTVERIP
LENLQIIRGNMYYENSYALAVLSNYDANKTGLKELPMRNLQGQKCDPSCP
NGSCWGAGEENCQKLTKIICAQQCSGRCRGKSPSDCCHNQCAAGCTGPRE
SDCLVCRKFRDEATCKDTCPPLMLYNPTTYQMDVNPEGKYSFGATCVKKC
PRNYVVTDHGSCVRACGADSYEMEEDGVRKCKKCEGPCRKVCNGIGIGEF
KDSLSINATNIKHFKNCTSISGDLHILPVAFRGDSFTHTPPLDPQELDIL
KTVKEITGFLLIQAWPENRTDLHAFENLEIIRGRTKQHGQFSLAVVSLNI
TSLGLRSLKEISDGDVIISGNKNLCYANTINWKKLFGTSGQKTKIISNRG
ENSCKATGQVCHALCSPEGCWGPEPRDCVSCRNVSRGRECVDKCNLLEGE
PREFVENSECIQCHPECLPQAMNITCTGRGPDNCIQCAHYIDGPHCVKTC
PAGVMGENNTLVWKYADAGHVCHLCHPNCTYGCTGPGLEGCPTNGPKIPS
IATGMVGALLLLLVVALGIGLFMRRRHIVRKRTLRRLLQERELVEPLTPS
GEAPNQALLRILKETEFKKIKVLGSGAFGTVYKGLWIPEGEKVKIPVAIK
ELREATSPKANKEILDEAYVMASVDNPHVCRLLGICLTSTVQLITQLMPF
GCLLDYVREHKDNIGSQYLLNWCVQIAKGMNYLEDRRLVHRDLAARNVLV
KTPQHVKITDFGLAKLLGAEEKEYHAEGGKVPIKWMALESILHRIYTHQS
DVWSYGVTVWELMTFGSKPYDGIPASEISSILEKGERLPQPPICTIDVYM
IMVKCWMIDADSRPKFRELIIEFSKMARDPQRYLVIQGDERMHLPSPTDS
NFYRALMDEEDMDDVVDADEYLIPQQGFFSSPSTSRTPLLSSLSATSNNS
TVACIDRNGLQSCPIKEDSFLQRYSSDPTGALTEDSIDDTFLPVPGEWLV
WKQSCSSTSSTHSAAASLQCPSQVLPPASPEGETVADLQTQ.

By “epidermal growth factor receptor (EGFR) polynucleotide” is meant a nucleic acid molecule encoding an EGFR polypeptide, as well as the introns, exons, and regulatory sequences associated with its expression, or fragments thereof. In embodiments, an EGFR polynucleotide is the genomic sequence, mRNA, or gene associated with and/or required for EGFR expression. An exemplary EGFR nucleotide sequence from Homo Sapiens is provided below (GenBank Accession No. BC094761.1, the protein-coding portion of which is provided below).

(SEQ ID NO: 458)
GTCCGGGCAGCCCCCGGCGCAGCGCGGCCGCAGCAGCCTCCTCCCCCCGCACGGTGTGAGCGCC
CGCCGCGGCCGAGGCGGCCGGAGTCCCGAGCTAGCCCCGGCGGCCGCCGCCGCCCAGACCGGAC
GACAGGCCACCTCGTCGGCGTCCGCCCGAGTCCCCGCCTCGCCGCCAACGCCACAACCACCGCG
CACGGCCCCCTGACTCCGTCCAGTATTGATCGGGAGAGCCGGAGCGAGCTCTTCGGGGAGCAGC
GATGCGACCCTCCGGGACGGCCGGGGCAGCGCTCCTGGCGCTGCTGGCTGCGCTCTGCCCGGCG
AGTCGGGCTCTGGAGGAAAAGAAAGTTTGCCAAGGCACGAGTAACAAGCTCACGCAGTTGGGCA
CTTTTGAAGATCATTTTCTCAGCCTCCAGAGGATGTTCAATAACTGTGAGGTGGTCCTTGGGAA
TTTGGAAATTACCTATGTGCAGAGGAATTATGATCTTTCCTTCTTAAAGACCATCCAGGAGGTG
GCTGGTTATGTCCTCATTGCCCTCAACACAGTGGAGCGAATTCCTTTGGAAAACCTGCAGATCA
TCAGAGGAAATATGTACTACGAAAATTCCTATGCCTTAGCAGTCTTATCTAACTATGATGCAAA
TAAAACCGGACTGAAGGAGCTGCCCATGAGAAATTTACAGGGACAAAAGTGTGATCCAAGCTGT
CCCAATGGGAGCTGCTGGGGTGCAGGAGAGGAGAACTGCCAGAAACTGACCAAAATCATCTGTG
CCCAGCAGTGCTCCGGGCGCTGCCGTGGCAAGTCCCCCAGTGACTGCTGCCACAACCAGTGTGC
TGCAGGCTGCACAGGCCCCCGGGAGAGCGACTGCCTGGTCTGCCGCAAATTCCGAGACGAAGCC
ACGTGCAAGGACACCTGCCCCCCACTCATGCTCTACAACCCCACCACGTACCAGATGGATGTGA
ACCCCGAGGGCAAATACAGCTTTGGTGCCACCTGCGTGAAGAAGTGTCCCCGTAATTATGTGGT
GACAGATCACGGCTCGTGCGTCCGAGCCTGTGGGGCCGACAGCTATGAGATGGAGGAAGACGGC
GTCCGCAAGTGTAAGAAGTGCGAAGGGCCTTGCCGCAAAGTGTGTAACGGAATAGGTATTGGTG
AATTTAAAGACTCACTCTCCATAAATGCTACGAATATTAAACACTTCAAAAACTGCACCTCCAT
CAGTGGCGATCTCCACATCCTGCCGGTGGCATTTAGGGGTGACTCCTTCACACATACTCCTCCT
CTGGATCCACAGGAACTGGATATTCTGAAAACCGTAAAGGAAATCACAGGGTTTTTGCTGATTC
AGGCTTGGCCTGAAAACAGGACGGACCTCCATGCCTTTGAGAACCTAGAAATCATACGCGGCAG
GACCAAGCAACATGGTCAGTTTTCTCTTGCAGTCGTCAGCCTGAACATAACATCCTTGGGATTA
CGCTCCCTCAAGGAGATAAGTGATGGAGATGTGATAATTTCAGGAAACAAAAATTTGTGCTATG
CAAATACAATAAACTGGAAAAAACTGTTTGGGACCTCCGGTCAGAAAACCAAAATTATAAGCAA
CAGAGGTGAAAACAGCTGCAAGGCCACAGGCCAGGTCTGCCATGCCTTGTGCTCCCCCGAGGGC
TGCTGGGGCCCGGAGCCCAGGGACTGCGTCTCTTGCCGGAATGTCAGCCGAGGCAGGGAATGCG
TGGACAAGTGCAACCTTCTGGAGGGTGAGCCAAGGGAGTTTGTGGAGAACTCTGAGTGCATACA
GTGCCACCCAGAGTGCCTGCCTCAGGCCATGAACATCACCTGCACAGGACGGGGACCAGACAAC
TGTATCCAGTGTGCCCACTACATTGACGGCCCCCACTGCGTCAAGACCTGCCCGGCAGGAGTCA
TGGGAGAAAACAACACCCTGGTCTGGAAGTACGCAGACGCCGGCCATGTGTGCCACCTGTGCCA
TCCAAACTGCACCTACGGATGCACTGGGCCAGGTCTTGAAGGCTGTCCAACGAATGGGCCTAAG
ATCCCGTCCATCGCCACTGGGATGGTGGGGGCCCTCCTCTTGCTGCTGGTGGTGGCCCTGGGGA
TCGGCCTCTTCATGCGAAGGCGCCACATCGTTCGGAAGCGCACGCTGCGGAGGCTGCTGCAGGA
GAGGGAGCTTGTGGAGCCTCTTACACCCAGTGGAGAAGCTCCCAACCAAGCTCTCTTGAGGATC
TTGAAGGAAACTGAATTCAAAAAGATCAAAGTGCTGGGCTCCGGTGCGTTCGGCACGGTGTATA
AGGGACTCTGGATCCCAGAAGGTGAGAAAGTTAAAATTCCCGTCGCTATCAAGGAATTAAGAGA
AGCAACATCTCCGAAAGCCAACAAGGAAATCCTCGATGAAGCCTACGTGATGGCCAGCGTGGAC
AACCCCCACGTGTGCCGCCTGCTGGGCATCTGCCTCACCTCCACCGTGCAGCTCATCACGCAGC
TCATGCCCTTCGGCTGCCTCCTGGACTATGTCCGGGAACACAAAGACAATATTGGCTCCCAGTA
CCTGCTCAACTGGTGTGTGCAGATCGCAAAGGGCATGAACTACTTGGAGGACCGTCGCTTGGTG
CACCGCGACCTGGCAGCCAGGAACGTACTGGTGAAAACACCGCAGCATGTCAAGATCACAGATT
TTGGGCTGGCCAAACTGCTGGGTGCGGAAGAGAAAGAATACCATGCAGAAGGAGGCAAAGTGCC
TATCAAGTGGATGGCATTGGAATCAATTTTACACAGAATCTATACCCACCAGAGTGATGTCTGG
AGCTACGGGGTGACCGTTTGGGAGTTGATGACCTTTGGATCCAAGCCATATGACGGAATCCCTG
CCAGCGAGATCTCCTCCATCCTGGAGAAAGGAGAACGCCTCCCTCAGCCACCCATATGTACCAT
CGATGTCTACATGATCATGGTCAAGTGCTGGATGATAGACGCAGATAGTCGCCCAAAGTTCCGT
GAGTTGATCATCGAATTCTCCAAAATGGCCCGAGACCCCCAGCGCTACCTTGTCATTCAGGGGG
ATGAAAGAATGCATTTGCCAAGTCCTACAGACTCCAACTTCTACCGTGCCCTGATGGATGAAGA
AGACATGGACGACGTGGTGGATGCCGACGAGTACCTCATCCCACAGCAGGGCTTCTTCAGCAGC
CCCTCCACGTCACGGACTCCCCTCCTGAGCTCTCTGAGTGCAACCAGCAACAATTCCACCGTGG
CTTGCATTGATAGAAATGGGCTGCAAAGCTGTCCCATCAAGGAAGACAGCTTCTTGCAGCGATA
CAGCTCAGACCCCACAGGCGCCTTGACTGAGGACAGCATAGACGACACCTTCCTCCCAGTGCCT
GGTGAGTGGCTTGTCTGGAAACAGTCCTGCTCCTCAACCTCCTCGACCCACTCAGCAGCAGCCA
GTCTCCAGTGTCCAAGCCAGGTGCTCCCTCCAGCATCTCCAGAGGGGGAAACAGTGGCAGATTT
GCAGACACAGTGAAGGGCGTAAGGAGCAGATAAACACATGACCGAGCCTGCACAAGCTCTTTGT
TGTGTCTGGTTGTTTGCTGTACCTCTGTTGTAAGAATGAATCTGCAAAATTTCTAGCTTATGAA
GCAAATCACGGACATACACATCTGTATGTGTGAGTGTTCATGATGTGTGTACATCTGTGTATGT
GTGTGTGTGTATGTGTGTGTTTGTGACAGATTTGATCCCTGTTCTCTCTGCTGGCTCTATCTTG
ACCTGTGAAACGTATATTTAACTAATTAAATATTAGTTAATATTAATAAATTTTAAGCTTTATC
CAGAAAAAAAAAAAAAAAA.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “adenine” or “9H-Purin-6-amine” is meant a purine nucleobase with the molecular formula C₅H₅N₅, having the structure

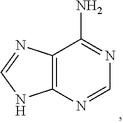

and corresponding to CAS No. 73-24-5. By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

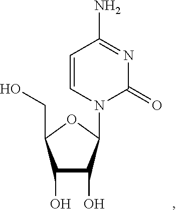

and corresponding to CAS No. 65-46-3. Its molecular formula is C₁₀H₁₃N₅O₄.

By “Adenosine A_{2A} Receptor (A2AR) polypeptide” is meant a protein having at least about 85% amino acid sequence identity to NCBI Accession No. NP_000666.2 or a fragment thereof that binds adenosine. An exemplary A2AR polypeptide sequence is provided below.

(SEQ ID NO: 370)

| | |
|---|---|
| 1 | MPIMGSSVYI TVELAIAVLA ILGNVLVCWA VWLNSNLQNV |
| | TNYFVVSLAA ADIAVGVLAI |
| 61 | PFAITISTGF CAACHGCLFI ACFVLVLTQS SIFSLLAIAI |
| | DRYIAIRIPL RYNGLVTGTR |
| 121 | AKGIIAICWV LSFAIGLTPM LGWNNCGQPK EGKNHSQGCG |
| | EGQVACLFED VVPMNYMVYF |
| 181 | NFFACVLVPL LLMLGVYLRI FLAARRQLKQ MESQPLPGER |
| | ARSTLQKEVH AAKSLAIIVG |
| 241 | LFALCWLPLH IINCFTFFCP DCSHAPLWLM YLAIVLSHTN |
| | SVVNPFIYAY RIREFRQTFR |
| 301 | KIIRSHVLRQ QEPFKAAGTS ARVLAAHGSD GEQVSLRING |
| | HPPGVWANGS APHPERRPNG |
| 361 | YALGLVSGGS AQESQGNTGL PDVELLSHEL KGVCPEPPGL |
| | DDPLAQDGAG VS |

By “Adenosine A_{2A} Receptor (A2AR) polynucleotide” is meant a nucleic acid molecule encoding an A2AR polypeptide, as well as the introns, exons, and regulatory sequences associated with its expression, or fragments thereof. In embodiments, an A2AR polynucleotide is the genomic sequence, mRNA, or gene associated with and/or required for A2AR expression. An exemplary A2AR polynucleotide sequence has about an 85% nucleic acid identity to Genbank Accession No. NM_000675.6, provided below, or a fragment thereof. A further exemplary embodiment of an A2AR polynucleotide sequence has about an 85% nucleic acid identity to the “ADORA2A gene sequence” provided in the Sequence Listing as SEQ ID NO: 371, or a fragment thereof.

(SEQ ID NO. 372)

| | |
|---|---|
| 1 | GAAGCTCTGC CTGGGCCTCA GGGACTGTGA CATGGAGCAG |
| | GAGCCGCCCC CAGCCAAGCT |
| 61 | GCTTTCAGCA CAGCGTGGGC CCCCAGCACC TTGGTGCGGG |
| | GTGCGGCCCC TCGGAGGAGG |
| 121 | GCTGTCAGGT GAAGCCTCGT GTGAGGGGGT GCCTCAGGAA |
| | CCCTGAAGCT GGGCTGAGCC |
| 181 | ATGATGCTGC TGCCAGAACC CCTGCAGAGG GCCTGGTTTC |
| | AGGAGACTCA GAGTCCTCTG |
| 241 | TGAAAAAGCC CTTGGAGAGC GCCCCAGCAG GGCTGCACTT |
| | GGCTCCTGTG AGGAAGGGGC |
| 301 | TCAGGGGTCT GGGCCCCTCC GCCTGGGCCG GGCTGGGAGC |
| | CAGGGGGGCG GCTGGGCTGC |
| 361 | AGCAATGGAC CGTGAGCTGG CCCAGCCCGC GTCCGTGCTG |
| | AGCCTGCCTG TCGTCTGTGG |
| 421 | CCATGCCCAT CATGGGCTCC TCGGTGTACA TCACGGTGGA |
| | GCTGGCCATT GCTGTGCTGG |
| 481 | CCATCCTGGG CAATGTGCTG GTGTGCTGGG CCGTGTGGCT |
| | CAACAGCAAC CTGCAGAACG |
| 541 | TCACCAACTA CTTTGTGGTG TCACTGGCGG CGGCCGACAT |
| | CGCAGTGGGT GTGCTCGCCA |
| 601 | TCCCCTTTGC CATCACCATC AGCACCGGGT TCTGCGCTGC |
| | CTGCCACGGC TGCCTCTTCA |
| 661 | TTGCCTGCTT CGTCCTGGTC CTCACGCAGA GCTCCATCTT |
| | CAGTCTCCTG GCCATCGCCA |
| 721 | TTGACCGCTA CATTGCCATC CGCATCCCGC TCCGGTACAA |
| | TGGCTTGGTG ACCGGCACGA |
| 781 | GGGCTAAGGG CATCATTGCC ATCTGCTGGG TGCTGTCGTT |
| | TGCCATCGGC CTGACTCCCA |
| 841 | TGCTAGGTTG GAACAACTGC GGTCAGCCAA AGGAGGGCAA |
| | GAACCACTCC CAGGGCTGCG |
| 901 | GGGAGGGCCA AGTGGCCTGT CTCTTTGAGG ATGTGGTCCC |
| | CATGAACTAC ATGGTGTACT |
| 961 | TCAACTTCTT TGCCTGTGTG CTGGTGCCCC TGCTGCTCAT |
| | GCTGGGTGTC TATTTGCGGA |
| 1021 | TCTTCCTGGC GGCGCGACGA CAGCTGAAGC AGATGGAGAG |
| | CCAGCCTCTG CCGGGGGAGC |
| 1081 | GGGCACGGTC CACACTGCAG AAGGAGGTCC ATGCTGCCAA |
| | GTCACTGGCC ATCATTGTGG |
| 1141 | GGCTCTTTGC CCTCTGCTGG CTGCCCCTAC ACATCATCAA |
| | CTGCTTCACT TTCTTCTGCC |
| 1201 | CCGACTGCAG CCACGCCCCT CTCTGGCTCA TGTACCTGGC |
| | CATCGTCCTC TCCCACACCA |
| 1261 | ATTCGGTTGT GAATCCCTTC ATCTACGCCT ACCGTATCCG |
| | CGAGTTCCGC CAGACCTTCC |
| 1321 | GCAAGATCAT TCGCAGCCAC GTCCTGAGGC AGCAAGAACC |
| | TTTCAAGGCA GCTGGCACCA |
| 1381 | GTGCCCGGGT CTTGGCAGCT CATGGCAGTG ACGGAGAGCA |
| | GGTCAGCCTC CGTCTCAACG |
| 1441 | GCCACCCGCC AGGAGTGTGG GCCAACGGCA GTGCTCCCCA |
| | CCCTGAGCGG AGGCCCAATG |
| 1501 | GCTATGCCCT GGGGCTGGTG AGTGGAGGGA GTGCCCAAGA |
| | GTCCCAGGGG AACACGGGCC |
| 1561 | TCCCAGACGT GGAGCTCCTT AGCCATGAGC TCAAGGGAGT |
| | GTGCCCAGAG CCCCCTGGCC |
| 1621 | TAGATGACCC CCTGGCCCAG GATGGAGCAG GAGTGTCCTG |
| | ATGATTCATG GAGTTTGCCC |
| 1681 | CTTCCTAAGG GAAGGAGATC TTTATCTTTC TGGTTGGCTT |
| | GACCAGTCAC GTTGGGAGAA |
| 1741 | GAGAGAGAGT GCCAGGAGAC CCTGAGGGCA GCCGGTTCCT |
| | ACTTTGGACT GAGAGAAGGG |
| 1801 | AGCCCCAGGC TGGAGCAGCA TGAGGCCCAG CAAGAAGGGC |
| | TTGGGTTCTG AGGAAGCAGA |
| 1861 | TGTTTCATGC TGTGAGGCCT TGCACCAGGT GGGGGCCACA |
| | GCACCAGCAG CATCTTTGCT |
| 1921 | GGGCAGGGCC CAGCCCTCCA CTGCAGAAGC ATCTGGAAGC |
| | ACCACCTTGT CTCCACAGAG |
| 1981 | CAGCTTGGGC ACAGCAGACT GGCCTGGCCC TGAGACTGGG |
| | GAGTGGCTCC AACAGCCTCC |
| 2041 | TGCCACCCAC ACACCACTCT CCCTAGACTC TCCTAGGGTT |
| | CAGGAGCTGC TGGGCCCAGA |
| 2101 | GGTGACATTT GACTTTTTTC CAGGAAAAAT GTAAGTGTGA |
| | GGAAACCCTT TTTATTTTAT |
| 2161 | TACCTTTCAC TCTCTGGCTG CTGGGTCTGC CGTCGGTCCT |
| | GCTGCTAACC TGGCACCAGA |
| 2221 | GCCTCTGCCC GGGGAGCCTC AGGCAGTCCT CTCCTGCTGT |
| | CACAGCTGCC ATCCACTTCT |
| 2281 | CAGTCCCAGG GCCATCTCTT GGAGTGACAA AGCTGGGATC |
| | AAGGACAGGG AGTTGTAACA |
| 2341 | GAGCAGTGCC AGAGCATGGG CCCAGGTCCC AGGGGAGAGG |
| | TTGGGGCTGG CAGGCCACTG |
| 2401 | GCATGTGCTG AGTAGCGCAG AGCTACCCAG TGAGAGGCCT |
| | TGTCTAACTG CCTTTCCTTC |
| 2461 | TAAAGGGAAT GTTTTTTTCT GAGATAAAAT AAAAACGAGC |
| | CACATCGTGT TTTAAGCTTG |
| 2521 | TCCAAATGA |

By “Adenosine A_{2B} Receptor (A2BR) polypeptide” is meant a protein having at least about 85% amino acid sequence identity to NCBI Accession No. NP_000667.1 or a fragment thereof that binds adenosine. An exemplary A2BR polypeptide sequence is provided below.

(SEQ ID NO: 373)

| | |
|---|---|
| 1 | MLLETQDALY VALELVIAAL SVAGNVLVCA AVGTANTLQT |
| | PTNYFLVSLA AADVAVGLFA |
| 61 | IPFAITISLG FCTDFYGCLF LACFVLVLTQ SSIFSLLAVA |
| | VDRYLAICVP LRYKSLVTGT |
| 121 | RARGVIAVLW VLAFGIGLTP FLGWNSKDSA TNNCTEPWDG |
| | TTNESCCLVK CLFENVVPMS |
| 181 | YMVYFNFFGC VLPPLLIMLV IYIKIFLVAC RQLQRTELMD |
| | HSRTTLQREI HAAKSLAMIV |
| 241 | GIFALCWLPV HAVNCVTLFQ PAQGKNKPKW AMNMAILLSH |
| | ANSVVNPIVY AYRNRDFRYT |
| 301 | FHKIISRYLL CQADVKSGNG QAGVQPALGV GL |

By “Adenosine A_{2B} Receptor (A2BR) polynucleotide” is meant a nucleic acid molecule encoding an A2AR polypeptide, as well as the introns, exons, and regulatory sequences associated with its expression, or fragments thereof. In embodiments, an A2BR polynucleotide is the genomic sequence, mRNA, or gene associated with and/or required for A2BR expression. An exemplary A2BR polynucleotide sequence has about an 85% nucleic acid identity to Genbank Accession No. NM_000676.4, provided below, or a fragment thereof. A further exemplary embodiment of an A2BR polynucleotide sequence has about an 85% nucleic acid identity to the “ADORA2B gene sequence” provided in the Sequence Listing as SEQ ID NO: 374, or a fragment thereof.

(SEQ ID NO: 375)

| | |
|---|---|
| 1 | AGAAGCGGCA GGCGGAGGCG CGGTCCGGGC GCTATGGCCA |
| | TGCCCGGCGG GTCTCACGCG |
| 61 | GCTGCCCCTC GCCCGGCGCG CCTTCGGTAG GGGGCGCCCG |
| | GGGCCCAGCT GGCCCGGCCA |
| 121 | TGCTGCTGGA GACACAGGAC GCGCTGTACG TGGCGCTGGA |
| | GCTGGTCATC GCCGCGCTTT |
| 181 | CGGTGGCGGG CAACGTGCTG GTGTGCGCCG CGGTGGGCAC |
| | GGCGAACACT CTGCAGACGC |
| 241 | CCACCAACTA CTTCCTGGTG TCCCTGGCTG CGGCCGACGT |
| | GGCCGTGGGG CTCTTCGCCA |
| 301 | TCCCCTTTGC CATCACCATC AGCCTGGGCT TCTGCACTGA |
| | CTTCTACGGC TGCCTCTTCC |
| 361 | TCGCCTGCTT CGTGCTGGTG CTCACGCAGA GCTCCATCTT |
| | CAGCCTTCTG GCCGTGGCAG |
| 421 | TCGACAGATA CCTGGCCATC TGTGTCCCGC TCAGGTATAA |
| | AAGTTTGGTC ACGGGGACCC |
| 481 | GAGCAAGAGG GGTCATTGCT GTCCTCTGGG TCCTTGCCTT |
| | TGGCATCGGA TTGACTCCAT |
| 541 | TCCTGGGGTG GAACAGTAAA GACAGTGCCA CCAACAACTG |
| | CACAGAACCC TGGGATGGAA |
| 601 | CCACGAATGA AAGCTGCTGC CTTGTGAAGT GTCTCTTTGA |
| | GAATGTGGTC CCCATGAGCT |
| 661 | ACATGGTATA TTTCAATTTC TTTGGGTGTG TTCTGCCCCC |
| | ACTGCTTATA ATGCTGGTGA |
| 721 | TCTACATTAA GATCTTCCTG GTGGCCTGCA GGCAGCTTCA |
| | GCGCACTGAG CTGATGGACC |
| 781 | ACTCGAGGAC CACCCTCCAG CGGGAGATCC ATGCAGCCAA |
| | GTCACTGGCC ATGATTGTGG |
| 841 | GGATTTTTGC CCTGTGCTGG TTACCTGTGC ATGCTGTTAA |
| | CTGTGTCACT CTTTTCCAGC |
| 901 | CAGCTCAGGG TAAAAATAAG CCCAAGTGGG CAATGAATAT |
| | GGCCATTCTT CTGTCACATG |
| 961 | CCAATTCAGT TGTCAATCCC ATTGTCTATG CTTACCGGAA |
| | CCGAGACTTC CGCTACACTT |
| 1021 | TTCACAAAAT TATCTCCAGG TATCTTCTCT GCCAAGCAGA |
| | TGTCAAGAGT GGGAATGGTC |
| 1081 | AGGCTGGGGT ACAGCCTGCT CTCGGTGTGG GCCTATGATC |
| | TAGGCTCTCG CCTCTTCCAG |
| 1141 | GAGAAGATAC AAATCCACAA GAAACAAAGA GGACACGGCT |
| | GGTTTTCATT GTGAAAGATA |
| 1201 | GCTACACCTC ACAAGGAAAT GGACTGCCTC TCTTGAGCAC |
| | TTCCCTGGAG CTACCACGTA |
| 1261 | TCTAGCTAAT ATGTATGTGT CAGTAGTAGG CTCCAAGGAT |
| | TGACAAATAT ATTTATGATC |
| 1321 | TATTCAGCTG CTTTTACTGT GTGGATTATG CCAACAGCTT |
| | GAATGGATTC TAACAGACTC |
| 1381 | TTTTGTTTTT AAAAGTCTGC CTTGTTTATG GTGGAAAATT |
| | ACTGAAACTA TTTTACTGTG |
| 1441 | AAACAGTGTG AACTATTATA ATGCAAATAC TTTTTAACTT |
| | AGAGGCAATG GAAAAATAAA |
| 1501 | AGTTGACTGT ACTAAAAATG TA. |

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). (e.g., eukaryotic, prokaryotic), including but not limited to algae, bacteria, fungi, plants, invertebrates (e.g., insects), and vertebrates (e.g., amphibians, mammals).

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor (ABE) polynucleotide” is meant a polynucleotide that encodes an ABE.

By “Adenosine Base Editor 8 (ABE8) polypeptide” or “ABE8” is meant a base editor as defined herein comprising one or more of the alterations listed in Table 15, one of the combinations of alterations listed in Table 15, or an alteration at any of the amino acid positions listed in Table 15, such alterations are relative to the following reference sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1) or at a corresponding position in another adenosine deaminase. In some embodiments, an ABE8 comprises alterations at amino acids 82 and/or 166 of SEQ ID NO. 1. In some embodiments, ABE8 comprises further alterations, as described herein, relative to the reference sequence.

By “Adenosine Base Editor 8 (ABE8) polynucleotide” is meant a polynucleotide encoding an ABE8.

“Administering” is referred to herein as providing one or more compositions described herein to a patient or a subject.

By “agent” is meant any small molecule chemical compound, antibody, nucleic acid molecule, or polypeptide, or fragments thereof.

“Allogeneic” as used herein, refers to cells taken from two non-identical individuals of the same species.

By “alteration” is meant a change (e.g., increase or decrease) in the level, structure, or activity of an analyte, gene or polypeptide as detected by standard art known methods such as those described herein. As used herein, an alteration includes a 10% change in expression levels, a 25% change, a 40% change, and a 50% or greater change in expression levels. In some embodiments, an alteration includes an insertion, deletion, or substitution of a nucleobase or amino acid.

By “ameliorate” is meant decrease, suppress, attenuate, diminish, arrest, or stabilize the development or progression of a disease.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

“Autologous,” as used herein, refers to cells obtained from the same individual.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1) in conjunction with a guide polynucleotide (e.g., guide RNA (gRNA)). Representative nucleic acid and protein sequences of base editors are provided in the Sequence Listing as SEQ ID NOs: 2-11.

By “AZD4635” is meant an agent with the structure

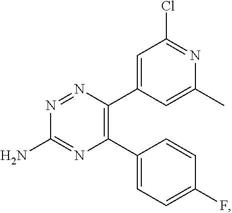 [SMILES: Cc1cc(-c2nnc(N)nc2-c2ccc(F)cc2)cc(Cl)n1]

and corresponding to CAS No. 1321514-06-0, or a pharmaceutically acceptable salt thereof, that inhibits A2AR signaling.

By “beta-2 microglobulin (02M; B2M) polypeptide” is meant a protein having at least about 85% amino acid sequence identity to UniProt Accession No. P61769, which is provided below, or a fragment thereof having immunomodulatory activity.
>sp|P61769|B2MG_HUMAN Beta-2-microglobulin OS=Homo sapiens OX=9606 GN=B2M PE=1 SV=1

(SEQ ID NO: 467)
MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDM.

By “beta-2-microglobulin (β2M; B2M) polynucleotide” is meant a nucleic acid molecule encoding an β2M polypeptide, as well as the introns, exons, 3′ untranslated regions, 5′ untranslated regions, and regulatory sequences associated with its expression, or fragments thereof. The beta-2-microglobulin gene encodes a serum protein associated with the major histocompatibility complex. β2M is involved in non-self-recognition by host CD8+ T cells. An exemplary 02M polynucleotide sequence is provided at GenBank Accession No. DQ217933.1, which is provided below.
>DQ217933.1 Homo sapiens beta-2-microglobin (β2M) gene, complete cds

(SEQ ID NO: 468)
CATGTCATAAATGGTAAGTCCAAGAAAAATACAGGTATTCCCCCCCAAAGAAAACTGTAAAATC
GACTTTTTTCTATCTGTACTGTTTTTTATTGGTTTTTAAATTGGTTTTCCAAGTGAGTAAATCA
GAATCTATCTGTAATGGATTTTAAATTTAGTGTTTCTCTGTGATGTAGTAAACAAGAAACTAGA
GGCAAAAATAGCCCTGTCCCTTGCTAAACTTCTAAGGCACTTTTCTAGTACAACTCAACACTAA
CATTTCAGGCCTTTAGTGCCTTATATGAGTTTTTAAAAGGGGGAAAAGGGAGGGAGCAAGAGTG
TCTTAACTCATACATTTAGGCATAACAATTATTCTCATATTTTAGTTATTGAGAGGGCTGGTAG
AAAAACTAGGTAAATAATATTAATAATTATAGCGCTTATTAAACACTACAGAACACTTACTATG
TACCAGGCATTGTGGGAGGCTCTCTCTTGTGCATTATCTCATTTCATTAGGTCCATGGAGAGTA
TTGCATTTTCTTAGTTTAGGCATGGCCTCCACAATAAAGATTATCAAAAGCCTAAAAATATGTA
AAAGAAACCTAGAAGTTATTTGTTGTGCTCCTTGGGGAAGCTAGGCAAATCCTTTCAACTGAAA
ACCATGGTGACTTCCAAGATCTCTGCCCCTCCCCATCGCCATGGTCCACTTCCTCTTCTCACTG
TTCCTCTTAGAAAAGATCTGTGGACTCCACCACCACGAAATGGCGGCACCTTATTTATGGTCAC
TTTAGAGGGTAGGTTTTCTTAATGGGTCTGCCTGTCATGTTTAACGTCCTTGGCTGGGTCCAAG
GCAGATGCAGTCCAAACTCTCACTAAAATTGCCGAGCCCTTTGTCTTCCAGTGTCTAAAATATT
AATGTCAATGGAATCAGGCCAGAGTTTGAATTCTAGTCTCTTAGCCTTTGTTTCCCCTGTCCAT
AAAATGAATGGGGGTAATTCTTTCCTCCTACAGTTTATTTATATATTCACTAATTCATTCATTC
ATCCATCCATTCGTTCATTCGGTTTACTGAGTACCTACTATGTGCCAGCCCCTGTTCTAGGGTG
GAAACTAAGAGAATGATGTACCTAGAGGGCGCTGGAAGCTCTAAAGCCCTAGCAGTTACTGCTT
TTACTATTAGTGGTCGTTTTTTTCTCCCCCCCGCCCCCCGACAAATCAACAGAACAAAGAAAAT
TACCTAAACAGCAAGGACATAGGGAGGAACTTCTTGGCACAGAACTTTCCAAACACTTTTTCCT
GAAGGGATACAAGAAGCAAGAAAGGTACTCTTTCACTAGGACCTTCTCTGAGCTGTCCTCAGGA
TGCTTTTGGGACTATTTTTCTTACCCAGAGAATGGAGAAACCCTGCAGGGAATTCCCAAGCTGT
AGTTATAAACAGAAGTTCTCCTTCTGCTAGGTAGCATTCAAAGATCTTAATCTTCTGGGTTTCC
GTTTTCTCGAATGAAAAATGCAGGTCCGAGCAGTTAACTGGCTGGGGCACCATTAGCAAGTCAC
TTAGCATCTCTGGGGCCAGTCTGCAAAGCGAGGGGGCAGCCTTAATGTGCCTCCAGCCTGAAGT
CCTAGAATGAGCGCCCGGTGTCCCAAGCTGGGGCGCGCACCCCAGATCGGAGGGCGCCGATGTA
CAGACAGCAAACTCACCCAGTCTAGTGCATGCCTTCTTAAACATCACGAGACTCTAAGAAAAGG
AAACTGAAAACGGGAAAGTCCCTCTCTCTAACCTGGCACTGCGTCGCTGGCTTGGAGACAGGTG
ACGGTCCCTGCGGGCCTTGTCCTGATTGGCTGGGCACGCGTTTAATATAAGTGGAGGCGTCGCG
CTGGCGGGCATTCCTGAAGCTGACAGCATTCGGGCCGAGATGTCTCGCTCCGTGGCCTTAGCTG
TGCTCGCGCTACTCTCTCTTTCTGGCCTGGAGGCTATCCAGCGTGAGTCTCTCCTACCCTCCCG
CTCTGGTCCTTCCTCTCCCGCTCTGCACCCTCTGTGGCCCTCGCTGTGCTCTCTCGCTCCGTGA
CTTCCCTTCTCCAAGTTCTCCTTGGTGGCCCGCCGTGGGGCTAGTCCAGGGCTGGATCTCGGGG
AAGCGGCGGGGTGGCCTGGGAGTGGGGAAGGGGGTGCGCACCCGGGACGCGCGCTACTTGCCCC
TTTCGGCGGGGAGCAGGGGAGACCTTTGGCCTACGGCGACGGGAGGGTCGGGACAAAGTTTAGG
GCGTCGATAAGCGTCAGAGCGCCGAGGTTGGGGGAGGGTTTCTCTTCCGCTCTTTCGCGGGGCC
TCTGGCTCCCCCAGCGCAGCTGGAGTGGGGGACGGGTAGGCTCGTCCCAAAGGCGCGGCGCTGA
GGTTTGTGAACGCGTGGAGGGGCGCTTGGGGTCTGGGGGAGGCGTCGCCCGGGTAAGCCTGTCT
GCTGCGGCTCTGCTTCCCTTAGACTGGAGAGCTGTGGACTTCGTCTAGGCGCCCGCTAAGTTCG
CATGTCCTAGCACCTCTGGGTCTATGTGGGGCCACACCGTGGGGAGGAAACAGCACGCGACGTT
TGTAGAATGCTTGGCTGTGATACAAAGCGGTTTCGAATAATTAACTTATTTGTTCCCATCACAT
GTCACTTTTAAAAAATTATAAGAACTACCCGTTATTGACATCTTTCTGTGTGCCAAGGACTTTA
TGTGCTTTGCGTCATTTAATTTTGAAAACAGTTATCTTCCGCCATAGATAACTACTATGGTTAT
CTTCTGCCTCTCACAGATGAAGAAACTAAGGCACCGAGATTTTAAGAAACTTAATTACACAGGG
GATAAATGGCAGCAATCGAGATTGAAGTCAAGCCTAACCAGGGCTTTTGCGGGAGCGCATGCCT
TTTGGCTGTAATTCGTGCATTTTTTTTTAAGAAAAACGCCTGCCTTCTGCGTGAGATTCTCCAG
AGCAAACTGGGCGGCATGGGCCCTGTGGTCTTTTCGTACAGAGGGCTTCCTCTTTGGCTCTTTG
CCTGGTTGTTTCCAAGATGTACTGTGCCTCTTACTTTCGGTTTTGAAAACATGAGGGGGTTGGG
CGTGGTAGCTTACGCCTGTAATCCCAGCACTTAGGGAGGCCGAGGCGGGAGGATGGCTTGAGGT
CCGTAGTTGAGACCAGCCTGGCCAACATGGTGAAGCCTGGTCTCTACAAAAAATAATAACAAAA
ATTAGCCGGGTGTGGTGGCTCGTGCCTGTGGTCCCAGCTGCTCCGGTGGCTGAGGCGGGAGGAT
CTCTTGAGCTTAGGCTTTTGAGCTATCATGGCGCCAGTGCACTCCAGCGTGGGCAACAGAGCGA
GACCCTGTCTCTCAAAAAAGAAAAAAAAAAAAAAAGAAAGAGAAAAGAAAAGAAAGAAAGAAGT
GAAGGTTTGTCAGTCAGGGGAGCTGTAAAACCATTAATAAAGATAATCCAAGATGGTTACCAAG
ACTGTTGAGGACGCCAGAGATCTTGAGCACTTTCTAAGTACCTGGCAATACACTAAGCGCGCTC
ACCTTTTCCTCTGGCAAAACATGATCGAAAGCAGAATGTTTTGATCATGAGAAAATTGCATTTA
ATTTGAATACAATTTATTTACAACATAAAGGATAATGTATATATCACCACCATTACTGGTATTT
GCTGGTTATGTTAGATGTCATTTTAAAAAATAACAATCTGATATTTAAAAAAAAATCTTATTTT
GAAAATTTCCAAAGTAATACATGCCATGCATAGACCATTTCTGGAAGATACCACAAGAAACATG
TAATGATGATTGCCTCTGAAGGTCTATTTTCCTCCTCTGACCTGTGTGTGGGTTTTGTTTTTGT
TTTACTGTGGGCATAAATTAATTTTTCAGTTAAGTTTTGGAAGCTTAAATAACTCTCCAAAAGT
CATAAAGCCAGTAACTGGTTGAGCCCAAATTCAAACCCAGCCTGTCTGATACTTGTCCTCTTCT
TAGAAAAGATTACAGTGATGCTCTCACAAAATCTTGCCGCCTTCCCTCAAACAGAGAGTTCCAG
GCAGGATGAATCTGTGCTCTGATCCCTGAGGCATTTAATATGTTCTTATTATTAGAAGCTCAGA
TGCAAAGAGCTCTCTTAGCTTTTAATGTTATGAAAAAAATCAGGTCTTCATTAGATTCCCCAAT
CCACCTCTTGATGGGGCTAGTAGCCTTTCCTTAATGATAGGGTGTTTCTAGAGAGATATATCTG
GTCAAGGTGGCCTGGTACTCCTCCTTCTCCCCACAGCCTCCCAGACAAGGAGGAGTAGCTGCCT
TTTAGTGATCATGTACCCTGAATATAAGTGTATTTAAAAGAATTTTATACACATATATTTAGTG
TCAATCTGTATATTTAGTAGCACTAACACTTCTCTTCATTTTCAATGAAAAATATAGAGTTTAT
AATATTTTCTTCCCACTTCCCCATGGATGGTCTAGTCATGCCTCTCATTTTGGAAAGTACTGTT
TCTGAAACATTAGGCAATATATTCCCAACCTGGCTAGTTTACAGCAATCACCTGTGGATGCTAA
TTAAAACGCAAATCCCACTGTCACATGCATTACTCCATTTGATCATAATGGAAAGTATGTTCTG
TCCCATTTGCCATAGTCCTCACCTATCCCTGTTGTATTTTATCGGGTCCAACTCAACCATTTAA
GGTATTTGCCAGCTCTTGTATGCATTTAGGTTTTGTTTCTTTGTTTTTTAGCTCATGAAATTAG
GTACAAAGTCAGAGAGGGGTCTGGCATATAAAACCTCAGCAGAAATAAAGAGGTTTTGTTGTTT
GGTAAGAACATACCTTGGGTTGGTTGGGCACGGTGGCTCGTGCCTGTAATCCCAACACTTTGGG
AGGCCAAGGCAGGCTGATCACTTGAAGTTGGGAGTTCAAGACCAGCCTGGCCAACATGGTGAAA
TCCCGTCTCTACTGAAAATACAAAAATTAACCAGGCATGGTGGTGTGTGCCTGTAGTCCCAGGA
ATCACTTGAACCCAGGAGGCGGAGGTTGCAGTGAGCTGAGATCTCACCACTGCACACTGCACTC
CAGCCTGGGCAATGGAATGAGATTCCATCCCAAAAAATAAAAAAATAAAAAAATAAAGAACATA
CCTTGGGTTGATCCACTTAGGAACCTCAGATAATAACATCTGCCACGTATAGAGCAATTGCTAT
GTCCCAGGCACTCTACTAGACACTTCATACAGTTTAGAAAATCAGATGGGTGTAGATCAAGGCA
GGAGCAGGAACCAAAAAGAAAGGCATAAACATAAGAAAAAAAATGGAAGGGGTGGAAACAGAGT
ACAATAACATGAGTAATTTGATGGGGGCTATTATGAACTGAGAAATGAACTTTGAAAAGTATCT
TGGGGCCAAATCATGTAGACTCTTGAGTGATGTGTTAAGGAATGCTATGAGTGCTGAGAGGGCA
TCAGAAGTCCTTGAGAGCCTCCAGAGAAAGGCTCTTAAAAATGCAGCGCAATCTCCAGTGACAG
AAGATACTGCTAGAAATCTGCTAGAAAAAAAACAAAAAAGGCATGTATAGAGGAATTATGAGGG
AAAGATACCAAGTCACGGTTTATTCTTCAAAATGGAGGTGGCTTGTTGGGAAGGTGGAAGCTCA
TTTGGCCAGAGTGGAAATGGAATTGGGAGAAATCGATGACCAAATGTAAACACTTGGTGCCTGA
TATAGCTTGACACCAAGTTAGCCCCAAGTGAAATACCCTGGCAATATTAATGTGTCTTTTCCCG
ATATTCCTCAGGTACTCCAAAGATTCAGGTTTACTCACGTCATCCAGCAGAGAATGGAAAGTCA
AATTTCCTGAATTGCTATGTGTCTGGGTTTCATCCATCCGACATTGAAGTTGACTTACTGAAGA
ATGGAGAGAGAATTGAAAAAGTGGAGCATTCAGACTTGTCTTTCAGCAAGGACTGGTCTTTCTA
TCTCTTGTACTACACTGAATTCACCCCCACTGAAAAAGATGAGTATGCCTGCCGTGTGAACCAT
GTGACTTTGTCACAGCCCAAGATAGTTAAGTGGGGTAAGTCTTACATTCTTTTGTAAGCTGCTG
AAAGTTGTGTATGAGTAGTCATATCATAAAGCTGCTTTGATATAAAAAAGGTCTATGGCCATAC
TACCCTGAATGAGTCCCATCCCATCTGATATAAACAATCTGCATATTGGGATTGTCAGGGAATG
TTCTTAAAGATCAGATTAGTGGCACCTGCTGAGATACTGATGCACAGCATGGTTTCTGAACCAG
TAGTTTCCCTGCAGTTGAGCAGGGAGCAGCAGCAGCACTTGCACAAATACATATACACTCTTAA
CACTTCTTACCTACTGGCTTCCTCTAGCTTTTGTGGCAGCTTCAGGTATATTTAGCACTGAACG
AACATCTCAAGAAGGTATAGGCCTTTGTTTGTAAGTCCTGCTGTCCTAGCATCCTATAATCCTG
GACTTCTCCAGTACTTTCTGGCTGGATTGGTATCTGAGGCTAGTAGGAAGGGCTTGTTCCTGCT
GGGTAGCTCTAAACAATGTATTCATGGGTAGGAACAGCAGCCTATTCTGCCAGCCTTATTTCTA
ACCATTTTAGACATTTGTTAGTACATGGTATTTTAAAAGTAAAACTTAATGTCTTCCTTTTTTT
TCTCCACTGTCTTTTTCATAGATCGAGACATGTAAGCAGCATCATGGAGGTAAGTTTTTGACCT
TGAGAAAATGTTTTTGTTTCACTGTCCTGAGGACTATTTATAGACAGCTCTAACATGATAACCC
TCACTATGTGGAGAACATTGACAGAGTAACATTTTAGCAGGGAAAGAAGAATCCTACAGGGTCA
TGTTCCCTTCTCCTGTGGAGTGGCATGAAGAAGGTGTATGGCCCCAGGTATGGCCATATTACTG
ACCCTCTACAGAGAGGGCAAAGGAACTGCCAGTATGGTATTGCAGGATAAAGGCAGGTGGTTAC
CCACATTACCTGCAAGGCTTTGATCTTTCTTCTGCCATTTCCACATTGGACATCTCTGCTGAGG
AGAGAAAATGAACCACTCTTTTCCTTTGTATAATGTTGTTTTATTCTTCAGACAGAAGAGAGGA
GTTATACAGCTCTGCAGACATCCCATTCCTGTATGGGGACTGTGTTTGCCTCTTAGAGGTTCCC
AGGCCACTAGAGGAGATAAAGGGAAACAGATTGTTATAACTTGATATAATGATACTATAATAGA
TGTAACTACAAGGAGCTCCAGAAGCAAGAGAGAGGGAGGAACTTGGACTTCTCTGCATCTTTAG
TTGGAGTCCAAAGGCTTTTCAATGAAATTCTACTGCCCAGGGTACATTGATGCTGAAACCCCAT
TCAAATCTCCTGTTATATTCTAGAACAGGGAATTGATTTGGGAGAGCATCAGGAAGGTGGATGA
TCTGCCCAGTCACACTGTTAGTAAATTGTAGAGCCAGGACCTGAACTCTAATATAGTCATGTGT
TACTTAATGACGGGGACATGTTCTGAGAAATGCTTACACAAACCTAGGTGTTGTAGCCTACTAC
ACGCATAGGCTACATGGTATAGCCTATTGCTCCTAGACTACAAACCTGTACAGCCTGTTACTGT
ACTGAATACTGTGGGCAGTTGTAACACAATGGTAAGTATTTGTGTATCTAAACATAGAAGTTGC
AGTAAAAATATGCTATTTTAATCTTATGAGACCACTGTCATATATACAGTCCATCATTGACCAA
AACATCATATCAGCATTTTTTCTTCTAAGATTTTGGGAGCACCAAAGGGATACACTAACAGGAT
ATACTCTTTATAATGGGTTTGGAGAACTGTCTGCAGCTACTTCTTTTAAAAAGGTGATCTACAC
AGTAGAAATTAGACAAGTTTGGTAATGAGATCTGCAATCCAAATAAAATAAATTCATTGCTAAC
CTTTTTCTTTTCTTTTCAGGTTTGAAGATGCCGCATTTGGATTGGATGAATTCCAAATTCTGCT
TGCTTGCTTTTTAATATTGATATGCTTATACACTTACACTTTATGCACAAAATGTAGGGTTATA
ATAATGTTAACATGGACATGATCTTCTTTATAATTCTACTTTGAGTGCTGTCTCCATGTTTGAT
GTATCTGAGCAGGTTGCTCCACAGGTAGCTCTAGGAGGGCTGGCAACTTAGAGGTGGGGAGCAG
AGAATTCTCTTATCCAACATCAACATCTTGGTCAGATTTGAACTCTTCAATCTCTTGCACTCAA
AGCTTGTTAAGATAGTTAAGCGTGCATAAGTTAACTTCCAATTTACATACTCTGCTTAGAATTT
GGGGGAAAATTTAGAAATATAATTGACAGGATTATTGGAAATTTGTTATAATGAATGAAACATT
TTGTCATATAAGATTCATATTTACTTCTTATACATTTGATAAAGTAAGGCATGGTTGTGGTTAA
TCTGGTTTATTTTTGTTCCACAAGTTAAATAAATCATAAAACTTGATGTGTTATCTCTTATATC
TCACTCCCACTATTACCCCTTTATTTTCAAACAGGGAAACAGTCTTCAAGTTCCACTTGGTAAA
AAATGTGAACCCCTTGTATATAGAGTTTGGCTCACAGTGTAAAGGGCCTCAGTGATTCACATTT
TCCAGATTAGGAATCTGATGCTCAAAGAAGTTAAATGGCATAGTTGGGGTGACACAGCTGTCTA
GTGGGAGGCCAGCCTTCTATATTTTAGCCAGCGTTCTTTCCTGCGGGCCAGGTCATGAGGAGTA
TGCAGACTCTAAGAGGGAGCAAAAGTATCTGAAGGATTTAATATTTTAGCAAGGAATAGATATA
CAATCATCCCTTGGTCTCCCTGGGGGATTGGTTTCAGGACCCCTTCTTGGACACCAAATCTATG
GATATTTAAGTCCCTTCTATAAAATGGTATAGTATTTGCATATAACCTATCCACATCCTCCTGT
ATACTTTAAATCATTTCTAGATTACTTGTAATACCTAATACAATGTAAATGCTATGCAAATAGT
TGTTATTGTTTAAGGAATAATGACAAGAAAAAAAAGTCTGTACATGCTCAGTAAAGACACAACC
ATCCCTTTTTTTCCCCAGTGTTTTTGATCCATGGTTTGCTGAATCCACAGATGTGGAGCCCCTG
GATACGGAAGGCCCGCTGTACTTTGAATGACAAATAACAGATTTAAA.

By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE).

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat) associated nuclease.

By “chimeric antigen receptor” or “CAR” is meant a synthetic or engineered receptor comprising an extracellular antigen binding domain joined to one or more intracellular signaling domains (e.g., T cell signaling domain) that confers specificity for an antigen onto an immune effector cell.

By “chimeric antigen receptor T cell” or “CAR-T cell” is meant a T cell expressing a CAR that has antigen specificity determined by the antibody-derived targeting domain of the CAR. As used herein, “CAR-T cells” includes T cells or NK cells. As used herein, “CAR-T cells” include cells engineered to express a CAR or a T cell receptor (TCR). Methods of making CARs (e.g., for treatment of cancer) are publicly available (see, e.g., Park et al., Trends Biotechnol., 29:550-557, 2011; Grupp et al., N Engl J Med., 368:1509-1518, 2013; Han et al., J. Hematol Oncol. 6:47, 2013; Haso et al., (2013) Blood, 121, 1165-1174; PCT Pubs. WO2012/079000, WO2013/059593; and U.S. Pub. 2012/0213783, each of which is incorporated by reference herein in its entirety).

By “class II, major histocompatibility complex, transactivator (CIITA) polypeptide” is meant a protein having at least about 85% amino acid sequence identity to NCBI Accession No. NP_001273331.1, which is provided below, or a fragment thereof having DNA binding activity.
>NP_001273331.1 MHC class II transactivator isoform 1 [Homo sapiens]

(SEQ ID NO: 469)
MRCLAPRPAGSYLSEPQGSSQCATMELGPLEGGYLELLNSDADPLCLYHF
YDQMDLAGEEEIELYSEPDTDTINCDQFSRLLCDMEGDEETREAYANIAE
LDQYVFQDSQLEGLSKDIFIEHIGPDEVIGESMEMPAEVGQKSQKRPFPE
ELPADLKHWKPAEPPTVVTGSLLVGPVSDCSTLPCLPLPALFNQEPASGQ
MRLEKTDQIPMPFSSSSLSCLNLPEGPIQFVPTISTLPHGLWQISEAGTG
VSSIFIYHGEVPQASQVPPPSGFTVHGLPTSPDRPGSTSPFAPSATDLPS
MPEPALTSRANMTEHKTSPTQCPAAGEVSNKLPKWPEPVEQFYRSLQDTY
GAEPAGPDGILVEVDLVQARLERSSSKSLERELATPDWAERQLAQGGLAE
VLLAAKEHRRPRETRVIAVLGKAGQGKSYWAGAVSRAWACGRLPQYDFVF
SVPCHCLNRPGDAYGLQDLLFSLGPQPLVAADEVFSHILKRPDRVLLILD
GFEELEAQDGFLHSTCGPAPAEPCSLRGLLAGLFQKKLLRGCTLLLTARP
RGRLVQSLSKADALFELSGFSMEQAQAYVMRYFESSGMTEHQDRALTLLR
DRPLLLSHSHSPTLCRAVCQLSEALLELGEDAKLPSTLTGLYVGLLGRAA
LDSPPGALAELAKLAWELGRRHQSTLQEDQFPSADVRTWAMAKGLVQHPP
RAAESELAFPSFLLQCFLGALWLALSGEIKDKELPQYLALTPRKKRPYDN
WLEGVPRFLAGLIFQPPARCLGALLGPSAAASVDRKQKVLARYLKRLQPG
TLRARQLLELLHCAHEAEEAGIWQHVVQELPGRLSFLGTRLTPPDAHVLG
KALEAAGQDFSLDLRSTGICPSGLGSLVGLSCVTRFRAALSDTVALWESL
QQHGETKLLQAAEEKFTIEPFKAKSLKDVEDLGKLVQTQRTRSSSEDTAG
ELPAVRDLKKLEFALGPVSGPQAFPKLVRILTAFSSLQHLDLDALSENKI
GDEGVSQLSATFPQLKSLETLNLSQNNITDLGAYKLAEALPSLAASLLRL
SLYNNCICDVGAESLARVLPDMVSLRVMDVQYNKFTAAGAQQLAASLRRC
PHVETLAMWTPTIPFSVQEHLQQQDSRISLR.

By “class II, major histocompatibility complex, transactivator (CIITA) polynucleotide” is meant a nucleic acid molecule encoding an CIITA polypeptide, as well as the introns, exons, 3′ untranslated regions, 5′ untranslated regions, and regulatory sequences associated with its expression, or fragments thereof. An exemplary CIITA polynucleotide is provided at NCBI Accession No. NM_001286402.1, which is provide below.
>NM_001286402.1 Homo sapiens class II major histocompatibility complex transactivator (CIITA), transcript variant 1, mRNA

(SEQ ID NO: 470)
GGTTAGTGATGAGGCTAGTGATGAGGCTGTGTGCTTCTGAGCTGGGCATCCGAAGGCATCCTTG
GGGAAGCTGAGGGCACGAGGAGGGGCTGCCAGACTCCGGGAGCTGCTGCCTGGCTGGGATTCCT
ACACAATGCGTTGCCTGGCTCCACGCCCTGCTGGGTCCTACCTGTCAGAGCCCCAAGGCAGCTC
ACAGTGTGCCACCATGGAGTTGGGGCCCCTAGAAGGTGGCTACCTGGAGCTTCTTAACAGCGAT
GCTGACCCCCTGTGCCTCTACCACTTCTATGACCAGATGGACCTGGCTGGAGAAGAAGAGATTG
AGCTCTACTCAGAACCCGACACAGACACCATCAACTGCGACCAGTTCAGCAGGCTGTTGTGTGA
CATGGAAGGTGATGAAGAGACCAGGGAGGCTTATGCCAATATCGCGGAACTGGACCAGTATGTC
TTCCAGGACTCCCAGCTGGAGGGCCTGAGCAAGGACATTTTCATAGAGCACATAGGACCAGATG
AAGTGATCGGTGAGAGTATGGAGATGCCAGCAGAAGTTGGGCAGAAAAGTCAGAAAAGACCCTT
CCCAGAGGAGCTTCCGGCAGACCTGAAGCACTGGAAGCCAGCTGAGCCCCCCACTGTGGTGACT
GGCAGTCTCCTAGTGGGACCAGTGAGCGACTGCTCCACCCTGCCCTGCCTGCCACTGCCTGCGC
TGTTCAACCAGGAGCCAGCCTCCGGCCAGATGCGCCTGGAGAAAACCGACCAGATTCCCATGCC
TTTCTCCAGTTCCTCGTTGAGCTGCCTGAATCTCCCTGAGGGACCCATCCAGTTTGTCCCCACC
ATCTCCACTCTGCCCCATGGGCTCTGGCAAATCTCTGAGGCTGGAACAGGGGTCTCCAGTATAT
TCATCTACCATGGTGAGGTGCCCCAGGCCAGCCAAGTACCCCCTCCCAGTGGATTCACTGTCCA
CGGCCTCCCAACATCTCCAGACCGGCCAGGCTCCACCAGCCCCTTCGCTCCATCAGCCACTGAC
CTGCCCAGCATGCCTGAACCTGCCCTGACCTCCCGAGCAAACATGACAGAGCACAAGACGTCCC
CCACCCAATGCCCGGCAGCTGGAGAGGTCTCCAACAAGCTTCCAAAATGGCCTGAGCCGGTGGA
GCAGTTCTACCGCTCACTGCAGGACACGTATGGTGCCGAGCCCGCAGGCCCGGATGGCATCCTA
GTGGAGGTGGATCTGGTGCAGGCCAGGCTGGAGAGGAGCAGCAGCAAGAGCCTGGAGCGGGAAC
TGGCCACCCCGGACTGGGCAGAACGGCAGCTGGCCCAAGGAGGCCTGGCTGAGGTGCTGTTGGC
TGCCAAGGAGCACCGGCGGCCGCGTGAGACACGAGTGATTGCTGTGCTGGGCAAAGCTGGTCAG
GGCAAGAGCTATTGGGCTGGGGCAGTGAGCCGGGCCTGGGCTTGTGGCCGGCTTCCCCAGTACG
ACTTTGTCTTCTCTGTCCCCTGCCATTGCTTGAACCGTCCGGGGGATGCCTATGGCCTGCAGGA
TCTGCTCTTCTCCCTGGGCCCACAGCCACTCGTGGCGGCCGATGAGGTTTTCAGCCACATCTTG
AAGAGACCTGACCGCGTTCTGCTCATCCTAGACGGCTTCGAGGAGCTGGAAGCGCAAGATGGCT
TCCTGCACAGCACGTGCGGACCGGCACCGGCGGAGCCCTGCTCCCTCCGGGGGCTGCTGGCCGG
CCTTTTCCAGAAGAAGCTGCTCCGAGGTTGCACCCTCCTCCTCACAGCCCGGCCCCGGGGCCGC
CTGGTCCAGAGCCTGAGCAAGGCCGACGCCCTATTTGAGCTGTCCGGCTTCTCCATGGAGCAGG
CCCAGGCATACGTGATGCGCTACTTTGAGAGCTCAGGGATGACAGAGCACCAAGACAGAGCCCT
GACGCTCCTCCGGGACCGGCCACTTCTTCTCAGTCACAGCCACAGCCCTACTTTGTGCCGGGCA
GTGTGCCAGCTCTCAGAGGCCCTGCTGGAGCTTGGGGAGGACGCCAAGCTGCCCTCCACGCTCA
CGGGACTCTATGTCGGCCTGCTGGGCCGTGCAGCCCTCGACAGCCCCCCCGGGGCCCTGGCAGA
GCTGGCCAAGCTGGCCTGGGAGCTGGGCCGCAGACATCAAAGTACCCTACAGGAGGACCAGTTC
CCATCCGCAGACGTGAGGACCTGGGCGATGGCCAAAGGCTTAGTCCAACACCCACCGCGGGCCG
CAGAGTCCGAGCTGGCCTTCCCCAGCTTCCTCCTGCAATGCTTCCTGGGGGCCCTGTGGCTGGC
TCTGAGTGGCGAAATCAAGGACAAGGAGCTCCCGCAGTACCTAGCATTGACCCCAAGGAAGAAG
AGGCCCTATGACAACTGGCTGGAGGGCGTGCCACGCTTTCTGGCTGGGCTGATCTTCCAGCCTC
CCGCCCGCTGCCTGGGAGCCCTACTCGGGCCATCGGCGGCTGCCTCGGTGGACAGGAAGCAGAA
GGTGCTTGCGAGGTACCTGAAGCGGCTGCAGCCGGGGACACTGCGGGCGCGGCAGCTGCTGGAG
CTGCTGCACTGCGCCCACGAGGCCGAGGAGGCTGGAATTTGGCAGCACGTGGTACAGGAGCTCC
CCGGCCGCCTCTCTTTTCTGGGCACCCGCCTCACGCCTCCTGATGCACATGTACTGGGCAAGGC
CTTGGAGGCGGCGGGCCAAGACTTCTCCCTGGACCTCCGCAGCACTGGCATTTGCCCCTCTGGA
TTGGGGAGCCTCGTGGGACTCAGCTGTGTCACCCGTTTCAGGGCTGCCTTGAGCGACACGGTGG
CGCTGTGGGAGTCCCTGCAGCAGCATGGGGAGACCAAGCTACTTCAGGCAGCAGAGGAGAAGTT
CACCATCGAGCCTTTCAAAGCCAAGTCCCTGAAGGATGTGGAAGACCTGGGAAAGCTTGTGCAG
ACTCAGAGGACGAGAAGTTCCTCGGAAGACACAGCTGGGGAGCTCCCTGCTGTTCGGGACCTAA
AGAAACTGGAGTTTGCGCTGGGCCCTGTCTCAGGCCCCCAGGCTTTCCCCAAACTGGTGCGGAT
CCTCACGGCCTTTTCCTCCCTGCAGCATCTGGACCTGGATGCGCTGAGTGAGAACAAGATCGGG
GACGAGGGTGTCTCGCAGCTCTCAGCCACCTTCCCCCAGCTGAAGTCCTTGGAAACCCTCAATC
TGTCCCAGAACAACATCACTGACCTGGGTGCCTACAAACTCGCCGAGGCCCTGCCTTCGCTCGC
TGCATCCCTGCTCAGGCTAAGCTTGTACAATAACTGCATCTGCGACGTGGGAGCCGAGAGCTTG
GCTCGTGTGCTTCCGGACATGGTGTCCCTCCGGGTGATGGACGTCCAGTACAACAAGTTCACGG
CTGCCGGGGCCCAGCAGCTCGCTGCCAGCCTTCGGAGGTGTCCTCATGTGGAGACGCTGGCGAT
GTGGACGCCCACCATCCCATTCAGTGTCCAGGAACACCTGCAACAACAGGATTCACGGATCAGC
CTGAGATGATCCCAGCTGTGCTCTGGACAGGCATGTTCTCTGAGGACACTAACCACGCTGGACC
TTGAACTGGGTACTTGTGGACACAGCTCTTCTCCAGGCTGTATCCCATGAGCCTCAGCATCCTG
GCACCCGGCCCCTGCTGGTTCAGGGTTGGCCCCTGCCCGGCTGCGGAATGAACCACATCTTGCT
CTGCTGACAGACACAGGCCCGGCTCCAGGCTCCTTTAGCGCCCAGTTGGGTGGATGCCTGGTGG
CAGCTGCGGTCCACCCAGGAGCCCCGAGGCCTTCTCTGAAGGACATTGCGGACAGCCACGGCCA
GGCCAGAGGGAGTGACAGAGGCAGCCCCATTCTGCCTGCCCAGGCCCCTGCCACCCTGGGGAGA
AAGTACTTCTTTTTTTTTATTTTTAGACAGAGTCTCACTGTTGCCCAGGCTGGCGTGCAGTGGT
GCGATCTGGGTTCACTGCAACCTCCGCCTCTTGGGTTCAAGCGATTCTTCTGCTTCAGCCTCCC
GAGTAGCTGGGACTACAGGCACCCACCATCATGTCTGGCTAATTTTTCATTTTTAGTAGAGACA
GGGTTTTGCCATGTTGGCCAGGCTGGTCTCAAACTCTTGACCTCAGGTGATCCACCCACCTCAG
CCTCCCAAAGTGCTGGGATTACAAGCGTGAGCCACTGCACCGGGCCACAGAGAAAGTACTTCTC
CACCCTGCTCTCCGACCAGACACCTTGACAGGGCACACCGGGCACTCAGAAGACACTGATGGGC
AACCCCCAGCCTGCTAATTCCCCAGATTGCAACAGGCTGGGCTTCAGTGGCAGCTGCTTTTGTC
TATGGGACTCAATGCACTGACATTGTTGGCCAAAGCCAAAGCTAGGCCTGGCCAGATGCACCAG
CCCTTAGCAGGGAAACAGCTAATGGGACACTAATGGGGCGGTGAGAGGGGAACAGACTGGAAGC
ACAGCTTCATTTCCTGTGTCTTTTTTCACTACATTATAAATGTCTCTTTAATGTCACAGGCAGG
TCCAGGGTTTGAGTTCATACCCTGTTACCATTTTGGGGTACCCACTGCTCTGGTTATCTAATAT
GTAACAAGCCACCCCAAATCATAGTGGCTTAAAACAACACTCACATTTA.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following:

| | |
|---|---|
| Glutamine | CAG → TAG Stop codon |
| | CAA → TAA |
| Arginine | CGA → TGA |
| Tryptophan | TGG → TGA |
| | TGG → TAG |
| | TGG → TAA |

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and π-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytotoxic T lymphocyte-associated 4 (CTLA4) polypeptide” is meant a protein having at least about 85% amino acid sequence identity to NCBI Ref. Seq. accession No. NP_005205.2, which is provided below, or a functional fragment thereof having immunomodulatory activity.
>NP_005205.2 cytotoxic T-lymphocyte protein 4 isoform CTLA4-TM precursor [Homo sapiens]

(SEQ ID NO: 472)
MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN.

By “cytotoxic T lymphocyte-associated 4 (CTLA4) polynucleotide” is meant a nucleic acid molecule encoding a CTLA4 polypeptide, as well as the introns, exons, 3′ untranslated regions, 5′ untranslated regions, and regulatory sequences associated with its expression, or fragments thereof. An exemplary CTLA4 polynucleotide sequence is provided at Ensembl Accession No. ENSG00000163599.

By “Cytidine Base Editor (CBE)” is meant a base editor that comprises a cytidine deaminase.

By “Cytidine Base Editor (CBE) polynucleotide” is meant a polynucleotide that encodes a CBE.

By “cytidine deaminase” or “cytosine deaminase” is meant a polypeptide or fragment thereof capable of catalyzing the deamination of cytidine or cytosine. In one embodiment, the cytidine deaminase converts cytosine to uracil or 5-methylcytosine to thymine. The terms “cytidine deaminase” and “cytosine deaminase” are used interchangeably throughout the application. A cytidine deaminase may be derived from a mammal (e.g., human, swine, bovine, horse, monkey etc.). Exemplary cytidine deaminases include but are not limited to Petromyzon marinus cytosine deaminase 1 (PmCDA1) (exemplary PmCDA1 polypeptide sequences are provided in the Sequence Listing as SEQ ID NOs: 13-14), Activation-induced cytidine deaminase (AID; AICDA) (exemplary AID polypeptide sequences are provided in the Sequence Listing as SEQ ID NOs: 15-21), and APOBEC (exemplary APOBEC polypeptide sequences are provided in the Sequence Listing as SEQ ID NOs: 12-61). Further exemplary cytidine deaminase sequences are provided in the Sequence Listing as SEQ ID NOs: 62-66 and SEQ ID NOs:67-189.

By “cytosine” or “4-Aminopyrimidin-2(1H)-one” is meant a purine nucleobase with the molecular formula C₄H₅N₃O, having the structure

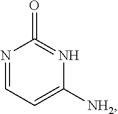

and corresponding to CAS No. 71-30-7.

By “cytidine” is meant a cytosine molecule attached to a ribose sugar via a glycosidic bond, having the structure

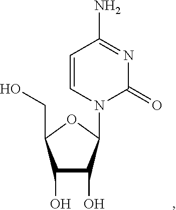

and corresponding to CAS No. 65-46-3. Its molecular formula is C₉H₁₃N₃O₅.

By “cytosine deaminase activity” is meant catalyzing the deamination of cytosine or cytidine. In one embodiment, a polypeptide having cytosine deaminase activity converts cytosine to uracil (i.e., C to U) or 5-methylcytosine to thymine (i.e., 5mC to T). In some embodiments, a cytosine deaminase variant as provided herein has an increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include diseases amenable to treatment with any of the modified immune cells or pharmaceutical compositions as provided herein. In some embodiments, a disease is a type of solid tumor. In some embodiments, the solid tumor is a lung solid tumor. In some embodiments, the solid tumor is an ovarian solid tumor. In embodiments, the disease is a cancer. In embodiments, the cancer and/or solid tumor is a glioma, thyroid cancer, lung cancer, colorectal cancer, esophageal cancer, head and neck (H&N) cancer, stomach cancer, liver cancer, carcinoid, pancreatic cancer, renal cancer, urothelial cancer, prostate cancer, a sarcoma, testis cancer, breast cancer, cervical cancer, endometrial cancer, ovarian cancer, a renal cell carcinoma (RCC), melanoma, skin cancer, uterine cancer, or lyphoma.

By “effective amount” is meant the amount of an agent or cell that is required to ameliorate the symptoms of a disease relative to an untreated patient or an individual without disease, i.e., a healthy individual, or is the amount of the agent or active compound sufficient to elicit a desired biological response. In some embodiments, the cell is a modified immune cell (e.g., T- or NK-cell), for example, an immune cell comprising an alteration that reduces or eliminates the expression of a polynucleotide or polypeptide of interest (e.g., a A2AR, A2BR, HIF1α, HIF1α.I3 polypeptide and/or polynucleotide). In some embodiments, the agent is a base editor as described herein, The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the invention sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is the amount of a modified immune cell (e.g., T- or NK-cell) required to achieve a therapeutic effect (e.g., reduce or stabilize cancer cell proliferation, tumor burden, or cancer cell survival). In one embodiment, an effective amount is sufficient to ameliorate one or more symptoms of a disease (e.g., solid tumor).

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule. In some embodiments the guide polynucleotide contains a sequence selected from those listed in Tables 1A and 1B.

By “Human Leukocyte Antigen-E (HLA-E) polypeptide” is meant a protein having at least about 85% amino acid sequence identity to NCBI Accession No. NP_005507.3, or a fragment thereof having immunomodulatory activity. An exemplary amino acid sequence is provided below.

(SEQ ID NO: 472)
MVDGTLLLLLSEALALTQTWAGSHSLKYFHTSVSRPGRGEPRFISVGYVD
DTQFVRFDNDAASPRMVPRAPWMEQEGSEYWDRETRSARDTAQIFRVNLR
TLRGYYNQSEAGSHTLQWMHGCELGPDGRFLRGYEQFAYDGKDYLTLNED
LRSWTAVDTAAQISEQKSNDASEAEHQRAYLEDTCVEWLHKYLEKGKETL
LHLEPPKTHVTHHPISDHEATLRCWALGFYPAEITLTWQQDGEGHTQDTE
LVETRPAGDGTFQKWAAVVVPSGEEQRYTCHVQHEGLPEPVTLRWKPASQ
PTIPIVGIIAGLVLLGSVVSGAVVAAVIWRKKSSGGKGGSYSKAEWSDSA
QGSESHISL.

By “Human Leukocyte Antigen-E (HLA-E) polynucleotide” is meant a nucleic acid molecule encoding an HLA-E polypeptide, as well as the introns, exons, 3′ untranslated regions, 5′ untranslated regions, and regulatory sequences associated with its expression, or fragments thereof. An exemplary HLA-E polynucleotide is provided at NCBI Accession No. NM_005516.6, which is provided below.

(SEQ ID NO: 473)
CTCAGGACTCAGAGGCTGGGATCATGGTAGATGGAACCCTCCTTTTACTC
CTCTCGGAGGCCCTGGCCCTTACCCAGACCTGGGCGGGCTCCCACTCCTT
GAAGTATTTCCACACTTCCGTGTCCCGGCCCGGCCGCGGGGAGCCCCGCT
TCATCTCTGTGGGCTACGTGGACGACACCCAGTTCGTGCGCTTCGACAAC
GACGCCGCGAGTCCGAGGATGGTGCCGCGGGCGCCGTGGATGGAGCAGGA
GGGGTCAGAGTATTGGGACCGGGAGACACGGAGCGCCAGGGACACCGCAC
AGATTTTCCGAGTGAATCTGCGGACGCTGCGCGGCTACTACAATCAGAGC
GAGGCCGGGTCTCACACCCTGCAGTGGATGCATGGCTGCGAGCTGGGGCC
CGACGGGCGCTTCCTCCGCGGGTATGAACAGTTCGCCTACGACGGCAAGG
ATTATCTCACCCTGAATGAGGACCTGCGCTCCTGGACCGCGGTGGACACG
GCGGCTCAGATCTCCGAGCAAAAGTCAAATGATGCCTCTGAGGCGGAGCA
CCAGAGAGCCTACCTGGAAGACACATGCGTGGAGTGGCTCCACAAATACC
TGGAGAAGGGGAAGGAGACGCTGCTTCACCTGGAGCCCCCAAAGACACAC
GTGACTCACCACCCCATCTCTGACCATGAGGCCACCCTGAGGTGCTGGGC
CCTGGGCTTCTACCCTGCGGAGATCACACTGACCTGGCAGCAGGATGGGG
AGGGCCATACCCAGGACACGGAGCTCGTGGAGACCAGGCCTGCAGGGGAT
GGAACCTTCCAGAAGTGGGCAGCTGTGGTGGTGCCTTCTGGAGAGGAGCA
GAGATACACGTGCCATGTGCAGCATGAGGGGCTACCCGAGCCCGTCACCC
TGAGATGGAAGCCGGCTTCCCAGCCCACCATCCCCATCGTGGGCATCATT
GCTGGCCTGGTTCTCCTTGGATCTGTGGTCTCTGGAGCTGTGGTTGCTGC
TGTGATATGGAGGAAGAAGAGCTCAGGTGGAAAAGGAGGGAGCTACTCTA
AGGCTGAGTGGAGCGACAGTGCCCAGGGGTCTGAGTCTCACAGCTTGTAA
AGCCTGAGACAGCTGCCTTGTGTGCGACTGAGATGCACAGCTGCCTTGTG
TGCGACTGAGATGCAGGATTTCCTCACGCCTCCCCTATGTGTCTTAGGGG
ACTCTGGCTTCTCTTTTTGCAAGGGCCTCTGAATCTGTCTGTGTCCCTGT
TAGCACAATGTGAGGAGGTAGAGAAACAGTCCACCTCTGTGTCTACCATG
ACCCCCTTCCTCACACTGACCTGTGTTCCTTCCCTGTTCTCTTTTCTATT
AAAAATAAGAACCTGGGCAGAGTGCGGCAGCTCATGCCTGTAATCCCAGC
ACTTAGGGAGGCCGAGGAGGGCAGATCACGAGGTCAGGAGATCGAAACCA
TCCTGGCTAACACGGTGAAACCCCGTCTCTACTAAAAAATACAAAAAATT
AGCTGGGCGCAGAGGCACGGGCCTGTAGTCCCAGCTACTCAGGAGGCGGA
GGCAGGAGAATGGCGTCAACCCGGGAGGCGGAGGTTGCAGTGAGCCAGGA
TTGTGCGACTGCACTCCAGCCTGGGTGACAGGGTGAAACGCCATCTCAAA
AAATAAAAATTGAAAAATAAAAAAAGAACCTGGATCTCAATTTAATTTTT
CATATTCTTGCAATGAAATGGACTTGAGGAAGCTAAGATCATAGCTAGAA
ATACAGATAATTCCACAGCACATCTCTAGCAAATTTAGCCTATTCCTATT
CTCTAGCCTATTCCTTACCACCTGTAATCTTGACCATATACCTTGGAGTT
GAATATTGTTTTCATACTGCTGTGGTTTGAATGTTCCCTCCAACACTCAT
GTTGAGACTTAATCCCTAATGTGGCAATACTGAAAGGTGGGGCCTTTGAG
ATGTGATTGGATCGTAAGGCTGTGCCTTCATTCATGGGTTAATGGATTAA
TGGGTTATCACAGGAATGGGACTGGTGGCTTTATAAGAAGAGGAAAAGAG
AACTGAGCTAGCATGCCCAGCCCACAGAGAGCCTCCACTAGAGTGATGCT
AAGTGGAAATGTGAGGTGCAGCTGCCACAGAGGGCCCCCACCAGGGAAAT
GTCTAGTGTCTAGTGGATCCAGGCCACAGGAGAGAGTGCCTTGTGGAGCG
CTGGGAGCAGGACCTGACCACCACCAGGACCCCAGAACTGTGGAGTCAGT
GGCAGCATGCAGCGCCCCCTTGGGAAAGCTTTAGGCACCAGCCTGCAACC
CATTCGAGCAGCCACGTAGGCTGCACCCAGCAAAGCCACAGGCACGGGGC
TACCTGAGGCCTTGGGGGCCCAATCCCTGCTCCAGTGTGTCCGTGAGGCA
GCACACGAAGTCAAAAGAGATTATTCTCTTCCCACAGATACCTTTTCTCT
CCCATGACCCTTTAACAGCATCTGCTTCATTCCCCTCACCTTCCCAGGCT
GATCTGAGGTAAACTTTGAAGTAAAATAAAAGCTGTGTTTGAGCATCA
The HLA-E gene corresponds to Ensembl:
ENSG00000116815.

By “Human Leukocyte Antigen-G (HLA-G) polypeptide” is meant a protein having at least about 85% amino acid sequence identity to NCBI Accession No. NP_001350496.1, which is provided below, or a fragment thereof having immunomodulatory activity.

(SEQ ID NO: 474)
MKTPRMVVMAPRTLFLLLSGALTLTETWAGSHSMRYFSAAVSRPGRGEPR
FIAMGYVDDTQFVRFDSDSACPRMEPRAPWVEQEGPEYWEEETRNTKAHA
QTDRMNLQTLRGYYNQSEASSHTLQWMIGCDLGSDGRLLRGYEQYAYDGK
DYLALNEDLRSWTAADTAAQISKRKCEAANVAEQRRAYLEGTCVEWLHRY
LENGKEMLQRADPPKTHVTHHPVFDYEATLRCWALGFYPAEIILTWQRDG
EDQTQDVELVETRPAGDGTFQKWAAVVVPSGEEQRYTCHVQHEGLPEPLM
LRWKQSSLPTIPIMGIVAGLVVLAAVVTGAAVAAVLWRKKSSD.

By “Human Leukocyte Antigen-G (HLA-G) polynucleotide” is meant a nucleic acid molecule encoding an HLA-G polypeptide, as well as the introns, exons, 3′ untranslated regions, 5′ untranslated regions, and regulatory sequences associated with its expression, or fragments thereof. An exemplary HLA-G polynucleotide is provided at NCBI Accession No. NM_001363567.2, which is provided below.

(SEQ ID NO: 475)
ATATAGTAACATAGTGTGGTACTTTGTCTTGAGGAGATGTCCTGGACTCA
CACGGAAACTTAGGGCTACGGAATGAAGACGCCAAGGATGGTGGTCATGG
CGCCCCGAACCCTCTTCCTGCTGCTCTCGGGGGCCCTGACCCTGACCGAG
ACCTGGGCGGGCTCCCACTCCATGAGGTATTTCAGCGCCGCCGTGTCCCG
GCCCGGCCGCGGGGAGCCCCGCTTCATCGCCATGGGCTACGTGGACGACA
CGCAGTTCGTGCGGTTCGACAGCGACTCGGCGTGTCCGAGGATGGAGCCG
CGGGCGCCGTGGGTGGAGCAGGAGGGGCCGGAGTATTGGGAAGAGGAGAC
ACGGAACACCAAGGCCCACGCACAGACTGACAGAATGAACCTGCAGACCC
TGCGCGGCTACTACAACCAGAGCGAGGCCAGTTCTCACACCCTCCAGTGG
ATGATTGGCTGCGACCTGGGGTCCGACGGACGCCTCCTCCGCGGGTATGA
ACAGTATGCCTACGATGGCAAGGATTACCTCGCCCTGAACGAGGACCTGC
GCTCCTGGACCGCAGCGGACACTGCGGCTCAGATCTCCAAGCGCAAGTGT
GAGGCGGCCAATGTGGCTGAACAAAGGAGAGCCTACCTGGAGGGCACGTG
CGTGGAGTGGCTCCACAGATACCTGGAGAACGGGAAGGAGATGCTGCAGC
GCGCGGACCCCCCCAAGACACACGTGACCCACCACCCTGTCTTTGACTAT
GAGGCCACCCTGAGGTGCTGGGCCCTGGGCTTCTACCCTGCGGAGATCAT
ACTGACCTGGCAGCGGGATGGGGAGGACCAGACCCAGGACGTGGAGCTCG
TGGAGACCAGGCCTGCAGGGGATGGAACCTTCCAGAAGTGGGCAGCTGTG
GTGGTGCCTTCTGGAGAGGAGCAGAGATACACGTGCCATGTGCAGCATGA
GGGGCTGCCGGAGCCCCTCATGCTGAGATGGAAGCAGTCTTCCCTGCCCA
CCATCCCCATCATGGGTATCGTTGCTGGCCTGGTTGTCCTTGCAGCTGTA
GTCACTGGAGCTGCGGTCGCTGCTGTGCTGTGGAGAAAGAAGAGCTCAGA
TTGAAAAGGAGGGAGCTACTCTCAGGCTGCAATGTGAAACAGCTGCCCTG
TGTGGGACTGAGTGGCAAGTCCCTTTGTGACTTCAAGAACCCTGACTCCT
CTTTGTGCAGAGACCAGCCCACCCCTGTGCCCACCATGACCCTCTTCCTC
ATGCTGAACTGCATTCCTTCCCCAATCACCTTTCCTGTTCCAGAAAAGGG
GCTGGGATGTCTCCGTCTCTGTCTCAAATTTGTGGTCCACTGAGCTATAA
CTTACTTCTGTATTAAAATTAGAATCTGAGTATAAA.
The HLA-G gene corresponds to ENSG00000230413,
ENSG00000233095, ENSG00000237216, ENSG00000276051
and ENSG00000204632.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “hypoxia” is meant a condition in which there is an oxygen deficiency that affects a cell, tissue, or biologic environment. In embodiments, the environment is a solid tumor microenvironment.

By “Hypoxia-Inducible Factor 1-alpha (HIF1a) polypeptide” is meant a protein having at least about 85% amino acid sequence identity to NCBI Accession No. NP_001521.1 or a fragment thereof, and having transcriptional regulatory and/or DNA binding activity. An exemplary amino acid sequence is provided below.

| (SEQ ID NO: 376) | |
|---|---|
| 1 | MEGAGGANDK KKISSERRKE KSRDAARSRR SKESEVFYEL AHQLPLPHNV SSHLDKASVM |
| 61 | RLTISYLRVR KLLDAGDLDI EDDMKAQMNC FYLKALDGFV MVLTDDGDMI YISDNVNKYM |
| 121 | GLTQFELTGH SVFDFTHPCD HEEMREMLTH RNGLVKKGKE QNTQRSFFLR MKCTLTSRGR |
| 181 | TMNIKSATWK VLHCTGHIHV YDTNSNQPQC GYKKPPMTCL VLICEPIPHP SNIEIPLDSK |
| 241 | TFLSRHSLDM KFSYCDERIT ELMGYEPEEL LGRSIYEYYH ALDSDHLTKT HHDMFTKGQV |
| 301 | TTGQYRMLAK RGGYVWVETQ ATVIYNTKNS QPQCIVCVNY VVSGIIQHDL IFSLQQTECV |
| 361 | LKPVESSDMK MTQLFTKVES EDTSSLFDKL KKEPDALTLL APAAGDTIIS LDFGSNDTET |
| 421 | DDQQLEEVPL YNDVMLPSPN EKLQNINLAM SPLPTAETPK PLRSSADPAL NQEVALKLEP |
| 481 | NPESLELSFT MPQIQDQTPS PSDGSTRQSS PEPNSPSEYC FYVDSDMVNE FKLELVEKLF |
| 541 | AEDTEAKNPF STQDTDLDLE MLAPYIPMDD DFQLRSFDQL SPLESSSASP ESASPQSTVT |
| 601 | VFQQTQIQEP TANATTTTAT TDELKTVTKD RMEDIKILIA SPSPTHIHKE TTSATSSPYR |
| 661 | DTQSRTASPN RAGKGVIEQT EKSHPRSPNV LSVALSQRTT VPEEELNPKI LALQNAQRKR |
| 721 | KMEHDGSLFQ AVGIGTLLQQ PDDHAATTSL SWKRVKGCKS SEQNGMEQKT IILIPSDLAC |
| 781 | RLLGQSMDES GLPQLTSYDC EVNAPIQGSR NLLQGEELLR ALDQVN |

In embodiments, the alpha subunit of transcription factor hypoxia-inducible factor-1 (HIF-1) polypeptide is a heterodimer composed of an alpha and a beta subunit. HIF-1 functions as a master regulator of cellular and systemic homeostatic response to hypoxia by activating transcription of many genes, including those involved in energy metabolism, angiogenesis, apoptosis, and other genes whose protein products increase oxygen delivery or facilitate metabolic adaptation to hypoxia. HIF-1 plays an important role in tumor angiogenesis.

By “Hypoxia-Inducible Factor 1-alpha (HIF1α) polynucleotide” is meant a nucleic acid encoding an HIF1α polypeptide. An exemplary HIF1α polynucleotide is provided at NCBI Accession No. NM_001530.4. An exemplary nucleic acid sequence is provided below.

| (SEQ ID NO: 378) | |
|---|---|
| 1 | AGTGCACAGT GCTGCCTCGT CTGAGGGGAC AGGAGGATCA CCCTCTTCGT CGCTTCGGCC |
| 61 | AGTGTGTCGG GCTGGGCCCT GACAAGCCAC CTGAGGAGAG GCTCGGAGCC GGGCCCGGAC |
| 121 | CCCGGCGATT GCCGCCCGCT TCTCTCTAGT CTCACGAGGG GTTTCCCGCC TCGCACCCCC |
| 181 | ACCTCTGGAC TTGCCTTTCC TTCTCTTCTC CGCGTGTGGA GGGAGCCAGC GCTTAGGCCG |
| 241 | GAGCGAGCCT GGGGGCCGCC CGCCGTGAAG ACATCGCGGG GACCGATTCA CCATGGAGGG |
| 301 | CGCCGGCGGC GCGAACGACA AGAAAAAGAT AAGTTCTGAA CGTCGAAAAG AAAAGTCTCG |
| 361 | AGATGCAGCC AGATCTCGGC GAAGTAAAGA ATCTGAAGTT TTTTATGAGC TTGCTCATCA |
| 421 | GTTGCCACTT CCACATAATG TGAGTTCGCA TCTTGATAAG GCCTCTGTGA TGAGGCTTAC |
| 481 | CATCAGCTAT TTGCGTGTGA GGAAACTTCT GGATGCTGGT GATTTGGATA TTGAAGATGA |
| 541 | CATGAAAGCA CAGATGAATT GCTTTTATTT GAAAGCCTTG GATGGTTTTG TTATGGTTCT |
| 601 | CACAGATGAT GGTGACATGA TTTACATTTC TGATAATGTG AACAAATACA TGGGATTAAC |
| 661 | TCAGTTTGAA CTAACTGGAC ACAGTGTGTT TGATTTTACT CATCCATGTG ACCATGAGGA |
| 721 | AATGAGAGAA ATGCTTACAC ACAGAAATGG CCTTGTGAAA AAGGGTAAAG AACAAAACAC |
| 781 | ACAGCGAAGC TTTTTTCTCA GAATGAAGTG TACCCTAACT AGCCGAGGAA GAACTATGAA |
| 841 | CATAAAGTCT GCAACATGGA AGGTATTGCA CTGCACAGGC CACATTCACG TATATGATAC |
| 901 | CAACAGTAAC CAACCTCAGT GTGGGTATAA GAAACCACCT ATGACCTGCT TGGTGCTGAT |
| 961 | TTGTGAACCC ATTCCTCACC CATCAAATAT TGAAATTCCT TTAGATAGCA AGACTTTCCT |
| 1021 | CAGTCGACAC AGCCTGGATA TGAAATTTTC TTATTGTGAT GAAAGAATTA CCGAATTGAT |
| 1081 | GGGATATGAG CCAGAAGAAC TTTTAGGCCG CTCAATTTAT GAATATTATC ATGCTTTGGA |
| 1141 | CTCTGATCAT CTGACCAAAA CTCATCATGA TATGTTTACT AAAGGACAAG TCACCACAGG |
| 1201 | ACAGTACAGG ATGCTTGCCA AAAGAGGTGG ATATGTCTGG GTTGAAACTC AAGCAACTGT |
| 1261 | CATATATAAC ACCAAGAATT CTCAACCACA GTGCATTGTA TGTGTGAATT ACGTTGTGAG |
| 1321 | TGGTATTATT CAGCACGACT TGATTTTCTC CCTTCAACAA ACAGAATGTG TCCTTAAACC |
| 1381 | GGTTGAATCT TCAGATATGA AAATGACTCA GCTATTCACC AAAGTTGAAT CAGAAGATAC |
| 1441 | AAGTAGCCTC TTTGACAAAC TTAAGAAGGA ACCTGATGCT TTAACTTTGC TGGCCCCAGC |
| 1501 | CGCTGGAGAC ACAATCATAT CTTTAGATTT TGGCAGCAAC GACACAGAAA CTGATGACCA |
| 1561 | GCAACTTGAG GAAGTACCAT TATATAATGA TGTAATGCTC CCCTCACCCA ACGAAAAATT |
| 1621 | ACAGAATATA AATTTGGCAA TGTCTCCATT ACCCACCGCT GAAACGCCAA AGCCACTTCG |
| 1681 | AAGTAGTGCT GACCCTGCAC TCAATCAAGA AGTTGCATTA AAATTAGAAC CAAATCCAGA |
| 1741 | GTCACTGGAA CTTTCTTTTA CCATGCCCCA GATTCAGGAT CAGACACCTA GTCCTTCCGA |
| 1801 | TGGAAGCACT AGACAAAGTT CACCTGAGCC TAATAGTCCC AGTGAATATT GTTTTTATGT |
| 1861 | GGATAGTGAT ATGGTCAATG AATTCAAGTT GGAATTGGTA GAAAAACTTT TTGCTGAAGA |
| 1921 | CACAGAAGCA AAGAACCCAT TTTCTACTCA GGACACAGAT TTAGACTTGG AGATGTTAGC |
| 1981 | TCCCTATATC CCAATGGATG ATGACTTCCA GTTACGTTCC TTCGATCAGT TGTCACCATT |
| 2041 | AGAAAGCAGT TCCGCAAGCC CTGAAAGCGC AAGTCCTCAA AGCACAGTTA CAGTATTCCA |
| 2101 | GCAGACTCAA ATACAAGAAC CTACTGCTAA TGCCACCACT ACCACTGCCA CCACTGATGA |
| 2161 | ATTAAAAACA GTGACAAAAG ACCGTATGGA AGACATTAAA ATATTGATTG CATCTCCATC |
| 2221 | TCCTACCCAC ATACATAAAG AAACTACTAG TGCCACATCA TCACCATATA GAGATACTCA |
| 2281 | AAGTCGGACA GCCTCACCAA ACAGAGCAGG AAAAGGAGTC ATAGAACAGA CAGAAAAATC |
| 2341 | TCATCCAAGA AGCCCTAACG TGTTATCTGT CGCTTTGAGT CAAAGAACTA CAGTTCCTGA |
| 2401 | GGAAGAACTA AATCCAAAGA TACTAGCTTT GCAGAATGCT CAGAGAAAGC GAAAAATGGA |
| 2461 | ACATGATGGT TCACTTTTTC AAGCAGTAGG AATTGGAACA TTATTACAGC AGCCAGACGA |
| 2521 | TCATGCAGCT ACTACATCAC TTTCTTGGAA ACGTGTAAAA GGATGCAAAT CTAGTGAACA |
| 2581 | GAATGGAATG GAGCAAAAGA CAATTATTTT AATACCCTCT GATTTAGCAT GTAGACTGCT |
| 2641 | GGGGCAATCA ATGGATGAAA GTGGATTACC ACAGCTGACC AGTTATGATT GTGAAGTTAA |
| 2701 | TGCTCCTATA CAAGGCAGCA GAAACCTACT GCAGGGTGAA GAATTACTCA GAGCTTTGGA |
| 2761 | TCAAGTTAAC TGAGCTTTTT CTTAATTTCA TTCCTTTTTT TGGACACTGG TGGCTCATTA |
| 2821 | CCTAAAGCAG TCTATTTATA TTTTCTACAT CTAATTTTAG AAGCCTGGCT ACAATACTGC |
| 2881 | ACAAACTTGG TTAGTTCAAT TTTGATCCCC TTTCTACTTA ATTTACATTA ATGCTCTTTT |
| 2941 | TTAGTATGTT CTTTAATGCT GGATCACAGA CAGCTCATTT TCTCAGTTTT TTGGTATTTA |
| 3001 | AACCATTGCA TTGCAGTAGC ATCATTTTAA AAAATGCACC TTTTTATTTA TTTATTTTTG |
| 3061 | GCTAGGGAGT TTATCCCTTT TTCGAATTAT TTTTAAGAAG ATGCCAATAT AATTTTTGTA |
| 3121 | AGAAGGCAGT AACCTTTCAT CATGATCATA GGCAGTTGAA AAATTTTTAC ACCTTTTTTT |
| 3181 | TCACATTTTA CATAAATAAT AATGCTTTGC CAGCAGTACG TGGTAGCCAC AATTGCACAA |
| 3241 | TATATTTTCT TAAAAAATAC CAGCAGTTAC TCATGGAATA TATTCTGCGT TTATAAAACT |
| 3301 | AGTTTTTAAG AAGAAATTTT TTTTGGCCTA TGAAATTGTT AAACCTGGAA CATGACATTG |
| 3361 | TTAATCATAT AATAATGATT CTTAAATGCT GTATGGTTTA TTATTTAAAT GGGTAAAGCC |
| 3421 | ATTTACATAA TATAGAAAGA TATGCATATA TCTAGAAGGT ATGTGGCATT TATTTGGATA |
| 3481 | AAATTCTCAA TTCAGAGAAA TCATCTGATG TTTCTATAGT CACTTTGCCA GCTCAAAAGA |
| 3541 | AAACAATACC CTATGTAGTT GTGGAAGTTT ATGCTAATAT TGTGTAACTG ATATTAAACC |
| 3601 | TAAATGTTCT GCCTACCCTG TTGGTATAAA GATATTTTGA GCAGACTGTA AACAAGAAAA |
| 3661 | AAAAAATCAT GCATTCTTAG CAAAATTGCC TAGTATGTTA ATTTGCTCAA AATACAATGT |
| 3721 | TTGATTTTAT GCACTTTGTC GCTATTAACA TCCTTTTTTT CATGTAGATT TCAATAATTG |
| 3781 | AGTAATTTTA GAAGCATTAT TTTAGGAATA TATAGTTGTC ACAGTAAATA TCTTGTTTTT |
| 3841 | TCTATGTACA TTGTACAAAT TTTTCATTCC TTTTGCTCTT TGTGGTTGGA TCTAACACTA |
| 3901 | ACTGTATTGT TTTGTTACAT CAAATAAACA TCTTCTGTGG ACCAGG |

By “Hypoxia-Inducible Factor 1-alpha isoform I.3 (HIF1α.I3) polypeptide” is meant a protein having at least about 85% amino acid sequence identity to Genbank Accession No. ACN88547.1 or a fragment thereof, and having transcriptional regulatory and/or DNA binding activity. An exemplary amino acid sequence is provided below.

| (SEQ ID NO: 379) | |
|---|---|
| 1 | MSSQCRSLEN KFVFLKEGLG NSKPEELEEI RIENGRISSE RRKEKSRDAA RSRRSKESEV |
| 61 | FYELAHQLPL PHNVSSHLDK ASVMRLTISY LRVRKLLDAG DLDIEDDMKA QMNCFYLKAL |
| 121 | DGFVMVLTDD GDMIYISDNV NKYMGLTQFE LTGHSVFDFT HPCDHEEMRE MLTHRNGLVK |
| 181 | KGKEQNTQRS FFLRMKCTLT SRGRTMNIKS ATWKVLHCTG HIHVYDTNSN QPQCGYKKPP |
| 241 | MTCLVLICEP IPHPSNIEIP LDSKTFLSRH SLDMKFSYCD ERITELMGYE PEELLGRSIY |
| 301 | EYYHALDSDH LTKTHHDMFT KGQVTTGQYR MLAKRGGYVW VETQATVIYN TKNSQPQCIV |
| 361 | CVNYVVSGII QHDLIFSLQQ TECVLKPVES SDMKMTQLFT KVESEDTSSL FDKLKKEPDA |
| 421 | LTLLAPAAGD TIISLDFGSN DTETDDQQLE EVPLYNDVML PSPNEKLQNI NLAMSPLPTA |
| 481 | ETPKPLRSSA DPALNQEVAL KLEPNPESLE LSFTMPQIQD QTPSPSDGST RQSSPEPNSP |
| 541 | SEYCFYVDSD MVNEFKLELV EKLFAEDTEA KNPFSTQDTD LDLEMLAPYI PMDDDFQLRS |
| 601 | FDQLSPLESS SASPESASPQ STVTVFQQTQ IQEPTANATT TTATTDELKT VTKDRMEDIK |
| 661 | ILIASPSPTH IHKETTSATS SPYRDTQSRT ASPNRAGKGV IEQTEKSHPR SPNVLSVALS |
| 721 | QRTTVPEEEL NPKILALQNA QRKRKMEHDG SLFQAVGIGT LLQQPDDHAA TTSLSWKRVK |
| 781 | GCKSSEQNGM EQKTIILIPS DLACRLLGQS MDESGLPQLT SYDCEVNAPI QGSRNLLQGE |
| 841 | ELLRALDQVN |

By “Hypoxia-Inducible Factor 1-alpha isoform I.3 (HIF1α.I3) polynucleotide,” “HIF-1α isoform 3,” or “HIF1,3” is meant a nucleic acid encoding an HIF1α.I3 polypeptide. An exemplary HIF1α.I3 polynucleotide is provided at Genbank Accession No. FJ790247.1, which is reproduced below:

| (SEQ ID NO: 380) | |
|---|---|
| 1 | ATTTGAAAAC TTGGCAACCT TGGATTGGAT GGATTCATAT TTCTTAGTAT AGAAGTTCTT |
| 61 | GATATAACTG AAAAATTAAG TTAAACACTT AATAAGTGGT GGTTACTCAG CACTTTTAGA |
| 121 | TGCTGTTTAT AATAGATGAC CTTTTCTAAC TAATTTACAG TTTTTTGAAA GATAACTGAG |
| 181 | AGGTTGAGGG ACGGAGATTT TCTTCAAGCA ATTTTTTTTT TTCATTTTAA ATGAGCTCCC |
| 241 | AATGTCGGAG TTTGGAAAAC AAATTTGTCT TTTTAAAAGA AGGTCTAGGA AACTCAAAAC |
| 301 | CTGAAGAATT GGAAGAAATC AGAATAGAAA ATGGTAGGAT AAGTTCTGAA CGTCGAAAAG |
| 361 | AAAAGTCTCG AGATGCAGCC AGATCTCGGC GAAGTAAAGA ATCTGAAGTT TTTTATGAGC |
| 421 | TTGCTCATCA GTTGCCACTT CCACATAATG TGAGTTCGCA TCTTGATAAG GCCTCTGTGA |
| 481 | TGAGGCTTAC CATCAGCTAT TTGCGTGTGA GGAAACTTCT GGATGCTGGT GATTTGGATA |
| 541 | TTGAAGATGA CATGAAAGCA CAGATGAATT GCTTTTATTT GAAAGCCTTG GATGGTTTTG |
| 601 | TTATGGTTCT CACAGATGAT GGTGACATGA TTTACATTTC TGATAATGTG AACAAATACA |
| 661 | TGGGATTAAC TCAGTTTGAA CTAACTGGAC ACAGTGTGTT TGATTTTACT CATCCATGTG |
| 721 | ACCATGAGGA AATGAGAGAA ATGCTTACAC ACAGAAATGG CCTTGTGAAA AAGGGTAAAG |
| 781 | AACAAAACAC ACAGCGAAGC TTTTTTCTCA GAATGAAGTG TACCCTAACT AGCCGAGGAA |
| 841 | GAACTATGAA CATAAAGTCT GCAACATGGA AGGTATTGCA CTGCACAGGC CACATTCACG |
| 901 | TATATGATAC CAACAGTAAC CAACCTCAGT GTGGGTATAA GAAACCACCT ATGACCTGCT |
| 961 | TGGTGCTGAT TTGTGAACCC ATTCCTCACC CATCAAATAT TGAAATTCCT TTAGATAGCA |
| 1021 | AGACTTTCCT CAGTCGACAC AGCCTGGATA TGAAATTTTC TTATTGTGAT GAAAGAATTA |
| 1081 | CCGAATTGAT GGGATATGAG CCAGAAGAAC TTTTAGGCCG CTCAATTTAT GAATATTATC |
| 1141 | ATGCTTTGGA CTCTGATCAT CTGACCAAAA CTCATCATGA TATGTTTACT AAAGGACAAG |
| 1201 | TCACCACAGG ACAGTACAGG ATGCTTGCCA AAAGAGGTGG ATATGTCTGG GTTGAAACTC |
| 1261 | AAGCAACTGT CATATATAAC ACCAAGAATT CTCAACCACA GTGCATTGTA TGTGTGAATT |
| 1321 | ACGTTGTGAG TGGTATTATT CAGCACGACT TGATTTTCTC CCTTCAACAA ACAGAATGTG |
| 1381 | TCCTTAAACC GGTTGAATCT TCAGATATGA AAATGACTCA GCTATTCACC AAAGTTGAAT |
| 1441 | CAGAAGATAC AAGTAGCCTC TTTGACAAAC TTAAGAAGGA ACCTGATGCT TTAACTTTGC |
| 1501 | TGGCCCCAGC CGCTGGAGAC ACAATCATAT CTTTAGATTT TGGCAGCAAC GACACAGAAA |
| 1561 | CTGATGACCA GCAACTTGAG GAAGTACCAT TATATAATGA TGTAATGCTC CCCTCACCCA |
| 1621 | ACGAAAAATT ACAGAATATA AATTTGGCAA TGTCTCCATT ACCCACCGCT GAAACGCCAA |
| 1681 | AGCCACTTCG AAGTAGTGCT GACCCTGCAC TCAATCAAGA AGTTGCATTA AAATTAGAAC |
| 1741 | CAAATCCAGA GTCACTGGAA CTTTCTTTTA CCATGCCCCA GATTCAGGAT CAGACACCTA |
| 1801 | GTCCTTCCGA TGGAAGCACT AGACAAAGTT CACCTGAGCC TAATAGTCCC AGTGAATATT |
| 1861 | GTTTTTATGT GGATAGTGAT ATGGTCAATG AATTCAAGTT GGAATTGGTA GAAAAACTTT |
| 1921 | TTGCTGAAGA CACAGAAGCA AAGAACCCAT TTTCTACTCA GGACACAGAT TTAGACTTGG |
| 1981 | AGATGTTAGC TCCCTATATC CCAATGGATG ATGACTTCCA GTTACGTTCC TTCGATCAGT |
| 2041 | TGTCACCATT AGAAAGCAGT TCCGCAAGCC CTGAAAGCGC AAGTCCTCAA AGCACAGTTA |
| 2101 | CAGTATTCCA GCAGACTCAA ATACAAGAAC CTACTGCTAA TGCCACCACT ACCACTGCCA |
| 2161 | CCACTGATGA ATTAAAAACA GTGACAAAAG ACCGTATGGA AGACATTAAA ATATTGATTG |
| 2221 | CATCTCCATC TCCTACCCAC ATACATAAAG AAACTACTAG TGCCACATCA TCACCATATA |
| 2281 | GAGATACTCA AAGTCGGACA GCCTCACCAA ACAGAGCAGG AAAAGGAGTC ATAGAACAGA |
| 2341 | CAGAAAAATC TCATCCAAGA AGCCCTAACG TGTTATCTGT CGCTTTGAGT CAAAGAACTA |
| 2401 | CAGTTCCTGA GGAAGAACTA AATCCAAAGA TACTAGCTTT GCAGAATGCT CAGAGAAAGC |
| 2461 | GAAAAATGGA ACATGATGGT TCACTTTTTC AAGCAGTAGG AATTGGAACA TTATTACAGC |
| 2521 | AGCCAGACGA TCATGCAGCT ACTACATCAC TTTCTTGGAA ACGTGTAAAA GGATGCAAAT |
| 2581 | CTAGTGAACA GAATGGAATG GAGCAAAAGA CAATTATTTT AATACCCTCT GATTTAGCAT |
| 2641 | GTAGACTGCT GGGGCAATCA ATGGATGAAA GTGGATTACC ACAGCTGACC AGTTATGATT |
| 2701 | GTGAAGTTAA TGCTCCTATA CAAGGCAGCA GAAACCTACT GCAGGGTGAA GAATTACTCA |
| 2761 | GAGCTTTGGA TCAAGTTAAC TGAGCTTTTT CTTAATTTCA TTCCTTTTTT TGGACACTGG |
| 2821 | TGGCTCACTA CCTAAAGCAG TCTATTTATA TTTTCTACAT CTAATTTTAG AAGCCTGGCT |
| 2881 | ACAATACTGC ACAAACTTGG TTAGTTCAAT TTTTGATCCC CTTTCTACTT AATTTACATT |
| 2941 | AATGCTCTTT TTTAGTATGT TCTTTAATGC TGGATCACAG ACAGCTCATT TTCTCAGTTT |
| 3001 | TTTGGTATTT AAACCATTGC ATTGCAGTAG CATCATTTTA AAAAATGCAC CTTTTTATTT |
| 3061 | ATTTATTTTT GGCTAGGGAG TTTATCCCTT TTTCGAATTA TTTTTAAGAA GATGCCAATA |
| 3121 | TAATTTTTGT AAGAAGGCAG TAACCTTTCA TCATGATCAT AGGCAGTTGA AAAATTTTTA |
| 3181 | CACCTTTTTT TTCACATTTT ACATAAATAA TAATGCTTTG CCAGCAGTAC GTGGTAGCCA |
| 3241 | CAATTGCACA ATATATTTTC TTAAAAAATA CCAGCAGTTA CTCATGGAAT ATATTCTGCG |
| 3301 | TTTATAAAAC TAGTTTTTAA GAAGAAATTT TTTTTGGCCT ATGAAATTGT TAAACCTGGA |
| 3361 | ACATGACATT GTTAATCATA TAATAATGAT TCTTAAATGC TGTATGGTTT ATTATTTAAA |
| 3421 | TGGGTAAAGC CATTTACATA ATATAGAAAG ATATGCATAT ATCTAGAAGG |

By “immune cell” is meant a cell of the immune system capable of generating an immune response. Exemplary immune cells include, but are not limited to, T cells, macrophages, and NK cells.

By “increases” is meant a positive alteration of at least 10%, 25%, 50%, 75%, or 100%. In embodiments, an increase in cytokine production is measured as an increase relative to an unmodified reference immune cell in an immunosuppressive environment (e.g., a hypoxic environment, such as a solid tumor microenvironment (sTME)).

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In some embodiments, a linker comprises amino acids, nucleic acids, or analogs thereof. In one embodiment, the term “linker” refers to a covalent linker (e.g., covalent bond) or a non-covalent linker.

By “lymphocyte activation gene 3 (LAG3) polypeptide” is meant a protein having at least about 85% amino acid sequence identity to GenBank Accession No. AAH52589.1, which is provided below, or a fragment thereof having immunomodulatory activity.
>AAH52589.1 LAG3 protein [Homo sapiens]

(SEQ ID NO: 476)
MWEAQFLGLLFLQPLWVAPVKPLQPGAEVPVVWAQEGAPAQLPCSPTIPL
QDLSLLRRAGVTWQHQPDSGPPAAAPGHPLAPGPHPAAPSSWGPRPRRYT
VLSVGPGGLRSGRLPLQPRVQLDERGRQRGDFSLWLRPARRADAGEYRAA
VHLRDRALSCRLRLRLGQASMTASPPGSLRASDWVILNCSFSRPDRPASV
HWFRNRGQGRVPVRESPHHHLAESFLFLPQVSPMDSGPWGCILTYRDGFN
VSIMYNLTVLGLEPPTPLTVYAGAGSRVGLPCRLPAGVGTRSFLTAKWTP
PGGGPDLLVTGDNGDFTLRLEDVSQAQAGTYTCHIHLQEQQLNATVTLAI
ITGQPQVGKE.

By “lymphocyte activation gene 3 (LAG3) polynucleotide” is meant a nucleic acid molecule encoding an x polypeptide, as well as the introns, exons, 3′ untranslated regions, 5′ untranslated regions, and regulatory sequences associated with its expression, or fragments thereof. An exemplary LAG3 polynucleotide sequence is provided at GenBank Accession No. BC052589.1:335-1417, which is provided below. An exemplary LAG3 polynucleotide sequence is also provided at Ensenbl accession no: ENSG00000089692.
>BC052589.1:335-1417 Homo sapiens lymphocyte-activation gene 3, mRNA (cDNA clone MGC:59698 IMAGE:6301931), complete cds

(SEQ ID NO: 477)
ATGTGGGAGGCTCAGTTCCTGGGCTTGCTGTTTCTGCAGCCGCTTTGGGT
GGCTCCAGTGAAGCCTCTCCAGCCAGGGGCTGAGGTCCCGGTGGTGTGGG
CCCAGGAGGGGGCTCCTGCCCAGCTCCCCTGCAGCCCCACAATCCCCCTC
CAGGATCTCAGCCTTCTGCGAAGAGCAGGGGTCACTTGGCAGCATCAGCC
AGACAGTGGCCCGCCCGCTGCCGCCCCCGGCCATCCCCTGGCCCCCGGCC
CTCACCCGGCGGCGCCCTCCTCCTGGGGGCCCAGGCCCCGCCGCTACACG
GTGCTGAGCGTGGGTCCCGGAGGCCTGCGCAGCGGGAGGCTGCCCCTGCA
GCCCCGCGTCCAGCTGGATGAGCGCGGCCGGCAGCGCGGGGACTTCTCGC
TATGGCTGCGCCCAGCCCGGCGCGCGGACGCCGGCGAGTACCGCGCCGCG
GTGCACCTCAGGGACCGCGCCCTCTCCTGCCGCCTCCGTCTGCGCCTGGG
CCAGGCCTCGATGACTGCCAGCCCCCCAGGATCTCTCAGAGCCTCCGACT
GGGTCATTTTGAACTGCTCCTTCAGCCGCCCTGACCGCCCAGCCTCTGTG
CATTGGTTCCGGAACCGGGGCCAGGGCCGAGTCCCTGTCCGGGAGTCCCC
CCATCACCACTTAGCGGAAAGCTTCCTCTTCCTGCCCCAAGTCAGCCCCA
TGGACTCTGGGCCCTGGGGCTGCATCCTCACCTACAGAGATGGCTTCAAC
GTCTCCATCATGTATAACCTCACTGTTCTGGGTCTGGAGCCCCCAACTCC
CTTGACAGTGTACGCTGGAGCAGGTTCCAGGGTGGGGCTGCCCTGCCGCC
TGCCTGCTGGTGTGGGGACCCGGTCTTTCCTCACTGCCAAGTGGACTCCT
CCTGGGGGAGGCCCTGACCTCCTGGTGACTGGAGACAATGGCGACTTTAC
CCTTCGACTAGAGGATGTGAGCCAGGCCCAGGCTGGGACCTACACCTGCC
ATATCCATCTGCAGGAACAGCAGCTCAATGCCACTGTCACATTGGCAATC
ATCACAGGTCAGCCTCAGGTGGGAAAGGAGTAG.

By “marker” is meant any protein or polynucleotide whose expression defines or is associated with a particular cell type or disease state. In some embodiments, a marker has an alteration in expression level or activity that is associated with a disease or disorder (e.g., solid tumor). In some cases, the marker is pCREB, which is suitable, for example, as a marker for expression of Hif1A and A2AR. pCREB is a secondary messenger downstream of A2AR. In some cases, a marker for A2AR or HIF is cytokine production, where higher levels of cytokine production indicate higher levels of A2AR or HIF (e.g., Hif1A) expression. Not intending to be bound by theory, when unedited cells are treated with adenosine, they utilize the A2AR signaling pathway and produce less cytokine. When cells edited to knock-out or reduce expression and/or activity of A2AR, the cells A2AR signaling is not activated and more cytokines are produced.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refers to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (2′—e.g., fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence

(SEQ ID NO: 190)
KRTADGSEFESPKKKRKV,
(SEQ ID NO: 191)
KRPAATKKAGQAKKKK,
(SEQ ID NO: 192)
KKTELQTTNAENKTKKL,
(SEQ ID NO: 193)
KRGINDRNFWRGENGRKTR,
(SEQ ID NO: 194)
RKSGKIAAIVVKRPRK,
(SEQ ID NO: 195)
PKKKRKV,
or
(SEQ ID NO: 196)
MDSLLMNRRKFLYQFKNVRWAKGRRETYLC.

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ. A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-230.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

A “patient” or “subject” as used herein refers to a mammalian subject or individual diagnosed with, at risk of having or developing, or suspected of having or developing a disease or a disorder. In some embodiments, the term “patient” refers to a mammalian subject having or having a propensity to develop a disease (e.g., cancer, solid tumor, neoplasia) or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease casing mutation”, “disease causing variant”, “deleterious mutation”, or “predisposing mutation” refers to a genetic alteration or mutation that increases an individual's susceptibility or predisposition to a certain disease or disorder (e.g., cancer, solid tumor, neoplasia). In some embodiments, the pathogenic mutation comprises at least one wild-type amino acid substituted by at least one pathogenic amino acid in a protein encoded by a gene.

The term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). The terms such as “excipient,” “carrier,” “pharmaceutically acceptable carrier,” “vehicle,” or the like are used interchangeably herein.

By “programmed cell death 1 (PD1) polypeptide” is meant a protein having at least about 85% amino acid sequence identity to GenBank Accession No. AJS10360.1, which is provided below, or a fragment thereof having immunomodulatory activity.
>AJS10360.1 programmed cell death 1 protein [Homo sapiens]

(SEQ ID NO: 478)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL.

By “programmed cell death 1 (PD1) polynucleotide” is meant a nucleic acid molecule encoding an x polypeptide, as well as the introns, exons, 3′ untranslated regions, 5′ untranslated regions, and regulatory sequences associated with its expression, or fragments thereof. An exemplary PD1 polynucleotide sequence is provided at GenBank Accession No. KJ865861.1, which is provided below. An exemplary PD1 polynucleotide sequence is also provided at Ensenbl accession no: ENSG00000188389.
>KJ865861.1 Homo sapiens cell-line G3361 programmed cell death 1 protein (PDCD1) mRNA, complete cds

(SEQ ID NO: 479)
ATGCAGATCCCACAGGCGCCCTGGCCAGTCGTCTGGGCGGTGCTACAACT
GGGCTGGCGGCCAGGATGGTTCTTAGACTCCCCAGACAGGCCCTGGAACC
CCCCCACCTTCTCCCCAGCCCTGCTCGTGGTGACCGAAGGGGACAACGCC
ACCTTCACCTGCAGCTTCTCCAACACATCGGAGAGCTTCGTGCTAAACTG
GTACCGCATGAGCCCCAGCAACCAGACGGACAAGCTGGCCGCCTTCCCCG
AGGACCGCAGCCAGCCCGGCCAGGACTGCCGCTTCCGTGTCACACAACTG
CCCAACGGGCGTGACTTCCACATGAGCGTGGTCAGGGCCCGGCGCAATGA
CAGCGGCACCTACCTCTGTGGGGCCATCTCCCTGGCCCCCAAGGCGCAGA
TCAAAGAGAGCCTGCGGGCAGAGCTCAGGGTGACAGAGAGAAGGGCAGAA
GTGCCCACAGCCCACCCCAGCCCCTCACCCAGGCCAGCCGGCCAGTTCCA
AACCCTGGTGGTTGGTGTCGTGGGCGGCCTGCTGGGCAGCCTGGTGCTGC
TAGTCTGGGTCCTGGCCGTCATCTGCTCCCGGGCCGCACGAGGGACAATA
GGAGCCAGGCGCACCGGCCAGCCCCTGAAGGAGGACCCCTCAGCCGTGCC
TGTGTTCTCTGTGGACTATGGGGAGCTGGATTTCCAGTGGCGAGAGAAGA
CCCCGGAGCCCCCCGTGCCCTGTGTCCCTGAGCAGACGGAGTATGCCACC
ATTGTCTTTCCTAGCGGAATGGGCACCTCATCCCCCGCCCGCAGGGGCTC
AGCCGACGGCCCTCGGAGTGCCCAGCCACTGAGGCCTGAGGATGGACACT
GCTCTTGGCCCCTCTGA.

The term “pharmaceutical composition” means a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

By “rBE4 polypeptide” is meant a polypeptide sharing at least 85% amino acid sequence identity to the below amino acid sequence and having cytidine base editor activity.

(SEQ ID NO: 453)
MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLKSGGSSGGSSGSETPGTSESAT
PESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKE
LGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMN
TKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMP
QVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEV
KKDLIIKLPKYSLFELENGRKRMLASAGELQKGNELALPSKYVNFLYLAS
HYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTK
EVLDATLIHQSITGLYETRIDLSQLGGDSGGSGGSGGSTNLSDIIEKETG
KQLVIQESILMLPEEVEEVIGNKPESDILVHTAYDESTDENVMLLTSDAP
EYKPWALVIQDSNGENKIKMLSGGSGGSGGSTNLSDIIEKETGKQLVIQE
SILMLPEEVEEVIGNKPESDILVHTAYDESTDENVMLLTSDAPEYKPWAL
VIQDSNGENKIKMLSGGSKRTADGSEFESPKKKRKVE.

By “rBE4 polynucleotide” is meant a polynucleotide encoding a rBE4 polypeptide.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 5%, 10%, 15%, 20%, 25%, 30%, 35%, 40%, 45%, 50%, 55%, 60%, 65%, 70%, 75%, 80%, 85%, 90%, 95% or 100%. In embodiments, the expression of a polypeptide or polynucleotide target is rendered virtually undetectable using standard methods for measuring polypeptides (e.g., flow cytometry, ELISA, Western Blot) and polynucleotides (e.g., qPCR, Northern blot). In embodiments, the negative alteration is of a marker (e.g., pCREB). In some cases, a reduction is measured using pCREB staining. In some cases, a reduction is measured using a functional readout. For example, cells can be placed under hypoxic stress (e.g., 1% oxygen) and a response to hypoxia evaluated. Under such hypoxic conditions, cells edited to be deficient in A2AR and/or HIF (e.g., HIF1a) expression and/or activity will produce more cytokine than unedited cells under similar conditions. HIF1α expression under hypoxic conditions is associated with reduced cytokine production.

By “reference” is meant a standard or control condition. In one embodiment, the reference is a wild-type or healthy cell (e.g., immune cell (e.g., T- or NK-cell)). In one embodiment, the reference is an unedited cell (e.g., immune cell (e.g., T- or NK-cell)). In other embodiments and without limitation, a reference is an untreated cell that is not subjected to a test condition, or is subjected to placebo or normal saline, medium, buffer, and/or a control vector that does not harbor a polynucleotide of interest. In some instances, the reference is an unedited cell and/or a wild type cell. In some cases the reference is a cell cultured in an immunosuppressive environment (e.g., hypoxic environment and/or a solid tumor microenvironment (sTME)).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” are used with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csn1) from Streptococcus pyogenes.

The term “single nucleotide polymorphism (SNP)” is a variation in a single nucleotide that occurs at a specific position in the genome, where each variation is present to some appreciable degree within a population (e.g., >1%). SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration.

By “specifically binds” is meant a nucleic acid molecule, polypeptide, polypeptide/polynucleotide complex, compound, or molecule that recognizes and binds a polypeptide and/or nucleic acid molecule of the invention, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least 60%, 80%, 85%, 90%, 95% or even 99% identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. COBALT is used, for example, with the following parameters:
a) alignment parameters: Gap penalties-11,-1 and End-Gap penalties-5,-1,
b) CDD Parameters: Use RPS BLAST on; Blast E-value 0.003; Find Conserved columns and Recompute on, and
c) Query Clustering Parameters: Use query clusters on; Word Size 4; Max cluster distance 0.8; Alphabet Regular.
   EMBOSS Needle is used, for example, with the following parameters:
a) Matrix: BLOSUM62;
b) GAP OPEN: 10;
c) GAP EXTEND: 0.5;
d) OUTPUT FORMAT: pair;
e) END GAP PENALTY: false;
f) END GAP OPEN: 10; and
g) END GAP EXTEND: 0.5.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In an embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “split” is meant divided into two or more fragments.

A “split Cas9 protein” or “split Cas9” refers to a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a “reconstituted” Cas9 protein.

The term “target site” refers to a sequence within a nucleic acid molecule that is deaminated by a deaminase (e.g., cytidine or cytosine deaminase; or adenine or adenosine deaminase), a fusion protein comprising a deaminase (e.g., a dCas9-adenosine deaminase fusion protein), or a base editor (e.g., adenine or adenosine base editor (ABE); or a cytidine or a cytosine base editor (CBE)) as disclosed herein).

By “T cell immunoglobulin mucin-3 (TIM3) polypeptide” is meant a protein having at least about 85% amino acid sequence identity to GenBank Accession No. AAL65157.1, which is provided below, or a fragment thereof having immunomodulatory activity.
>AAL65157.1 T cell immuno lobulin mucin-3 [Homo sapiens]

(SEQ ID NO: 480)
MFSHLPFDCVLLLLLLLLTRSSEVEYRAEVGQNAYLPCFYTPAAPGNLVPVCWGKGACPVFECG
NVVLRTDERDVNYWTSRYWLNGDFRKGDVSLTIENVTLADSGIYCCRIQIPGIMNDEKFNLKLV
IKPAKVTPAPTLQRDFTAAFPRMLTTRGHGPAETQTLGSLPDINLTQISTLANELRDSRLANDL
RDSGATIRIGIYIGAGICAGLALALIFGALIFKWYSHSKEKIQNLSLISLANLPPSGLANAVAE
GIRSEENIYTIEENVYEVEEPNEYYCYVSSRQQPSQPLGCRFAMP.

By “T cell immunoglobulin mucin-3 (TIM3) polynucleotide” is meant a nucleic acid molecule encoding an x polypeptide, as well as the introns, exons, 3′ untranslated regions, 5′ untranslated regions, and regulatory sequences associated with its expression, or fragments thereof. An exemplary TIM3 polynucleotide sequence is provided at GenBank Accession No. AF450242.1:58-963, which is provided below. An exemplary TIM3 polynucleotide sequence is also provided at Ensenbl accession no: ENSG00000135077.
>AF450242.1:58-963 Homo sapiens clone 1 T cell immunoglobulin mucin-3 (TIM3) mRNA, complete cds

(SEQ ID NO: 481)
ATGTTTTCACATCTTCCCTTTGACTGTGTCCTGCTGCTGCTGCTGCTACTACTTACAAGGTCCT
CAGAAGTGGAATACAGAGCGGAGGTCGGTCAGAATGCCTATCTGCCCTGCTTCTACACCCCAGC
CGCCCCAGGGAACCTCGTGCCCGTCTGCTGGGGCAAAGGAGCCTGTCCTGTGTTTGAATGTGGC
AACGTGGTGCTCAGGACTGATGAAAGGGATGTGAATTATTGGACATCCAGATACTGGCTAAATG
GGGATTTCCGCAAAGGAGATGTGTCCCTGACCATAGAGAATGTGACTCTAGCAGACAGTGGGAT
CTACTGCTGCCGGATCCAAATCCCAGGCATAATGAATGATGAAAAATTTAACCTGAAGTTGGTC
ATCAAACCAGCCAAGGTCACCCCTGCACCGACTCTGCAGAGAGACTTCACTGCAGCCTTTCCAA
GGATGCTTACCACCAGGGGACATGGCCCAGCAGAGACACAGACACTGGGGAGCCTCCCTGATAT
AAATCTAACACAAATATCCACATTGGCCAATGAGTTACGGGACTCTAGATTGGCCAATGACTTA
CGGGACTCTGGAGCAACCATCAGAATAGGCATCTACATCGGAGCAGGGATCTGTGCTGGGCTGG
CTCTGGCTCTTATCTTCGGCGCTTTAATTTTCAAATGGTATTCTCATAGCAAAGAGAAGATACA
GAATTTAAGCCTCATCTCTTTGGCCAACCTCCCTCCCTCAGGATTGGCAAATGCAGTAGCAGAG
GGAATTCGCTCAGAAGAAAACATCTATACCATTGAAGAGAACGTATATGAAGTGGAGGAGCCCA
ATGAGTATTATTGCTATGTCAGCAGCAGGCAGCAACCCTCACAACCTTTGGGTTGTCGCTTTGC
AATGCCATAG.

By “T cell receptor beta constant 1 (TRBC1) polypeptide” is meant a protein having at least about 85% amino acid sequence identity to UniProtKB/Swiss-Prot Accession No. P01850.4, which is provided below, or a fragment thereof having immunomodulatory activity.
>sp|P01850.4|TRBC1_HUMAN RecName: Full=T cell receptor beta constant 1

(SEQ ID NO: 482)
DLNKVFPPEVAVFEPSEAEISHTQKATLVCLATGFFPDHVELSWWVNGKEVHSGVSTDPQPLKE
QPALNDSRYCLSSRLRVSATFWQNPRNHFRCQVQFYGLSENDEWTQDRAKPVTQIVSAEAWGRA
DCGFTSVSYQQGVLSATILYEILLGKATLYAVLVSALVLMAMVKRKDF.

By “T cell receptor beta constant 1 (TRBC1) polynucleotide” is meant a nucleic acid molecule encoding a TRBC1 polypeptide, as well as the introns, exons, 3′ untranslated regions, 5′ untranslated regions, and regulatory sequences associated with its expression, or fragments thereof. An exemplary TRBC1 polynucleotide sequence is provided at Ensenbl accession no: ENSG00000211751.

By “transforming growth factor-beta type I receptor (TGFbetaR1; TGFbR1) polypeptide” is meant a protein having at least about 85% amino acid sequence identity to GenBank Accession No. AAD02042.1, which is provided below, or a fragment thereof having signal transduction activity.
>AAD02042.1 transforming growth factor-beta type I receptor [Homo sapiens]

(SEQ ID NO: 483)
MEAAVAAPRPRLLLLVLAAAAAAAAALLPGATALQCFCHLCTKDNFTCVTDGLCFVSVTETTDK
VIHNSMCIAEIDLIPRDRPFVCAPSSKTGSVTTTYCCNQDHCNKIELPTTVKSSPGLGPVELAA
VIAGPVCFVCISLMLMVYICHNRTVIHHRVPNEEDPSLDRPFISEGTTLKDLIYDMTTSGSGSG
LPLLVQRTIARTIVLQESIGKGRFGEVWRGKWRGEEVAVKIFSSREERSWFREAEIYQTVMLRH
ENILGFIAADNKDNGTWTQLWLVSDYHEHGSLFDYLNRYTVTVEGMIKLALSTASGLAHLHMEI
VGTQGKPAIAHRDLKSKNILVKKNGTCCIADLGLAVRHDSATDTIDIAPNHRVGTKRYMAPEVL
DDSINMKHFESFKRADIYAMGLVFWEIARRCSIGGIHEDYQLPYYDLVPSDPSVEEMRKVVCEQ
KLRPNIPNRWQSCEALRVMAKIMRECWYANGAARLTALRIKKTLSQLSQQEGIKM.

By “transforming growth factor-beta type I receptor (TGFbetaR1; TGFbR1) polynucleotide” is meant a nucleic acid molecule encoding an x polypeptide, as well as the introns, exons, 3′ untranslated regions, 5′ untranslated regions, and regulatory sequences associated with its expression, or fragments thereof. An exemplary TGFbetaR1 polynucleotide sequence is provided at GenBank Accession No. AH007196.2:71-167,467-712,1161-1391,1856-2086,2589-2756,3257-3413,3915-4039,4543-4673,5174-5299, which is provided below. An exemplary TGFbetaR1 polynucleotide sequence is also provided at Ensenbl accession no: ENSG00000106799.
>AH007196.2:71-167,467-712,1161-1391,1856-2086,2589-2756,3257-3413,3915-4039,4543-4673,5174-5299 Homo sapiens chromosome 9 transforming growth factor-beta type I receptor gene, complete cds (transforming growth factor-beta type I receptor)

(SEQ ID NO: 484)
ATGGAGGCGGCGGTCGCTGCTCCGCGTCCCCGGCTGCTCCTCCTCGTGCTGGCGGCGGCGGCGG
CGGCGGCGGCGGCGCTGCTCCCGGGGGCGACGGCGTTACAGTGTTTCTGCCACCTCTGTACAAA
AGACAATTTTACTTGTGTGACAGATGGGCTCTGCTTTGTCTCTGTCACAGAGACCACAGACAAA
GTTATACACAACAGCATGTGTATAGCTGAAATTGACTTAATTCCTCGAGATAGGCCGTTTGTAT
GTGCACCCTCTTCAAAAACTGGGTCTGTGACTACAACATATTGCTGCAATCAGGACCATTGCAA
TAAAATAGAACTTCCAACTACTGTAAAGTCATCACCTGGCCTTGGTCCTGTGGAACTGGCAGCT
GTCATTGCTGGACCAGTGTGCTTCGTCTGCATCTCACTCATGTTGATGGTCTATATCTGCCACA
ACCGCACTGTCATTCACCATCGAGTGCCAAATGAAGAGGACCCTTCATTAGATCGCCCTTTTAT
TTCAGAGGGTACTACGTTGAAAGACTTAATTTATGATATGACAACGTCAGGTTCTGGCTCAGGT
TTACCATTGCTTGTTCAGAGAACAATTGCGAGAACTATTGTGTTACAAGAAAGCATTGGCAAAG
GTCGATTTGGAGAAGTTTGGAGAGGAAAGTGGCGGGGAGAAGAAGTTGCTGTTAAGATATTCTC
CTCTAGAGAAGAACGTTCGTGGTTCCGTGAGGCAGAGATTTATCAAACTGTAATGTTACGTCAT
GAAAACATCCTGGGATTTATAGCAGCAGACAATAAAGACAATGGTACTTGGACTCAGCTCTGGT
TGGTGTCAGATTATCATGAGCATGGATCCCTTTTTGATTACTTAAACAGATACACAGTTACTGT
GGAAGGAATGATAAAACTTGCTCTGTCCACGGCGAGCGGTCTTGCCCATCTTCACATGGAGATT
GTTGGTACCCAAGGAAAGCCAGCCATTGCTCATAGAGATTTGAAATCAAAGAATATCTTGGTAA
AGAAGAATGGAACTTGCTGTATTGCAGACTTAGGACTGGCAGTAAGACATGATTCAGCCACAGA
TACCATTGATATTGCTCCAAACCACAGAGTGGGAACAAAAAGGTACATGGCCCCTGAAGTTCTC
GATGATTCCATAAATATGAAACATTTTGAATCCTTCAAACGTGCTGACATCTATGCAATGGGCT
TAGTATTCTGGGAAATTGCTCGACGATGTTCCATTGGTGGAATTCATGAAGATTACCAACTGCC
TTATTATGATCTTGTACCTTCTGACCCATCAGTTGAAGAAATGAGAAAAGTTGTTTTGAACAGA
AGTTAAGGCCAAATATCCCAAACAGATGGCAGAGCTGTGAAGCCTTGAGAGTAATGGCTAAAAT
TAGAGAGAATGTTGGTATGCCAATGGAGCAGCTAGGCTTACAGCATTGCGGATTAAGAAAACAT
TATCGCAACTCATCAACAGGAAGGCATCAAAATGTAA.

By “transforming growth factor-beta type II receptor (TGFbetaR2; TGFbR2) polypeptide” is meant a protein having at least about 85% amino acid sequence identity to GenBank Accession No. AAA61164.1, which is provided below, or a fragment thereof having signal transduction activity.
>AAA61164.1 TGF-beta type II receptor [Homo sapiens]

(SEQ ID NO: 485)
MGRGLLRGLWPLHIVLWTRIASTIPPHVQKSVNNDMIVTDNNGAVKFPQLCKFCDVRFSTCDNQ
KSCMSNCSITSICEKPQEVCVAVWRKNDENITLETVCHDPKLPYHDFILEDAASPKCIMKEKKK
PGETFFMCSCSSDECNDNIIFSEEYNTSNPDLLLVIFQVTGISLLPPLGVAISVIIIFYCYRVN
RQQKLSSTWETGKTRKLMEFSEHCAIILEDDRSDISSTCANNINHNTELLPIELDTLVGKGRFA
EVYKAKLKQNTSEQFETVAVKIFPYEEYASWKTEKDIFSDINLKHENILQFLTAEERKTELGKQ
YWLITAFHAKGNLQEYLTRHVISWEDLRKLGSSLARGIAHLHSDHTPCGRPKMPIVHRDLKSSN
ILVKNDLTCCLCDFGLSLRLDPTLSVDDLANSGQVGTARYMAPEVLESRMNLENAESFKQTDVY
SMALVLWEMTSRCNAVGEVKDYEPPFGSKVREHPCVESMKDNVLRDRGRPEIPSFWLNHQGIQM
VCETLTECWDHDPEARLTAQCVAERFSELEHLDRLSGRSCSEEKIPEDGSLNTTK.

By “transforming growth factor-beta type II receptor (TGFbetaR2; TGFbR2) polynucleotide” is meant a nucleic acid molecule encoding an x polypeptide, as well as the introns, exons, 3′ untranslated regions, 5′ untranslated regions, and regulatory sequences associated with its expression, or fragments thereof. An exemplary x polynucleotide sequence is provided at GenBank Accession No. M85079.1:336-2039, which is provided below. An exemplary TGFbetaR2 polynucleotide sequence is also provided at Ensenbl accession no: ENSG00000163513.
>M85079.1:336-2039 Human TGF-beta type II receptor mRNA, complete cds

(SEQ ID NO: 486)
ATGGGTCGGGGGCTGCTCAGGGGCCTGTGGCCGCTGCACATCGTCCTGTGGACGCGTATCGCCA
GCACGATCCCACCGCACGTTCAGAAGTCGGTTAATAACGACATGATAGTCACTGACAACAACGG
TGCAGTCAAGTTTCCACAACTGTGTAAATTTTGTGATGTGAGATTTTCCACCTGTGACAACCAG
AAATCCTGCATGAGCAACTGCAGCATCACCTCCATCTGTGAGAAGCCACAGGAAGTCTGTGTGG
CTGTATGGAGAAAGAATGACGAGAACATAACACTAGAGACAGTTTGCCATGACCCCAAGCTCCC
CTACCATGACTTTATTCTGGAAGATGCTGCTTCTCCAAAGTGCATTATGAAGGAAAAAAAAAAG
CCTGGTGAGACTTTCTTCATGTGTTCCTGTAGCTCTGATGAGTGCAATGACAACATCATCTTCT
CAGAAGAATATAACACCAGCAATCCTGACTTGTTGCTAGTCATATTTCAAGTGACAGGCATCAG
CCTCCTGCCACCACTGGGAGTTGCCATATCTGTCATCATCATCTTCTACTGCTACCGCGTTAAC
CGGCAGCAGAAGCTGAGTTCAACCTGGGAAACCGGCAAGACGCGGAAGCTCATGGAGTTCAGCG
AGCACTGTGCCATCATCCTGGAAGATGACCGCTCTGACATCAGCTCCACGTGTGCCAACAACAT
CAACCACAACACAGAGCTGCTGCCCATTGAGCTGGACACCCTGGTGGGGAAAGGTCGCTTTGCT
GAGGTCTATAAGGCCAAGCTGAAGCAGAACACTTCAGAGCAGTTTGAGACAGTGGCAGTCAAGA
TCTTTCCCTATGAGGAGTATGCCTCTTGGAAGACAGAGAAGGACATCTTCTCAGACATCAATCT
GAAGCATGAGAACATACTCCAGTTCCTGACGGCTGAGGAGCGGAAGACGGAGTTGGGGAAACAA
TACTGGCTGATCACCGCCTTCCACGCCAAGGGCAACCTACAGGAGTACCTGACGCGGCATGTCA
TCAGCTGGGAGGACCTGCGCAAGCTGGGCAGCTCCCTCGCCCGGGGGATTGCTCACCTCCACAG
TGATCACACTCCATGTGGGAGGCCCAAGATGCCCATCGTGCACAGGGACCTCAAGAGCTCCAAT
ATCCTCGTGAAGAACGACCTAACCTGCTGCCTGTGTGACTTTGGGCTTTCCCTGCGTCTGGACC
CTACTCTGTCTGTGGATGACCTGGCTAACAGTGGGCAGGTGGGAACTGCAAGATACATGGCTCC
AGAAGTCCTAGAATCCAGGATGAATTTGGAGAATGCTGAGTCCTTCAAGCAGACCGATGTCTAC
TCCATGGCTCTGGTGCTCTGGGAAATGACATCTCGCTGTAATGCAGTGGGAGAAGTAAAAGATT
ATGAGCCTCCATTTGGTTCCAAGGTGCGGGAGCACCCCTGTGTCGAAAGCATGAAGGACAACGT
GTTGAGAGATCGAGGGCGACCAGAAATTCCCAGCTTCTGGCTCAACCACCAGGCATCCAGATGG
TGTGTGAGACGTTGACTGAGTGCTGGGACCACGACCCAGAGGCCCGTCTCACAGCCCAGTGTGT
GGCAGAACGCTTCAGTGAGCTGGAGCATCTGGACAGGCTCTCGGGGAGGAGCTGCTCGGAGGAG
AAGATTCCTGAAGACGGCTCCCTAAACACTACCAAATAG.

By “T Cell Receptor Alpha Constant (TRAC) polypeptide” is meant a protein having at least about 85% amino acid sequence identity to NCBI Accession No. P01848.2, or a fragment thereof having immunomodulatory activity. An exemplary amino acid sequence is provided below.
>sp|P01848.2|TRAC_HUMAN RecName: Full=T cell receptor alpha constant

(SEQ ID NO: 487)
IQNPDPAVYQLRDSKSSDKSVCLFTDFDSQTNVSQSKDSDVYITDKTVLDMRSMDFKSNSAVAW
SNKSDFACANAFNNSIIPEDTFFPSPESSCDVKLVEKSFETDTNLNFQNLSVIGFRILLLKVAG
FNLLMTLRLWSS.

By “T Cell Receptor Alpha Constant (TRAC) polynucleotide” is meant a nucleic acid molecule encoding a TRAC polypeptide, as well as the introns, exons, 3′ untranslated regions, 5′ untranslated regions, and regulatory sequences associated with its expression, or fragments thereof. An exemplary TRAC polynucleotide is provided at Gene ENSG00000277734.8, which is provided below.
>UCSC human genome database, Gene ENSG00000277734.8 Human T-cell receptor alpha chain (TCR-alpha)

(SEQ ID NO: 488)
catgctaatcctccggcaaacctctgtttcctcctcaaaaggcaggaggtcggaaagaataaacaatgag
agtcacattaaaaacacaaaatcctacggaaatactgaagaatgagtctcagcactaaggaaaagcctcc
agcagctcctgctttctgagggtgaaggatagacgctgtggctctgcatgactcactagcactctatcac
ggccatattctggcagggtcagtggctccaactaacatttgtttggtactttacagtttattaaatagat
gtttatatggagaagctctcatttctttctcagaagagcctggctaggaaggtggatgaggcaccatatt
cattttgcaggtgaaattcctgagatgtaaggagctgctgtgacttgctcaaggccttatatcgagtaaa
cggtagtgctggggcttagacgcaggtgttctgatttatagttcaaaacctctatcaatgagagagcaat
ctcctggtaatgtgatagatttcccaacttaatgccaacataccataaacctcccattctgctaatgccc
agcctaagttggggagaccactccagattccaagatgtacagtttgctttgctgggcctttttcccatgc
ctgcctttactctgccagagttatattgctggggttttgaagaagatcctattaaataaaagaataagca
gtattattaagtagccctgcatttcaggtttccttgagtggcaggccaggcctggccgtgaacgttcact
gaaatcatggcctcttggccaagattgatagcttgtgcctgtccctgagtcccagtccatcacgagcagc
tggtttctaagatgctatttcccgtataaagcatgagaccgtgacttgccagccccacagagccccgccc
ttgtccatcactggcatctggactccagcctgggttggggcaaagagggaaatgagatcatgtcctaacc
ctgatcctcttgtcccacagATATCCAGAACCCTGACCCTGCCGTGTACCAGCTGAGAGACTCTAAATCC
AGTGACAAGTCTGTCTGCCTATTCACCGATTTTGATTCTCAAACAAATGTGTCACAAAGTAAGGATTCTG
ATGTGTATATCACAGACAAAACTGTGCTAGACATGAGGTCTATGGACTTCAAGAGCAACAGTGCTGTGGC
CTGGAGCAACAAATCTGACTTTGCATGTGCAAACGCCTTCAACAACAGCATTATTCCAGAAGACACCTTC
TTCCCCAGCCCAGgtaagggcagctttggtgccttcgcaggctgtttccttgcttcaggaatggccaggt
tctgcccagagctctggtcaatgatgtctaaaactcctctgattggtggtctcggccttatccattgcca
ccaaaaccctctttttactaagaaacagtgagccttgttctggcagtccagagaatgacacgggaaaaaa
gcagatgaagagaaggtggcaggagagggcacgtggcccagcctcagtctctccaactgagttcctgcct
gcctgcctttgctcagactgtttgccccttactgctcttctaggcctcattctaagccccttctccaagt
tgcctctccttatttctccctgtctgccaaaaaatctttcccagctcactaagtcagtctcacgcagtca
ctcattaacccaccaatcactgattgtgccggcacatgaatgcaccaggtgttgaagtggaggaattaaa
aagtcagatgaggggtgtgcccagaggaagcaccattctagttgggggagcccatctgtcagctgggaaa
agtccaaataacttcagattggaatgtgttttaactcagggttgagaaaacagctaccttcaggacaaaa
gtcagggaagggctctctgaagaaatgctacttgaagataccagccctaccaagggcagggagaggaccc
tatagaggcctgggacaggagctcaatgagaaaggagaagagcagcaggcatgagttgaatgaaggaggc
agggccgggtcacagggccttctaggccatgagagggtagacagtattctaaggacgccagaaagctgtt
gatcggcttcaagcaggggagggacacctaatttgcttttcttttttttttttttttttttttttttttt
tgagatggagttttgctcttgttgcccaggctggagtgcaatggtgcatcttggctcactgcaacctccg
cctcccaggttcaagtgattctcctgcctcagcctcccgagtagctgagattacaggcacccgccaccat
gcctggctaattttttgtatttttagtagagacagggtttcactatgttggccaggctggtctcgaactc
ctgacctcaggtgatccacccgcttcagcctcccaaagtgctgggattacaggcgtgagccaccacaccc
ggcctgcttttcttaaagatcaatctgagtgctgtacggagagtgggttgtaagccaagagtagaagcag
aaagggagcagttgcagcagagagatgatggaggcctgggcagggtggtggcagggaggtaaccaacacc
attcaggtttcaaaggtagaaccatgcagggatgagaaagcaaagaggggatcaaggaaggcagctggat
tttggcctgagcagctgagtcaatgatagtgccgtttactaagaagaaaccaaggaaaaaatttggggtg
cagggatcaaaactttttggaacatatgaaagtacgtgtttatactctttatggcccttgtcactatgta
tgcctcgctgcctccattggactctagaatgaagccaggcaagagcagggtctatgtgtgatggcacatg
tggccagggtcatgcaacatgtactttgtacaaacagtgtatattgagtaaatagaaatggtgtccagga
gccgaggtatcggtcctgccagggccaggggctctccctagcaggtgctcatatgctgtaagttccctcc
agatctctccacaaggaggcatggaaaggctgtagttgttcacctgcccaagaactaggaggtctggggt
gggagagtcagcctgctctggatgctgaaagaatgtctgtttttccttttagAAAGTTCCTGTGATGTCA
AGCTGGTCGAGAAAAGCTTTGAAACAGgtaagacaggggtctagcctgggtttgcacaggattgcggaag
tgatgaacccgcaataaccctgcctggatgagggagtgggaagaaattagtagatgtgggaatgaatgat
gaggaatggaaacagcggttcaagacctgcccagagctgggtggggtctctcctgaatccctctcaccat
ctctgactttccattctaagcactttgaggatgagtttctagcttcaatagaccaaggactctctcctag
gcctctgtattcctttcaacagctccactgtcaagagagccagagagagcttctgggtggcccagctgtg
aaatttctgagtcccttagggatagccctaaacgaaccagatcatcctgaggacagccaagaggttttgc
cttctttcaagacaagcaacagtactcacataggctgtgggcaatggtcctgtctctcaagaatcccctg
ccactcctcacacccaccctgggcccatattcatttccatttgagttgttcttattgagtcatccttcct
gtggtagcggaactcactaaggggcccatctggacccgaggtattgtgatgataaattctgagcacctac
cccatccccagaagggctcagaaataaaataagagccaagtctagtcggtgtttcctgtcttgaaacaca
atactgttggccctggaagaatgcacagaatctgtttgtaaggggatatgcacagaagctgcaagggaca
ggaggtgcaggagctgcaggcctcccccacccagcctgctctgccttggggaaaaccgtgggtgtgtcct
gcaggccatgcaggcctgggacatgcaagcccataaccgctgtggcctcttggttttacagATACGAACC
TAAACTTTCAAAACCTGTCAGTGATTGGGTTCCGAATCCTCCTCCTGAAAGTGGCCGGGTTTAATCTGCT
CATGACGCTGCGGCTGTGGTCCAGCTGAGgtgaggggccttgaagctgggagtggggtttagggacgcgg
gtctctgggtgcatcctaagctctgagagcaaacctccctgcagggtcttgcttttaagtccaaagcctg
agcccaccaaactctcctacttcttcctgttacaaattcctcttgtgcaataataatggcctgaaacgct
gtaaaatatcctcatttcagccgcctcagttgcacttctcccctatgaggtaggaagaacagttgtttag
aaacgaagaaactgaggccccacagctaatgagtggaggaagagagacacttgtgtacaccacatgcctt
gtgttgtacttctctcaccgtgtaacctcctcatgtcctctctccccagtacggctctcttagctcagta
gaaagaagacattacactcatattacaccccaatcctggctagagtctccgcaccctcctcccccagggt
ccccagtcgtcttgctgacaactgcatcctgttccatcaccatcaaaaaaaaactccaggctgggtgcgg
gggctcacacctgtaatcccagcactttgggaggcagaggcaggaggagcacaggagctggagaccagcc
tgggcaacacagggagaccccgcctctacaaaaagtgaaaaaattaaccaggtgtggtgctgcacacctg
tagtcccagctacttaagaggctgagatgggaggatcgcttgagccctggaatgttgaggctacaatgag
ctgtgattgcgtcactgcactccagcctggaagacaaagcaagatcctgtctcaaataataaaaaaaata
agaactccagggtacatttgctcctagaactctaccacatagccccaaacagagccatcaccatcacatc
cctaacagtcctgggtcttcctcagtgtccagcctgacttctgttcttcctcattccagATCTGCAAGAT
TGTAAGACAGCCTGTGCTCCCTCGCTCCTTCCTCTGCATTGCCCCTCTTCTCCCTCTCCAAACAGAGGGA
ACTCTCCTACCCCCAAGGAGGTGAAAGCTGCTACCACCTCTGTGCCCCCCCGGCAATGCCACCAACTGGA
TCCTACCCGAATTTATGATTAAGATTGCTGAAGAGCTGCCAAACACTGCTGCCACCCCCTCTGTTCCCTT
ATTGCTGCTTGTCACTGCCTGACATTCACGGCAGAGGCAAGGCTGCTGCAGCCTCCCCTGGCTGTGCACA
TTCCCTCCTGCTCCCCAGAGACTGCCTCCGCCATCCCACAGATGATGGATCTTCAGTGGGTTCTCTTGGG
CTCTAGGTCCTGCAGAATGTTGTGAGGGGTTTATTTTTTTTTAATAGTGTTCATAAAGAAATACATAGTA
TTCTTCTTCTCAAGACGTGGGGGGAAATTATCTCATTATCGAGGCCCTGCTATGCTGTGTATCTGGGCGT
GTTGTATGTCCTGCTGCCGATGCCTTCATTAAAATGATTTGGAAGAGCAGA.

Nucleotides in lower case above are untranslated regions or introns, and nucleotides in upper cases are exons.
>X02592.1 Human mRNA for T-cell receptor alpha chain (TCR-alpha)

(SEQ ID NO: 489)
TTTTGAAACCCTTCAAAGGCAGAGACTTGTCCAGCCTAACCTGCCTGCTGCTCCTAGCTCCTGA
GGCTCAGGGCCCTTGGCTTCTGTCCGCTCTGCTCAGGGCCCTCCAGCGTGGCCACTGCTCAGCC
ATGCTCCTGCTGCTCGTCCCAGTGCTCGAGGTGATTTTTACCCTGGGAGGAACCAGAGCCCAGT
CGGTGACCCAGCTTGGCAGCCACGTCTCTGTCTCTGAAGGAGCCCTGGTTCTGCTGAGGTGCAA
CTACTCATCGTCTGTTCCACCATATCTCTTCTGGTATGTGCAATACCCCAACCAAGGACTCCAG
CTTCTCCTGAAGTACACATCAGCGGCCACCCTGGTTAAAGGCATCAACGGTTTTGAGGCTGAAT
TTAAGAAGAGTGAAACCTCCTTCCACCTGACGAAACCCTCAGCCCATATGAGCGACGCGGCTGA
GTACTTCTGTGCTGTGAGTGATCTCGAACCGAACAGCAGTGCTTCCAAGATAATCTTTGGATCA
GGGACCAGACTCAGCATCCGGCCAAATATCCAGAACCCTGACCCTGCCGTGTACCAGCTGAGAG
ACTCTAAATCCAGTGACAAGTCTGTCTGCCTATTCACCGATTTTGATTCTCAAACAAATGTGTC
ACAAAGTAAGGATTCTGATGTGTATATCACAGACAAAACTGTGCTAGACATGAGGTCTATGGAC
TTCAAGAGCAACAGTGCTGTGGCCTGGAGCAACAAATCTGACTTTGCATGTGCAAACGCCTTCA
ACAACAGCATTATTCCAGAAGACACCTTCTTCCCCAGCCCAGAAAGTTCCTGTGATGTCAAGCT
GGTCGAGAAAAGCTTTGAAACAGATACGAACCTAAACTTTCAAAACCTGTCAGTGATTGGGTTC
CGAATCCTCCTCCTGAAAGTGGCCGGGTTTAATCTGCTCATGACGCTGCGGCTGTGGTCCAGCT
GAGATCTGCAAGATTGTAAGACAGCCTGTGCTCCCTCGCTCCTTCCTCTGCATTGCCCCTCTTC
TCCCTCTCCAAACAGAGGGAACTCTCCTACCCCCAAGGAGGTGAAAGCTGCTACCACCTCTGTG
CCCCCCCGGTAATGCCACCAACTGGATCCTACCCGAATTTATGATTAAGATTGCTGAAGAGCTG
CCAAACACTGCTGCCACCCCCTCTGTTCCCTTATTGCTGCTTGTCACTGCCTGACATTCACGGC
AGAGGCAAGGCTGCTGCAGCCTCCCCTGGCTGTGCACATTCCCTCCTGCTCCCCAGAGACTGCC
TCCGCCATCCCACAGATGATGGATCTTCAGTGGGTTCTCTTGGGCTCTAGGTCCTGGAGAATGT
TGTGAGGGGTTTATTTTTTTTTAATAGTGTTCATAAAGAAATACATAGTATTCTTCTTCTCAAG
ACGTGGGGGGAAATTATCTCATTATCGAGGCCCTGCTATGCTGTGTGTCTGGGCGTGTTGTATG
TCCTGCTGCCGATGCCTTCATTAAAATGATTTGGAA.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. Including an inhibitor of uracil DNA glycosylase (UGI) in the base editor prevents base excision repair which changes the U back to a C. An exemplary UGI comprises an amino acid sequence as follows:
>sp1P14739IUNGI_BPPB2 Uracil-DNA glycosylase inhibitor

(SEQ ID NO: 231)
MTNLSDIIEKETGKQLVIQESILMLPEEVEEVIGNKPESDILVHTAYDE
STDENVMLLTSDAPEYKPWALVIQDSNGENKIKML.

The term “vector” refers to a means of introducing a nucleic acid sequence into a cell, resulting in a transformed cell. Vectors include plasmids, transposons, phages, viruses, liposomes, and episome. “Expression vectors” are nucleic acid sequences comprising the nucleotide sequence to be expressed in the recipient cell. Expression vectors may include additional nucleic acid sequences to promote and/or facilitate the expression of the of the introduced sequence such as start, stop, enhancer, promoter, and secretion sequences.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, e.g., within 5-fold, within 2-fold of a value. Where particular values are described in the application and claims, unless otherwise stated, the term “about” means within an acceptable error range for the particular value should be assumed.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIG. 1 is a schematic depicting the hypoxic and adenosinergic pathways. As depicted in FIG. 1, the two pathways are intertwined and pay synergistic roles in suppressing T cells in the tumor microenvironment.

FIGS. 2A and 2B present schematics depicting the role of hypoxic and adenosinergic pathways in suppressing T cells in the tumor microenvironment. As shown in FIGS. 2A and 2B, adenosine receptor antagonists alleviate T cell immunosuppression in the tumor microenvironment.

FIG. 3 provides a graph depicting next generation sequencing-based (NGS) measurements of editing at A2AR, HIF1α (alternatively referred to as HIF1α), and A2BR target sites in CAR-T cells using guides for CBE (g145, g222), ABE (g155, g170, g173, g221), and Cas9 (A2A Cas9, A2B Cas9). Electroporation only (EP) was used as a control. High molecular editing (i.e., base editing efficiencies>90%) was seen across all guides at every target site in the CAR-T cells.

FIGS. 4A-4C provide flow cytometry graphs and bar graphs. FIGS. 4A and 4B depict CAR expression after editing A2A, HIF1α, and A2B target sites in CAR-T cells using guides. FIG. 4A provides flow cytometry graphs depicting CAR expression controls for electroporation (EP) only and untransduced (UTD) CAR-T cells. FIG. 4B are flow cytometry graphs depicting CAR expression of edited A2AR target sites using guides g145, g155, and Cas9 (top), edited HIF1α target sites using guides g168, g170, and g173 (middle), and edited A2B target sites using guides g221, g222, and Cas9 (bottom). FIG. 4C is a bar graph quantifying the CAR expression observed in the flow cytometry graphs of FIG. 4B. Consistent 60% CAR expression was seen across all guides and editing targets. There was no observed impact of editing on CAR expression.

FIG. 5 is a bar graph depicting next generation sequencing-based (NGS) measurements of editing at HIF1α target sites in CAR-T cells using guide RNAs for CBE (sgRNA162, sgRNA163) and ABE (sgRNA158, sgRNA168, sgRNA169, sgRNA170, sgRNA171, sgRNA172, sgRNA173). Electroporation (EP) only was used as a control. Next generation sequencing (NGS) showed high molecular editing (i.e., high base editing efficiencies) for three guides (see arrows in FIG. 5).

FIG. 6A-6D are scatter plots depicting the expression of HIF1α using RNA sequencing (RNA-seq). Untreated cells were compared to cells treated with guides 158, 170 and 173. m1 (H1F1a missense mutation control) was used as a control. RNAseq indicated a decrease in HIF1α mRNA in two of the guide candidates (Guide 170 and Guide 173).

FIG. 7 is a schematic depicting an exemplary target sequence for base editing HIF1α isoform 3 (HIF1α.I3). The sequences in order of occurrence from top-to-bottom in FIG. 7 are provided in the Sequence Listing as SEQ ID NOs: 381 and 382. The lower sequence shown in FIG. 7 is the reverse complement of SEQ ID NO. 381.

FIGS. 8A-8B provide schematics showing that the HIF1α guide RNAs (Guide 170 and Guide 173) target different intron/exon splice sites across the HIF1α gene (SEQ ID NO: 377). Targeting conserved sequences at intron-exon boundaries results in improper splicing, which led to effective protein knockout.

FIGS. 9A and 9B provide schematics depicting the evaluation of gene-splicing sites resulting from editing of the HIF1α gene using Guides 170 and 173. Significant intron retention was observed. More robust editing was observed with Guide 173, which is an observation consistent with this guide being the top HIF1α guide RNA. The sequences shown in FIGS. 9A and 9B in order of occurrence correspond to SEQ ID NOs: 383 and 384.

FIG. 10 provides bar graphs depicting IFNγ production (pg/mL) after EGFR CAR-Ts with HIF1α edits were co-cultured at 1 to 1 E:T ratio with either SKOV3 or H226 cells in 1% O₂ for 48 hours. HIF1α was edited using guides g170 and g173. Electroporation (EP) only was used as a control. EGFR CAR-T cells with HIF1α knockout edits produced more cytokine under hypoxic stress than unedited EGFR CAR-T cells.

FIG. 11 provides a schematic depicting the effect of hypoxia on cytotoxic T cell function via HIF1α, cAMP and pCREB signaling. Not being bound by theory, the hypoxia-adenosinergic axis suppresses cytotoxic T cell function via HIF1α, cAMP and pCREB signaling.

FIG. 12 provides a schematic showing that A2A and A2B adenosine receptor subtypes both play an inhibitory role in suppressing T cell function. Without being bound by theory, A2A is the high affinity inhibitory adenosine receptor, and A2B is the low affinity inhibitory adenosine receptor.

FIG. 13 provides a bar graph depicting next generation sequencing-based (NGS) measurements of editing at A2AR target sites in CAR-T cells using guide RNAs for CBE (sgRNA144, sgRNA145, sgRNA146, sgRNA147, sgRNA148, sgRNA149, sgRNA150, sgRNA151, sgRNA152, sgRNA153, sgRNA154, sgRNA155) and ABE (sgRNA155). Electroporation (EP) only was used as a control.

FIG. 14 provides histograms depicting expression of pCREB in A2AR knockout T cells using guides g145 and g155. Cas9 and Electroporation (EP) only were used as controls. In the plots, the darker-grey histogram corresponds to DMSO and the lighter-grey histogram corresponds to 30 μM CADO. A2AR knockout abrogated adenosine signaling resulting in no upregulation of downstream pCREB. Throughout the figures, the term “CADO” represents 2-chloroadenosine.

FIG. 15 presents bar graphs depicting IFNγ production after 48 hours in A2AR knockout T cells using guides g145 and g155. The data shown in FIG. 15 is normalized to IFNγ production in 0 μM CADO (DMSO only treatment). A2A knockout protected CAR-T cells from adenosine-mediated cytokine suppression.

FIG. 16 provides a bar graph depicting next generation sequencing-based (NGS) measurements of editing at A2BR target sites in CAR-T cells using guide RNAs for CBE (sgRNA222, sgRNA223, sgRNA225, sgRNA226) and ABE (sgRNA221, sgRNA224). Cas9 and Electroporation (EP) only were used as controls. The gRNA screen revealed two top guides with >95% editing.

FIGS. 17A-17C provide graphs showing tumor volume plotted as a function of time in mice administered 2×10⁶, 4×10⁶, or 8×10⁶ edited anti-EGFR CAR T cells having an adenosine receptor (A2AR) knock-out, control anti-EGFR CAR T cells expressing the adenosine receptor, or untransduced (UTD) control cells. The anti-EGFR CAR T cells were adenosine-resistant CAR-T cell (ARC T cells), which are T cells with expression of TCR, HLA Class I, HLA Class II, and A2AR knocked out. The ARC T cells demonstrated robust, dose-dependent anti-tumor efficacy compared to unedited CAR-T cells in a subcutaneous xenograft tumor model (H226 lung carcinoma) in NCG mice.

FIGS. 18A-18C provide schematics. FIG. 18A provides a schematic showing how signaling through A2A adenosine receptors (A2AR) on T cells can significantly inhibit effector functions, including cytokine selection and anti-tumor cytotoxicity. FIGS. 18B and 18C provide schematics showing how an adenosine base editor binds to target DNA to expose a narrow editing window and deaminate an adenosine base to produce inosine, which is read as G by DNA polymerase.

FIGS. 19A-19D provide bar graphs, flow cytometry scatter plots, and a schematic showing highly efficient base editing and preparation of adenosine-resistant CAR-T cells (ARC T cells). FIG. 19A provides a bar graph showing base editing of the ADORA2A gene quantified via next generation sequencing (NGS). FIG. 19B provides a bar graph presenting flow cytometry data showing reduction of cell surface protein, thereby confirming allogeneic gene editing. FIG. 19C provides a set of three flow cytometry scatter plots showing high expression of an EGFR-specific CAR in primary human T cells detected with anti-CAR idiotype antibody. FIG. 19D provides a schematic providing an overview of a process for generating multiplex base edited EGFR-targeting ARC T cells. In FIG. 19B, each set of three bars correspond to, from left-to-right, TCR, HLA Class I, and HLA Class II. In FIGS. 19A-19D, “Base Editing Guide 1” (BE2) indicates cells edited using the base editor ABE8.20 in combination with the guide sgRNA145 (see Table 1A), “Base Editing Guide 2” (BE2) indicates cells edited using the base editor ABE8.20 in combination with the guide sgRNA155, and “CRISPR Guide” indicates cells edited using a CRISPR guide and editor known to be effective in knocking out expression of A2AR.

FIGS. 20A-20E provide flow cytometry histograms, bar graphs, a plot, and images showing adenosine-resistant CAR-T cells (CAR-T cells) were protected from adenosine-mediated suppression in vitro. FIG. 20A provides flow cytometry histograms showing downstream signaling of A2AR (light grey curves) was prevented in ARC T cells, as indicated by a reduction in phosphorylated CREB staining. FIG. 20B provides a bar graph showing that adenosine-resistant CAR-T cells (ARC T) maintained capacity to produce interferon-gamma (IFNγ) in the presence of extracellular adenosine. FIG. 20C shows live cell images of GFP+H226 spheroids treated with untransduced (UTD) T cells or EGFR-targeted CAR-T or ARC T cells 9 days after co-culture. FIG. 20D provides a plot showing that EGFR-targeted ARC T cells retained cytotoxicity against tumor spheroids in the presence of exogenous adenosine. H266 cells expressing GFP were plated in ultra-low adherent 96-well plates and incubated at 36° C. for 3 days to allow for spheroid formation. Then, untransduced cells (UTD), ARC T cells, or CAR T cells expressing TCR, HLA Class I, HLA Class II, and ADAR were added to the wells at a 1:2 effector to target ratio (E:T) with 0 μM adenosine. Plates were then placed in an Incucyte live imaging analysis instrument and cytotoxicity was measured via reduction in GFP+ spheroid volume over time. FIG. 20E provides a bar graph showing quantification of ARC T cell cytotoxicity from the assay shown in FIGS. 20C and 20D. In FIGS. 19A-19D, “BE KO1” indicates cells edited using the base editor ABE8.20 in combination with the guide sgRNA145 (see Table 1A), “BE KO2” indicates cells edited using the base editor ABE8.20 in combination with the guide sgRNA155, and “CRISPR KO” indicates cells edited using a CRISPR guide and editor known to be effective in knocking out expression of A2AR.

FIGS. 21A and 21B provide a schematic and images showing that adenosine-resistant CAR-T cells (ARC T cells) exhibited superior anti-tumor activity in vivo. FIG. 21A provides a schematic showing the experimental schema to test ARC T cell functionality in NCG xenograft mice. FIG. 21B shows ex vivo immunofluorescent staining of hypoxia and adenosine-producing ectoenzyme CD73 in the tumor microenvironment (TME).

FIG. 22 provides a flow cytometry histogram showing phosphor SMAD2/3 (pSMAD2/3) expression in controls. The pSMAD assay was completed to determine functional TGFbR signaling. T cells were stimulated with 100 ng/mL of rhTGFb1 or DMSO for 20 mins at 37° C. Cells were then fixed and permeabilized and stained with phosphor-SMAD2/3 antibody. In FIG. 22, the darker-shaded curve corresponds to cells treated with DMSO and the lightly-shaded curve corresponds to cells contacted with 10 ng/mL TGFb1 for 20 minutes. TGFbR signaling was determined via upregulation of pSMAD 2/3 protein.

FIG. 23 provides a collection of flow cytometry histograms showing phosphor-SMAD2/3 (pSMAD2/3) expression in T cells base edited using the base editors (i.e., ABE) or nuclease (i.e., Cas12b) listed to the left of each row of plots in combination with the indicated guides (e.g., g258; guide spacer sequences are provided in Table 1i), which are listed in each corresponding plot. Stars indicate plots corresponding to base edited cells that showed reduced pSMAD2/3 signaling in the presence of 10 ng/mL TGFb1. The pSMAD2/3 expression assays were conducted as described for FIG. 22. TGFbR knock-out (KO) was confirmed by a reduction in pSMAD2/3 signaling. In FIG. 23, the lightly-shaded curves correspond to edited cells and the darkly-shaded curves correspond to unedited cells.

FIG. 24 provides a series of flow cytometry histograms showing a comparison of TGFbR knockout guides in a cytokine suppression assay. Cells were contacted with dimethyl sulfoxide (DMSO) and measurements were made to determine background levels of phosphor-SMAD (pSMAD) signaling. Unedited cells were stimulated with 100 ng/mL TGFb1 for 20 mins as a control to show upregulation of pSMAD2/3 (dark-grey shaded curves). Cells edited using the guide polynucleotide g260, g262, g272, or g273 (guide spacer sequences are provided in Table 1B) in combination with an editor showed varying levels of TGFbR knock-out (KO), as shown by reduction of pSMAD signaling. In FIG. 24, all of the plots contain the same curves corresponding to unedited cells and cells contacted with DMSO, so that the location of the curves corresponding to the unedited cells and the cells contacted with DMSO are the same across all of the plots.

FIG. 25 provides a set of flow cytometry scatter plots showing chimeric antigen receptor expression in EGFR-targeting CAR-T cells edited to knock out expression of the indicated polypeptides (i.e., A2AR, PD1, TGFbRII, or combinations thereof). At the end of culturing, T cells were stained with an anti-idiotype antibody. No differences were observed in CAR expression across various edits. ADAR expression was knocked out using the guide TSBTx2043, PD1 expression was knocked out using the guide TSBTx025, TGFbR expression was knocked out using the guide TSBTx5277, and “Triple KO” cells were editing using all three guides (see Table 1B).

FIGS. 26A-26C provide a flow cytometry scatter plot and bar graphs demonstrating high efficiency base editing of an HIF-1a isoform 3 polynucleotide (HIF1,3) in EGFR-targeting chimeric antigen receptor (CAR) T cells using the guide polynucleotide TSBTx4470 in combination with an adenosine base editor (ABE). The base editing resulted in knock-out of the HIF-1a isoform 3 gene in the cells. FIG. 26A provides flow cytometry scatter plots showing that T cells obtained from two donors (Donor 1; Donor 2) were effectively transduced with a polynucleotide encoding a chimeric antigen receptor (CAR) targeting EGFR and surface-expressed the CAR. FIG. 26B provides a bar graph showing base editing efficiencies measured in T cells from Donors 1, 2, and 3 that were base edited using the guide polynucleotide TSBTx4470, which targeted an HIF-1a isoform 3 polynucleotide (HIF1,3), and an adenosine base editor (ABE8.20). FIG. 26C provides bar graphs showing levels of the indicated cytokines (GM-CSF, IL-2, TNF-alpha, INF-gamma) produced by EGFR-targeting T cells base edited to knock out expression of HIF-1a isoform 3 when co-cultured with H226 tumor cells at an effector to target ratio of 1:2 for 48 hours in normoxia (20% oxygen) or hypoxia (1% oxygen) conditions. Cytokine levels were measured using an enzyme-linked immunosorbent assay. The base edited CAR T cells showed superior cytokine secretion relative to EGFR-targeting CAR T cells that were not base edited. In FIG. 26C, “CAR” refers to EGFR-targeting CAR T cells that were not base edited to knock-out expression of HIF-1a isoform 3 and “1,3 KO” refers to EGFR-targeting CAR T cells that were base edited to knock out expression of HIF-1a isoform 3.

FIG. 27 provides a plot showing tumor volume in mice administered about 5e6 H226 cells subcutaneously and subsequently intravenously administered 2e6 of the indicated anti-EGFR CAR T cells once H226 tumors reached a volume, on average, of about 150 mm³. In FIG. 27, “Control” indicates mice administered no CAR T cells, “CAR” indicates anti-EGFR CAR T cells base edited to knock-out expression of CD3e (CD3ε), B2M, and CIITA, “A2AR” indicates anti-EGFR CAR T cells base edited to knock-out expression of CD3ε, B2M, CIITA, and A2AR, and “TKO” indicates anti-EGFR CAR T cells base edited to knock-out expression of CD3ε, B2M, CIITA, A2AR, TGFbR2, and PD1. TGFbR2 was knocked-out using the guide polynucleotide TSBTx5277 in combination with Cas12b. Knock-out of all other targets (A2AR, CD3ε, B2M, CIITA, and PD1) was carried out using base editing. A2AR was base edited using ABE8.20 in combination with the guide polynucleotide TSBTx2043. CD3ε was base edited using ABE8.20 in combination with the guide polynucleotide TSBTx4073. B2M was base edited using ABE8.20 in combination with the guide polynucleotide TSBTx760. CIITA was base edited using ABE8.20 in combination with the guide polynucleotide TSBTx763. PD1 was base edited using ABE8.20 in combination with the guide polynucleotide TSBTx025.

FIG. 28 provides a plot showing tumor volume in mice administered H226 cells subcutaneously and subsequently administered 4e6 of the indicated anti-EGFR CAR T cells. In FIG. 28, “Control” indicates mice administered no CAR T cells, “CAR” indicates anti-EGFR CAR T cells base edited to knock-out expression of CD3ε, B2M, and CIITA, “A2AR” indicates anti-EGFR CAR T cells base edited to knock-out expression of CD3ε, B2M, CIITA, and A2AR, and “TKO” indicates anti-EGFR CAR T cells base edited to knock-out expression of CD3ε, B2M, CIITA, A2AR, TGFbR2, and PD1. TGFbR2 was knocked-out using the guide polynucleotide TSBTx5277 in combination with Cas12b. Knock-out of all other targets (A2AR, CD3ε, B2M, CIITA, and PD1) was carried out using base editing. A2AR was base edited using ABE8.20 in combination with the guide polynucleotide TSBTx2043. CD3ε was base edited using ABE8.20 in combination with the guide polynucleotide TSBTx4073. B2M was base edited using ABE8.20 in combination with the guide polynucleotide TSBTx760. CIITA was base edited using ABE8.20 in combination with the guide polynucleotide TSBTx763. PD1 was base edited using ABE8.20 in combination with the guide polynucleotide TSBTx025.

### DETAILED DESCRIPTION OF THE INVENTION

The invention features genetically modified immune cells (e.g., T- or NK-cells), and methods for producing and using these modified immune cells (e.g., T cells, NK cells, or macrophages).

The invention is based, at least in part, on the discovery that generating base edits in one or more genes encoding proteins that function in or regulate hypoxic and adenosinergic pathways (e.g., A2AR, A2BR, HIF1α, HIF1α.I3 in an immune cell (e.g., T- or NK-cell) increases resistance to hypoxic-adenosinergic immunosuppression. The modification of immune cells (e.g., T- or NK-cells) to reduce the expression of A2AR, A2BR, HIF1α, HIF1α.I3 polypeptides and/or polynucleotides is accomplished using a base editor system as described herein.

### Car-T Cell Therapies

Base editors (BEs) are a class of emerging gene editing reagents that enable highly efficient, user-defined modification of target genomic DNA without the creation of double-stranded breaks (DSBs). In contrast to a nuclease-only editing strategy, concurrent modification of one or more genetic loci by base editing produces highly efficient gene knockouts with no detectable translocation events. Multiplex editing of genes is likely to be useful in the creation of CAR-T cell therapies with improved therapeutic properties. The methods described herein address known limitations of immune cell (e.g., CAR-T cell) products and are a promising development towards the next generation of precision cell-based therapies.

The present invention provides modified immune cells (e.g., T- or NK-cells) that have increased resistance to hypoxia-adenosinergic immunosuppression. In some embodiments, the modified immune cell described herein is a modified CAR-T cell. In some embodiments, the CAR-T cell is a T cell that expresses a desired CAR, and can be universally applicable, irrespective of the donor and the recipient's immunogenic compatibility. An immune cell may be derived from one or more subjects or donors. In certain embodiments, the immune cell is derived from a single human subject or donor. For example, the T cell may be derived from PBMCs of a single healthy human donor. In certain embodiments, the immune cell is derived from multiple human donors. In some embodiments, the immune cell is derived from a subject with a disease or disorder (e.g., solid tumor).

In some embodiments, an immune cell (e.g., T- or NK-cell) may be generated, as described herein by using gene modification to introduce concurrent edits at one or more genetic loci. A modification, or concurrent modifications as described herein may be a genetic editing, such as a base editing, generated by a base editor. The base editor may be a C base editor or A base editor. As is discussed herein, base editing may be used to achieve a gene disruption, such that the gene is not expressed. A modification by base editing may be used to achieve a reduction in gene expression. In some embodiments base editor may be used to introduce a genetic modification such that the edited gene does not generate a structurally or functionally viable protein product. In some embodiments, a modification, such as the concurrent modifications described herein may comprise a genetic editing, such as base editing, such that the expression or functionality of the gene product is altered in any way. For example, the expression of the gene product may be enhanced or upregulated as compared to baseline expression levels. In some embodiments the activity or functionality of the gene product may be upregulated as a result of the base editing, or multiple base editing events acting in concert. In some embodiments, a base editor and sgRNAs that provide for multiplex editing are introduced in a single electroporation event, thereby reducing electroporation event associated toxicity. Any known methods for incorporation of exogenous genetic material into a cell may be used to replace electroporation, and such methods known in the art are contemplated for use in any of the methods described herein.

The present invention provides an alternative means of producing modified immune cells (e.g., T- or NK-cells) by using base editing technology to increase resistance to hypoxia-adenosinergic immunosuppression. In some embodiments, at least one or more genes (e.g., A2AR, A2BR, HIF1α, HIF1α.I3), or regulatory elements thereof, are modified in an immune cell (e.g., T- or NK-cell) with the base editing compositions and methods provided herein. In some embodiments, the base editor alters a polynucleotide encoding a polypeptide (e.g., A2AR, A2BR, HIF1α, and/or HIF1α.I3) that functions in or regulates a hypoxic and/or adenosinergic pathways.

In some embodiments, the immune cell (or immune cell equivalent) is obtained from a immune precursor cell (e.g., an induced pluripotent stem cell (iPSC) or an embryonic stem cell (ESC)). In some embodiments, the immune precursor cell is modified by the methods disclosed herein to produce the modified immune cells disclosed herein.

The modified immune cells and methods provided herein address known limitations of CAR-T therapy and is a promising development towards the next generation of precision cell-based therapies.

### Modified Immune Cells

The disclosure provides immune cells (e.g., T- or NK-cells) modified using nucleobase editors described herein. The modified immune cells may express chimeric antigen receptors (CARs) (e.g., CAR-T cells). Modification of immune cells to express a chimeric antigen receptor can enhance an immune cell's immunoreactive activity, wherein the chimeric antigen receptor has an affinity for an epitope on an antigen, wherein the antigen is associated with an altered fitness of an organism. For example, the chimeric antigen receptor can have an affinity for an epitope on a protein expressed in a diseased cell. Because the CAR-T cells can act independently of major histocompatibility complex (MHC), activated CAR-T cells can kill the diseased cell expressing the antigen. The direct action of the CAR-T cell evades defensive mechanisms that have evolved in response to MHC presentation of antigens to immune cells.

In an embodiment, the invention provides T cells that have been altered according to the methods provided herein to reduce or eliminate expression of one or more of the following polypeptides: HIF1α, A2AR, PD1, CTLA4, LAG3, TIM3, TGFbetaR1, TGFbetaR2, HIF1α, and A2AR. In an embodiment, the invention provides T cells that have been altered according to the methods provided herein to reduce or eliminate expression of one or more of the following polypeptides: CD3ε, B2M, CIITA. In an embodiment, the invention provides T cells that have been altered according to the methods provided herein to reduce or eliminate expression of one or more of the following polypeptides: CD3ε, B2M, CIITA, A2AR. In an embodiment, the invention provides T cells that have been altered according to the methods provided herein to reduce or eliminate expression of one or more of the following polypeptides: CD3ε, B2M, CIITA, A2AR, TGFbR2, PD1. In some cases, the T cells have been altered according to the methods provided herein to reduce or eliminate expression of HIF1α and A2AR. In some embodiments, the invention provides T cells that have been altered according to the methods provided herein to reduce or eliminate expression of one or more of the following polypeptides: CD3ε, CD3δ, CD3γ, B2M, CIITA, TRAC, or TRBC. In some cases, the invention provides T cells that have been altered according to the methods provided herein to reduce or eliminate expression of one or more of HIF1a, A2AR, PD1, CTLA4, LAG3, TIM3, TGFbetaR1, TGFbetaR2, dual HIF1α/A2AR and, additionally, to reduce or eliminate expression of one or more of CD3ε, CD3δ, CD3γ, B2M, CIITA, TRAC, or TRBC. In various instances, the invention provides T cells that over-express HLA-E and/or HLA-G. In some cases, the invention provides T cells have been altered according to the methods provided herein to reduce or eliminate expression of HLA Class I polypeptides, HLA Class II polypeptides, and TCR. The present disclosure also provides methods for producing such T cells.

Some embodiments comprise autologous immune cell immunotherapy, wherein immune cells are obtained from a subject having a disease or altered fitness characterized by cancerous or otherwise altered cells expressing a surface marker. The obtained immune cells are genetically modified to express a chimeric antigen receptor and are effectively redirected against specific antigens. Thus, in some embodiments, immune cells are obtained from a subject in need of CAR-T immunotherapy. In some embodiments, these autologous immune cells are cultured and modified shortly after they are obtained from the subject. In other embodiments, the autologous cells are obtained and then stored for future use. This practice may be advisable for individuals who may be undergoing parallel treatment that will diminish immune cell counts in the future.

Some embodiments comprise allogeneic immune cell immunotherapy. In allogeneic immune cell immunotherapy, immune cells are obtained from a donor other than the subject who will be receiving treatment. In some embodiments, immune cells are obtained from a healthy subject or donor and are genetically modified to express a chimeric antigen receptor and are effectively redirected against specific antigens. The immune cells, after modification to express a chimeric antigen receptor (CAR), are administered to a subject for treating a disease. In some embodiments, immune cells to be modified to express a chimeric antigen receptor (CAR) can be obtained from pre-existing stock cultures of immune cells.

Immune cells and/or immune effector cells can be isolated or purified from a sample collected from a subject or a donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD4⁺ is used as a marker to select T cells. In one embodiment, CD8⁺ is used as a marker to select T cells. In one embodiment, CD4⁺ and CD8⁺ are used as a marker to select regulatory T cells.

In another embodiment, the invention provides T cells that have targeted gene knockouts at the TCR constant region (TRAC), which is responsible for TCRαβ surface expression. TCRαβ-deficient CAR T cells are compatible with allogeneic immunotherapy (Qasim et al., Sci. Transl. Med. 9, eaaj2013 (2017); Valton et al., Mol Ther. 2015 September; 23(9): 1507-1518). If desired, residual TCRαβ T cells are removed using CliniMACS magnetic bead depletion to minimize the risk of GVHD. In another embodiment, the invention provides donor T cells selected ex vivo to recognize minor histocompatibility antigens expressed on recipient hematopoietic cells, thereby minimizing the risk of graft-versus-host disease (GVHD), which is the main cause of morbidity and mortality after transplantation (Warren et al., Blood 2010; 115(19):3869-3878).

Another technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4, CD8, CD28, CD45) and corresponding gating strategy. In one embodiment, a CD4 gating strategy is employed. In one embodiment, a CD8 gating strategy is employed. In one embodiment, a CD4 and CD8 gating strategy is employed. In some embodiments, a gating strategy for other markers specific to an immune effector cell is employed instead of, or in combination with, the CD4 and/or CD8 gating strategy.

In some embodiments, the immune effector cells contemplated in the invention are effector T cells. In some embodiments, the effector T cell is a naïve CD8⁺ T cell, a cytotoxic T cell, a natural killer T (NKT) cell, a natural killer (NK) cell, or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4⁺CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, immune effector cells are effector NK cells. In some embodiments, the immune effector cell is any other subset of T cells. The modified immune effector cell may express, in addition to the chimeric antigen receptor (CAR), an exogenous cytokine, a different chimeric receptor, or any other agent that would enhance immune effector cell signaling or function. For example, co-expression of the chimeric antigen receptor and a cytokine may enhance the CAR-T cell's ability to lyse a target cell.

Chimeric antigen receptors (CARs) as contemplated in the present invention may comprise an extracellular binding domain, a transmembrane domain, and an intracellular domain. Binding of an antigen to the extracellular binding domain can activate the CAR-T cell and generate an effector response, which includes CAR-T cell proliferation, cytokine production, and other processes that lead to the death of the antigen expressing cell. Exemplary CARs include those described in the following publications: WO 2020/168300 A1; WO 2020/150534; Li, et al., “Improving the anti-solid tumor efficacy of CAR-T cells by inhibiting adenosine signaling pathway,” Oncoimmunology, 9:e1824643 (2020), DOI: 10.1080/2162402X.2020.1824643; Masoumi, et al., “Genetic and pharmacological targeting of A2a receptor improves function of anti-mesothelin CAR T cells,” Journal of Experimental & Clinical Cancer Research, 39:49 (2020), DOI: 10.1186/s13046-020-01546-6; Xia, et al. “EGFR-targeted CAR-T cells are potent and specific in suppressing triple-negative breast cancer both in vitro and in vivo,” Clinical and Translational Immunology, e1135 (2020), DOI: 10.1002/cti2.1135; Zhou, et al., “Cellular Immunotherapy for Carcinoma Using Genetically Modified EGFR-Specific T-lymphocytes,” NeoPlasia, 15:544-553 (2013), DOI: 10.1593/neo.13168; Li, et al., “Antitumor activity of EGFR-specific CAR T cells against non-small-cell lung cancer cells in vitro and in mice,” Cell Death and Disease, 9:177 (2018), DOI: 10.1038/s41419-017-0238-6; Liu, et al., “Anti-EGFR chimeric antigen receptor-modified T cells in metastatic pancreatic carcinoma: A phase I clinical trial,” Cytotherapy, 22:573-580 (2020), DOI: 10.1016/j.jcyt.2020.04.088; Guo, et al., “Phase I Study of Chimeric Antigen Receptor-Modified T Cells in Patients with EGFR-Positive Advanced Biliary Tract Cancers,” Clinical Cancer Research, 24:1277-1286 (2017), DOI: 10.1158/1078-0432.CCR-17-0432; the entire contents of each of which are incorporated herein in their entireties by reference for all purposes.

In some embodiments of the present invention, the chimeric antigen receptor further comprises a linker. In some embodiments, the linker is a (GGGGS)ₙ linker (SEQ ID NO: 247). In some embodiments, the linker is a (GGGGS)₃ linker (SEQ ID NO: 385). In some embodiments, a CAR of the present invention includes a leader peptide sequence (e.g., N-terminal to the antigen binding domain). An exemplary leader peptide amino acid sequence is: METDTLLLWVLLLWVPGSTG (SEQ ID NO: 386).

Provided herein are also nucleic acid molecules that encode the chimeric antigen receptors (CARs) described herein. In some embodiments, the nucleic acid molecule is isolated or purified. Delivery of the nucleic acid molecules ex vivo can be accomplished using methods known in the art. For example, immune cells obtained from a subject may be transformed with a nucleic acid vector encoding the chimeric antigen receptor. The vector may then be used to transform recipient immune cells so that these cells will then express the chimeric antigen receptor. Efficient means of transforming immune cells include transfection and transduction. Such methods are well known in the art. For example, applicable methods for delivery the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

Some aspects of the present disclosure provide for immune cells comprising a chimeric antigen receptor (CAR) and an altered endogenous gene (e.g., A2AR, A2BR, HIF1α, and HIF1α.I3), whose alteration increases resistance to immunosuppression, or an altered endogenous gene that provides increased cytokine production, persistence, resistance to fratricide, enhances immune cell function, resistance to immunosuppression or inhibition, or a combination thereof. In some embodiments, immune cells described herein comprise a chimeric antigen receptor (CAR) and an altered endogenous gene that provides increased resistance to hypoxia-adenosinergic immunosuppression. In some embodiments, the altered endogenous gene may be created by base editing. In some embodiments, the base editing may reduce or attenuate the gene expression. In some embodiments, the base editing may reduce or attenuate the gene activation. In some embodiments, the base editing may reduce or attenuate the functionality of the gene product. In some other embodiments, the base editing may activate or enhance the gene expression. In some embodiments, the base editing may increase the functionality of the gene product. In some embodiments, the altered endogenous gene may be modified or edited in an exon, an intron, an exon-intron injunction, or a regulatory element thereof. The modification may be edit to a single nucleobase in a gene or a regulatory element thereof. The modification may be in a exon, more than one exons, an intron, or more than one introns, or a combination thereof. The modification may be in an open reading frame of a gene. The modification may be in an untranslated region of the gene, for example, a 3′-UTR or a 5′-UTR. In some embodiments, the modification is in a regulatory element of an endogenous gene. In some embodiments, the modification is in a promoter, an enhancer, an operator, a silencer, an insulator, a terminator, a transcription initiation sequence, a translation initiation sequence (e.g. a Kozak sequence), or any combination thereof.

Allogeneic immune cells expressing an endogenous immune cell receptor as well as a chimeric antigen receptor (CAR) may recognize and attack host cells, a circumstance termed graft versus host disease (GVHD). The alpha component of the immune cell receptor complex is encoded by the TRAC gene, and in some embodiments, this gene is edited such that the alpha subunit of the TCR complex is nonfunctional or absent. Because this subunit is necessary for endogenous immune cell signaling, editing this gene can reduce the risk of graft versus host disease caused by allogeneic immune cells.

In some embodiments, editing of genes to provide increased persistence, fratricide resistance, increased cytokine production, increased resistance to immunosuppression, enhance the function of the immune cell or to reduce immunosuppression or inhibition can occur in the immune cell before the cell is transformed to express a chimeric antigen receptor (CAR). In some embodiments, editing of genes to provide increased resistance to hypoxia-adenosinergic immunosuppression can occur in the immune cell before, during, or after the cell is transformed to express a chimeric antigen receptor (CAR). In some embodiments, editing of genes to provide increased cytokine production can occur in the immune cell before, during, or after the cell is transformed to express a chimeric antigen receptor (CAR). In other aspects, editing of genes to increase persistence, provide fratricide resistance, enhance the function of the immune cell or to reduce immunosuppression or inhibition can occur in a CAR-T cell, i.e., after the immune cell has been transformed to express a chimeric antigen receptor (CAR). In some embodiments, editing of genes to provide increased resistance to hypoxia-adenosinergic immunosuppression can occur in a CAR-T cell, i.e., after the immune cell has been transformed to express a chimeric antigen receptor (CAR)

In some embodiments, the immune cell may comprise one or more edited genes, one or more regulatory elements thereof, or combinations thereof, wherein expression of the edited gene is either knocked out or knocked down. In some embodiments, the immune cell may comprise one or more edited genes, one or more regulatory elements thereof, or combinations thereof, wherein expression of the edited gene is increased. In some embodiments, the immune cell may comprise a chimeric antigen receptor (CAR) and one or more edited genes, one or more regulatory elements thereof, or combinations thereof, wherein expression of the edited gene is either knocked out or knocked down. In some embodiments, the immune cell may comprise a chimeric antigen receptor (CAR) and one or more edited genes, one or more regulatory elements thereof, or combinations thereof, wherein expression of the edited gene is increased.

In some embodiments, the CAR-T cells have reduced or inactivated surface HLA class-I expression as compared to a similar CAR-T cell, but without further having the one or more edited genes as described herein. In some embodiments, the CAR-T cells have increased persistence as compared to a similar CAR-T cell but without further having the one or more edited genes as described herein. In some embodiments, the CAR-T cells have increased fratricide resistance as compared to a similar CAR-T cell but without further having the one or more edited genes as described herein. In some embodiments, the CAR-T cells have reduced immunogenicity as compared to a similar CAR-T cell but without further having the one or more edited genes as described herein. In some embodiments, the CAR-T cells have lower activation threshold as compared to a similar CAR-T but without further having the one or more edited genes as described herein. In some embodiments, the CAR-T cells have increased anti-neoplasia activity as compared to a similar CAR-T cell but without further having the one or more edited genes as described herein. In some embodiments, the CAR-T cells have increased T- and/or NK-cell resistance as compared to a similar CAR-T cell but without further having the one or more edited genes as described herein. The one or more genes may be edited by base editing. In some embodiments the one or more genes are components of hypoxic and/or adenosinergic pathways or regulatory components thereof. In some embodiments the one or more genes are selected from A2AR, A2BR, HIF1α, and HIF1α.I3.

In some embodiments, a cell having an alteration in in a polynucleotide (e.g., gene) encoding one or more of A2AR, A2BR, HIF1a, and HIF1α.I3 further comprises an alteration in a polypeptide selected from one or more of the following: 02M, TAP1, TAP2, and Tapasin; TRAC, CD52, CIITA, HLA-E, HLA-G, PD-L1, PD1, and CD47; TRAC, CD52, and CIITA; HLA-E, HLA-G, PD-L1, PD1, and CD47; one or more of 02M, TAP1, TAP2, and Tapasin and one or more of HLA-E, HLA-G, PD-L1, PD1, and CD47.

In embodiments, a cell of the present disclosure is edited according to methods provided herein and/or those available in the art to alter a nucleobase in one or more genes (e.g., using a base editor), one or more regulatory elements thereof, or combinations thereof. In some instances, the alteration is associated with a reduction in expression and/or activity of a polypeptide encoded by the one or more genes. In some embodiments the one or more genes, or one or more regulatory elements thereof, or combinations thereof, may be selected from a group consisting of: BRINP1, JNK1, PRKCQ, CHIP, CD70, CD58, PD-1, SIRT1, and RNF20. In some embodiments, the one or more genes, or regulatory elements thereof, comprise a combination of targets including one or more of SIRT1 and RNF20, and one or more of PD-1, CD70, and CD58. In embodiments, the combination of targets further includes β2M (β2M). In some embodiments, the one or more genes comprise a combination of targets selected from the following: SIRT1, PD-1, CD70, and CD58; SIRT1, PD-1, and CD70; SIRT1, PD-1, and CD58; SIRT1, CD70, and CD58; SIRT1 and PD-1; SIRT1 and CD70; SIRT1 and CD58; SIRT1, PD-1, CD70, CD58, and B2M; SIRT1, PD-1, CD70, and B2M; SIRT1, PD-1, CD58 and B2M; SIRT1, CD70, CD58, and B2M; SIRT1, PD-1, and B2M; SIRT1, CD70, and B2M; SIRT1, CD58, and B2M; RNF20, PD-1, CD70, and CD58; RNF20, PD-1, and CD70; RNF20, PD-1, and CD58; RNF20, CD70, and CD58; RNF20 and PD-1; RNF20 and CD70; RNF20 and CD58; RNF20, PD-1, CD70, CD58, and B2M; RNF20, PD-1, CD70, and B2M; RNF20, PD-1, CD58 and B2M; RNF20, CD70, CD58, and B2M; RNF20, PD-1, and B2M; RNF20, CD70, and B2M; RNF20, CD58, and B2M; SIRT1, RNF20, PD-1, CD70, and CD58; SIRT1, RNF20, PD-1, and CD70; SIRT1, RNF20, PD-1, and CD58; SIRT1, RNF20, CD70, and CD58; SIRT1, RNF20, and PD-1; SIRT1, RNF20, and CD70; SIRT1, RNF20, and CD58; SIRT1, RNF20, PD-1, CD70, CD58, and B2M; SIRT1, RNF20, PD-1, CD70, and B2M; SIRT1, RNF20, PD-1, CD58, and B2M; SIRT1, RNF20, CD70, CD58, and B2M; SIRT1, RNF20, PD-1, and B2M; SIRT1, RNF20, CD70, and B2M; and SIRT1, RNF20, CD58, and B2M. In embodiments, the one or more genes or regulatory elements thereof include one or more of the following: TAP1, TAP2, Tapasin, NLRC5, CD155, HLA-A, HLA-B, HLA-C, MICA, MICB, Nectin-2, TRAC, ULBP, CIITA, TRBC1, TRBC2, and CD52.

In some embodiments, the at least one or more genes or regulatory elements thereof include one or more of the following: B cell leukemia/lymphoma 11b (Bcl11b); B cell leukemia/lymphoma 2 related protein A1d (Bcl2a1d); B cell leukemia/lymphoma 6 (Bcl6); butyrophilin-like 6 (Btn16); CD151 antigen (Cd151); chemokine (C-C motif) receptor 7 (Ccr7); discs large MAGUK scaffold protein 5 (Dlg5); erythropoietin (Epo); G protein-coupled receptor 18 (Gpr18); interferon alpha 15 (Ifna15); interleukin 6 signal transducer (I16st); interleukin 7 receptor (117r); Janus kinase 3 (Jak3); membrane associated ring-CH-type finger 7 (Marchf7); NCK associated protein 1 like (Nckap11); phospholipase A2, group IIF (Pla2g2f); runt related transcription factor 3 (Runx3); Signal-regulatory protein beta 1B (Sirpb1b); transforming growth factor, beta 1 (Tgfb1); tumor necrosis factor (ligand) superfamily, member 14 (Tnfsf14); tumor necrosis factor (ligand) superfamily, member 18 (Tnfsf18); tumor necrosis factor (ligand) superfamily, member 8 (Tnfsf8); zinc finger CCCH type containing 8 (Zc3h8); (Rac family small GTPase 2); (Slc4a1); 5-azacytidine induced gene 2 (Azi2); a disintegrin and metalloprotease domain 17 (Adam 17); a disintegrin and metalloprotease domain 8 (Adam8); Acetyl-CoA Acetyltransferase 1 (ACAT1); ACLY; adapter related protein complex 3 beta 1 sububit (Ap3b1); adapter related protein complex 3 delta 1 sububit (Ap3d1); adenosine A2a receptor (Adora2a); adenosine deaminase (Ada); adenosine kinase (Adk); adenosine regulating molecule 1 (Adrm1); advanced glycosylation end product-specific receptor (Ager) allograft inflammatory factor 1 (Aif1); AKT1; AKT2; amyloid beta (A4) precursor protein-binding family B member 1 interacting protein (Apbb1ip); ankyrin repeat and LEM domain (Ankle1); annecin A1 (Anxa1); arginase liver (Arg 1); arginase type II (Arg 2); AtPase Cu++ transporting, alpha polypeptide (Atp7a); autoimmune regulator (Aire); autophagy related 5 (Atg5); AXL; B and T Lymphocyte Associated (BTLA); B and T lymphocyte associated (Btla); B cell leukemia/lymphoma 10 (Bcl10); B cell leukemia/lymphoma 11a (Bcl11a); B cell leukemia/lymphoma 2 (Bcl2); B cell leukemia/lymphoma 3 (Bcl3); basic leucine zipper transcription factor, ATF-like (Batf); BCL2-associated X protein (Bax); BCL2L11; beta 2 microglobulin (B2m); BL2-associated agonist of cell dealth (Bad); BLIMP1; Bloom syndrome, RecQ like helicase (Blm); Bmi1 polycomb ring finger oncogene (Bmi1); Bone morphogenic protein 4 (Bmp4); Braf transforming gene (Braf); butyrophilin, subfamily 2, member A1 (Btn2al); butyrophilin, subfamily 2, member A2 (Btn2a2); butyrophilin-like 1 (Btnll); butyrophilin-like 2 (Btnl2); c-abl oncogene 1 (Abl1); c-abl oncogene 2 (Abl2); cadherin-like 26(Cdh26); calcium channel, voltage dependent, beta 4 subunit (Cacnb4); CAMK2D; capping protein regulator and myosin 1 linker 2 (Carmil2); carcinoembryonic antigen-related cell adhesion molecule (Ceacam1); Casitas B-lineage lymphoma b (Cblb); CASP8; Caspase 3 (Casp3); caspase recruitment domain family member 11 (Card11); catenin (cadherin associated protein), beta 1 (Ctnnb1); caveolin 1 (Cav1); CBL-B; CCAAT/enhancer binding protein (C/EBP), beta (Cebpb); CCR10; CCR4; CCR5; CCR6; CCR9; CD103; CD11a; CD122; CD123; CD127; CD130; CD132; CD160 antigen (Cd160); CD161; CD19; CD1d1 antigen (Cd1d1); CD1d2 antigen (CD1d2); CD2 antigen (CD2); CD209e antigen (Cd209e); CD23; CD244 molecule A (Cd244a); CD24a antigen (Cd24a); CD27 antigen (CD27); CD274 antigen (Cd274); CD276 antigen (Cd276); CD28 antigen (Cd28); CD3 delta; CD3 epsilon; CD3 gamma; CD30; CD300A molecule (Cd300a); CD33; CD38; CD4 antigen (Cd4); CD40 ligand (Cd40lg); CD44 antigen (Cd44); CD46 antigen, complement regulatory protein (Cd46); CD47 antigen (Rh-related antigen, integrin-associated signal transducer) (Cd47); CD48 antigen (Cd48); CD5 antigen (Cd5); CD52; CD58; CD59b antigen (Cd59b); CD6 antigen (Cd6); CD69; CD7; CD70; CD74 antigen (Cd74); CD8; CD8 antigen (Cd8); CD80 antigen (Cd80); CD81 antigen (Cd81); CD82; CD83 antigen (Cd83); CD86; CD86 antigen (Cd86); CD8A; CD96; CD99; CDK4; CDK8; CDKN1B; chemokine (C motif) ligand 1 (Xc11); chemokine (C-C motif) ligand 19 (Ccl19); chemokine (C-C motif) ligand 2 (Ccl2); chemokine (C-C motif) ligand 20 (Ccl20); chemokine (C-C motif) ligand 5 (Ccl5); chemokine (C-C motif) receptor 2 (Ccr2); chemokine (C-C motif) receptor 6 (Ccr6); chemokine (C-C motif) receptor 9 (Ccr9); chemokine (C-X-C motif) ligand 12 (Cxcl12); chemokine (C-X-C motif) receptor (Cxcr4); Chitinase 3 Like 1 (Chi311); cholinergic receptor, nicotinic, alpha polypeptide 7 (Chrna7); chromodomain helicase DNA binding protein 7 (Chd7); CLA; Class II Major Histocompatibility Complex Transactivator (CIITA); cleft lip and palate associated transmembrane protein 1 (Clptm1); Cluster of Differentiation 123 (CD123); Cluster of Differentiation 3 (CD3); Cluster of Differentiation 33 (CD33); Cluster of Differentiation 52 (CD52); Cluster of Differentiation 7 (CD7); Cluster of Differentiation 96 (CD96); coagulation factor II (thrombin) receptor-like 1 (F2rl1); coil-coil domain containing 88B (Ccdc88b); core-binding factor beta (Cbfb); coronin, actin binding protein 1A (Coro1a); coxsackie virus and adenovirus receptor (Cxadr); CS-1; CSF2CSK; c-src tyrosine kinase (Csk); C-type lectin domain family 2, member i (Clec2i); C-type lectin domain family 4, member a2 (Clec4a2); C-type lectin domain family 4, member d (Clec4d); C-type lectin domain family 4, member e (Clec4e); C-type lectin domain family 4, member f (Clec4f); C-type lectin domain family 4, member g (Clec4g); CUL3; CXCR3; cyclic GMP-AMP synthase (Cgas); cyclin D3 (Ccnd3); cyclin dependent kinase inhibitor 2A (Cdkn2a); cyclin-dependent kinase (Cdk6); CYLD lysine 63 deubiquitinase (Cyld); cysteine-rich protein 3 (Crip3); cytidine 5′-triphosphate synthase (Ctps); Cytochrome P450 Family 11 Subfamily A Member 1 (Cyp11a1); cytochrome P450, family 26, subfamily b, polypeptide (Cyp26b1); Cytokine Inducible SH2 Containing Protein (CISH); cytotoxic T lymphocyte-associated protein 2 alpha (Ctla2a); Cytotoxic T-Lymphocyte Associated Protein 4 (CTLA-4); DCK; dedicator of cytokinesis 2 (Dock2); dedicator of cytokinesis 8 (Dock8); delta like canonical Notch ligand 4 (D114); deltex 1, E3 ubiquitin ligase (Dtx1); deoxyhypusine synthase (Dhps); DGKA; DGKZ; DHX37; dicer 1, ribonuclease type III (Dicer1); dipeptidylpeptidase 4 (Dpp4); discs large MAGUK scaffold protein 1 (Dlg1); DnaJ heat shock protein family (Hsp40) member A3 (Dnaja3); dolichyl-di-phosphooligosaccharide-protein glycotransferase (Ddost); double homeobox B-like 1 (Duxbl1); drosha, ribonuclease type III (Drosha); dual specificity phosphatase 10 (Dusp10); dual specificity phosphatase 22 (Dusp22); dual specificity phosphatase 3 (Dusp3); E74-like factor 4 (Elf4); early growth response 1(Egr1); early growth response 3 (Egr3); ELOB (TCEB2); ENTPD1 (CD39); eomesodermin (Eomes); Eph receptor B4 (Ephb4); Eph receptor B6 (Ephb6); ephrin B1 (Efnb1); ephrin B2 (Efnb2); ephrin B3 (Efnb3); Epstein-Barr virus induced gene 3 (Ebi3); erb-b2 receptor tyrosine kinase (Erbb2); eukaryotic translation initiation factor 2 alpha kinase 4 (Eif2ak4); FADD; family with sequence similarity 49, member B (Fam49b); Fanconi anemia, complementation group A (Fanca); Fanconi anemia, complementation group D2 (Fancd2); Fas (TNF receptor superfamily member 6) (Fas); Fas (TNFRSF6)-associated via death domain (Fadd); Fas Cell Surface Death Receptor (FAS); Fc receptor, IgE, high affinity I, gamma polypeptide (Fcer1g); fibrinogen-like protein 1 (Fgl1); fibrinogen-like protein 2 (Fgl2); FK506 binding protein 1a (Fkbp1a); FK506 binding protein 1b ((Fkbp1b); flotillin 2 (Flot2); FMS-like tyrosine kinase (Flt3); forkhead box J1 (Foxj1); forkhead box N1 (Foxn1); forkhead box P1 (Foxp1); forkhead box P3 (Foxp3); frizzled class receptor 5 (Fzd5); frizzled class receptor 7 (Fzd7); frizzled class receptor 8 (Fzd8); fucosyltransferase 7 (Fut7); Fyn proto-oncogene (Fyn); gap junction protein, alpha 1 (Gja1); GATA binding protein 3 (GATA3); GCN2 kinase (IDO pathway); gelsolin (Gsn); GLI-Kruppel family member GLI3 (Gli3); glycerol-3-phosphate acyltransferase, mitochondrial (Gpam); growth arrest and DNA-damage-inducible 45 gamma (Gadd45g); GTPase, IMAP family member 1 (Gimap1); H1TET2; H2.0-like homeobox (Hlx); haematopoietic 1(hem1); HCLS1 binding protein 3 (Hs1bp3); heat shock 105 kDa/110 kDa protein 1(Hsph1); heat shock protein 1 (chaperonin) (Hspd1); heat shock protein 90, alpha (cytosolic), class A member 1 (Hsp90aa1); hematopoietic SH2 domain containing (Hsh2d); hepatitis A virus cellular receptor 2 (Havcr2); hes family bHLH transcription factor 1 (Hes1); histocompatibility 2, class II antigen A, alpha (H2-Aa); histocompatibility 2, class II antigen A, beta 1 (H2-Ab1); histocompatibility 2, class II, locus DMa (H2-DMa); histocompatibility 2, M region locus 3(H3-M3); histocompatibility 2, O region alpha locus (H2-Oa); histocompatibility 2, T region locus 23 (H2-T23); HLA-DR; homeostatic iron regulator (Hfe); icos ligand (Icos1); IKAROS family zinc finger 1 (Ikzf1); IL10; IL10RA; IL2 inducible T cell kinase (Itk); IL6R; Indian hedgehog (Ihh); indoleamine 2,3-dioxygenase 1 (Idol); inducible T cell co-stimulator (Icos); inositol 1,4,5-trisphosphate 3-kinase B (Itpkb); insulin II (Ins2); insulin-like growth factor 1 (Igf1); insulin-like growth factor 2 (Igf2); insulin-like growth factor binding protein 2 (Igfbp2); integrin alpha L (Itgal); integrin alpha M (Itgam); integrin alpha V (Itgav); integrin alpha X (Itgax); integrin beta 2 (Itgb2); integrin, alpha D (Itgad); intercellular adhesion molecule 1 (Icam1); interferon (alpha and beta) receptor 1(Ifnar1); interferon alpha 1 (Ifna1); interferon alpha 11 (Ifna11); interferon alpha 12 (Ifna12); interferon alpha 13 (Ifna13); interferon alpha 14 (Ifna14); interferon alpha 16 (Ifna16); interferon alpha 2 (Ifna2); interferon alpha 4 (Ifna4); interferon alpha 5 (Ifna5); interferon alpha 6 (Ifna6); interferon alpha 7 (Ifna7); interferon alpha 9 (Ifna9); interferon alpha B (Ifnab); interferon beta 1 (Ifnb1); interferon gamma (Ifng); interferon kappa (Ifnk); interferon regulatory factor 1 (Irf1); interferon regulatory factor 4 (Irf4); interferon zeta (Ifnz); interleukin 1 beta (Il1b; interleukin 1 family, member 8 (Il1f8); interleukin 1 receptor-like 2 (Il1r12); interleukin 12 receptor, beta1 (Il12rb1); interleukin 12a (12a); interleukin 12b (12b); interleukin 15 (Il15); interleukin 18 (Il18); interleukin 18 receptor 1 (Il18r1); interleukin 2 (Il2); interleukin 2 receptor, alpha chain (I12ra); interleukin 2 receptor, gamma chain (Il2rg); interleukin 20 receptor beta (Il20rb); interleukin 21 (Il21); interleukin 23, alpha subunit p19 (Il23a); interleukin 27 (Il27); interleukin 4 (Il4); interleukin 4 receptor, alpha (Il4ra); interleukin 6 (Il6); interleukin 7 (Il7); IRF8; itchy, E3 ubiquitin protein ligase (Itch); jagged 2 (Jag2); jumonji domain containing 6 (Jmjd6); J UNB; junction adhesion molecule like 9 (Jam9); K(lysine) acetyltransferase 2A (Kat2a); KDEL (Lys-Asp-Glu-Leu) endoplasmic reticulum protein retention receptor 1 (Kdelr1); KIT proto-oncogene receptor tyrosine kinase (Kit); LAG-3; LAIR-1 (CD305); LDHA; lectin, galactose binding, soluble 1 (Lgals1); lectin, galactose binding, soluble 3 (Lgals3); lectin, galactose binding, soluble 8 (Lgals8); lectin, galactose binding, soluble 9 (Lgals9); leptin (Lep); leptin receptor (Lepr); leucine rich repeat containing 32 (Lrrc32); leukocyte immunoglobulin-like receptor, subfamily B, member 4A (Lilrb4a); LFNG O-fucosylpeptide 3-beta-N-acetylglucosaminyltransferase (Lfng); LIF; ligase IV, DNA, ATP-dependent (Lig4); LIM domain only 1 (Lmo1); limb region 1 like (Lmbr1); linker for activation of T cells (Lat); lymphocyte antigen 9 (Ly9); lymphocyte cytosolic protein 1 (Lcp1); lymphocyte protein tyrosine kinase (Lck); lymphocyte transmembrane adaptor 1 (Lax1); lymphocyte-activation gene 3 (Lag3); lymphoid enhancer binding factor 1 (Lef1); LYN; lysyl oxidase-like 3 (Lox13); MAD1 mitotic arrest deficient 1-like 1 (Mad1l1); MALT1 paracaspase (Malt1); MAP4K4; MAPK14; MCJ; mechanistic target of rapamycin kinase (Mtor); MEF2D; Methylation-Controlled J Protein (MCJ); methyltransferase like 3 (Mettl3); MGAT5; MHC I like leukocyte 2 (Mill2); midkine (Mdk); mitogen-activated protein kinase 8 interacting protein 1 (Mapk8ip10); moesin (Msn); myelin protein zero-like 2 (Mpzl2); myeloblastosis oncogene (Myb); myosin, heavy polypeptide 9, non-muscle (Myh9); Nedd4 family interacting protein 1 (Ndfip1); neural precursor cell expressed, developmentally down-regulated 4 (Nedd4); NFATc1; NFATC2; NFATC4; NFKB activating protein (Nkap); nicastrin (Ncstn); NK2 homeobox 3 (Nkx2-3); NLR family, CARD domain containing 3 (Nlrc3); NLR family, pyrin domain containing 3 (Nlrp3); non-catalytic region of tyrosine kinase adaptor protein 1 (Nck1); non-catalytic region of tyrosine kinase adaptor protein 2 (Nck2); non-homologous end joining factor 1 (Nhej 1); non-SMC condensin II complex, subunit H2 (Ncaph2); Notch-regulated ankyrin repeat protein (Nrarp); NT5E (CD73); nuclear factor of activated T cells, cytoplasmic, calcineurin dependent (Nfatc3); nuclear factor of kappa light polypeptide gene enhancer in B cells inhibitor, delta (Nfkbid); nuclear receptor co-repressor 1 (Ncor1); Nuclear Receptor Subfamily 4 Group A Member 1 (NR4A1); Nuclear Receptor Subfamily 4 Group A Member 2 (NR4A2); Nuclear Receptor Subfamily 4 Group A Member 3 (NR4A3); ODC1; OTU domain containing 5 (Otud5); OTULINL (FAM105A); paired box 1 (Pax1); PDCD1 (PD1; PD-1); PDIA3; pellino 1 (Peli1); peroxiredoxin 2 (Prdx2); PHD1 (EGLN2); PHD2 (EGLN1); PHD3 (EGLN3); phosphodiesterase 5A, cGMP-specific (Pde5a); phosphoinositide-3-kinase regulatory subunit (Pik3r6); phospholipase A2, group IIA (Pla2g2a); phospholipase A2, group IID (Pla2g2d); phospholipase A2, group IE (Pla2g2e); phosphoprotein associated with glycosphingolipid microdomains 1 (Pag1); PIK3CD; PIKFYVE; POZ (BTB) and AT hook containing zinc finger 1 (Patz1); PPARa; PPARd; PR domain containing 1, with ZNF domain (Prdm1); presenilin 1 (Psen1); presenilin 2 (Psen2); PRKACA; PRKC, apoptosis, WT1, regulator (Pawr); programmed cell death 1 ligand 2 (Pdcd1lg2); prosaposin (Psap); prostaglandin E receptor 4 (subtype EP4) (Ptger4); protein kinase C, theta 2 (Prkcq); protein kinase C, zeta (Prkcz); protein kinase, cAMP dependent regulatory, type I, alpha (Prkar1a); protein kinase, DNA activated, catalytic polypeptide (Prkdc); protein phosphatase 3, catalytic subunit, beta isoform (Ppp3cb); protein tyrosine phosphatase, non-receptor type 2 (Ptpn2); protein tyrosine phosphatase, non-receptor type 22 (lymphoid) (Ptpn22); protein tyrosine phosphatase, non-receptor type 6 (Ptpn6); protein tyrosine phosphatase, receptor type, C (Ptprc); PTEN; PTPN11; purine-nucleoside phosphorylase (Pnp); purinergic receptor P2X, ligand-gated ion channel, 7 (P2rx7); PVR Related Immunoglobulin Domain Containing (PVRIG; CD112R); PYD and CARD domain containing 7 (Pycard); RAB27A, member RAS oncogene family (Rab27a); RAB29, member RAS oncogene family (Rab29); radical S-adenosyl methionine domain containing 2 (Rsad2); RAR-related orphan receptor alpha (Rora); RAR-related orphan receptor gamma (Ror); RAS guanyl releasing protein 1 (Rasgrp1); ras homolog family member A (Rhoa); ras homolog family member H (Rhoh); RAS protein activator like 3 (Rasal3); RASA2; receptor (TNFRSF)-interacting serine-threonine kinase 2 (Ripk2); recombination activating gene 1 (Rag1); recombination activating gene 2 (Rag2); Regulatory Factor X Associated Ankyrin Containing Protein (RFXANK); RHO family interacting cell polarization regulator 2 (Ripor2); ribosomal protein L22 (Rpl 22); ribosomal protein S6 (Rps6); RING CCCH (C3H) domains 1 (Rc3hl); ring finger and CCCH-type zinc finger domains 2 (Rc3h2); RNF2; runt related transcription factor 1 (Runx1); runt related transcription factor 2 (Runx2); SAM and SH3 domain containing 3 (Sash3); schlafen 1; Selectin P Ligand/P-Selectin Glycoprotein Ligand-1 (SELPG/PSGL1) polypeptide; selenoprotein K (Selenok); sema domain immunoglobulin domain (Ig), transmembrane Domain (TM) and short cytoplasmic domain, (semaphorin) 4A (Sema4a); serine/threonine kinase 11 (Stk11); SH3 domain containing ring finger 1 (Sh3rf1); SHP1; sialophorin (Spn); SIGLEC15; signal transducer and activator of transcription 3 (Stat3); signal transducer and activator of transcription 5A (Stat5A); signal transducer and activator of transcription 5B (Stat5B); signal-regulatory protein alpha (Sirpa); Signal-regulatory protein beta 1A (Sirpb1a); Signal-regulatory protein beta 1C (Sirpb1c); SLA; SLAM family member 6 (Slamf6); SLAMF7; SMAD family member 3 (Smad3); SMAD family member 7 (Smad7); SMARCA4; solute carrier family 11 (proton-coupled divalent metal ion transporters), member 1 (Slc11a1); solute carrier family 4 (anion exchanger), member 1; solute carrier family 46, member 2 (Slc46a2); sonic hedgehog (Shh); SOS Ras/Rac guanine nucleotide exchange factor 1 (Sos1); SOS Ras/Rac guanine nucleotide exchange factor 2 (Sos2); special AT-rich sequence binding protein 1 (Satb1); spleen tyrosine kinase (Syk); Sprouty RTK Signaling Antagonist 1 (Spry1); Sprouty RTK Signaling Antagonist 2 (Spry2); squamous cell carcinoma antigen recognized by T cells (Sart1); src homology 2 domain-containing transforming protein B (Shb); Src-like-adaptor 2 (Sla2); SRY (sex determining region Y)-box 4 (Sox4); STK4; suppression inducing transmembrane adaptor 1 (Sit1); suppressor of cytokine signaling 1 (Socs1); suppressor of cytokine signaling 5 (Socs5); suppressor of cytokine signaling 6 (Socs6); surfactant associated protein D (Sftpd); SUV39; syndecan 4 (Sdc4); syntaxin 11 (Stx11); T Cell Immunoglobulin Mucin 3 (Tim-3); T cell immunoreceptor with Ig and ITIM domains (Tigit); T cell receptor alpha joining 18 (Traj18); T Cell Receptor Beta Constant 1 (TRBC1); T Cell Receptor Beta Constant 2 (TRBC2); T cell, immune regulator 1, ATPase, H+ transporting, lysosomal V0 protein A3 (Tcirg1); T cell-interacting, activating receptor on myeloid cells 1 (Tarm1); T-box 21 (Tbx21); TCR; TCR alpha; TCR beta; TCR complex gene sequence; Tet Methylcytosine Dioxygenase 2 (TET2); TGFbRII; TGFbRII (TGFBR2); three prime repair exonuclease 1 (Trex1); thymocyte selection associated (Themis); thymus cell antigen 1, theta (Thy1); TMEM222; TNF receptor-associated factor 6 (Traf6); TNFAIP3; TNFRSF10B; TNFRSF8 (CD30); TOX; TOX2; TRAC; transformation related protein 53 (Trp53); Transforming Growth Factor Beta Receptor II (TGFbRII); transforming growth factor, beta receptor II (Tgfbr2); transmembrane 131 like (Tmem131l); transmembrane protein 98 (Tmem98); triggering receptor expressed on myeloid cells-like 2 (Treml2); TSC complex subunit 1 (Tsc1); tumor necrosis factor (ligand) superfamily, member 11 (Tnfsf11); tumor necrosis factor (ligand) superfamily, member 13b (Tnfsf13b); tumor necrosis factor (ligand) superfamily, member 4 (Tnfsf4); tumor necrosis factor (ligand) superfamily, member 9 (Tnfsf9); tumor necrosis factor receptor superfamily, member 13c (Tnfrsf13c); tumor necrosis factor receptor superfamily, member 4 (Tnfrsf4); tumor necrosis factor, alpha-induced protein 8-like 2 (Tnfa1p812); twisted gastrulation BMP signaling modulator 1 (Twsg1); UBASH3A; vanin 1 (Vnn1); vascular cell adhesion molecule 1 (Vcam1); VHL; v-maf musculoaponeurotic fibrosarcoma oncogene family, protein B (avian) (Mafb); V-set and immunoglobulin domain containing 4 (Vsig4); V-Set Immunoregulatory Receptor (VISTA); WD repeat and FYVE domain containing 4 (Wdfy4); wingless-type MMTV integration site family, member 1 (Wnt1); wingless-type MMTV integration site family, member 4 (Wnt4); WNT signaling pathway regulator (Apc); WW domain containing E3 ubiquitin protein ligase 1 (Wwp1); XBP1; YAP1; ZAP70; ZC3H12A; zfp35; zinc finger and BTB domain containing 1 (Zbtb1); zinc finger and BTB domain containing 7B (Zbtb7B); zinc finger CCCH type containing 12A (Zc3h12a); zinc finger CCCH type containing 12D (Zc3h12d); zinc finger E-box binding homeobox 1 (Zeb1); zinc finger protein 36, C3H type (Zfp36); zinc finger protein 36, C3H type-like 1 (Zfp36L1); zinc finger protein 36, C3H type-like 2 (Zfp36L2); and zinc finger protein 683 (Zfp683). Further non-limiting examples of the one or more genes, or one or more regulatory elements thereof, or combinations thereof include those described in PCT/US20/13964, PCT/US20/52822, PCT/US20/18178, and/or PCT/US21/52035.

In some embodiments, an immune cell comprises a chimeric antigen receptor (CAR) and one or more additional edited genes, a regulatory element thereof, or combinations thereof. An edited gene may be an immune response regulation gene, an immunogenic gene, a checkpoint inhibitor gene, a gene involved in immune responses, a cell surface marker, e.g. a T cell surface marker, or any combination thereof. In some embodiments, an immune cell comprises a chimeric antigen receptor and an edited gene that is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof. In some embodiments, the edited gene may be a checkpoint inhibitor gene, for example, such as a PD1 gene, a PDC1 gene, or a member related to or regulating the pathway of their formation or activation.

In some embodiments, provided herein is an immune cell with an edited gene in a hypoxic and/or adenosinergic pathway component or a regulatory element thereof, such that the immune cell has an increased resistance to hypoxia-adenosinergic immunosuppression. In some embodiments, provided herein is an immune cell with an edited gene in a hypoxic and/or adenosinergic pathway component or a regulatory element thereof, such that the immune cell has an increased cytokine production. In some embodiments, the immune cell comprises an edited gene in a hypoxic and/or adenosinergic pathway component or a regulatory element thereof, and additionally, at least one edited gene.

In some embodiments, provided herein is an immune cell (e.g., T- or NK-cell) with an edited Adenosine A_{2A} Receptor (A2AR) gene, such that the immune cell does not express or expresses at reduced levels an endogenous functional A2AR. In some embodiments, provided herein is an immune cell with an edited A2AR gene, such that the immune cell has increased resistance to hypoxia-adenosinergic immunosuppression. In some embodiments, provided herein is an immune cell with an edited A2AR gene, such that the immune cell has an increased cytokine production. In some embodiments, the immune cell comprises an edited A2AR gene, and additionally, at least one edited gene.

In some embodiments, provided herein is an immune cell (e.g., T- or NK-cell) with an edited Adenosine A_{2B} Receptor (A2BR) gene, such that the immune cell does not express or expresses at reduced levels an endogenous functional A2BR. In some embodiments, provided herein is an immune cell with an edited A2BR gene, such that the immune cell has increased resistance to hypoxia-adenosinergic immunosuppression. In some embodiments, provided herein is an immune cell with an edited A2BR gene, such that the immune cell has an increased cytokine production. In some embodiments, the immune cell comprises an edited A2BR gene, and additionally, at least one edited gene.

In some embodiments, provided herein is an immune cell (e.g., T- or NK-cell) with an edited Hypoxia-Inducible Factor 1-alpha (HIF1α) gene, such that the immune cell does not express or expresses at reduced levels an endogenous functional HIF1α. In some embodiments, provided herein is an immune cell with an edited HIF1α gene, such that the immune cell has increased resistance to hypoxia-adenosinergic immunosuppression. In some embodiments, provided herein is an immune cell with an edited HIF1α gene, such that the immune cell has an increased cytokine production. In some embodiments, the immune cell comprises an edited HIF1α gene, and additionally, at least one edited gene.

In some embodiments, provided herein is an immune cell (e.g., T- or NK-cell) with an edited Hypoxia-Inducible Factor 1-alpha isoform I3 (HIF1α.I3) gene, such that the immune cell does not express or expresses at reduced levels an endogenous functional HIF1α.I3. In some embodiments, provided herein is an immune cell with an edited HIF1α.I3gene, such that the immune cell has increased resistance to hypoxia-adenosinergic immunosuppression. In some embodiments, provided herein is an immune cell with an edited HIF1α.I3 gene, such that the immune cell has an increased cytokine production. In some embodiments, the immune cell comprises an edited HIF1α.I3gene, and additionally, at least one edited gene.

In some embodiments, each edited gene may comprise a single base edit. In some embodiments, each edited gene may comprise multiple base edits at different regions of the gene. In some embodiments, a single modification event (such as electroporation), may introduce one or more gene edits. In some embodiments at least one, two, three, four, five, six, seven, eight, nine, ten, eleven, twelve, thirteen, fourteen, fifteen, sixteen, seventeen, eighteen, nineteen, twenty or more edits may be introduced in one or more genes simultaneously. In some embodiments, an immune cell, including but not limited to any immune cell comprising an edited gene selected from any of the aforementioned gene edits, can be edited to generate mutations in other genes that enhance the CAR-T's function or reduce immunosuppression or inhibition of the cell.

### Editing of Target Polynucleotides in Immune Cells

In general, base editing is carried out to induce therapeutic changes in the genome of a cell (e.g., immune cell (e.g., T- or NK-cell)), such changes include reducing the expression of a polypeptide or polynucleotide of interest (e.g., A2AR, A2BR, HIF1α, and HIF1α.I3) to reduce immunesuppression. In some instances, a system containing a base editor and/or a nucleic acid programmable DNA binding protein with nuclease activity (e.g., Cas12b) and one or more guide polynucleotides is used to induce changes in the genome of a cell that result in reduced or undetectable levels (e.g., knock-out) of expression relative to an unedited cell of each of the following polypeptides: CD3ε, B2M, CIITA, A2AR, TGFbR2, and PD1. In embodiments, base editing is carried out to induce any of the changes described above into the genome of a cell. In some embodiments, the base edit introduces a stop codon, or alteration in a splice acceptor and/or splice donor site that reduces, eliminates, and/or renders protein expression undetectable. Base editing can be carried out in vitro or in vivo. In some embodiments, cells (e.g., immune cell (e.g., T- or NK-cell)) are collected from a subject or a donor. In some embodiments, base editing is carried out to induce therapeutic changes in the genome of an immune cell (e.g., T- or NK-cell). In some embodiments, base editing is carried out to induce therapeutic changes in the genome of an allogeneic immune cell (e.g., T- or NK-cell) of a subject. In some embodiments, base editing is carried out to induce therapeutic changes in the genome of an allogeneic CAR-T cell.

In some embodiments, immune cells (e.g., T- or NK-cell) of the present invention, are contacted with one or more guide RNAs and a nucleobase editor polypeptide comprising a nucleic acid programmable DNA binding protein (napDNAbp) (e.g., Cas9) domain and a deaminase (e.g., cytidine deaminase and/or adenosine deaminase) domain. In some embodiments, immune cells (e.g., T- or NK-cell) of the present invention, are contacted with one or more guide RNAs and a nucleobase editor polypeptide comprising a nucleic acid programmable DNA binding protein (napDNAbp) (e.g., Cas9) domain and an adenosine deaminase domain. In some embodiments, immune cells (e.g., T- or NK-cell) of the present invention, are contacted with one or more guide RNAs and a nucleobase editor polypeptide comprising a nucleic acid programmable DNA binding protein (napDNAbp) (e.g., Cas9) domain and a cytidine deaminase domain. In some embodiments, immune cells (e.g., T- or NK-cell) of the present invention, are contacted with one or more guide RNAs and a nucleobase editor polypeptide comprising a nucleic acid programmable DNA binding protein (napDNAbp) (e.g., Cas9) domain and an adenosine/cytidine deaminase domain. In some embodiments, the at least one nucleic acid molecule encoding one or more guide RNAs and a nucleobase editor polypeptide is delivered to cells by one or more vectors (e.g., AAV vector).

In some embodiments, one or more vectors (e.g., AAV vector) comprise at least one nucleic acid molecule encoding one or more guide RNAs and a nucleobase editor polypeptide, which comprises a nucleic acid programmable DNA binding protein (napDNAbp) (e.g., Cas9) domain and a deaminase (e.g., cytidine deaminase and/or adenosine deaminase) domain. In some embodiments, one or more vectors (e.g., AAV vector) comprise at least one nucleic acid molecule encoding one or more guide RNAs, which direct a nucleobase editor polypeptide to edit a site in the genome of a cell (e.g., immune cell (e.g., T- or NK-cell)).

The present disclosure provides one or more guide RNAs that direct a nucleobase editor polypeptide to edit a site in the genome of the cell (e.g., immune cell (e.g., T- or NK-cell)). In some embodiments, the present invention provides guide RNAs that target one or more genes in an immune cell (e.g., T- or NK-cell) involved in hypoxic and/or adenosinergic pathways or regulatory components thereof. In some embodiments, the present invention provides guide RNAs that target one or more genes selected from A2AR, A2BR, HIF1α, and HIF1α.I3. In some embodiments, the nucleobase editor polypeptide comprises a deaminase that introduces a stop codon or alters a splice donor or splice acceptor site in a target gene. In some embodiments, the gRNA comprises nucleotide analogs. These nucleotide analogs can inhibit degradation of the gRNA from cellular processes.

In embodiments, a guide polynucleotide of the present disclosure includes a scaffold capable of binding a nucleic acid programmable DNA binding protein (e.g., Cas9 or Cas12b). Non-limiting examples of scaffold sequences include the following:

(Cas9 scaffold; SEQ ID NO: 317)
GUUUUAGAGCUAGAAAUAGCAAGUUAAAAUAAGGCUAGUCCGUUAUCAA
CUUGAAAAAGUGGCACCGAGUCGGUGCUUUU
and
(Cas12b scaffold; SEQ ID NO: 321)
GUUCUGUCUUUUGGUCAGGACAACCGUCUAGCUAUAAGUGCUGCAGGGU
GUGAGAAACUCCUAUUGCUGGACGAUGUCUCUUACGAGGCAUUAGCAC.

Exemplary guide RNA sequences are provided in the following Tables 1A and 1B.

TABLE 1A
Guide RNA Sequences (in Table 1A “SD” represents “splice donor,” “SA” represents “splice acceptor”, “Ex” represents “exon”,
and “Pos” represents “position” within the target sequence, “STOP” indicates a mutation introducing a new stop codon, “START” indicates a
mutation editing a start site codon (e.g., an initial ATG codon))

| | | Target | | Guide | | | | |
|---|---|---|---|---|---|---|---|---|
| | | SEQ ID | | SEQ ID | | | Base | |
| Guide No. | Target Sequence | NO | Spacer Sequence | NO: | PAM | Gene | Editor | Base Edit Position |
| sgRNA | TCACCGGAGCGGGATGCGGA | 420 | UCACCGGAGCGGGAUGCGGA | 387 | NGG | A2AR | CBE | Ex1 SD (Pos 4) |
| 144 | | | | | | | | |
| sgRNA | CTGCTCACCGGAGCGGGATG | 421 | CUGCUCACCGGAGCGGGAUG | 388 | NGG | A2AR | CBE | Ex1 SD (Pos 8) |
| 145 | | | | | | | | |
| sgRNA | CACTCCCAGGGCTGCGGGGA | 422 | CACUCCCAGGGCUGCGGGGA | 389 | NGG | A2AR | CBE | Ex2 STOP (Pos 7) |
| 146 | | | | | | | | |
| sgRNA | CCACTCCCAGGGCTGCGGGG | 423 | CCACUCCCAGGGCUGCGGGG | 390 | NGG | A2AR | CBE | Ex2 STOP (Pos 8) |
| 147 | | | | | | | | |
| sgRNA | GCGACGACAGCTGAAGCAGA | 424 | GCGACGACAGCUGAAGCAGA | 391 | NGG | A2AR | CBE | Ex2 STOP (Pos 8) |
| 148 | | | | | | | | |
| sgRNA | TGGAGAGCCAGCCTCTGCCG | 425 | UGGAGAGCCAGCCUCUGCCG | 392 | NGG | A2AR | CBE | Ex2 STOP (Pos 9) |
| 149 | | | | | | | | |
| sgRNA | GGAGAGCCAGCCTCTGCCGG | 426 | GGAGAGCCAGCCUCUGCCGG | 393 | NGG | A2AR | CBE | Ex2 STOP (Pos 8) |
| 150 | | | | | | | | |
| sgRNA | ACATGAGCCAGAGAGGGGCG | 427 | ACAUGAGCCAGAGAGGGGCG | 394 | NGG | A2AR | CBE | Ex2 STOP (Pos 8/9) |
| 151 | | | | | | | | |
| sgRNA | GAGGCAGCAAGAACCTTTCA | 428 | GAGGCAGCAAGAACCUUUCA | 395 | NGG | A2AR | CBE | Ex2 STOP (Pos 5/8) |
| 152 | | | | | | | | |
| sgRNA | TGGCCCACACTCCTGGCGGG | 429 | UGGCCCACACUCCUGGCGGG | 396 | NGG | A2AR | CBE | Ex2 STOP (Pos 5/6) |
| 153 | | | | | | | | |
| sgRNA | CGTTGGCCCACACTCCTGGC | 430 | CGUUGGCCCACACUCCUGGC | 397 | NGG | A2AR | CBE | Ex2 STOP (Pos 8/9) |
| 154 | | | | | | | | |
| sgRNA | TCTCCCCAGGTACAATGGCT | 431 | UCUCCCCAGGUACAAUGGCU | 398 | NGG | A2AR | ABE | Ex2 SA (Pos 8) |
| 155 | | | | | | | | |
| sgRNA | CAGTTGTTCCAACCTAGCAT | 432 | CAGUUGUUCCAACCUAGCAU | 399 | NGG | A2AR | | Ex2 STOP (Pos 8/9) |
| 201 | | | | | | | | |
| sgRNA | GGCCATGCTGCTGGAGACAC | 433 | GGCCAUGCUGCUGGAGACAC | 400 | NGG | A2BR | ABE | Ex1 START (Pos 5) |
| 221 | | | | | | | | |
| sgRNA | TCACCTGAGCGGGACACAGA | 434 | UCACCUGAGCGGGACACAGA | 401 | NGG | A2BR | | Ex1 SD (Pos 4) |
| 222 | | | | | | | | |
| sgRNA | TTACTGTTCCACCCCAGGAA | 435 | UUACUGUUCCACCCCAGGAA | 402 | NGG | A2BR | | Ex2 STOP (Pos 9) |
| 223 | | | | | | | | |
| sgRNA | TTTAAACAGGTATAAAAGTT | 436 | UUUAAACAGGUAUAAAAGUU | 403 | NGG | A2BR | ABE | Ex2 SA (Pos 8) |
| 224 | | | | | | | | |
| sgRNA | GCTTCAGCGCACTGAGCTGA | 437 | GCUUCAGCGCACUGAGCUGA | 404 | NGG | A2BR | | Ex2 STOP (Pos 5) |
| 225 | | | | | | | | |
| sgRNA | TGCCAAGCAGATGTCAAGAG | 438 | UGCCAAGCAGAUGUCAAGAG | 405 | NGG | A2BR | | Ex2 STOP (Pos 4) |
| 226 | | | | | | | | |
| sgRNA | CTTACTATCATGATGAGTTT | 439 | CUUACUAUCAUGAUGAGUUU | 406 | NGG | HIF1α | CBE/ABE | Ex7 SD (Pos 5) |
| 158 | | | | | | | | |
| sgRNA | CATATACCTGAGTAGAAAAT | 440 | CAUAUACCUGAGUAGAAAAU | 407 | NGG | HIF1α | CBE | Ex11 SD (Pos 7) |
| 162 | | | | | | | | |
| sgRNA | TCATATACCTGAGTAGAAAA | 441 | UCAUAUACCUGAGUAGAAAA | 408 | NGG | HIF1α | CBE | Ex11 SD (Pos 8) |
| 163 | | | | | | | | |
| sgRNA | TGTTTACAGTTTGAACTAAC | 442 | UGUUUACAGUUUGAACUAAC | 409 | NGG | HIF1α | ABE | Ex4 SA (Pos 8) |
| 168 | | | | | | | | |
| sgRNA | TCATTAGGCCTTGTGAAAAA | 443 | UCAUUAGGCCUUGUGAAAAA | 410 | NGG | HIF1α | ABE | Ex5 SA (Pos 6) |
| 169 | | | | | | | | |
| sgRNA | ACACAGGTATTGCACTGCAC | 444 | ACACAGGUAUUGCACUGCAC | 411 | NGG | HIF1α | ABE | Ex6 SA (Pos 5) |
| 170 | | | | | | | | |
| sgRNA | TAACAGAATTACCGAATTGA | 445 | UAACAGAAUUACCGAAUUGA | 412 | NGG | HIF1α | ABE | Ex7 SA (Pos 5) |
| 171 | | | | | | | | |
| sgRNA | AACAGAATTACCGAATTGAT | 446 | AACAGAAUUACCGAAUUGAU | 413 | NGG | HIF1α | ABE | Ex7 SA (Pos 4) |
| 172 | | | | | | | | |
| sgRNA | TTTCAGAACTACAGTTCCTG | 447 | UUUCAGAACUACAGUUCCUG | 414 | NGG | HIF1α | ABE | Ex13 SA (Pos 5) |
| 173 | | | | | | | | |
| sgRNA | AGCTCCCAATGTCGGAGTTT | 448 | AGCUCCCAAUGUCGGAGUUU | 415 | NGA | HIF1α. | | Ex1.5 STOP (Pos 7) |
| 227 | | | | | | I3 | | |
| sgRNA | GAGCTCCCAATGTCGGAGTT | 449 | GAGCUCCCAAUGUCGGAGUU | 416 | NGG | HIF1α. | | Ex1.5 STOP (Pos 8) |
| 228 | | | | | | I3 | | |
| sgRNA | TTAAATGAGCTCCCAATGTC | 450 | UUAAAUGAGCUCCCAAUGUC | 417 | NGA | HIF1α. | ABE | Ex1.5 START (Pos 5) |
| 229 | | | | | | I3 | | |
| sgRNA | TTTAAATGAGCTCCCAATGT | 451 | UUUAAAUGAGCUCCCAAUGU | 418 | NGG | HIF1α. | ABE | Ex1.5 START (Pos 6) |
| 230 | | | | | | I3 | | |
| (TSBTx4470) | | | | | | | | |
| sgRNA | ACCATACCCATTTTCTATTC | 452 | ACCAUACCCAUUUUCUAUUC | 419 | NGA | HIF1α. | | Ex1.5 SD (Pos 8) |
| 231 | | | | | | I3 | | |

TABLE 1B
Spacer Sequences.

| | | SEQ | | | |
|---|---|---|---|---|---|
| Guide ID | Guide Sequence | ID NO | PAM | Gene | Notes |
| sgRNA 258 | CCAUGGGUCGGGGGCUGCUC | 490 | NGG | TGFbR2 ABE | |
| sgRNA 259 | UUACCUGCCCACUGUUAGCC | 491 | NGG | TGFbR2 ABE | |
| sgRNA 260 | CCCGCUACAGGGCAUCCAGA | 492 | NGG | TGFbR2 ABE | |
| sgRNA 261 | GUUUUACAGACAAUGGUACU | 493 | NGG | TGFbR1 ABE | |
| sgRNA 262 | CUUGUAGGCCUUGAGAGUAA | 494 | NGG | TGFbR1 ABE | |
| sgRNA 263 | UGAAGCGUUCUGCCACACAC | 495 | NGG | TGFbR2 | |
| | | | | neighboring | |
| | | | | Kinase domain | |
| sgRNA 264 | UGGAAGAUGCUGCUUCUCCAAA | 496 | ATTN | TGFbR2 | Ex2 |
| | | | | | Cas12b |
| sgRNA 265 | UGAAGGAAAAAAAAAAGCCUGG | 497 | ATTN | TGFbR2 | Ex 2 |
| | | | | | Cas12b |
| sgRNA 266 | CACUCAUCAGAGCUACAGGAAC | 498 | ATTN | TGFbR2 | Ex2 |
| | | | | | Cas12b |
| sgRNA 267 | CAAGUGACAGGCAUCAGCCUCC | 499 | ATTN | TGFbR2 | Ex3 |
| | | | | | Cas12b |
| sgRNA 268 | CUCACCUCCACAGUGAUCACAC | 500 | ATTN | TGFbR2 | Ex3 |
| | | | | | Cas12b |
| sgRNA 269 | AUGUCUGAGAAGAUGUCCUUCU | 501 | ATTN | TGFbR2 | Ex3 |
| | | | | | Cas12b |
| sgRNA 270 | UAGGACUUCUGGAGCCAUGUAU | 502 | ATTN | TGFbR2 | Ex 4 |
| | | | | | Cas12b |
| sgRNA 271 | GGAGAAUGUUGAGUCCUUCAAG | 503 | ATTN | TGFbR2 | Ex4 |
| | | | | | Cas12b |
| sgRNA 272 | CCCAGAGCACCAGAGCCAUGGA | 504 | ATTN | TGFbR2 | Ex4 |
| | | | | | Cas12b |
| sgRNA 273 | UGAGCCUCCAUUUGGUUCCAAG | 505 | ATTN | TGFbR2 | Ex5 |
| | | | | | Cas12b |
| sgRNA 274 | CUGGUCGCCCUCGAUCUCUCAA | 506 | ATTN | TGFbR2 | Ex5 |
| | | | | | Cas12b |
| sgRNA 275 | CCAGCUUCUGGCUCAACCACCA | 507 | ATTN | TGFbR2 | Ex5 |
| | | | | | Cas12b |
| TSBTx2043 | UCUCCCCAGGUACAAUGGCU | 508 | TGG | A2AR | ABE8.20 |
| (sgRNA 155) | | | | | |
| TSBTx2042 | CUGCUCACCGGAGCGGGAUG | 509 | CGG | A2AR | CBE |
| (sgRNA 145) | | | | | |
| TSBTx2040 | UUUCAGAACUACAGUUCCUG | 510 | AGG | HIF1a | ABE8.20 |
| (sgRNA 173) | | | | | |
| TSBTx4073 | CUGGAUUACCUCUUGCCCUC | 511 | AGG | CD3e (CD3ϵ) | ABE8.20 |
| (gRNA 566) | | | | | |
| TSBTx760 | CUUACCCCACUUAACUAUCU | 512 | TGG | B2M | ABE8.20 |
| TSBTx763 | CACUCACCUUAGCCUGAGCA | 513 | GGG | CIITA | ABE8.20 |
| | | | | (exon 16 SD) | |
| TSBTx025 | CACCUACCUAAGAACCAUCC | 514 | TGG | PD1 (Exon 1) | ABE8.20 |
| TSBTx5277 | CCCAGAGCACCAGAGCCAUGGA | 515 | ATTT | TGFbR2 | Cas12b |
| (sgRCM272) | | | | | |

In some embodiments, provided herein is an immune cell with at least one modification in an endogenous gene (e.g., A2AR, A2BR, HIF1α, and HIF1α.I3) or regulatory elements thereof. In some embodiments, the immune cell may comprise a further modification in at least one, two, three, four, five, six, seven, eight, nine, ten, eleven, twelve, thirteen, fourteen, fifteen, sixteen, seventeen, eighteen, nineteen, twenty or more endogenous genes or regulatory elements thereof. In some embodiments, the at least one modification is a single nucleobase modification. In some embodiments, the at least one modification is implemented by base editing. The base editing may be positioned at any suitable position of the gene, or in a regulatory element of the gene. Thus, it may be appreciated that a single base editing at a start codon, for example, can completely abolish the expression of the gene. In some embodiments, the base editing may be performed at a splice donor and/or splice acceptor site. In some embodiments, the base editing is performed at multiple target sites. In some embodiments, the base editing may be performed at any exon of the multiple exons in a gene. In some embodiments, base editing may introduce a premature STOP codon into an exon, resulting in either lack of a translated product or in a truncated that may be misfolded and thereby eliminated by degradation, or may produce an unstable mRNA that is readily degraded. In some embodiments, the immune cell is a T cell. In some embodiments, the immune cell is a CAR-T cell. In some embodiments, the immune cell is a NK cell.

In some embodiments, a cell comprises not only alterations that reduce the expression of A2AR, A2BR, HIF1α, and HIF1α.I3, but also comprises an edited gene that is an immune response regulation gene, an immunogenic gene, a checkpoint inhibitor gene, a gene involved in immune responses, a cell surface marker, e.g. a T cell surface marker, or any combination thereof. In some embodiments, the edited gene is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof. In some embodiments, the edited gene may be a checkpoint inhibitor gene.

In some embodiments, the editing of the endogenous gene reduces expression of the gene. In some embodiments, the editing of the endogenous gene reduces expression of the gene by at least 50% as compared to a control cell without the modification. In some embodiments, the editing of the endogenous gene reduces expression of the gene by at least 60% as compared to a control cell without the modification. In some embodiments, the editing of the endogenous gene reduces expression of the gene by at least 70% as compared to a control cell without the modification. In some embodiments, the editing of the endogenous gene reduces expression of the gene by at least 80% as compared to a control cell without the modification. In some embodiments, the editing of the endogenous gene reduces expression of the gene by at least 90% as compared to a control cell without the modification. In some embodiments, the editing of the endogenous gene reduces expression of the gene by at least 100% as compared to a control cell without the modification. In some embodiments, the editing of the endogenous gene eliminates gene expression.

In some embodiments, base editing may be performed on an intron. For example, base editing may be performed on an intron of an A2AR, A2BR, HIF1α, and HIF1α.I3 gene. In some embodiments, the base editing may be performed at a site within an intron. In some embodiments, the base editing may be performed at sites in one or more introns. In some embodiments, the base editing may be performed at any exon of the multiple introns in a gene. In some embodiments, one or more base editing may be performed on an exon, an intron or any combination of exons and introns.

In some embodiments, the modification or base edit may be within a promoter site. In some embodiments, the base edit may be introduced within an alternative promoter site. In some embodiments, the base edit may be in a 5′ regulatory element, such as an enhancer. In some embodiment, base editing may be introduced to disrupt the binding site of a nucleic acid binding protein. Exemplary nucleic acid binding proteins may be a polymerase, nuclease, gyrase, topoisomerase, methylase or methyl transferase, transcription factors, enhancer, PABP, zinc finger proteins, among many others.

In some embodiments, base editing may be used for splice disruption to silence target protein expression (e.g., A2AR, A2BR, HIF1α, and HIF1α.I3 expression). In some embodiments, base editing may generate a splice acceptor-splice donor (SA-SD) site. Targeted base editing generating a SA-SD, or at a SA-SD site can result in reduced expression of a gene or polypeptide (e.g., A2AR, A2BR, HIF1α, and HIF1α.I3). In some embodiments, base editors (e.g., ABE, CBE) are used to target dinucleotide motifs that constitute splice acceptor and splice donor sites, which are the first and last two nucleotides of each intron. In some embodiments, splice disruption is achieved with an adenosine base editor (ABE). In some embodiments, splice disruption is achieved with a cytidine base editor (CBE). In some embodiments, base editors (e.g., ABE, CBE) are used to edit exons by creating STOP codons.

In some embodiments, provided herein is an immune cell with at least one modification in one or more endogenous genes. In some embodiments, the immune cell may have at least one modification in one, two, three, four, five, six, seven, eight, nine, ten, eleven, twelve, thirteen, fourteen, fifteen, sixteen, seventeen, eighteen, nineteen, twenty or more endogenous genes. In some embodiments, the modification generates a premature stop codon in the endogenous genes. In some embodiments, the STOP codon silences target protein expression. In some embodiments, the modification is a single base modification. In some embodiments, the modification is generated by base editing. The premature stop codon may be generated in an exon, an intron, or an untranslated region. In some embodiments, base editing may be used to introduce more than one STOP codon, in one or more alternative reading frames. In some embodiments, the stop codon is generated by a adenosine base editor (ABE). In some embodiments, the stop codon is generated by a cytidine base editor (CBE). In some embodiments, the CBE generates any one of the following edits (shown in underlined font) to generate a STOP codon: CAG→TAG; CAA→TAA; CGA→TGA; TGG→TGA; TGG→TAG; or TGG→TAA.

In some embodiments, the modification is a missense mutation. In some embodiments, the modification is in a peptide binding site, ATP binding site, splice site, promoter, enhancer, or in an untranslated region (UTR). In some embodiments, modification/base edits may be introduced at a 3′-UTR, for example, in a poly adenylation (poly-A) site. In some embodiments, base editing may be performed on a 5′-UTR region.

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase or cytidine deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

In certain embodiments, the nucleobase editors provided herein comprise one or more features that improve base editing activity. For example, any of the nucleobase editors provided herein may comprise a Cas9 domain that has reduced nuclease activity. In some embodiments, any of the nucleobase editors provided herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand opposite the targeted nucleobase. Mutation of the catalytic residue (e.g., D10 to A10) prevents cleavage of the edited (e.g., deaminated) strand containing the targeted residue (e.g., A or C). Such Cas9 variants can generate a single-strand DNA break (nick) at a specific location based on the gRNA-defined target sequence, leading to repair of the non-edited strand, ultimately resulting in a nucleobase change on the non-edited strand.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain can comprise an endonuclease or an exonuclease. An endonuclease can cleave a single strand of a double-stranded nucleic acid or both strands of a double-stranded nucleic acid molecule. In some embodiments, a nuclease domain of a polynucleotide programmable nucleotide binding domain can cut zero, one, or two strands of a target polynucleotide.

Non-limiting examples of a polynucleotide programmable nucleotide binding domain which can be incorporated into a base editor include a CRISPR protein-derived domain, a restriction nuclease, a meganuclease, TAL nuclease (TALEN), and a zinc finger nuclease (ZFN). In some embodiments, a base editor comprises a polynucleotide programmable nucleotide binding domain comprising a natural or modified protein or portion thereof which via a bound guide nucleic acid is capable of binding to a nucleic acid sequence during CRISPR (i.e., Clustered Regularly Interspaced Short Palindromic Repeats)-mediated modification of a nucleic acid. Such a protein is referred to herein as a “CRISPR protein.” Accordingly, disclosed herein is a base editor comprising a polynucleotide programmable nucleotide binding domain comprising all or a portion of a CRISPR protein (i.e. a base editor comprising as a domain all or a portion of a CRISPR protein, also referred to as a “CRISPR protein-derived domain” of the base editor). A CRISPR protein-derived domain incorporated into a base editor can be modified compared to a wild-type or natural version of the CRISPR protein. For example, as described below a CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Cas9 nuclease sequences and structures are well known to those of skill in the art (See, e.g., “Complete genome sequence of an Ml strain of Streptococcus pyogenes.” Ferretti et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607(2011); and “A programmable dual-RNA-guided DNA endonuclease in adaptive bacterial immunity.” Jinek M., et al., Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

### High Fidelity Cas9 Domains

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233. In some embodiments, high fidelity Cas9 domains are engineered Cas9 domains comprising one or more mutations that decrease electrostatic interactions between the Cas9 domain and the sugar-phosphate backbone of a DNA, relative to a corresponding wild-type Cas9 domain. High fidelity Cas9 domains that have decreased electrostatic interactions with the sugar-phosphate backbone of DNA have less off-target effects. In some embodiments, the Cas9 domain (e.g., a wild type Cas9 domain (SEQ ID NOs: 197 and 200) comprises one or more mutations that decrease the association between the Cas9 domain and the sugar-phosphate backbone of a DNA. In some embodiments, a Cas9 domain comprises one or more mutations that decreases the association between the Cas9 domain and the sugar-phosphate backbone of DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, or at least 70%.

In some embodiments, any of the Cas9 fusion proteins provided herein comprise one or more of a D10A, N497X, a R661X, a Q695X, and/or a Q926X mutation, or a corresponding mutation in any of the amino acid sequences provided herein, wherein X is any amino acid. In some embodiments, the high fidelity Cas9 enzyme is SpCas9(K855A), eSpCas9(1.1), SpCas9-HF1, or hyper accurate Cas9 variant (HypaCas9). In some embodiments, the modified Cas9 eSpCas9(1.1) contains alanine substitutions that weaken the interactions between the HNH/RuvC groove and the non-target DNA strand, preventing strand separation and cutting at off-target sites. Similarly, SpCas9-HF1 lowers off-target editing through alanine substitutions that disrupt Cas9's interactions with the DNA phosphate backbone. HypaCas9 contains mutations (SpCas9 N692A/M694A/Q695A/H698A) in the REC3 domain that increase Cas9 proofreading and target discrimination. All three high fidelity enzymes generate less off-target editing than wildtype Cas9.

Cas9 Domains with Reduced Exclusivity

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. This may limit the ability to edit desired bases within a genome. In some embodiments, the base editing fusion proteins provided herein may need to be placed at a precise location, for example a region comprising a target base that is upstream of the PAM. See e.g., Komor, A. C., et al., “Programmable editing of a target base in genomic DNA without double-stranded DNA cleavage” Nature 533, 420-424 (2016), the entire contents of which are hereby incorporated by reference. Exemplary polypeptide sequences for spCas9 proteins capable of binding a PAM sequence are provided in the Sequence Listing as SEQ ID NOs: 197, 201, and 234-237. Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

### Nickases

In some embodiments, the polynucleotide programmable nucleotide binding domain can comprise a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). In some embodiments, a nickase can be derived from a fully catalytically active (e.g., natural) form of a polynucleotide programmable nucleotide binding domain by introducing one or more mutations into the active polynucleotide programmable nucleotide binding domain. For example, where a polynucleotide programmable nucleotide binding domain comprises a nickase domain derived from Cas9, the Cas9-derived nickase domain can include a D10A mutation and a histidine at position 840. In such embodiments, the residue H840 retains catalytic activity and can thereby cleave a single strand of the nucleic acid duplex. In another example, a Cas9-derived nickase domain can comprise an H840A mutation, while the amino acid residue at position 10 remains a D. In some embodiments, a nickase can be derived from a fully catalytically active (e.g., natural) form of a polynucleotide programmable nucleotide binding domain by removing all or a portion of a nuclease domain that is not required for the nickase activity. For example, where a polynucleotide programmable nucleotide binding domain comprises a nickase domain derived from Cas9, the Cas9-derived nickase domain can comprise a deletion of all or a portion of the RuvC domain or the HNH domain.

In some embodiments, wild-type Cas9 corresponds to, or comprises the following amino acid sequence:

(SEQ ID NO: 197)
MDKKYSIGLDIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD

(single underline: HNH domain; double underline: RuvC domain).

In some embodiments, the strand of a nucleic acid duplex target polynucleotide sequence that is cleaved by a base editor comprising a nickase domain (e.g., Cas9-derived nickase domain, Cas12-derived nickase domain) is the strand that is not edited by the base editor (i.e., the strand that is cleaved by the base editor is opposite to a strand comprising a base to be edited). In other embodiments, a base editor comprising a nickase domain (e.g., Cas9-derived nickase domain, Cas12-derived nickase domain) can cleave the strand of a DNA molecule which is being targeted for editing. In such embodiments, the non-targeted strand is not cleaved.

In some embodiments, a Cas9 nuclease has an inactive (e.g., an inactivated) DNA cleavage domain, that is, the Cas9 is a nickase, referred to as an “nCas9” protein (for “nickase” Cas9). The Cas9 nickase may be a Cas9 protein that is capable of cleaving only one strand of a duplexed nucleic acid molecule (e.g., a duplexed DNA molecule). In some embodiments the Cas9 nickase cleaves the target strand of a duplexed nucleic acid molecule, meaning that the Cas9 nickase cleaves the strand that is base paired to (complementary to) a gRNA (e.g., an sgRNA) that is bound to the Cas9. In some embodiments, a Cas9 nickase comprises a D10A mutation and has a histidine at position 840. In some embodiments the Cas9 nickase cleaves the non-target, non-base-edited strand of a duplexed nucleic acid molecule, meaning that the Cas9 nickase cleaves the strand that is not base paired to a gRNA (e.g., an sgRNA) that is bound to the Cas9. In some embodiments, a Cas9 nickase comprises an H840A mutation and has an aspartic acid residue at position 10, or a corresponding mutation. In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field, and are within the scope of this disclosure.

The amino acid sequence of an exemplary catalytically Cas9 nickase (nCas9) is as follows:

(SEQ ID NO: 201)
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKRYTSTKEVLD
ATLIHQSITGLYETRIDLSQLGGD

The Cas9 nuclease has two functional endonuclease domains: RuvC and HNH. Cas9 undergoes a conformational change upon target binding that positions the nuclease domains to cleave opposite strands of the target DNA. The end result of Cas9-mediated DNA cleavage is a double-strand break (DSB) within the target DNA (˜3-4 nucleotides upstream of the PAM sequence). The resulting DSB is then repaired by one of two general repair pathways: (1) the efficient but error-prone non-homologous end joining (NHEJ) pathway; or (2) the less efficient but high-fidelity homology directed repair (HDR) pathway.

The “efficiency” of non-homologous end joining (NHEJ) and/or homology directed repair (HDR) can be calculated by any convenient method. For example, in some embodiments, efficiency can be expressed in terms of percentage of successful HDR. For example, a surveyor nuclease assay can be used to generate cleavage products and the ratio of products to substrate can be used to calculate the percentage. For example, a surveyor nuclease enzyme can be used that directly cleaves DNA containing a newly integrated restriction sequence as the result of successful HDR. More cleaved substrate indicates a greater percent HDR (a greater efficiency of HDR). As an illustrative example, a fraction (percentage) of HDR can be calculated using the following equation [(cleavage products)/(substrate plus cleavage products)] (e.g., (b+c)/(a+b+c), where “a” is the band intensity of DNA substrate and “b” and “c” are the cleavage products).

In some embodiments, efficiency can be expressed in terms of percentage of successful NHEJ. For example, a T7 endonuclease I assay can be used to generate cleavage products and the ratio of products to substrate can be used to calculate the percentage NHEJ. T7 endonuclease I cleaves mismatched heteroduplex DNA which arises from hybridization of wild-type and mutant DNA strands (NHEJ generates small random insertions or deletions (indels) at the site of the original break). More cleavage indicates a greater percent NHEJ (a greater efficiency of NHEJ). As an illustrative example, a fraction (percentage) of NHEJ can be calculated using the following equation: (1−(1−(b+c)/(a+b+c))^{1/2})×100, where “a” is the band intensity of DNA substrate and “b” and “c” are the cleavage products (Ran et. al., Cell. 2013 Sep. 12; 154(6):1380-9; and Ran et al., Nat Protoc. 2013 November; 8(11): 2281-2308).

The NHEJ repair pathway is the most active repair mechanism, and it frequently causes small nucleotide insertions or deletions (indels) at the DSB site. The randomness of NHEJ-mediated DSB repair has important practical implications, because a population of cells expressing Cas9 and a gRNA or a guide polynucleotide can result in a diverse array of mutations. In most embodiments, NHEJ gives rise to small indels in the target DNA that result in amino acid deletions, insertions, or frameshift mutations leading to premature stop codons within the open reading frame (ORF) of the targeted gene. The ideal end result is a loss-of-function mutation within the targeted gene.

While NHEJ-mediated DSB repair often disrupts the open reading frame of the gene, homology directed repair (HDR) can be used to generate specific nucleotide changes ranging from a single nucleotide change to large insertions like the addition of a fluorophore or tag.

In order to utilize HDR for gene editing, a DNA repair template containing the desired sequence can be delivered into the cell type of interest with the gRNA(s) and Cas9 or Cas9 nickase. The repair template can contain the desired edit as well as additional homologous sequence immediately upstream and downstream of the target (termed left & right homology arms). The length of each homology arm can be dependent on the size of the change being introduced, with larger insertions requiring longer homology arms. The repair template can be a single-stranded oligonucleotide, double-stranded oligonucleotide, or a double-stranded DNA plasmid. The efficiency of HDR is generally low (<10% of modified alleles) even in cells that express Cas9, gRNA and an exogenous repair template. The efficiency of HDR can be enhanced by synchronizing the cells, since HDR takes place during the S and G2 phases of the cell cycle. Chemically or genetically inhibiting genes involved in NHEJ can also increase HDR frequency.

In some embodiments, Cas9 is a modified Cas9. A given gRNA targeting sequence can have additional sites throughout the genome where partial homology exists. These sites are called off-targets and need to be considered when designing a gRNA. In addition to optimizing gRNA design, CRISPR specificity can also be increased through modifications to Cas9. Cas9 generates double-strand breaks (DSBs) through the combined activity of two nuclease domains, RuvC and HNH. Cas9 nickase, a D10A mutant of SpCas9, retains one nuclease domain and generates a DNA nick rather than a DSB. The nickase system can also be combined with HDR-mediated gene editing for specific gene edits.

### Catalytically Dead Nucleases

Also provided herein are base editors comprising a polynucleotide programmable nucleotide binding domain which is catalytically dead (i.e., incapable of cleaving a target polynucleotide sequence). Herein the terms “catalytically dead” and “nuclease dead” are used interchangeably to refer to a polynucleotide programmable nucleotide binding domain which has one or more mutations and/or deletions resulting in its inability to cleave a strand of a nucleic acid. In some embodiments, a catalytically dead polynucleotide programmable nucleotide binding domain base editor can lack nuclease activity as a result of specific point mutations in one or more nuclease domains. For example, in the case of a base editor comprising a Cas9 domain, the Cas9 can comprise both a D10A mutation and an H840A mutation. Such mutations inactivate both nuclease domains, thereby resulting in the loss of nuclease activity. In other embodiments, a catalytically dead polynucleotide programmable nucleotide binding domain can comprise one or more deletions of all or a portion of a catalytic domain (e.g., RuvC1 and/or HNH domains). In further embodiments, a catalytically dead polynucleotide programmable nucleotide binding domain comprises a point mutation (e.g., D10A or H840A) as well as a deletion of all or a portion of a nuclease domain. dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference).

In some embodiments, dCas9 corresponds to, or comprises in part or in whole, a Cas9 amino acid sequence having one or more mutations that inactivate the Cas9 nuclease activity. In some embodiments, the nuclease-inactive dCas9 domain comprises a D10X mutation and a H840X mutation of the amino acid sequence set forth herein, or a corresponding mutation in any of the amino acid sequences provided herein, wherein X is any amino acid change. In some embodiments, the nuclease-inactive dCas9 domain comprises a D10A mutation and a H840A mutation of the amino acid sequence set forth herein, or a corresponding mutation in any of the amino acid sequences provided herein. In some embodiments, a nuclease-inactive Cas9 domain comprises the amino acid sequence set forth in Cloning vector pPlatTET-gRNA2 (Accession No. BAV54124).

In some embodiments, a variant Cas9 protein can cleave the complementary strand of a guide target sequence but has reduced ability to cleave the non-complementary strand of a double stranded guide target sequence. For example, the variant Cas9 protein can have a mutation (amino acid substitution) that reduces the function of the RuvC domain. As a non-limiting example, in some embodiments, a variant Cas9 protein has a D10A (aspartate to alanine at amino acid position 10) and can therefore cleave the complementary strand of a double stranded guide target sequence but has reduced ability to cleave the non-complementary strand of a double stranded guide target sequence (thus resulting in a single strand break (SSB) instead of a double strand break (DSB) when the variant Cas9 protein cleaves a double stranded target nucleic acid) (see, for example, Jinek et al., Science. 2012 Aug. 17; 337(6096):816-21).

In some embodiments, a variant Cas9 protein can cleave the non-complementary strand of a double stranded guide target sequence but has reduced ability to cleave the complementary strand of the guide target sequence. For example, the variant Cas9 protein can have a mutation (amino acid substitution) that reduces the function of the HNH domain (RuvC/HNH/RuvC domain motifs). As a non-limiting example, in some embodiments, the variant Cas9 protein has an H840A (histidine to alanine at amino acid position 840) mutation and can therefore cleave the non-complementary strand of the guide target sequence but has reduced ability to cleave the complementary strand of the guide target sequence (thus resulting in a SSB instead of a DSB when the variant Cas9 protein cleaves a double stranded guide target sequence). Such a Cas9 protein has a reduced ability to cleave a guide target sequence (e.g., a single stranded guide target sequence) but retains the ability to bind a guide target sequence (e.g., a single stranded guide target sequence).

As another non-limiting example, in some embodiments, the variant Cas9 protein harbors W476A and W1126A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some embodiments, the variant Cas9 protein harbors P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some embodiments, the variant Cas9 protein harbors H840A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors H840A, D10A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, the variant Cas9 has restored catalytic His residue at position 840 in the Cas9 HNH domain (A840H).

As another non-limiting example, in some embodiments, the variant Cas9 protein harbors, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors D10A, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, when a variant Cas9 protein harbors W476A and W1126A mutations or when the variant Cas9 protein harbors P475A, W476A, N477A, D1125A, W1126A, and D1127A mutations, the variant Cas9 protein does not bind efficiently to a PAM sequence. Thus, in some such embodiments, when such a variant Cas9 protein is used in a method of binding, the method does not require a PAM sequence. In other words, in some embodiments, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a variant Cas9 protein that has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the variant Cas9 protein can still bind to target DNA in a site-specific manner (because it is still guided to a target DNA sequence by a guide RNA) as long as it retains the ability to interact with the guide RNA.

In some embodiments, the variant Cas protein can be spCas9, spCas9-VRQR, spCas9-VRER, xCas9 (sp), saCas9, saCas9-KKH, spCas9-MQKSER, spCas9-LRKIQK, or spCas9-LRVSQL.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises a N579A mutation, or a corresponding mutation in any of the amino acid sequences provided in the Sequence Listing submitted herewith.

In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT or a NNGRRV PAM sequence. In some embodiments, the SaCas9 domain comprises one or more of a E781X, a N967X, and a R1014X mutation, or a corresponding mutation in any of the amino acid sequences provided herein, wherein X is any amino acid. In some embodiments, the SaCas9 domain comprises one or more of a E781K, a N967K, and a R1014H mutation, or one or more corresponding mutation in any of the amino acid sequences provided herein. In some embodiments, the SaCas9 domain comprises a E781K, a N967K, or a R1014H mutation, or corresponding mutations in any of the amino acid sequences provided herein.

In some embodiments, one of the Cas9 domains present in the fusion protein may be replaced with a guide nucleotide sequence-programmable DNA-binding protein domain that has no requirements for a PAM sequence. In some embodiments, the Cas9 is an SaCas9. Residue A579 of SaCas9 can be mutated from N579 to yield a SaCas9 nickase. Residues K781, K967, and H1014 can be mutated from E781, N967, and R1014 to yield a SaKKH Cas9.

In some embodiments, a modified SpCas9 including amino acid substitutions D1135M, S1136Q, G1218K, E1219F, A1322R, D1332A, R1335E, and T1337R (SpCas9-MQKFRAER) and having specificity for the altered PAM 5′-NGC-3′ was used.

Alternatives to S. pyogenes Cas9 can include RNA-guided endonucleases from the Cpf1 family that display cleavage activity in mammalian cells. CRISPR from Prevotella and Francisella 1 (CRISPR/Cpf1) is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. Unlike Cas9 nucleases, the result of Cpfi-mediated DNA cleavage is a double-strand break with a short 3′ overhang. Cpf1's staggered cleavage pattern can open up the possibility of directional gene transfer, analogous to traditional restriction enzyme cloning, which can increase the efficiency of gene editing. Like the Cas9 variants and orthologues described above, Cpf1 can also expand the number of sites that can be targeted by CRISPR to AT-rich regions or AT-rich genomes that lack the NGG PAM sites favored by SpCas9. The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9.

Furthermore, Cpf1, unlike Cas9, does not have a HNH endonuclease domain, and the N-terminal of Cpf1 does not have the alpha-helical recognition lobe of Cas9. Cpf1 CRISPR-Cas domain architecture shows that Cpf1 is functionally unique, being classified as Class 2, type V CRISPR system. The Cpf1 loci encode Cas1, Cas2 and Cas4 proteins that are more similar to types I and III than type II systems. Functional Cpf1 does not require the trans-activating CRISPR RNA (tracrRNA), therefore, only CRISPR (crRNA) is required. This benefits genome editing because Cpf1 is not only smaller than Cas9, but also it has a smaller sgRNA molecule (approximately half as many nucleotides as Cas9). The Cpf1-crRNA complex cleaves target DNA or RNA by identification of a protospacer adjacent motif 5′-YTN-3′ or 5′-TTN-3′ in contrast to the G-rich PAM targeted by Cas9. After identification of PAM, Cpf1 introduces a sticky-end-like DNA double-stranded break having an overhang of 4 or 5 nucleotides.

In some embodiments, the Cas9 is a Cas9 variant having specificity for an altered PAM sequence. In some embodiments, the Additional Cas9 variants and PAM sequences are described in Miller, S. M., et al. Continuous evolution of SpCas9 variants compatible with non-G PAMs, Nat. Biotechnol. (2020), the entirety of which is incorporated herein by reference. in some embodiments, a Cas9 variate have no specific PAM requirements. In some embodiments, a Cas9 variant, e.g. a SpCas9 variant has specificity for a NRNH PAM, wherein R is A or G and H is A, C, or T. In some embodiments, the SpCas9 variant has specificity for a PAM sequence AAA, TAA, CAA, GAA, TAT, GAT, or CAC. In some embodiments, the SpCas9 variant comprises an amino acid substitution at position 1114, 1134, 1135, 1137, 1139, 1151, 1180, 1188, 1211, 1218, 1219, 1221, 1249, 1256, 1264, 1290, 1318, 1317, 1320, 1321, 1323, 1332, 1333, 1335, 1337, or 1339 or a corresponding position thereof. In some embodiments, the SpCas9 variant comprises an amino acid substitution at position 1114, 1135, 1218, 1219, 1221, 1249, 1320, 1321, 1323, 1332, 1333, 1335, or 1337 or a corresponding position thereof. In some embodiments, the SpCas9 variant comprises an amino acid substitution at position 1114, 1134, 1135, 1137, 1139, 1151, 1180, 1188, 1211, 1219, 1221, 1256, 1264, 1290, 1318, 1317, 1320, 1323, 1333 ora corresponding position thereof. In some embodiments, the SpCas9 variant comprises an amino acid substitution at position 1114, 1131, 1135, 1150, 1156, 1180, 1191, 1218, 1219, 1221, 1227, 1249, 1253, 1286, 1293, 1320, 1321, 1332, 1335, 1339 or a corresponding position thereof. In some embodiments, the SpCas9 variant comprises an amino acid substitution at position 1114, 1127, 1135, 1180, 1207, 1219, 1234, 1286, 1301, 1332, 1335, 1337, 1338, 1349 or a corresponding position thereof. Exemplary amino acid substitutions and PAM specificity of SpCas9 variants are shown in Tables 3A-3D.

TABLE 3A
SpCas9 Variants

| | SpCas9 amino acid position | | | | | | | | | | | | |
|---|---|---|---|---|---|---|---|---|---|---|---|---|---|
| | 1114 | 1135 | 1218 | 1219 | 1221 | 1249 | 1320 | 1321 | 1323 | 1332 | 1333 | 1335 | 1337 |
| PAM | R | D | G | E | Q | P | A | P | A | D | R | R | T |
| AAA | | N | | V | H | | | | | | G | | |
| AAA | | N | | V | H | | | | | | G | | |
| AAA | | | | V | | | | | | | G | | |
| TAA | G | N | | V | | | | | | | I | | |
| TAA | | N | | V | | | | | | | I | | A |
| TAA | G | N | | V | | | | | | | I | | A |
| CAA | | | | V | | | | | | | K | | |
| CAA | | N | | V | | | | | | | K | | |
| CAA | | N | | V | | | | | | | K | | |
| GAA | | | | V | H | | V | | | | K | | |
| GAA | | N | | V | | | V | | | | K | | |
| GAA | | | | V | H | | V | | | | K | | |
| TAT | | | S | V | H | S | | S | | | | L | |
| TAT | | | S | V | H | S | | S | | | | L | |
| TAT | | | S | V | H | S | | S | | | | L | |
| GAT | | | | V | | | | | | | I | | |
| GAT | | | | V | | | | | D | | | Q | |
| GAT | | | | V | | | | | D | | | Q | |
| CAC | | | | V | | | | | | N | | Q | N |
| CAC | | N | | V | | | | | | | | Q | N |
| CAC | | | | V | | | | | | N | | Q | N |

TABLE 3B

| | SpCas9 amino acid position | | | | | | | | | | | | | | | | | | |
|---|---|---|---|---|---|---|---|---|---|---|---|---|---|---|---|---|---|---|---|
| | 1114 | 1134 | 1135 | 1137 | 1139 | 1151 | 1180 | 1188 | 1211 | 1219 | 1221 | 1256 | 1264 | 1290 | 1318 | 1317 | 1320 | 1323 | 1333 |
| PAM | R | F | D | P | V | K | D | K | K | E | Q | Q | H | V | L | N | A | A | R |
| GAA | | | | | | | | | | V | H | | | | | | V | | K |
| GAA | | | N | S | | | | | | V | | | | | | | V | D | K |
| GAA | | | N | | | | | | | V | H | | Y | | | | V | | K |
| CAA | | | N | | | | | | | V | H | | Y | | | | V | | K |
| CAA | G | | N | S | | | | | | V | H | | Y | | | | V | | K |
| CAA | | | N | | | | | R | | V | H | | | | | | V | | K |
| CAA | | | N | | | | G | | R | V | H | | Y | | | | V | | K |
| CAA | | | N | | | | | | | V | H | | Y | | | | V | | K |
| AAA | | | N | | | | G | | | V | H | R | Y | | | | V | D | K |
| CAA | G | | N | | | | G | | | V | H | | Y | | | | V | D | K |
| CAA | | L | N | | | | G | | | V | H | | Y | | | T | V | D | K |
| TAA | G | | N | | | | G | | | V | H | | Y | G | S | | V | D | K |
| TAA | G | | N | | | E | G | | | V | H | | Y | | S | | V | | K |
| TAA | G | | N | | | | G | | | V | H | | Y | | S | | V | D | K |
| TAA | G | | N | | | | G | | R | V | H | | | | | | V | | K |
| TAA | | | N | | | | G | | R | V | H | | Y | | | | V | | K |
| TAA | G | | N | | A | | G | | | V | H | | | | | | V | | K |
| TAA | G | | N | | | | | | | V | H | | | | | | V | | K |

TABLE 3C

| | SpCas9 amino acid position | | | | | | | | | |
|---|---|---|---|---|---|---|---|---|---|---|
| | 1114 | 1131 | 1135 | 1150 | 1156 | 1180 | 1191 | 1218 | 1219 | 1221 |
| PAM | R | Y | D | E | K | D | K | G | E | Q |
| SacB.TAT | | | N | | | | N | | V | H |
| SacB.TAT | | | N | | | | | S | V | H |
| AAT | | | N | | | | | S | V | H |
| TAT | G | | N | | | G | | S | V | H |
| TAT | G | | N | | | G | | S | V | H |
| TAT | G | C | N | | | G | | S | V | H |
| TAT | G | C | N | | | G | | S | V | H |
| TAT | G | C | N | | | G | | S | V | H |
| TAT | G | C | N | | E | G | | S | V | H |
| TAT | G | C | N | V | | G | | S | V | H |
| TAT | | C | N | | | G | | S | V | H |
| TAT | G | C | N | | | G | | S | V | H |

| | SpCas9 amino acid position | | | | | | | | | |
|---|---|---|---|---|---|---|---|---|---|---|
| | 1227 | 1249 | 1253 | 1286 | 1293 | 1320 | 1321 | 1332 | 1335 | 1339 |
| PAM | A | P | E | N | A | A | P | D | R | T |
| SacB.TAT | | | | | | V | S | | L | |
| SacB.TAT | | S | | | | | S | G | L | |
| AAT | V | S | | K | T | | S | G | L | I |
| TAT | | S | K | | | | S | G | L | |
| TAT | | S | | | | | S | G | L | |
| TAT | | S | | | | | S | G | L | |
| TAT | | S | | | | | S | G | L | |
| TAT | | S | | | | | S | G | L | |
| TAT | | S | | | | | S | G | L | |
| TAT | | S | | | | | S | G | L | |
| TAT | | S | | | | | S | G | L | |
| TAT | | S | | | | | S | G | L | |

TABLE 3D

| | SpCas9 amino acid position | | | | | | | | | | | | | |
|---|---|---|---|---|---|---|---|---|---|---|---|---|---|---|
| | 1114 | 1127 | 1135 | 1180 | 1207 | 1219 | 1234 | 1286 | 1301 | 1332 | 1335 | 1337 | 1338 | 1349 |
| PAM | R | D | D | D | E | E | N | N | P | D | R | T | S | H |

| | | | | | | | | | | | | | |
|---|---|---|---|---|---|---|---|---|---|---|---|---|---|
| SacB.CAC | | | N | | | V | | | N | Q | N | | |
| AAC | G | | N | | | V | | | N | Q | N | | |
| AAC | G | | N | | | V | | | N | Q | N | | |
| TAC | G | | N | | | V | | | N | Q | N | | |
| TAC | G | | N | | | V | | H | N | Q | N | | |
| TAC | G | | N | | G | V | D | H | N | Q | N | | |
| TAC | G | | N | | | V | | | N | O | N | | |
| TAC | G | G | N | E | | V | | H | N | Q | N | | |
| TAC | G | | N | | | V | | H | N | Q | N | | |
| TAC | G | | N | | | V | | | N | Q | N | T | R |

Further exemplary Cas9 (e.g., SaCas9) polypeptides with modified PAM recognition are described in Kleinstiver, et al. “Broadening the targeting range of Staphylococcus aureus CRISPR-Cas9 by modifying PAM recognition,” Nature Biotechnology, 33:1293-1298 (2015) DOI: 10.1038/nbt.3404, the disclosure of which is incorporated herein by reference in its entirety for all purposes. In some embodiments, a Cas9 variant (e.g., a SaCas9 variant) comprising one or more of the alterations E782K, N929R, N968K, and/or R1015H has specificity for, or is associated with increased editing activities relative to a reference polypeptide (e.g., SaCas9) at an NNNRRT or NNHRRT PAM sequence, where N represents any nucleotide, H represents any nucleotide other than G (i.e., “not G”), and R represents a purine. In embodiments, the Cas9 variant (e.g., a SaCas9 variant) comprises the alterations E782K, N968K, and R1015H or the alterations E782K, K929R, and R1015H.

In some embodiments, the nucleic acid programmable DNA binding protein (napDNAbp) is a single effector of a microbial CRISPR-Cas system. Single effectors of microbial CRISPR-Cas systems include, without limitation, Cas9, Cpf1, Cas12b/C2c1, and Cas12c/C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (Cas12b/C2c1, and Cas12c/C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, Cas12b/C2c1, and Cas12c/C2c3, contain RuvC-like endonuclease domains related to Cpf1. A third system contains an effector with two predicated HEPN RNase domains. Production of mature CRISPR RNA is tracrRNA-independent, unlike production of CRISPR RNA by Cas12b/C2c1. Cas12b/C2c1 depends on both CRISPR RNA and tracrRNA for DNA cleavage.

In some embodiments, the napDNAbp is a circular permutant (e.g., SEQ ID NO: 238).

The crystal structure of Alicyclobaccillus acidoterrastris Cas12b/C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with Cas12b/C2c1-mediated cleavage resulting in a staggered seven-nucleotide break of target DNA. Structural comparisons between Cas12b/C2c1 ternary complexes and previously identified Cas9 and Cpf1 counterparts demonstrate the diversity of mechanisms used by CRISPR-Cas9 systems.

In some embodiments, the nucleic acid programmable DNA binding protein (napDNAbp) of any of the fusion proteins provided herein may be a Cas12b/C2c1, or a Cas12c/C2c3 protein. In some embodiments, the napDNAbp is a Cas12b/C2c1 protein. In some embodiments, the napDNAbp is a Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12b/C2c1 or Cas12c/C2c3 protein. In some embodiments, the napDNAbp is a naturally-occurring Cas12b/C2c1 or Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of the napDNAbp sequences provided herein. It should be appreciated that Cas12b/C2c1 or Cas12c/C2c3 from other bacterial species may also be used in accordance with the present disclosure.

In some embodiments, a napDNAbp refers to Cas12c. In some embodiments, the Cas12c protein is a Cas12c1 (SEQ ID NO: 239) or a variant of Cas12c1. In some embodiments, the Cas12 protein is a Cas12c2 (SEQ ID NO: 240) or a variant of Cas12c2. In some embodiments, the Cas12 protein is a Cas12c protein from Oleiphilus sp. HI0009 (i.e., OspCas12c; SEQ ID NO: 241) or a variant of OspCas12c. These Cas12c molecules have been described in Yan et al., “Functionally Diverse Type V CRISPR-Cas Systems,” Science, 2019 Jan. 4; 363: 88-91; the entire contents of which is hereby incorporated by reference. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12c1, Cas12c2, or OspCas12c protein. In some embodiments, the napDNAbp is a naturally-occurring Cas12c1, Cas12c2, or OspCas12c protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any Cas12c1, Cas12c2, or OspCas12c protein described herein. It should be appreciated that Cas12c1, Cas12c2, or OspCas12c from other bacterial species may also be used in accordance with the present disclosure.

In some embodiments, a napDNAbp refers to Cas12g, Cas12h, or Cas12i, which have been described in, for example, Yan et al., “Functionally Diverse Type V CRISPR-Cas Systems,” Science, 2019 Jan. 4; 363: 88-91; the entire contents of each is hereby incorporated by reference. Exemplary Cas12g, Cas12h, and Cas12i polypeptide sequences are provided in the Sequence Listing as SEQ ID NOs: 242-245. By aggregating more than 10 terabytes of sequence data, new classifications of Type V Cas proteins were identified that showed weak similarity to previously characterized Class V protein, including Cas12g, Cas12h, and Cas12i. In some embodiments, the Cas12 protein is a Cas12g or a variant of Cas12g. In some embodiments, the Cas12 protein is a Cas12h or a variant of Cas12h. In some embodiments, the Cas12 protein is a Cas12i or a variant of Cas12i. It should be appreciated that other RNA-guided DNA binding proteins may be used as a napDNAbp, and are within the scope of this disclosure. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12g, Cas12h, or Cas12i protein. In some embodiments, the napDNAbp is a naturally-occurring Cas12g, Cas12h, or Cas12i protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any Cas12g, Cas12h, or Cas12i protein described herein. It should be appreciated that Cas12g, Cas12h, or Cas12i from other bacterial species may also be used in accordance with the present disclosure. In some embodiments, the Cas12i is a Cas12i1 or a Cas12i2.

In some embodiments, the nucleic acid programmable DNA binding protein (napDNAbp) of any of the fusion proteins provided herein may be a Cas12j/CasΦ protein. Cas12j/CasΦ is described in Pausch et al., “CRISPR-CasΦ from huge phages is a hypercompact genome editor,” Science, 17 Jul. 2020, Vol. 369, Issue 6501, pp. 333-337, which is incorporated herein by reference in its entirety. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12j/CasΦ protein. In some embodiments, the napDNAbp is a naturally-occurring Cas12j/CasΦ protein. In some embodiments, the napDNAbp is a nuclease inactive (“dead”) Cas12j/CasΦ protein. It should be appreciated that Cas12j/CasΦ from other species may also be used in accordance with the present disclosure.

Fusion Proteins with Internal Insertions

Provided herein are fusion proteins comprising a heterologous polypeptide fused to a nucleic acid programmable nucleic acid binding protein, for example, a napDNAbp. A heterologous polypeptide can be a polypeptide that is not found in the native or wild-type napDNAbp polypeptide sequence. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine of adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide. In some embodiments, the cytidine deaminase is an APOBEC deaminase (e.g., APOBEC1). In some embodiments, the adenosine deaminase is a TadA (e.g., TadA*7.10 or TadA*8). In some embodiments, the TadA is a TadA*8 or a TadA*9. TadA sequences (e.g., TadA7.10 or TadA*8) as described herein are suitable deaminases for the above-described fusion proteins.

In some embodiments, the fusion protein comprises the structure:
NH2-[N-terminal fragment of a napDNAbp]-[deaminase]-[C-terminal fragment of a napDNAbp]-COOH;
NH2-[N-terminal fragment of a Cas9]-[adenosine deaminase]-[C-terminal fragment of a Cas9]-COOH;
NH2-[N-terminal fragment of a Cas12]-[adenosine deaminase]-[C-terminal fragment of a Cas12]-COOH;
NH2-[N-terminal fragment of a Cas9]-[cytidine deaminase]-[C-terminal fragment of a Cas9]-COOH;
NH2-[N-terminal fragment of a Cas12]-[cytidine deaminase]-[C-terminal fragment of a Cas12]-COOH;
wherein each instance of “]-[” is an optional linker.

The deaminase can be a circular permutant deaminase. For example, the deaminase can be a circular permutant adenosine deaminase. In some embodiments, the deaminase is a circular permutant TadA, circularly permutated at amino acid residue 116, 136, or 65 as numbered in the TadA reference sequence.

The fusion protein can comprise more than one deaminase. The fusion protein can comprise, for example, 1, 2, 3, 4, 5 or more deaminases. In some embodiments, the fusion protein comprises one or two deaminase. The two or more deaminases in a fusion protein can be an adenosine deaminase, a cytidine deaminase, or a combination thereof. The two or more deaminases can be homodimers or heterodimers. The two or more deaminases can be inserted in tandem in the napDNAbp. In some embodiments, the two or more deaminases may not be in tandem in the napDNAbp.

In some embodiments, the napDNAbp in the fusion protein is a Cas9 polypeptide or a fragment thereof. The Cas9 polypeptide can be a variant Cas9 polypeptide. In some embodiments, the Cas9 polypeptide is a Cas9 nickase (nCas9) polypeptide or a fragment thereof. In some embodiments, the Cas9 polypeptide is a nuclease dead Cas9 (dCas9) polypeptide or a fragment thereof. The Cas9 polypeptide in a fusion protein can be a full-length Cas9 polypeptide. In some cases, the Cas9 polypeptide in a fusion protein may not be a full length Cas9 polypeptide. The Cas9 polypeptide can be truncated, for example, at a N-terminal or C-terminal end relative to a naturally-occurring Cas9 protein. The Cas9 polypeptide can be a circularly permuted Cas9 protein. The Cas9 polypeptide can be a fragment, a portion, or a domain of a Cas9 polypeptide, that is still capable of binding the target polynucleotide and a guide nucleic acid sequence.

In some embodiments, the Cas9 polypeptide is a Streptococcus pyogenes Cas9 (SpCas9), Staphylococcus aureus Cas9 (SaCas9), Streptococcus thermophilus 1 Cas9 (St1Cas9), or fragments or variants of any of the Cas9 polypeptides described herein.

In some embodiments, the fusion protein comprises an adenosine deaminase domain and a cytidine deaminase domain inserted within a Cas9. In some embodiments, an adenosine deaminase is fused within a Cas9 and a cytidine deaminase is fused to the C-terminus. In some embodiments, an adenosine deaminase is fused within Cas9 and a cytidine deaminase fused to the N-terminus. In some embodiments, a cytidine deaminase is fused within Cas9 and an adenosine deaminase is fused to the C-terminus. In some embodiments, a cytidine deaminase is fused within Cas9 and an adenosine deaminase fused to the N-terminus.

Exemplary structures of a fusion protein with an adenosine deaminase and a cytidine deaminase and a Cas9 are provided as follows:
NH2-[Cas9(adenosine deaminase)]-[cytidine deaminase]-COOH;
NH2-[cytidine deaminase]-[Cas9(adenosine deaminase)]-COOH;
NH2-[Cas9(cytidine deaminase)]-[adenosine deaminase]-COOH; or
NH2-[adenosine deaminase]-[Cas9(cytidine deaminase)]-COOH.

In some embodiments, the “-” used in the general architecture above indicates the presence of an optional linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA*8. In some embodiments, a TadA*8 is fused within Cas9 and a cytidine deaminase is fused to the C-terminus. In some embodiments, a TadA*8 is fused within Cas9 and a cytidine deaminase fused to the N-terminus. In some embodiments, a cytidine deaminase is fused within Cas9 and a TadA*8 is fused to the C-terminus. In some embodiments, a cytidine deaminase is fused within Cas9 and a TadA*8 fused to the N-terminus. Exemplary structures of a fusion protein with a TadA*8 and a cytidine deaminase and a Cas9 are provided as follows:
NH2-[Cas9(TadA*8)]-[cytidine deaminase]-COOH;
NH2-[cytidine deaminase]-[Cas9(TadA*8)]-COOH;
NH2-[Cas9(cytidine deaminase)]-[TadA*8]-COOH; or
NH2-[TadA*8]-[Cas9(cytidine deaminase)]-COOH.

In some embodiments, the “-” used in the general architecture above indicates the presence of an optional linker.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase) can be inserted into a napDNAbp without compromising function of the deaminase (e.g., base editing activity) or the napDNAbp (e.g., ability to bind to target nucleic acid and guide nucleic acid). A deaminase (e.g., adenosine deaminase, cytidine deaminase, or adenosine deaminase and cytidine deaminase) can be inserted in the napDNAbp at, for example, a disordered region or a region comprising a high temperature factor or B-factor as shown by crystallographic studies. Regions of a protein that are less ordered, disordered, or unstructured, for example solvent exposed regions and loops, can be used for insertion without compromising structure or function. A deaminase (e.g., adenosine deaminase, cytidine deaminase, or adenosine deaminase and cytidine deaminase) can be inserted in the napDNAbp in a flexible loop region or a solvent-exposed region. In some embodiments, the deaminase (e.g., adenosine deaminase, cytidine deaminase, or adenosine deaminase and cytidine deaminase) is inserted in a flexible loop of the Cas9 or the Cas12b/C2c1 polypeptide.

In some embodiments, the insertion location of a deaminase (e.g., adenosine deaminase, cytidine deaminase, or adenosine deaminase and cytidine deaminase) is determined by B-factor analysis of the crystal structure of Cas9 polypeptide. In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). B-factor or temperature factor can indicate the fluctuation of atoms from their average position (for example, as a result of temperature-dependent atomic vibrations or static disorder in a crystal lattice). A high B-factor (e.g., higher than average B-factor) for backbone atoms can be indicative of a region with relatively high local mobility. Such a region can be used for inserting a deaminase without compromising structure or function. A deaminase (e.g., adenosine deaminase, cytidine deaminase, or adenosine deaminase and cytidine deaminase) can be inserted at a location with a residue having a Ca atom with a B-factor that is 50%, 60%, 70%, 80%, 90%, 100%, 110%, 120%, 130%, 140%, 150%, 160%, 170%, 180%, 190%, 200%, or greater than 200% more than the average B-factor for the total protein. A deaminase (e.g., adenosine deaminase, cytidine deaminase, or adenosine deaminase and cytidine deaminase) can be inserted at a location with a residue having a Ca atom with a B-factor that is 50%, 60%, 70%, 80%, 90%, 100%, 110%, 120%, 130%, 140%, 150%, 160%, 170%, 180%, 190%,200% or greater than 200% more than the average B-factor for a Cas9 protein domain comprising the residue. Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

A heterologous polypeptide (e.g., deaminase) can be inserted in the napDNAbp at an amino acid residue selected from the group consisting of: 768, 791, 792, 1015, 1016, 1022, 1023, 1026, 1029, 1040, 1052, 1054, 1067, 1068, 1069, 1246, 1247, and 1248 as numbered in the above Cas9 reference sequence, or a corresponding amino acid residue in another Cas9 polypeptide. In some embodiments, the heterologous polypeptide is inserted between amino acid positions 768-769, 791-792, 792-793, 1015-1016, 1022-1023, 1026-1027, 1029-1030, 1040-1041, 1052-1053, 1054-1055, 1067-1068, 1068-1069, 1247-1248, or 1248-1249 as numbered in the above Cas9 reference sequence or corresponding amino acid positions thereof. In some embodiments, the heterologous polypeptide is inserted between amino acid positions 769-770, 792-793, 793-794, 1016-1017, 1023-1024, 1027-1028, 1030-1031, 1041-1042, 1053-1054, 1055-1056, 1068-1069, 1069-1070, 1248-1249, or 1249-1250 as numbered in the above Cas9 reference sequence or corresponding amino acid positions thereof. In some embodiments, the heterologous polypeptide replaces an amino acid residue selected from the group consisting of: 768, 791, 792, 1015, 1016, 1022, 1023, 1026, 1029, 1040, 1052, 1054, 1067, 1068, 1069, 1246, 1247, and 1248 as numbered in the above Cas9 reference sequence, or a corresponding amino acid residue in another Cas9 polypeptide. It should be understood that the reference to the above Cas9 reference sequence with respect to insertion positions is for illustrative purposes. The insertions as discussed herein are not limited to the Cas9 polypeptide sequence of the above Cas9 reference sequence, but include insertion at corresponding locations in variant Cas9 polypeptides, for example a Cas9 nickase (nCas9), nuclease dead Cas9 (dCas9), a Cas9 variant lacking a nuclease domain, a truncated Cas9, or a Cas9 domain lacking partial or complete HNH domain.

A heterologous polypeptide (e.g., deaminase) can be inserted in the napDNAbp at an amino acid residue selected from the group consisting of: 768, 792, 1022, 1026, 1040, 1068, and 1247 as numbered in the above Cas9 reference sequence, or a corresponding amino acid residue in another Cas9 polypeptide. In some embodiments, the heterologous polypeptide is inserted between amino acid positions 768-769, 792-793, 1022-1023, 1026-1027, 1029-1030, 1040-1041, 1068-1069, or 1247-1248 as numbered in the above Cas9 reference sequence or corresponding amino acid positions thereof. In some embodiments, the heterologous polypeptide is inserted between amino acid positions 769-770, 793-794, 1023-1024, 1027-1028, 1030-1031, 1041-1042, 1069-1070, or 1248-1249 as numbered in the above Cas9 reference sequence or corresponding amino acid positions thereof. In some embodiments, the heterologous polypeptide replaces an amino acid residue selected from the group consisting of: 768, 792, 1022, 1026, 1040, 1068, and 1247 as numbered in the above Cas9 reference sequence, or a corresponding amino acid residue in another Cas9 polypeptide.

A heterologous polypeptide (e.g., deaminase) can be inserted in the napDNAbp at an amino acid residue as described herein, or a corresponding amino acid residue in another Cas9 polypeptide. In an embodiment, a heterologous polypeptide (e.g., deaminase) can be inserted in the napDNAbp at an amino acid residue selected from the group consisting of: 1002, 1003, 1025, 1052-1056, 1242-1247, 1061-1077, 943-947, 686-691, 569-578, 530-539, and 1060-1077 as numbered in the above Cas9 reference sequence, or a corresponding amino acid residue in another Cas9 polypeptide. The deaminase (e.g., adenosine deaminase, cytidine deaminase, or adenosine deaminase and cytidine deaminase) can be inserted at the N-terminus or the C-terminus of the residue or replace the residue. In some embodiments, the deaminase (e.g., adenosine deaminase, cytidine deaminase, or adenosine deaminase and cytidine deaminase) is inserted at the C-terminus of the residue.

In some embodiments, an adenosine deaminase (e.g., TadA) is inserted at an amino acid residue selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, an adenosine deaminase (e.g., TadA) is inserted in place of residues 792-872, 792-906, or 2-791 as numbered in the above Cas9 reference sequence, or a corresponding amino acid residue in another Cas9 polypeptide. In some embodiments, the adenosine deaminase is inserted at the N-terminus of an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, the adenosine deaminase is inserted at the C-terminus of an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, the adenosine deaminase is inserted to replace an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide.

In some embodiments, a cytidine deaminase (e.g., APOBECI) is inserted at an amino acid residue selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted at the N-terminus of an amino acid selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted at the C-terminus of an amino acid selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted to replace an amino acid selected from the group consisting of: 1016, 1023, 1029, 1040, 1069, and 1247 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 768 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 791 or is inserted at amino acid residue 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid 791, or is inserted to replace amino acid 792, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1016 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1022, or is inserted at amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1022 or is inserted at the N-terminus of amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1022 or is inserted at the C-terminus of amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1022, or is inserted to replace amino acid residue 1023, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1026, or is inserted at amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1026 or is inserted at the N-terminus of amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1026 or is inserted at the C-terminus of amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1026, or is inserted to replace amino acid residue 1029, as numbered in the above Cas9 reference sequence, or corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1040 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1052, or is inserted at amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1052 or is inserted at the N-terminus of amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1052 or is inserted at the C-terminus of amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1052, or is inserted to replace amino acid residue 1054, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1067, or is inserted at amino acid residue 1068, or is inserted at amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1067 or is inserted at the N-terminus of amino acid residue 1068 or is inserted at the N-terminus of amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1067 or is inserted at the C-terminus of amino acid residue 1068 or is inserted at the C-terminus of amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1067, or is inserted to replace amino acid residue 1068, or is inserted to replace amino acid residue 1069, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1246, or is inserted at amino acid residue 1247, or is inserted at amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1246 or is inserted at the N-terminus of amino acid residue 1247 or is inserted at the N-terminus of amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1246 or is inserted at the C-terminus of amino acid residue 1247 or is inserted at the C-terminus of amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1246, or is inserted to replace amino acid residue 1247, or is inserted to replace amino acid residue 1248, as numbered in the above Cas9 reference sequence, or a corresponding amino acid residue in another Cas9 polypeptide.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. In some embodiments, the deleted region corresponds to residues 792-872 as numbered in the above Cas9 reference sequence, or a corresponding amino acid residue in another Cas9 polypeptide. In some embodiments, the deleted region corresponds to residues 792-906 as numbered in the above Cas9 reference sequence, or a corresponding amino acid residue in another Cas9 polypeptide. In some embodiments, the deleted region corresponds to residues 2-791 as numbered in the above Cas9 reference sequence, or a corresponding amino acid residue in another Cas9 polypeptide. In some embodiments, the deleted region corresponds to residues 1017-1069 as numbered in the above Cas9 reference sequence, or corresponding amino acid residues thereof.

Exemplary internal fusions base editors are provided in Table 4 below:

TABLE 4
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

In some embodiments, the Cas9 polypeptide lacks one or more domains selected from the group consisting of: RuvC I, RuvC II, RuvC III, Recd, Rec2, PI, or HNH domain. In some embodiments, the Cas9 polypeptide lacks a nuclease domain. In some embodiments, the Cas9 polypeptide lacks an HNH domain. In some embodiments, the Cas9 polypeptide lacks a portion of the HNH domain such that the Cas9 polypeptide has reduced or abolished HNH activity. In some embodiments, the Cas9 polypeptide comprises a deletion of the nuclease domain, and the deaminase is inserted to replace the nuclease domain. In some embodiments, the HNH domain is deleted and the deaminase is inserted in its place. In some embodiments, one or more of the RuvC domains is deleted and the deaminase is inserted in its place.

A fusion protein comprising a heterologous polypeptide can be flanked by a N-terminal and a C-terminal fragment of a napDNAbp. In some embodiments, the fusion protein comprises a deaminase flanked by a N-terminal fragment and a C-terminal fragment of a Cas9 polypeptide. The N terminal fragment or the C terminal fragment can bind the target polynucleotide sequence. The C-terminus of the N terminal fragment or the N-terminus of the C terminal fragment can comprise a part of a flexible loop of a Cas9 polypeptide. The C-terminus of the N terminal fragment or the N-terminus of the C terminal fragment can comprise a part of an alpha-helix structure of the Cas9 polypeptide. The N-terminal fragment or the C-terminal fragment can comprise a DNA binding domain. The N-terminal fragment or the C-terminal fragment can comprise a RuvC domain. The N-terminal fragment or the C-terminal fragment can comprise an HNH domain. In some embodiments, neither of the N-terminal fragment and the C-terminal fragment comprises an HNH domain.

In some embodiments, the C-terminus of the N terminal Cas9 fragment comprises an amino acid that is in proximity to a target nucleobase when the fusion protein deaminates the target nucleobase. In some embodiments, the N-terminus of the C terminal Cas9 fragment comprises an amino acid that is in proximity to a target nucleobase when the fusion protein deaminates the target nucleobase. The insertion location of different deaminases can be different in order to have proximity between the target nucleobase and an amino acid in the C-terminus of the N terminal Cas9 fragment or the N-terminus of the C terminal Cas9 fragment. For example, the insertion position of an deaminase can be at an amino acid residue selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide.

The N-terminal Cas9 fragment of a fusion protein (i.e. the N-terminal Cas9 fragment flanking the deaminase in a fusion protein) can comprise the N-terminus of a Cas9 polypeptide. The N-terminal Cas9 fragment of a fusion protein can comprise a length of at least about: 100, 200, 300, 400, 500, 600, 700, 800, 900, 1000, 1100, 1200, or 1300 amino acids. The N-terminal Cas9 fragment of a fusion protein can comprise a sequence corresponding to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide.

The C-terminal Cas9 fragment of a fusion protein (i.e. the C-terminal Cas9 fragment flanking the deaminase in a fusion protein) can comprise the C-terminus of a Cas9 polypeptide. The C-terminal Cas9 fragment of a fusion protein can comprise a length of at least about: 100, 200, 300, 400, 500, 600, 700, 800, 900, 1000, 1100, 1200, or 1300 amino acids. The C-terminal Cas9 fragment of a fusion protein can comprise a sequence corresponding to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide.

The N-terminal Cas9 fragment and C-terminal Cas9 fragment of a fusion protein taken together may not correspond to a full-length naturally occurring Cas9 polypeptide sequence, for example, as set forth in the above Cas9 reference sequence.

The fusion protein described herein can effect targeted deamination with reduced deamination at non-target sites (e.g., off-target sites), such as reduced genome wide spurious deamination. The fusion protein described herein can effect targeted deamination with reduced bystander deamination at non-target sites. The undesired deamination or off-target deamination can be reduced by at least 30%, at least 40%, at least 50%, at least 60%, at least 70%, at least 80%, at least 90%, at least 95%, or at least 99% compared with, for example, an end terminus fusion protein comprising the deaminase fused to a N terminus or a C terminus of a Cas9 polypeptide. The undesired deamination or off-target deamination can be reduced by at least one-fold, at least two-fold, at least three-fold, at least four-fold, at least five-fold, at least tenfold, at least fifteen fold, at least twenty fold, at least thirty fold, at least forty fold, at least fifty fold, at least 60 fold, at least 70 fold, at least 80 fold, at least 90 fold, or at least hundred fold, compared with, for example, an end terminus fusion protein comprising the deaminase fused to a N terminus or a C terminus of a Cas9 polypeptide.

In some embodiments, the deaminase (e.g., adenosine deaminase, cytidine deaminase, or adenosine deaminase and cytidine deaminase) of the fusion protein deaminates no more than two nucleobases within the range of an R-loop. In some embodiments, the deaminase of the fusion protein deaminates no more than three nucleobases within the range of the R-loop. In some embodiments, the deaminase of the fusion protein deaminates no more than 2, 3, 4, 5, 6, 7, 8, 9, or 10 nucleobases within the range of the R-loop. An R-loop is a three-stranded nucleic acid structure including a DNA:RNA hybrid, a DNA:DNA or an RNA: RNA complementary structure and the associated with single-stranded DNA. As used herein, an R-loop may be formed when a target polynucleotide is contacted with a CRISPR complex or a base editing complex, wherein a portion of a guide polynucleotide, e.g. a guide RNA, hybridizes with and displaces with a portion of a target polynucleotide, e.g. a target DNA. In some embodiments, an R-loop comprises a hybridized region of a spacer sequence and a target DNA complementary sequence. An R-loop region may be of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobase pairs in length. In some embodiments, the R-loop region is about 20 nucleobase pairs in length. It should be understood that, as used herein, an R-loop region is not limited to the target DNA strand that hybridizes with the guide polynucleotide. For example, editing of a target nucleobase within an R-loop region may be to a DNA strand that comprises the complementary strand to a guide RNA, or may be to a DNA strand that is the opposing strand of the strand complementary to the guide RNA. In some embodiments, editing in the region of the R-loop comprises editing a nucleobase on non-complementary strand (protospacer strand) to a guide RNA in a target DNA sequence.

The fusion protein described herein can effect target deamination in an editing window different from canonical base editing. In some embodiments, a target nucleobase is from about 1 to about 20 bases upstream of a PAM sequence in the target polynucleotide sequence. In some embodiments, a target nucleobase is from about 2 to about 12 bases upstream of a PAM sequence in the target polynucleotide sequence. In some embodiments, a target nucleobase is from about 1 to 9 base pairs, about 2 to 10 base pairs, about 3 to 11 base pairs, about 4 to 12 base pairs, about 5 to 13 base pairs, about 6 to 14 base pairs, about 7 to 15 base pairs, about 8 to 16 base pairs, about 9 to 17 base pairs, about 10 to 18 base pairs, about 11 to 19 base pairs, about 12 to 20 base pairs, about 1 to 7 base pairs, about 2 to 8 base pairs, about 3 to 9 base pairs, about 4 to 10 base pairs, about 5 to 11 base pairs, about 6 to 12 base pairs, about 7 to 13 base pairs, about 8 to 14 base pairs, about 9 to 15 base pairs, about 10 to 16 base pairs, about 11 to 17 base pairs, about 12 to 18 base pairs, about 13 to 19 base pairs, about 14 to 20 base pairs, about 1 to 5 base pairs, about 2 to 6 base pairs, about 3 to 7 base pairs, about 4 to 8 base pairs, about 5 to 9 base pairs, about 6 to 10 base pairs, about 7 to 11 base pairs, about 8 to 12 base pairs, about 9 to 13 base pairs, about 10 to 14 base pairs, about 11 to 15 base pairs, about 12 to 16 base pairs, about 13 to 17 base pairs, about 14 to 18 base pairs, about 15 to 19 base pairs, about 16 to 20 base pairs, about 1 to 3 base pairs, about 2 to 4 base pairs, about 3 to 5 base pairs, about 4 to 6 base pairs, about 5 to 7 base pairs, about 6 to 8 base pairs, about 7 to 9 base pairs, about 8 to 10 base pairs, about 9 to 11 base pairs, about 10 to 12 base pairs, about 11 to 13 base pairs, about 12 to 14 base pairs, about 13 to 15 base pairs, about 14 to 16 base pairs, about 15 to 17 base pairs, about 16 to 18 base pairs, about 17 to 19 base pairs, about 18 to 20 base pairs away or upstream of the PAM sequence. In some embodiments, a target nucleobase is about 1, 2, 3, 4, 5, 6, 7, 8, 9, 10, 11, 12, 13, 14, 15, 16, 17, 18, 19, 20, or more base pairs away from or upstream of the PAM sequence. In some embodiments, a target nucleobase is about 1, 2, 3, 4, 5, 6, 7, 8, or 9 base pairs upstream of the PAM sequence. In some embodiments, a target nucleobase is about 2, 3, 4, or 6 base pairs upstream of the PAM sequence.

The fusion protein can comprise more than one heterologous polypeptide. For example, the fusion protein can additionally comprise one or more UGI domains and/or one or more nuclear localization signals. The two or more heterologous domains can be inserted in tandem. The two or more heterologous domains can be inserted at locations such that they are not in tandem in the NapDNAbp.

A fusion protein can comprise a linker between the deaminase and the napDNAbp polypeptide. The linker can be a peptide or a non-peptide linker. For example, the linker can be an XTEN, (GGGS)n (SEQ ID NO: 246), (GGGGS)n (SEQ ID NO: 247), (G)n, (EAAAK)n (SEQ ID NO: 248), (GGS)n, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase, but does not comprise a linker between the C-terminal Cas9 fragment and the deaminase. In some embodiments, the fusion protein comprises a linker between the C-terminal Cas9 fragment and the deaminase, but does not comprise a linker between the N-terminal Cas9 fragment and the deaminase.

In some embodiments, the napDNAbp in the fusion protein is a Cas12 polypeptide, e.g., Cas12b/C2c1, or a fragment thereof. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253).

Fusion proteins comprising a heterologous catalytic domain flanked by N- and C-terminal fragments of a Cas12 polypeptide are also useful for base editing in the methods as described herein. Fusion proteins comprising Cas12 and one or more deaminase domains, e.g., adenosine deaminase, or comprising an adenosine deaminase domain flanked by Cas12 sequences are also useful for highly specific and efficient base editing of target sequences. In an embodiment, a chimeric Cas12 fusion protein contains a heterologous catalytic domain (e.g., adenosine deaminase, cytidine deaminase, or adenosine deaminase and cytidine deaminase) inserted within a Cas12 polypeptide. In some embodiments, the fusion protein comprises an adenosine deaminase domain and a cytidine deaminase domain inserted within a Cas12. In some embodiments, an adenosine deaminase is fused within Cas12 and a cytidine deaminase is fused to the C-terminus. In some embodiments, an adenosine deaminase is fused within Cas12 and a cytidine deaminase fused to the N-terminus. In some embodiments, a cytidine deaminase is fused within Cas12 and an adenosine deaminase is fused to the C-terminus. In some embodiments, a cytidine deaminase is fused within Cas12 and an adenosine deaminase fused to the N-terminus. Exemplary structures of a fusion protein with an adenosine deaminase and a cytidine deaminase and a Cas12 are provided as follows:
NH2-[Cas12(adenosine deaminase)]-[cytidine deaminase]-COOH;
NH2-[cytidine deaminase]-[Cas12(adenosine deaminase)]-COOH;
NH2-[Cas12(cytidine deaminase)]-[adenosine deaminase]-COOH; or
NH2-[adenosine deaminase]-[Cas12(cytidine deaminase)]-COOH;

In some embodiments, the “-” used in the general architecture above indicates the presence of an optional linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA*8. In some embodiments, a TadA*8 is fused within Cas12 and a cytidine deaminase is fused to the C-terminus. In some embodiments, a TadA*8 is fused within Cas12 and a cytidine deaminase fused to the N-terminus. In some embodiments, a cytidine deaminase is fused within Cas12 and a TadA*8 is fused to the C-terminus. In some embodiments, a cytidine deaminase is fused within Cas12 and a TadA*8 fused to the N-terminus. Exemplary structures of a fusion protein with a TadA*8 and a cytidine deaminase and a Cas12 are provided as follows:
N-[Cas12(TadA*8)]-[cytidine deaminase]-C;
N-[cytidine deaminase]-[Cas12(TadA*8)]-C;
N-[Cas12(cytidine deaminase)]-[TadA*8]-C; or
N-[TadA*8]-[Cas12(cytidine deaminase)]-C.

In some embodiments, the “-” used in the general architecture above indicates the presence of an optional linker.

In other embodiments, the fusion protein contains one or more catalytic domains. In other embodiments, at least one of the one or more catalytic domains is inserted within the Cas12 polypeptide or is fused at the Cas12 N-terminus or C-terminus. In other embodiments, at least one of the one or more catalytic domains is inserted within a loop, an alpha helix region, an unstructured portion, or a solvent accessible portion of the Cas12 polypeptide. In other embodiments, the Cas12 polypeptide is Cas12a, Cas12b, Cas12c, Cas12d, Cas12e, Cas12g, Cas12h, Cas12i, or Cas12j/CasΦ. In other embodiments, the Cas12 polypeptide has at least about 85% amino acid sequence identity to Bacillus hisashii Cas12b, Bacillus thermoamylovorans Cas12b, Bacillus sp. V3-13 Cas12b, or Alicyclobacillus acidiphilus Cas12b (SEQ ID NO: 254). In other embodiments, the Cas12 polypeptide has at least about 90% amino acid sequence identity to Bacillus hisashii Cas12b (SEQ ID NO: 255), Bacillus thermoamylovorans Cas12b, Bacillus sp. V3-13 Cas12b, or Alicyclobacillus acidiphilus Cas12b. In other embodiments, the Cas12 polypeptide has at least about 95% amino acid sequence identity to Bacillus hisashii Cas12b, Bacillus thermoamylovorans Cas12b (SEQ ID NO: 256), Bacillus sp. V3-13 Cas12b (SEQ ID NO: 257), or Alicyclobacillus acidiphilus Cas12b. In other embodiments, the Cas12 polypeptide contains or consists essentially of a fragment of Bacillus hisashii Cas12b, Bacillus thermoamylovorans Cas12b, Bacillus sp. V3-13 Cas12b, or Alicyclobacillus acidiphilus Cas12b. In embodiments, the Cas12 polypeptide contains BvCas12b (V4), which in some embodiments is expressed as 5′ mRNA Cap---5′ UTR---bhCas12b---STOP sequence---3′ UTR---120polyA tail (SEQ ID NOs: 258-260).

In other embodiments, the catalytic domain is inserted between amino acid positions 153-154, 255-256, 306-307, 980-981, 1019-1020, 534-535, 604-605, or 344-345 of BhCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P153 and S154 of BhCas12b. In other embodiments, the catalytic domain is inserted between amino acids K255 and E256 of BhCas12b. In other embodiments, the catalytic domain is inserted between amino acids D980 and G981 of BhCas12b. In other embodiments, the catalytic domain is inserted between amino acids K1019 and L1020 of BhCas12b. In other embodiments, the catalytic domain is inserted between amino acids F534 and P535 of BhCas12b. In other embodiments, the catalytic domain is inserted between amino acids K604 and G605 of BhCas12b. In other embodiments, the catalytic domain is inserted between amino acids H344 and F345 of BhCas12b. In other embodiments, catalytic domain is inserted between amino acid positions 147 and 148, 248 and 249, 299 and 300, 991 and 992, or 1031 and 1032 of BvCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P147 and D148 of BvCas12b. In other embodiments, the catalytic domain is inserted between amino acids G248 and G249 of BvCas12b. In other embodiments, the catalytic domain is inserted between amino acids P299 and E300 of BvCas12b. In other embodiments, the catalytic domain is inserted between amino acids G991 and E992 of BvCas12b. In other embodiments, the catalytic domain is inserted between amino acids K1031 and M1032 of BvCas12b. In other embodiments, the catalytic domain is inserted between amino acid positions 157 and 158, 258 and 259, 310 and 311, 1008 and 1009, or 1044 and 1045 of AaCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P157 and G158 of AaCas12b. In other embodiments, the catalytic domain is inserted between amino acids V258 and G259 of AaCas12b. In other embodiments, the catalytic domain is inserted between amino acids D310 and P311 of AaCas12b. In other embodiments, the catalytic domain is inserted between amino acids G1008 and E1009 of AaCas12b. In other embodiments, the catalytic domain is inserted between amino acids G1044 and K1045 at of AaCas12b.

In other embodiments, the fusion protein contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

(SEQ ID NO: 262)
ATGGCCCCAAAGAAGAAGCGGAAGGTCGGTATCCACGGAGTCCCAGCAG
CC.

In other embodiments, the Cas12b polypeptide contains a mutation that silences the catalytic activity of a RuvC domain. In other embodiments, the Cas12b polypeptide contains D574A, D829A and/or D952A mutations. In other embodiments, the fusion protein further contains a tag (e.g., an influenza hemagglutinin tag).

In some embodiments, the fusion protein comprises a napDNAbp domain (e.g., Cas12-derived domain) with an internally fused nucleobase editing domain (e.g., all or a portion of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 5 below.

TABLE 5
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

By way of nonlimiting example, an adenosine deaminase (e.g., TadA*8.13) may be inserted into a BhCas12b to produce a fusion protein (e.g., TadA*8.13-BhCas12b) that effectively edits a nucleic acid sequence.

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

In some embodiments, adenosine base editors were generated to insert TadA or variants thereof into the Cas9 polypeptide at the identified positions.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a deoxyadenosine residue in deoxyribonucleic acid (DNA). In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In certain embodiments, a base editor comprising an adenosine deaminase can deaminate a target A of a polynucleotide comprising RNA. For example, the base editor can comprise an adenosine deaminase domain capable of deaminating a target A of an RNA polynucleotide and/or a DNA-RNA hybrid polynucleotide. In an embodiment, an adenosine deaminase incorporated into a base editor comprises all or a portion of adenosine deaminase acting on RNA (ADAR, e.g., ADAR1 or ADAR2) or tRNA (ADAT). A base editor comprising an adenosine deaminase domain can also be capable of deaminating an A nucleobase of a DNA polynucleotide. In an embodiment an adenosine deaminase domain of a base editor comprises all or a portion of an ADAT comprising one or more mutations which permit the ADAT to deaminate a target A in DNA. For example, the base editor can comprise all or a portion of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

It should be appreciated that any of the mutations provided herein (e.g., based on the TadA reference sequence) can be introduced into other adenosine deaminases, such as E. coli TadA (ecTadA), S. aureus TadA (saTadA), or other adenosine deaminases (e.g., bacterial adenosine deaminases). It would be apparent to the skilled artisan that additional deaminases may similarly be aligned to identify homologous amino acid residues that can be mutated as provided herein. Thus, any of the mutations identified in the TadA reference sequence can be made in other adenosine deaminases (e.g., ecTada) that have homologous amino acid residues. It should also be appreciated that any of the mutations provided herein can be made individually or in any combination in the TadA reference sequence or another adenosine deaminase.

In some embodiments, the adenosine deaminase comprises a D108X mutation in the TadA reference sequence, or a corresponding mutation in another adenosine deaminase, where X indicates any amino acid other than the corresponding amino acid in the wild-type adenosine deaminase. In some embodiments, the adenosine deaminase comprises a D108G, D108N, D108V, D108A, or D108Y mutation in TadA reference sequence, or a corresponding mutation in another adenosine deaminase. It should be appreciated, however, that additional deaminases may similarly be aligned to identify homologous amino acid residues that can be mutated as provided herein.

In some embodiments, the adenosine deaminase comprises an A106X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A106V mutation in TadA reference sequence, or a corresponding mutation in another adenosine deaminase (e.g., ecTadA).

In some embodiments, the adenosine deaminase comprises a E155X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a E155D, E155G, or E155V mutation in TadA reference sequence, or a corresponding mutation in another adenosine deaminase (e.g., ecTadA).

In some embodiments, the adenosine deaminase comprises a D147X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a D147Y, mutation in TadA reference sequence, or a corresponding mutation in another adenosine deaminase (e.g., ecTadA).

In some embodiments, the adenosine deaminase comprises an A106X, E155X, or D147X, mutation in the TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an E155D, E155G, or E155V mutation. In some embodiments, the adenosine deaminase comprises a D147Y.

It should also be appreciated that any of the mutations provided herein may be made individually or in any combination in ecTadA or another adenosine deaminase. For example, an adenosine deaminase may contain a D108N, a A106V, a E155V, and/or a D147Y mutation in TadA reference sequence, or a corresponding mutation in another adenosine deaminase (e.g., ecTadA). In some embodiments, an adenosine deaminase comprises the following group of mutations (groups of mutations are separated by a “;”) in a TadA reference sequence, or corresponding mutations in another adenosine deaminase: D108N and A106V; D108N and E155V; D108N and D147Y; A106V and E155V; A106V and D147Y; E155V and D147Y; D108N, A106V, and E155V; D108N, A106V, and D147Y; D108N, E155V, and D147Y; A106V, E155V, and D147Y; and D108N, A106V, E155V, and D147Y. It should be appreciated, however, that any combination of corresponding mutations provided herein may be made in an adenosine deaminase (e.g., ecTadA).

In some embodiments, the adenosine deaminase comprises a combination of mutations in a TadA reference sequence (e.g., TadA*7.10), or corresponding mutations in another adenosine deaminase: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; or L36H+I76Y+V82G+Y147D+F149Y+Q154S+N157K+D167N.

In some embodiments, the adenosine deaminase comprises one or more of a H8X, T17X, L18X, W23X, L34X, W45X, R51X, A56X, E59X, E85X, M94X, I95X, V102X, F104X, A106X, R107X, D108X, K110X, M118X, N127X, A138X, F149X, M151X, R153X, Q154X, I156X, and/or K157X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H8Y, T17S, L18E, W23L, L34S, W45L, R51H, A56E, or A56S, E59G, E85K, or E85G, M94L, I95L, V102A, F104L, A106V, R107C, or R107H, or R107P, D108G, or D108N, or D108V, or D108A, or D108Y, K110I, M118K, N127S, A138V, F149Y, M151V, R153C, Q154L, I156D, and/or K157R mutation in TadA reference sequence, or one or more corresponding mutations in another adenosine deaminase.

In some embodiments, the adenosine deaminase comprises one or more of a H8X, D108X, and/or N127X mutation in TadA reference sequence, or one or more corresponding mutations in another adenosine deaminase, where X indicates the presence of any amino acid. In some embodiments, the adenosine deaminase comprises one or more of a H8Y, D108N, and/or N127S mutation in TadA reference sequence, or one or more corresponding mutations in another adenosine deaminase.

In some embodiments, the adenosine deaminase comprises one or more of H8X, R26X, M61X, L68X, M70X, A106X, D108X, A109X, N127X, D147X, R152X, Q154X, E155X, K161X, Q163X, and/or T166X mutation in TadA reference sequence, or one or more corresponding mutations in another adenosine deaminase, where X indicates the presence of any amino acid other than the corresponding amino acid in the wild-type adenosine deaminase. In some embodiments, the adenosine deaminase comprises one or more of H8Y, R26W, M61I, L68Q, M70V, A106T, D108N, A109T, N127S, D147Y, R152C, Q154H or Q154R, E155G or E155V or E155D, K161Q, Q163H, and/or T166P mutation in TadA reference sequence, or one or more corresponding mutations in another adenosine deaminase.

In some embodiments, the adenosine deaminase comprises one, two, three, four, five, or six mutations selected from the group consisting of H8X, D108X, N127X, D147X, R152X, and Q154X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, M61X, M70X, D108X, N127X, Q154X, E155X, and Q163X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, N127X, E155X, and T166X in TadA reference sequence, or a corresponding mutation or mutations in another adenosine deaminase (e.g., ecTadA), where X indicates the presence of any amino acid other than the corresponding amino acid in the wild-type adenosine deaminase.

In some embodiments, the adenosine deaminase comprises one, two, three, four, five, or six mutations selected from the group consisting of H8X, A106X, and D108X, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, R26X, L68X, D108X, N127X, D147X, and E155X, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of H8X, R126X, L68X, D108X, N127X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, D108X, A109X, N127X, and E155X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, or six mutations selected from the group consisting of H8Y, D108N, N127S, D147Y, R152C, and Q154H in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, M61I, M70V, D108N, N127S, Q154R, E155G and Q163H in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, N127S, E155V, and T166P in TadA reference sequence, or a corresponding mutation or mutations in another adenosine deaminase (e.g., ecTadA). In some embodiments, the adenosine deaminase comprises one, two, three, four, five, or six mutations selected from the group consisting of H8Y, A106T, D108N, N127S, E155D, and K161Q in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, R26W, L68Q, D108N, N127S, D147Y, and E155V in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, A109T, N127S, and E155G in TadA reference sequence, or a corresponding mutation or mutations in another adenosine deaminase (e.g., ecTadA).

In some embodiments, the adenosine deaminase comprises one or more of the or one or more corresponding mutations in another adenosine deaminase. In some embodiments, the adenosine deaminase comprises a D108N, D108G, or D108V mutation in TadA reference sequence, or corresponding mutations in another adenosine deaminase. In some embodiments, the adenosine deaminase comprises a A106V and D108N mutation in TadA reference sequence, or corresponding mutations in another adenosine deaminase. In some embodiments, the adenosine deaminase comprises R107C and D108N mutations in TadA reference sequence, or corresponding mutations in another adenosine deaminase. In some embodiments, the adenosine deaminase comprises a H8Y, D108N, N127S, D147Y, and Q154H mutation in TadA reference sequence, or corresponding mutations in another adenosine deaminase. In some embodiments, the adenosine deaminase comprises a H8Y, D108N, N127S, D147Y, and E155V mutation in TadA reference sequence, or corresponding mutations in another adenosine deaminase. In some embodiments, the adenosine deaminase comprises a D108N, D147Y, and E155V mutation in TadA reference sequence, or corresponding mutations in another adenosine deaminase. In some embodiments, the adenosine deaminase comprises a H8Y, D108N, and N127S mutation in TadA reference sequence, or corresponding mutations in another adenosine deaminase. In some embodiments, the adenosine deaminase comprises a A106V, D108N, D147Y, and E155V mutation in TadA reference sequence, or corresponding mutations in another adenosine deaminase (e.g., ecTadA).

In some embodiments, the adenosine deaminase comprises one or more of S2X, H8X, I49X, L84X, H123X, N127X, I156X, and/or K160X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of S2A, H8Y, I49F, L84F, H123Y, N127S, I156F, and/or K160S mutation in TadA reference sequence, or one or more corresponding mutations in another adenosine deaminase (e.g., ecTadA).

In some embodiments, the adenosine deaminase comprises an L84X mutation adenosine deaminase, where X indicates any amino acid other than the corresponding amino acid in the wild-type adenosine deaminase. In some embodiments, the adenosine deaminase comprises an L84F mutation in TadA reference sequence, or a corresponding mutation in another adenosine deaminase (e.g., ecTadA).

In some embodiments, the adenosine deaminase comprises an H123X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H123Y mutation in TadA reference sequence, or a corresponding mutation in another adenosine deaminase.

In some embodiments, the adenosine deaminase comprises an I156X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an I156F mutation in TadA reference sequence, or a corresponding mutation in another adenosine deaminase.

In some embodiments, the adenosine deaminase comprises one, two, three, four, five, six, or seven mutations selected from the group consisting of L84X, A106X, D108X, H123X, D147X, E155X, and I156X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, or six mutations selected from the group consisting of S2X, I49X, A106X, D108X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, A106X, D108X, N127X, and K160X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and I156F in TadA reference sequence, or a corresponding mutation or mutations in another adenosine deaminase. In some embodiments, the adenosine deaminase comprises one, two, three, four, five, or six mutations selected from the group consisting of S2A, I49F, A106V, D108N, D147Y, and E155V in TadA reference sequence.

In some embodiments, the adenosine deaminase comprises one, two, three, four, or five mutations selected from the group consisting of H8Y, A106T, D108N, N127S, and K160S in TadA reference sequence, or a corresponding mutation or mutations in another adenosine deaminase.

In some embodiments, the adenosine deaminase comprises one or more of a E25X, R26X, R107X, A142X, and/or A143X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of E25M, E25D, E25A, E25R, E25V, E25S, E25Y, R26G, R26N, R26Q, R26C, R26L, R26K, R107P, R107K, R107A, R107N, R107W, R107H, R107S, A142N, A142D, A142G, A143D, A143G, A143E, A143L, A143W, A143M, A143S, A143Q, and/or A143R mutation in TadA reference sequence, or one or more corresponding mutations in another adenosine deaminase. In some embodiments, the adenosine deaminase comprises one or more of the mutations described herein corresponding to TadA reference sequence, or one or more corresponding mutations in another adenosine deaminase.

In some embodiments, the adenosine deaminase comprises an E25X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an E25M, E25D, E25A, E25R, E25V, E25S, or E25Y mutation in TadA reference sequence, or a corresponding mutation in another adenosine deaminase (e.g., ecTadA).

In some embodiments, the adenosine deaminase comprises an R26X mutation in TadA reference sequence, or a corresponding mutation in another adenosine deaminase, where X indicates any amino acid other than the corresponding amino acid in the wild-type adenosine deaminase. In some embodiments, the adenosine deaminase comprises R26G, R26N, R26Q, R26C, R26L, or R26K mutation in TadA reference sequence, or a corresponding mutation in another adenosine deaminase (e.g., ecTadA).

In some embodiments, the adenosine deaminase comprises an R107X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R107P, R107K, R107A, R107N, R107W, R107H, or R107S mutation in TadA reference sequence, or a corresponding mutation in another adenosine deaminase (e.g., ecTadA).

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A142N, A142D, A142G, mutation in TadA reference sequence, or a corresponding mutation in another adenosine deaminase (e.g., ecTadA).

In some embodiments, the adenosine deaminase comprises an A143X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A143D, A143G, A143E, A143L, A143W, A143M, A143S, A143Q, and/or A143R mutation in TadA reference sequence, or a corresponding mutation in another adenosine deaminase (e.g., ecTadA).

In some embodiments, the adenosine deaminase comprises one or more of a H36X, N37X, P48X, I49X, R51X, M70X, N72X, D77X, E134X, S146X, Q154X, K157X, and/or K161X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H36L, N37T, N37S, P48T, P48L, I49V, R51H, R51L, M70L, N72S, D77G, E134G, S146R, S146C, Q154H, K157N, and/or K161T mutation in TadA reference sequence, or one or more corresponding mutations in another adenosine deaminase (e.g., ecTadA).

In some embodiments, the adenosine deaminase comprises an H36X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H36L mutation in TadA reference sequence, or a corresponding mutation in another adenosine deaminase.

In some embodiments, the adenosine deaminase comprises an N37X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an N37T or N37S mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an P48T or P48L mutation in TadA reference sequence, or a corresponding mutation in another adenosine deaminase.

In some embodiments, the adenosine deaminase comprises an R51X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R51H or R51L mutation in TadA reference sequence, or a corresponding mutation in another adenosine deaminase.

In some embodiments, the adenosine deaminase comprises an S146X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an S146R or S146C mutation in TadA reference sequence, or a corresponding mutation in another adenosine deaminase.

In some embodiments, the adenosine deaminase comprises an K157X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a K157N mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a P48S, P48T, or P48A mutation in TadA reference sequence, or a corresponding mutation in another adenosine deaminase.

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a A142N mutation in TadA reference sequence, or a corresponding mutation in another adenosine deaminase.

In some embodiments, the adenosine deaminase comprises an W23X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a W23R or W23L mutation in TadA reference sequence, or a corresponding mutation in another adenosine deaminase.

In some embodiments, the adenosine deaminase comprises an R152X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a R152P or R52H mutation in TadA reference sequence, or a corresponding mutation in another adenosine deaminase.

In one embodiment, the adenosine deaminase may comprise the mutations H36L, R51L, L84F, A106V, D108N, H123Y, S146C, D147Y, E155V, I156F, and K157N. In some embodiments, the adenosine deaminase comprises the following combination of mutations relative to TadA reference sequence, where each mutation of a combination is separated by a “_” and each combination of mutations is between parentheses:
(A106V_D108N),
(R107C_D108N),
(H8Y_D108N_N127S_D147Y_Q154H),
(H8Y_D108N_N127S_D147Y_E155V),
(D108N_D147Y_E155V),
(H8Y_D108N_N127S),
(H8Y_D108N_N127S_D147Y_Q154H),
(A106V_D108N_D147Y_E155V),
(D108Q_D147Y_E155V),
(D108M_D147Y_E155V),
(D108L_D147Y_E155V),
(D108K_D147Y_E155V),
(D108I_D147Y_E155V),
(D108F_D147Y_E155V),
(A106V_D108N_D147Y),
(A106V_D108M_D147Y_E155V),
(E59A_A106V_D108N_D147Y_E155V),
(E59A cat dead_A106V_D108N_D147Y_E155V),
(L84F_A106V_D108N_H123Y_D147Y_E155V_I156Y),
(L84F_A106V_D108N_H123Y_D147Y_E155V_I156F),
(D103A_D104N),
(G22P_D103A_D104N),
(D103A_D104N_S138A),
(R26G_L84F_A106V_R107H_D108N_H123Y_A142N_A143D_D147Y_E155V_I156F),
(E25G_R26G_L84F_A106V_R107H_D108N_H123Y_A142N_A143D_D147Y_E155V_I156F)
(E25D_R26G_L84F_A106V_R107K_D108N_H123Y_A142N_A143G_D147Y_E155V_I156F),
(R26Q_L84F_A106V_D108N_H123Y_A142N_D147Y_E155V_I156F),
(E25M_R26G_L84F_A106V_R107P_D108N_H123Y_A142N_A143D_D147Y_E155V_I156F),
(R26C_L84F_A106V_R107H_D108N_H123Y_A142N_D147Y_E155V_I156F),
(L84F_A106V_D108N_H123Y_A142N_A143L_D147Y_E155V_I156F),
(R26G_L84F_A106V_D108N_H123Y_A142N_D147Y_E155V_I156F),
(E25A_R26G_L84F_A106V_R107N_D108N_H123Y_A142N_A143E_D147Y_E155V_I156F),
(R26G_L84F_A106V_R107H_D108N_H123Y_A142N_A143D_D147Y_E155V_I156F),
(A106V_D108N_A142N_D147Y_E155V),
(R26G_A106V_D108N_A142N_D147Y_E155V),
(E25D_R26G_A106V_R107K_D108N_A142N_A143G_D147Y_E155V),
(R26G_A106V_D108N_R107H_A142N_A143D_D147Y_E155V),
(E25D_R26G_A106V_D108N_A142N_D147Y_E155V),
(A106V_R107K_D108N_A142N_D147Y_E155V),
(A106V_D108N_A142N_A143G_D147Y_E155V),
(A106V_D108N_A142N_A143L_D147Y_E155V),
(H36L_R51L_L84F_A106V_D108N_H123Y_S146C_D147Y_E155V_I156F_K157N),
(N37T_P48T_M70L_L84F_A106V_D108N_H123Y_D147Y_I49V_E155V_I156F),
(N37S_L84F_A106V_D108N_H123Y_D147Y_E155V_I156F_K161T),
(H36L_L84F_A106V_D108N_H123Y_D147Y_Q154H_E155V_I156F),
(N72S_L84F_A106V_D108N_H123Y_S146R_D147Y_E155V_I156F),
(H36L_P48L_L84F_A106V_D108N_H123Y_E134G_D147Y_E155V_I156F),
(H36L_L84F_A106V_D108N_H123Y_D147Y_E155V_I156F_K157N)
(H36L_L84F_A106V_D108N_H123Y_S146C_D147Y_E155V_I156F),
(L84F_A106V_D108N_H123Y_S146R_D147Y_E155V_I156F_K161T),
(N37S_R51H_D77G_L84F_A106V_D108N_H123Y_D147Y_E155V_I156F),
(R51L_L84F_A106V_D108N_H123Y_D147Y_E155V_I156F_K157N),
(D24G_Q71R_L84F_H96L_A106V_D108N_H123Y_D147Y_E155V_I156F_K160E),
(H36L_G67V_L84F_A106V_D108N_H123Y_S146T_D147Y_E155V_I156F),
(Q71L_L84F_A106V_D108N_H123Y_L137M_A143E_D147Y_E155V_I156F),
(E25G_L84F_A106V_D108N_H123Y_D147Y_E155V_156F_Q159L),
(L84F_A91T_F104I_A106V_D108N_H123Y_D147Y_E155V_I156F),
(N72D_L84F_A106V_D108N_H123Y_G125A_D147Y_E155V_I156F),
(P48S_L84F_S97C_A106V_D108N_H123Y_D147Y_E155V_I156F),
(W23G_L84F_A106V_D108N_H123Y_D147Y_E155V_I156F),
(D24G_P48L_Q71R_L84F_A106V_D108N_H123Y_D147Y_E155V_I156F_Q159L),
(L84F_A106V_D108N_H123Y_A142N_D147Y_E155V_I156F),
(H36L_R51L_L84F_A106V_D108N_H123Y_A142N_S146C_D147Y_E155V_I156F_K157N),
(N37S_L84F_A106V_D108N_H123Y_A142N_D147Y_E155V_I156F_K161T),
(L84F_A106V_D108N_D147Y_E155V_I156F),
(R51L_L84F_A106V_D108N_H123Y_S146C_D147Y_E155V_I156F_K157N_K161T),
(L84F_A106V_D108N_H123Y_S146C_D147Y_E155V_I156F_K161T),
(L84F_A106V_D108N_H123Y_S146C_D147Y_E155V_I156F_K157N_K160E_K161T),
(L84F_A106V_D108N_H123Y_S146C_D147Y_E155V_I156F_K157N_K160E),
(R74Q_L84F_A106V_D108N_H123Y_D147Y_E155V_I156F),
(R74A_L84F_A106V_D108N_H123Y_D147Y_E155V_I156F),
(L84F_A106V_D108N_H123Y_D147Y_E155V_I156F),
(R74Q_L84F_A106V_D108N_H123Y_D147Y_E155V_I156F),
(L84F_R98Q_A106V_D108N_H123Y_D147Y_E155V_I156F),
(L84F_A106V_D108N_H123Y_R129Q_D147Y_E155V_1156F),
(P48S_L84F_A106V_D108N_H123Y_A142N_D147Y_E155V_I156F),
(P48S_A142N),
(P48T_I49V_L84F_A106V_D108N_H123Y_A142N_D147Y_E155V_I156F_L157N),
(P48T_I49V_A142N),
(H36L_P48S_R51L_L84F_A106V_D108N_H123Y_S146C_D147Y_E155V_I156F_K157N),
(H36L_P48S_R51L_L84F_A106V_D108N_H123Y_S146C_A142N_D147Y_E155V_I156F
(H36L_P48T_I49V_R51L_L84F_A106V_D108N_H123Y_S146C_D147Y_E155V_I156F_K157N),
(H36L_P48T_I49V_R51L_L84F_A106V_D108N_H123Y_A142N_S146C_D147Y_E155V_I156F_K157N),
(H36L_P48A_R51L_L84F_A106V_D108N_H123Y_S146C_D147Y_E155V_I156F_K157N),
(H36L_P48A_R51L_L84F_A106V_D108N_H123Y_A142N_S146C_D147Y_E155V_I156F_K157N),
(H36L_P48A_R51L_L84F_A106V_D108N_H123Y_S146C_A142N_D147Y_E155V_I156F_K157N),
(W23L_H36L_P48A_R51L_L84F_A106V_D108N_H123Y_S146C_D147Y_E155V_I156F_K157N),
(W23R_H36L_P48A_R51L_L84F_A106V_D108N_H123Y_S146C_D147Y_E155V_I156F_K157N),
(W23L_H36L_P48A_R51L_L84F_A106V_D108N_H123Y_S146R_D147Y_E155V_I156F_K161T),
(H36L_P48A_R51L_L84F_A106V_D108N_H123Y_S146C_D147Y_R152H_E155V_I156F_K157N),
(H36L_P48A_R51L_L84F_A106V_D108N_H123Y_S146C_D147Y_R152P_E155V_I156F_K157N),
(W23L_H36L_P48A_R51L_L84F_A106V_D108N_H123Y_S146C_D147Y_R152P_E155V_I156F_K157N),
(W23L_H36L_P48A_R51L_L84F_A106V_D108N_H123Y_A142A_S146C_D147Y_E155V_I 156F_K157N),
(W23L_H36L_P48A_R51L_L84F_A106V_D108N_H123Y_A142A_S146C_D147Y_R152P_E155V_I156F_K157N),
(W23L_H36L_P48A_R51L_L84F_A106V_D108N_H123Y_S146R_D147Y_E155V_I156F_K161T),
(W23R_H36L_P48A_R51L_L84F_A106V_D108N_H123Y_S146C_D147Y_R152P_E155V_I156F_K157N),
(H36L_P48A_R51L_L84F_A106V_D108N_H123Y_A142N_S146C_D147Y_R152P_E155 V_I156F_K157N).

In some embodiments, the TadA deaminase is TadA variant. In some embodiments, the TadA variant is TadA*7.10. In particular embodiments, the fusion proteins or complexes comprise a single TadA*7.10 domain (e.g., provided as a monomer). In other embodiments, the fusion protein comprises TadA*7.10 and TadA(wt), which are capable of forming heterodimers. In one embodiment, a fusion protein of the invention comprises a wild-type TadA linked to TadA*7.10, which is linked to Cas9 nickase.

In some embodiments, TadA*7.10 comprises at least one alteration. In some embodiments, the adenosine deaminase comprises an alteration in the following sequence:

TadA*7.10
(SEQ ID NO: 1)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVFNAQKKAQSSTD

In some embodiments, TadA*7.10 comprises an alteration at amino acid 82 and/or 166. In particular embodiments, TadA*7.10 comprises one or more of the following alterations: Y147T, Y147R, Q154S, Y123H, V82S, T166R, and/or Q154R. In other embodiments, a variant of TadA*7.10 comprises a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R.

In some embodiments, a variant of TadA*7.10 comprises one or more of alterations selected from the group of L36H, I76Y, V82G, Y147T, Y147D, F149Y, Q154S, N157K, and/or D167N. In some embodiments, a variant of TadA*7.10 comprises V82G, Y147T/D, Q154S, and one or more of L36H, I76Y, F149Y, N157K, and D167N. In other embodiments, a variant of TadA*7.10 comprises a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N.

In some embodiments, an adenosine deaminase variant (e.g., TadA*8) comprises a deletion. In some embodiments, an adenosine deaminase variant comprises a deletion of the C terminus. In particular embodiments, an adenosine deaminase variant comprises a deletion of the C terminus beginning at residue 149, 150, 151, 152, 153, 154, 155, 156, and 157, relative to TadA*7.10, the TadA reference sequence, or a corresponding mutation in another TadA.

In other embodiments, an adenosine deaminase variant (e.g., TadA*8) is a monomer comprising one or more of the following alterations: Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant (TadA*8) is a monomer comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, the adenosine deaminase variant is a homodimer comprising two adenosine deaminase domains (e.g., TadA*8) each having one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a homodimer comprising two adenosine deaminase domains (e.g., TadA*8) each having a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor of the disclosure comprising an adenosine deaminase variant (e.g., TadA*8) monomer comprising one or more of the following alterations: R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant (TadA*8) monomer comprises a combination of alterations selected from the group of: R26C+A109S+T111R+D119N+H122N+Y147D+F149Y+T1661+D167N; V88A+A109S+T111R+D119N+H122N+F149Y+T1661+D167N; R26C+A109S+T111R+D119N+H122N+F149Y+T1661+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In some embodiments, an adenosine deaminase variant (e.g., MSP828) is a monomer comprising one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant (e.g., MSP828) is a monomer comprising V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant (TadA variant) is a monomer comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In other embodiments, the adenosine deaminase variant is a heterodimer of a wild-type adenosine deaminase domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a wild-type adenosine deaminase domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor of the disclosure comprising an adenosine deaminase variant (e.g., TadA*8) homodimer comprising two adenosine deaminase domains (e.g., TadA*8) each having one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant is a homodimer comprising two adenosine deaminase domains (e.g., TadA*8) each having a combination of alterations selected from the group of: R26C+A109S+T111R+D119N+H122N+Y147D+F149Y+T166I+D167N; V88A+A109S+T111R+Dl19N+H122N+F149Y+T1661+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In some embodiments, an adenosine deaminase variant is a homodimer comprising two adenosine deaminase domains (e.g., TadA*7.10) each having one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a homodimer comprising two adenosine deaminase variant domains (e.g., MSP828) each having the following alterations V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant is a homodimer comprising two adenosine deaminase domains (e.g., TadA*7.10) each having a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor comprises a heterodimer of a wild-type adenosine deaminase domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a wild-type adenosine deaminase domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In other embodiments, the adenosine deaminase variant is a heterodimer of a wild-type adenosine deaminase domain and an adenosine deaminase variant domain (e.g., TadA*7.10) comprising one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a heterodimer comprising a wild-type adenosine deaminase domain and an adenosine deaminase variant domain (e.g., MSP828) having the following alterations V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant is a heterodimer of a wild-type adenosine deaminase domain and an adenosine deaminase variant domain (e.g., TadA*7.10) comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, an adenosine deaminase is a TadA*8. In one embodiment, an adenosine deaminase is a TadA*8 that comprises or consists essentially of the following sequence or a fragment thereof having adenosine deaminase activity: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCTFFRMPRQVFNAQKKAQSSTD (SEQ ID NO: 316)

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In some embodiments the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

In other embodiments, a base editor of the disclosure comprising an adenosine deaminase variant (e.g., TadA*8) monomer comprising one or more of the following alterations: R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant (TadA*8) monomer comprises a combination of alterations selected from the group of: R26C+A109S+Ti 11R+D119N+H122N+Y147D+F149Y+T1661+D167N; V88A+A109S+T111R+D119N+H122N+F149Y+T1661+D167N; R26C+A109S+T111R+D119N+H122N+F149Y+T1661+D167N; V88A+T111R+D119N+F149Y; and A109S+T111R+D119N+H122N+Y147D+F149Y+T1661+D167N, relative to TadA*7.10, the TadA reference sequence, or a corresponding mutation in another TadA.

In other embodiments, a base editor comprises a heterodimer of a wild-type adenosine deaminase domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a wild-type adenosine deaminase domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T1661+D167N; R26C+A109S+T111R+D119N+H122N+F149Y+T166I+D167N; V88A+T111R+D119N+F149Y; and A109S+T111R+D119N+H122N+Y147D+F149Y+T1661+D167N, relative to TadA*7.10, the TadA reference sequence, or a corresponding mutation in another TadA.

In other embodiments, a base editor comprises a heterodimer of a TadA*7.10 domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a TadA*7.10 domain and an adenosine deaminase variant domain (e.g., TadA*8) comprising a combination of alterations selected from the group of: R26C+A109S+Ti 11R+D119N+H122N+Y147D+F149Y+T1661+D167N; V88A+A109S+T111R+D119N+H122N+F149Y+T1661+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*7.10) comprising one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a heterodimer comprising a TadA*7.10 domain and an adenosine deaminase variant domain (e.g., MSP828) having the following alterations V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*7.10) comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In some embodiments, the TadA*8 is a variant as shown in Table 6. Table 6 shows certain amino acid position numbers in the TadA amino acid sequence and the amino acids present in those positions in the TadA-7.10 adenosine deaminase. Table 6 also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e.

TABLE 6
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 6.1. Table 6.1 shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 6.1
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In one embodiment, a fusion protein of the invention comprises a wild-type TadA is linked to an adenosine deaminase variant described herein (e.g., TadA*8), which is linked to Cas9 nickase. In particular embodiments, the fusion proteins comprise a single TadA*8 domain (e.g., provided as a monomer). In other embodiments, the fusion protein comprises TadA*8 and TadA(wt), which are capable of forming heterodimers.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

In particular embodiments, a TadA*8 comprises one or more mutations at any of the following positions shown in bold. In other embodiments, a TadA*8 comprises one or more mutations at any of the positions shown with underlining:

| (SEQ ID NO: 1) | |
|---|---|
| MSEVEFSHEY WMRHALTLAK RARDEREVPV GAVLVLNNRV IGEGWNRAIG | 50 |
| LHDPTAHAEI MALRQGGLVM QNYRLIDATL YVTFEPCVMC AGAMIHSRIG | 100 |
| RVVFGVRNAK TGAAGSLMDV LHYPGMNHRV EITEGILADE CAALLCYFFR | 150 |
| MPRQVFNAQK KAQSSTD | |

For example, the TadA*8 comprises alterations at amino acid position 82 and/or 166 (e.g., V82S, T166R) alone or in combination with any one or more of the following Y147T, Y147R, Q154S, Y123H, and/or Q154R, relative to TadA*7.10, the TadA reference sequence, or a corresponding mutation in another TadA. In particular embodiments, a combination of alterations is selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In one embodiment, a fusion protein of the invention comprises a wild-type TadA is linked to an adenosine deaminase variant described herein (e.g., TadA*8), which is linked to Cas9 nickase. In particular embodiments, the fusion proteins comprise a single TadA*8 domain (e.g., provided as a monomer). In other embodiments, the base editor comprises TadA*8 and TadA(wt), which are capable of forming heterodimers.

In particular embodiments, the fusion proteins comprise a single (e.g., provided as a monomer) TadA*8. In some embodiments, the TadA*8 is linked to a Cas9 nickase. In some embodiments, the fusion proteins of the invention comprise as a heterodimer of a wild-type TadA (TadA(wt)) linked to a TadA*8. In other embodiments, the fusion proteins of the invention comprise as a heterodimer of a TadA*7.10 linked to a TadA*8. In some embodiments, the base editor is ABE8 comprising a TadA*8 variant monomer. In some embodiments, the base editor is ABE8 comprising a heterodimer of a TadA*8 and a TadA(wt). In some embodiments, the base editor is ABE8 comprising a heterodimer of a TadA*8 and TadA*7.10. In some embodiments, the base editor is ABE8 comprising a heterodimer of a TadA*8. In some embodiments, the TadA*8 is selected from Table 6, 12, or 13. In some embodiments, the ABE8 is selected from Table 12, 13, or 15.

In some embodiments, the adenosine deaminase is a TadA*9 variant. In some embodiments, the adenosine deaminase is a TadA*9 variant selected from the variants described below and with reference to the following sequence (termed TadA*7.10):

(SEQ ID NO: 1)
MSEVEFSHEY WMRHALTLAK RARDEREVPV GAVLVLNNRV
IGEGWNRAIG LHDPTAHAEI MALRQGGLVM QNYRLIDATL
YVTFEPCVMC AGAMIHSRIG RVVFGVRNAK TGAAGSLMDV
LHYPGMNHRV EITEGILADE CAALLCYFFR MPRQVFNAQK
KAQSSTD

In some embodiments, an adenosine deaminase comprises one or more of the following alterations: R21N, R23H, E25F, N38G, L51W, P54C, M70V, Q71M, N72K, Y73S, V82T, M94V, P124W, T133K, D139L, D139M, C146R, and A158K. The one or more alternations are shown in the sequence above in underlining and bold font.

In some embodiments, an adenosine deaminase comprises one or more of the following combinations of alterations: V82S+Q154R+Y147R; V82S+Q154R+Y123H; V82S+Q154R+Y147R+Y123H; Q154R+Y147R+Y123H+I76Y+V82S; V82S+I76Y; V82S+Y147R; V82S+Y147R+Y123H; V82S+Q154R+Y123H; Q154R+Y147R+Y123H+I76Y; V82S+Y147R; V82S+Y147R+Y123H; V82S+Q154R+Y123H; V82S+Q154R+Y147R; V82S+Q154R+Y147R; Q154R+Y147R+Y123H+176Y; Q154R+Y147R+Y123H+I76Y+V82S; I76Y_V82S_Y123H_Y147R_Q154R; Y147R+Q154R+H123H; and V82S+Q154R.

In some embodiments, an adenosine deaminase comprises one or more of the following combinations of alterations: E25F+V82S+Y123H, T133K+Y147R+Q154R; E25F+V82S+Y123H+Y147R+Q154R; L51W+V82S+Y123H+C146R+Y147R+Q154R; Y73S+V82S+Y123H+Y147R+Q154R; P54C+V82S+Y123H+Y147R+Q154R; N38G+V82T+Y123H+Y147R+Q154R; N72K+V82S+Y123H+D139L+Y147R+Q154R; E25F+V82S+Y123H+D139M+Y147R+Q154R; Q71M+V82S+Y123H+Y147R+Q154R; E25F+V82S+Y123H+T133K+Y147R+Q154R; E25F+V82S+Y123H+Y147R+Q154R; V82S+Y123H+P124W+Y147R+Q154R; L51W+V82S+Y123H+C146R+Y147R+Q154R; P54C+V82S+Y123H+Y147R+Q154R; Y73S+V82S+Y123H+Y147R+Q154R; N38G+V82T+Y123H+Y147R+Q154R; R23H+V82S+Y123H+Y147R+Q154R; R21N+V82S+Y123H+Y147R+Q154R; V82S+Y123H+Y147R+Q154R+A158K; N72K+V82S+Y123H+D139L+Y147R+Q154R; E25F+V82S+Y123H+D139M+Y147R+Q154R; and M70V+V82S+M94V+Y123H+Y147R+Q154R

In some embodiments, an adenosine deaminase comprises one or more of the following combinations of alterations: Q71M+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D139M+Y147R+Q154R; Y73S+I76Y+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D139M+Y147R+Q154R; Y73 S+I76Y+V82S+Y123H+Y147R+Q154R; and V82S+Q154R; N72K_V82S+Y123H+Y147R+Q154R; Q71M_V82S+Y123H+Y147R+Q154R; V82S+Y123H+T133K+Y147R+Q154R; V82S+Y123H+T133K+Y147R+Q154R+A158K; M70V+Q71M+N72K+V82S+Y123H+Y147R+Q154R; N72K_V82S+Y123H+Y147R+Q154R; Q71M_V82S+Y123H+Y147R+Q154R; M70V+V82S+M94V+Y123H+Y147R+Q154R; V82S+Y123H+T133K+Y147R+Q154R; V82S+Y123H+T133K+Y147R+Q154R+A158K; and M70V+Q71M+N72K+V82S+Y123H+Y147R+Q154R. In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation, e.g., Y73S and Y72S and D139M and D138M.

In some embodiments, the TadA*9 variant comprises the alterations described in Table 16 as described herein. In some embodiments, the TadA*9 variant is a monomer. In some embodiments, the TadA*9 variant is a heterodimer with a wild-type TadA adenosine deaminase. In some embodiments, the TadA*9 variant is a heterodimer with another TadA variant (e.g., TadA*8, TadA*9). Additional details of TadA*9 adenosine deaminases are described in International PCT Application No. PCT/2020/049975, which is incorporated herein by reference for its entirety.

Any of the mutations provided herein and any additional mutations (e.g., based on the ecTadA amino acid sequence) can be introduced into any other adenosine deaminases. Any of the mutations provided herein can be made individually or in any combination in TadA reference sequence or another adenosine deaminase (e.g., ecTadA).

Details of A to G nucleobase editing proteins are described in International PCT Application No. PCT/2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

### C to T Editing

In some embodiments, a base editor disclosed herein comprises a fusion protein comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids. Typically, a cytidine deaminase catalyzes a C nucleobase that is positioned in the context of a single-stranded portion of a polynucleotide. In some embodiments, the entire polynucleotide comprising a target C can be single-stranded. For example, a cytidine deaminase incorporated into the base editor can deaminate a target C in a single-stranded RNA polynucleotide. In other embodiments, a base editor comprising a cytidine deaminase domain can act on a double-stranded polynucleotide, but the target C can be positioned in a portion of the polynucleotide which at the time of the deamination reaction is in a single-stranded state. For example, in embodiments where the NAGPB domain comprises a Cas9 domain, several nucleotides can be left unpaired during formation of the Cas9-gRNA-target DNA complex, resulting in formation of a Cas9 “R-loop complex”. These unpaired nucleotides can form a bubble of single-stranded DNA that can serve as a substrate for a single-strand specific nucleotide deaminase enzyme (e.g., cytidine deaminase).

In some embodiments, a cytidine deaminase of a base editor can comprise all or a portion of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase. In some embodiments, a deaminase incorporated into a base editor comprises all or a portion of an APOBEC1 deaminase. In some embodiments, a deaminase incorporated into a base editor comprises all or a portion of APOBEC2 deaminase. In some embodiments, a deaminase incorporated into a base editor comprises all or a portion of is an APOBEC3 deaminase. In some embodiments, a deaminase incorporated into a base editor comprises all or a portion of an APOBEC3A deaminase. In some embodiments, a deaminase incorporated into a base editor comprises all or a portion of APOBEC3B deaminase. In some embodiments, a deaminase incorporated into a base editor comprises all or a portion of APOBEC3C deaminase. In some embodiments, a deaminase incorporated into a base editor comprises all or a portion of APOBEC3D deaminase. In some embodiments, a deaminase incorporated into a base editor comprises all or a portion of APOBEC3E deaminase. In some embodiments, a deaminase incorporated into a base editor comprises all or a portion of APOBEC3F deaminase. In some embodiments, a deaminase incorporated into a base editor comprises all or a portion of APOBEC3G deaminase. In some embodiments, a deaminase incorporated into a base editor comprises all or a portion of APOBEC3H deaminase. In some embodiments, a deaminase incorporated into a base editor comprises all or a portion of APOBEC4 deaminase. In some embodiments, a deaminase incorporated into a base editor comprises all or a portion of activation-induced deaminase (AID). In some embodiments a deaminase incorporated into a base editor comprises all or a portion of cytidine deaminase 1 (CDA1). It should be appreciated that a base editor can comprise a deaminase from any suitable organism (e.g., a human or a rat). In some embodiments, a deaminase domain of a base editor is from a human, chimpanzee, gorilla, monkey, cow, dog, rat, or mouse. In some embodiments, the deaminase domain of the base editor is derived from rat (e.g., rat APOBEC1). In some embodiments, the deaminase domain of the base editor is human APOBEC1. In some embodiments, the deaminase domain of the base editor is pmCDA1.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins described herein, for example by making point mutations in the deaminase domain, affect the processivity of the fusion proteins (e.g., base editors). For example, mutations that reduce, but do not eliminate, the catalytic activity of a deaminase domain within a base editing fusion protein can make it less likely that the deaminase domain will catalyze the deamination of a residue adjacent to a target residue, thereby narrowing the deamination window. The ability to narrow the deamination window can prevent unwanted deamination of residues adjacent to specific target residues, which can decrease or prevent off-target effects.

For example, in some embodiments, an APOBEC deaminase incorporated into a base editor can comprise one or more mutations selected from the group consisting of H121X, H122X, R126X, R126X, R118X, W90X, W90X, and R132X of rAPOBEC1, or one or more corresponding mutations in another APOBEC deaminase, wherein X is any amino acid. In some embodiments, an APOBEC deaminase incorporated into a base editor can comprise one or more mutations selected from the group consisting of H121R, H122R, R126A, R126E, R118A, W90A, W90Y, and R132E of rAPOBEC1, or one or more corresponding mutations in another APOBEC deaminase.

In some embodiments, an APOBEC deaminase incorporated into a base editor can comprise one or more mutations selected from the group consisting of D316X, D317X, R320X, R320X, R313X, W285X, W285X, R326X of hAPOBEC3G, or one or more corresponding mutations in another APOBEC deaminase, wherein X is any amino acid. In some embodiments, any of the fusion proteins provided herein comprise an APOBEC deaminase comprising one or more mutations selected from the group consisting of D316R, D317R, R320A, R320E, R313A, W285A, W285Y, R326E of hAPOBEC3G, or one or more corresponding mutations in another APOBEC deaminase.

In some embodiments, an APOBEC deaminase incorporated into a base editor can comprise a H121R and a H122R mutation of rAPOBEC1, or one or more corresponding mutations in another APOBEC deaminase. In some embodiments an APOBEC deaminase incorporated into a base editor can comprise an APOBEC deaminase comprising a R126A mutation of rAPOBEC1, or one or more corresponding mutations in another APOBEC deaminase. In some embodiments, an APOBEC deaminase incorporated into a base editor can comprise an APOBEC deaminase comprising a R126E mutation of rAPOBEC1, or one or more corresponding mutations in another APOBEC deaminase. In some embodiments, an APOBEC deaminase incorporated into a base editor can comprise an APOBEC deaminase comprising a R118A mutation of rAPOBEC1, or one or more corresponding mutations in another APOBEC deaminase. In some embodiments, an APOBEC deaminase incorporated into a base editor can comprise an APOBEC deaminase comprising a W90A mutation of rAPOBEC1, or one or more corresponding mutations in another APOBEC deaminase. In some embodiments, an APOBEC deaminase incorporated into a base editor can comprise an APOBEC deaminase comprising a W90Y mutation of rAPOBEC1, or one or more corresponding mutations in another APOBEC deaminase. In some embodiments, an APOBEC deaminase incorporated into a base editor can comprise an APOBEC deaminase comprising a R132E mutation of rAPOBEC1, or one or more corresponding mutations in another APOBEC deaminase. In some embodiments an APOBEC deaminase incorporated into a base editor can comprise an APOBEC deaminase comprising a W90Y and a R126E mutation of rAPOBEC1, or one or more corresponding mutations in another APOBEC deaminase. In some embodiments, an APOBEC deaminase incorporated into a base editor can comprise an APOBEC deaminase comprising a R126E and a R132E mutation of rAPOBEC1, or one or more corresponding mutations in another APOBEC deaminase. In some embodiments, an APOBEC deaminase incorporated into a base editor can comprise an APOBEC deaminase comprising a W90Y and a R132E mutation of rAPOBEC1, or one or more corresponding mutations in another APOBEC deaminase. In some embodiments, an APOBEC deaminase incorporated into a base editor can comprise an APOBEC deaminase comprising a W90Y, R126E, and R132E mutation of rAPOBEC1, or one or more corresponding mutations in another APOBEC deaminase.

In some embodiments, an APOBEC deaminase incorporated into a base editor can comprise an APOBEC deaminase comprising a D316R and a D317R mutation of hAPOBEC3G, or one or more corresponding mutations in another APOBEC deaminase. In some embodiments, any of the fusion proteins provided herein comprise an APOBEC deaminase comprising a R320A mutation of hAPOBEC3G, or one or more corresponding mutations in another APOBEC deaminase. In some embodiments, an APOBEC deaminase incorporated into a base editor can comprise an APOBEC deaminase comprising a R320E mutation of hAPOBEC3G, or one or more corresponding mutations in another APOBEC deaminase. In some embodiments, an APOBEC deaminase incorporated into a base editor can comprise an APOBEC deaminase comprising a R313A mutation of hAPOBEC3G, or one or more corresponding mutations in another APOBEC deaminase. In some embodiments, an APOBEC deaminase incorporated into a base editor can comprise an APOBEC deaminase comprising a W285A mutation of hAPOBEC3G, or one or more corresponding mutations in another APOBEC deaminase. In some embodiments, an APOBEC deaminase incorporated into a base editor can comprise an APOBEC deaminase comprising a W285Y mutation of hAPOBEC3G, or one or more corresponding mutations in another APOBEC deaminase. In some embodiments, an APOBEC deaminase incorporated into a base editor can comprise an APOBEC deaminase comprising a R326E mutation of hAPOBEC3G, or one or more corresponding mutations in another APOBEC deaminase. In some embodiments, an APOBEC deaminase incorporated into a base editor can comprise an APOBEC deaminase comprising a W285Y and a R320E mutation of hAPOBEC3G, or one or more corresponding mutations in another APOBEC deaminase. In some embodiments, an APOBEC deaminase incorporated into a base editor can comprise an APOBEC deaminase comprising a R320E and a R326E mutation of hAPOBEC3G, or one or more corresponding mutations in another APOBEC deaminase. In some embodiments, an APOBEC deaminase incorporated into a base editor can comprise an APOBEC deaminase comprising a W285Y and a R326E mutation of hAPOBEC3G, or one or more corresponding mutations in another APOBEC deaminase. In some embodiments, an APOBEC deaminase incorporated into a base editor can comprise an APOBEC deaminase comprising a W285Y, R320E, and R326E mutation of hAPOBEC3G, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion of an APOBEC1 deaminase.

In some embodiments, the fusion proteins of the invention comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

The disclosure provides any deaminase domains with a certain percent identity plus any of the mutations or combinations thereof described herein. In some embodiments, the cytidine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the cytidine deaminases provided herein. In some embodiments, the cytidine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs). Representative CABEs include those described in PCT/US22/22050, the disclosure of which is incorporated herein by reference in its entirety for all purposes. In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, the adenosine deaminase variants of the invention comprise one or more alterations. In some embodiments, an adenosine deaminase variant of the invention is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some embodiments, the adenosine deaminase variant is a bacterial TadA deaminase variant (e.g., ecTadA). In some embodiments, the adenosine deaminase variant is a truncated TadA deaminase variant. In some embodiments, the adenosine deaminase variant is a fragment of a TadA deaminase variant. In some embodiments, an adenosine deaminase variant is a TadA*8 variant comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity of at least about 30%, 40%, 50% or more of the adenosine deaminase activity of a reference adenosine deaminase (e.g., TadA*8.20 or TadA*8.19). In some embodiments, an adenosine deaminase variant is a TadA*8.20 adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity of at least 30%, 40%, 50% of the activity of a reference adenosine deaminase (e.g., TadA*8.20 or TadA*8.19). In some embodiments, an adenosine deaminase variant as provided herein has an increased cytosine deaminase activity of at least about 10-fold, 20-fold, 30-fold, 40-fold, 50-fold, 60-fold, 70-fold, 80-fold, 90-fold, 100-fold or more relative to a reference adenosine deaminase (e.g., TadA*8.20 or TadA*8.19). In some embodiments, an adenosine deaminase variant as provided herein maintains a level of adenosine deaminase activity that is at least about 30%, 40%, 50%, 60%, 70% of the activity of a reference adenosine deaminase (e.g., TadA*8.20 or TadA*8.19). In some embodiments, the reference adenosine deaminase is TadA*8.20 or TadA*8.19.

In some embodiments, the adenosine deaminase variant is an adenosine deaminase comprising one or more alterations that increase cytosine deaminase activity and has an amino acid sequence that is at least about 70%, 75%, 80%, 85%, 90%, 95%, 98%, 99% or greater identity to SEQ ID NO: 1 below:

(SEQ ID NO: 1)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVFNAQKKAQSSTD

In some embodiments, the adenosine deaminase variant is an adenosine deaminase comprising the amino acid sequence of SEQ ID NO: 1 and one or more alterations that increase cytosine deaminase activity. In various embodiments, the one or more alterations of the invention do not include a R amino acid at position 48 of SEQ ID NO: 1, or a corresponding mutation in another adenosine deaminase.

In some embodiments, the adenosine deaminase variant is an adenosine deaminase comprising one or more alterations at an amino acid position selected from 2, 4, 6, 13, 27, 29, 100, 112, 114, 115, 162, and 165 of an amino acid sequence having at least about 70%, 75%, 80%, 85%, 90%, 95%, 98%, 99% or greater identity to SEQ ID NO: 1, or a corresponding alteration in another deaminase. In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase. In some embodiments, the two or more alterations are at an amino acid position selected from the group consisting of S2X, V4X, F6X, H8X, R13X, T17X, R23X, E27X, P29X, V30X, R47X, A48X, I49X, G67X, Y76X, D77X, S82X, F84X, H96X, G100X, R107X, G112X, A114X, G115X, M118X, D119X, H122X, N127X, A142X, A143X, R147X, Y147X, F149X, A158X, Q159X, A162X, S165X, T166X, and D167X of an amino acid sequence having at least about 70%, 75%, 80%, 85%, 90%, 95%, 98%, 99% or greater identity to SEQ ID NO: 1, or a corresponding alteration in another deaminase. In various embodiments, the alterations of the invention do not include a 48R mutation. In some embodiments, the adenosine deaminase variant is an adenosine deaminase comprising one or more alterations at an amino acid position selected from 2, 4, 6, 13, 27, 29, 100, 112, 114, 115, 162, and 165 of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, I49K, I49M, I49N, I49Q, I49T, G67W, I76H, I76R, I76W, Y76H, Y76R, Y76W, F84A, F84M, H96N, G100A, G100K, T111H, G112H, A114C, G115M, M118L, H122G, H122R, H122T, N1271, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising a combination of amino acid alterations selected from: E27H, Y76I, and F84M; E27H, I49K, and Y76I; E27S, I49K, Y76I, and A162N; E27K and D119N; E27H and Y76I; E27S, I49K, and G67W; E27S, 149K, and Y761; 149T, G67W, and H96N; E27C, Y76I, and D119N; R13G, E27Q, and N127K; T17A, E27H, 149M, Y761, and M118L; I49Q, Y76I, and G115M; S2H, I49K, Y76I, and G112H; R47S and R107C; H8Q, I49Q, and Y76I; T17A, A48G, S82T, and A142E; E27G and I49N; E27G, D77G, and S165P; E27S, I49K, and S82T; E27S, I49K, S82T, and G115M; E27S, V301, 149K, and S82T; E27S, V30F, I49K, S82T, F84A, R107C, and A142E; E27S, V30F, I49K, S82T, F84A, G112H, and A142E; E27S, V30F, I49K, S82T, F84A, G115M, and A142E; E27S, I49K, S82T, F84L, and R107C; E27S, I49K, S82T, F84L, and G112H; E27S, I49K, S82T, F84L, and G115M; E27S, I49K, S82T, F84L, R107C, and G112H; E27S, I49K, S82T, F84L, R107C, and G115M; E27S, I49K, S82T, F84L, R107C, and A142E; E27S, I49K, S82T, F84L, G112H, and A142E; E27S, I49K, S82T, F84L, G115M, and A142E; E27S, I49K, S82T, F84L, R107C, G112H, GI15M, and A142E; E27S, V301, 149K, S82T, and F84L; E27S, P29G, I49K, and S82T; E27S, P29G, I49K, S82T, and G115M; E27S, P29G, I49K, S82T, and A142E; P29G, I49K, and S82T; E27G, I49K, and S82T; E27G, I49K, S82T, R107C, and A142E; V4K, E27H, 149K, Y761, and A114C; V4K, E27H, I49K, Y76I, and D77G; F6Y, E27H, I49K, Y76I, G100A, and H122R; V4T, E27H, I49K, Y76R, and H122G; F6Y, E27H, I49K, and Y76W; F6Y, E27H, 149K, Y761, and D119N; F6Y, E27H, I49K, Y76I, and A114C; F6Y, E27H, I49K, and Y76I; V4K, E27H, 149K, Y76W, and H122T; F6G, E27H, I49K, Y76R, and G100K; F6H, E27H, I49K, Y76I, and H122N; E27H, I49K, Y76I, and A114C; F6Y, E27H, I49K, Y76H, H122R, and T1661; E27H, 149K, Y761, and N127P; R23Q, E27H, I49K, and Y76R; E27H, I49K, Y76H, H122R, and A158V; F6Y, E27H, I49K, Y76I, and T111H; E27H, I49K, Y76I, and R147H; E27H, I49K, Y76I, and A143E; F6Y, E27H, I49K, and Y76R; T17W, E27H, I49K, Y76H, H122G, and A158V; V4S, E27H, I49K, A143E, and Q159S; E27H, I49K, Y76I, N127I, and A162Q; T17A, E27H, and A48G; T17A, E27K, and A48G; T17A, E27S, and A48G; T17A, E27S, A48G, and I49K; T17A, E27G, and A48G; T17A, A48G, and I49N; T17A, E27G, A48G, and I49N; T17A, E27Q, and A48G; E27S, I49K, S82T, and R107C; E27S, I49K, S82T, and G112H; E27S, I49K, S82T, and A142E; E27S, I49K, S82T, R107C, and G112H; E27S, I49K, S82T, R107C, and G115M; E27S, 149K, S82T, R107C, and A142E; E27S, I49K, S82T, G112H, and A142E; E27S, 149K, S82T, G115M, and A142E; E27S, I49K, S82T, R107C, G112H, G115M, and A142E; E27S, V301, 149K, S82T, and R107C; E27S, V301, 149K, S82T, and G112H; E27S, V301, 149K, S82T, and G115M; E27S, V30I, I49K, S82T, and A142E; E27S, V301, 149K, S82T, R107C, and G112H; E27S, V301, I49K, S82T, R107C, and G115M; E27S, V301, 149K, S82T, R107C, and A142E; E27S, V30I, I49K, S82T, G112H, and A142E; E27S, V301, 149K, S82T, G115M, and A142E; E27S, V30I, I49K, S82T, R107C, G112H, G115M, and A142E; E27S, V30L, I49K, and S82T; E27S, V30L, I49K, S82T, and R107C; E27S, V30L, I49K, S82T, and G112H; E27S, V30L, 149K, S82T, and G115M; E27S, V30L, I49K, S82T, and A142E; E27S, V30L, I49K, S82T, R107C, and G112H; E27S, V30L, I49K, S82T, R107C, and G115M; E27S, V30L, I49K, S82T, R107C, and A142E; E27S, V30L, I49K, S82T, G112H, and A142E; E27S, V30L, I49K, S82T, G115M, and A142E; E27S, V30L, I49K, S82T, R107C, G112H, G115M, and A142E; E27S, V30F, 149K, S82T, and F84A; E27S, V30F, I49K, S82T, F84A, and R107C; E27S, V30F, I49K, S82T, F84A, and G112H; E27S, V30F, I49K, S82T, F84A, and G115M; E27S, V30F, I49K, S82T, F84A, and A142E; E27S, V30F, I49K, S82T, F84A, R107C, and G112H; E27S, V30F, I49K, S82T, F84A, R107C, and G115M; E27S, V30F, I49K, S82T, F84A, R107C, G112H, G115M, and A142E; E27S, I49K, S82T, and F84L; E27S, I49K, S82T, F84L, and A142E; E27S, V301, 149K, S82T, F84L, and R107C; E27S, V301, 149K, S82T, F84L, and G112H; E27S, V301, 149K, S82T, F84L, and G115M; E27S, V301, 149K, S82T, F84L, and A142E; E27S, V301, 149K, S82T, F84L, R107C, and G112H; E27S, V301, 149K, S82T, F84L, R107C, and G115M; E27S, V301, 149K, S82T, F84L, R107C, and A142E; E27S, V301, 149K, S82T, F84L, G112H, and A142E; E27S, V301, 149K, S82T, F84L, G115M, and A142E; E27S, V301, 149K, S82T, F84L, R107C, G112H, G115M, and A142E; E27S, P29G, I49K, S82T, and R107C; E27S, P29G, 149K, S82T, and G112H; E27S, P29G, I49K, S82T, R107C, and G112H; E27S, P29G, 149K, S82T, R107C, and G115M; E27S, P29G, I49K, S82T, R107C, and A142E; E27S, P29G, 149K, S82T, G112H, and A142E; E27S, P29G, I49K, S82T, G115M, and A142E; E27S, P29G, I49K, S82T, R107C, G112H, G115M, and A142E; P29G, I49K, S82T, and R107C; P29G, 149K, S82T, and G112H; P29G, I49K, S82T, and G115M; P29G, I49K, S82T, and A142E; P29G, I49K, S82T, R107C, and G112H; P29G, I49K, S82T, R107C, and G115M; P29G, I49K, S82T, R107C, and A142E; P29G, I49K, S82T, G112H, and A142E; P29G, I49K, S82T, G115M, and A142E; P29G, I49K, S82T, R107C, G112H, G115M, and A142E; P29K, I49K, and S82T; P29K, 149K, S82T, and R107C; P29K, I49K, S82T, and G112H; P29K, I49K, S82T, and G115M; P29K, I49K, S82T, and A142E; P29K, I49K, S82T, R107C, and G112H; P29K, I49K, S82T, R107C, and G115M; P29K, I49K, S82T, R107C, and A142E; P29K, I49K, S82T, G112H, and A142E; P29K, I49K, S82T, G115M, and A142E; P29K, I49K, S82T, R107C, G112H, G115M, and A142E; P29K, V301, 149K, and S82T; P29K, V301, 149K, S82T, and R107C; P29K, V301, 149K, S82T, and G112H; P29K, V301, 149K, S82T, and G115M; P29K, V301, 149K, S82T, and A142E; P29K, V301, 149K, S82T, R107C, and G112H; P29K, V301, 149K, S82T, R107C, and G115M; P29K, V301, 149K, S82T, R107C, and A142E; P29K, V301, 149K, S82T, G112H, and A142E; P29K, V301, 149K, S82T, G115M, and A142E; P29K, V301, 149K, S82T, R107C, G112H, G115M, and A142E; P29K, I49K, S82T, and F84L; P29K, I49K, S82T, F84L, and R107C; P29K, I49K, S82T, F84L, and G112H; P29K, I49K, S82T, F84L, and G115M; P29K, I49K, S82T, F84L, and A142E; P29K, I49K, S82T, F84L, R107C, and G112H; P29K, I49K, S82T, F84L, R107C, and G115M; P29K, I49K, S82T, F84L, R107C, and A142E; P29K, I49K, S82T, F84L, G112H, and A142E; P29K, I49K, S82T, F84L, G115M, and A142E; P29K, I49K, S82T, F84L, R107C, G112H, G115M, and A142E; P29K, V301, 149K, S82T, and F84L; P29K, V301, 149K, S82T, F84L, and R107C; P29K, V301, 149K, S82T, F84L, and G112H; P29K, V301, 149K, S82T, F84L, and G115M; P29K, V301, 149K, S82T, F84L, and A142E; P29K, V301, 149K, S82T, F84L, R107C, and G112H; P29K, V301, 149K, S82T, F84L, R107C, and G115M; P29K, V301, I49K, S82T, F84L, R107C, and A142E; P29K, V301, 149K, S82T, F84L, G112H, and A142E; P29K, V301, 149K, S82T, F84L, G115M, and A142E; P29K, V301, 149K, S82T, F84L, R107C, G112H, G115M, and A142E; E27G, I49K, S82T, and R107C; E27G, 149K, S82T, and G112H; E27G, I49K, S82T, and G115M; E27G, I49K, S82T, and A142E; E27G, I49K, S82T, R107C, and G112H; E27G, I49K, S82T, R107C, and G115M; E27G, I49K, S82T, G112H, and A142E; E27G, I49K, S82T, G115M, and A142E; E27G, I49K, S82T, R107C, G112H, G115M, and A142E; E27H, I49K, and S82T; E27H, I49K, S82T, and R107C; E27H, 149K, S82T, and G112H; E27H, I49K, S82T, and G115M; E27H, I49K, S82T, and A142E; E27H, I49K, S82T, R107C, and G112H; E27H, I49K, S82T, R107C, and G115M; E27H, 149K, S82T, R107C, and A142E; E27H, I49K, S82T, G112H, and A142E; E27H, I49K, S82T, G115M, and A142E; E27H, I49K, S82T, R107C, G112H, G115M, and A142E; E27S, and S82T; E27S, S82T, and R107C; E27S, S82T, and G112H; E27S, S82T, and G115M; E27S, S82T, and A142E; E27S, S82T, R107C, and G112H; E27S, S82T, R107C, and G115M; E27S, S82T, R107C, and A142E; E27S, S82T, G112H, and A142E; E27S, S82T, G115M, and A142E; E27S, S82T, R107C, G112H, G115M, and A142E; P29A, and S82T; P29A, S82T, and R107C; P29A, S82T, and G112H; P29A, S82T, and G115M; P29A, S82T, and A142E; P29A, S82T, R107C, and G112H; P29A, S82T, R107C, and G115M; P29A, S82T, R107C, and A142E; P29A, S82T, G112H, and A142E; P29A, S82T, G115M, and A142E; P29A, S82T, R107C, G112H, G115M, and A142E; E27S, V30I, and S82T; E27S, V30I, S82T, and R107C; E27S, V30I, S82T, and G112H; E27S, V30I, S82T, and G115M; E27S, V30I, S82T, and A142E; E27S, V30I, S82T, R107C, and G112H; E27S, V30I, S82T, R107C, and G115M; E27S, V30I, S82T, R107C, and A142E; E27S, V30I, S82T, G112H, and A142E; E27S, V30I, S82T, G115M, and A142E; E27S, V30I, S82T, R107C, G112H, G115M, and A142E; P29A, V30I, S82T, and F84L; P29A, V30I, S82T, F84L, and R107C; P29A, V30I, S82T, F84L, and G112H; P29A, V30I, S82T, F84L, and G115M; P29A, V30I, S82T, F84L, and A142E; P29A, V30I, S82T, F84L, R107C, and G112H; P29A, V30I, S82T, F84L, R107C, and G115M; P29A, V30I, S82T, F84L, R107C, and A142E; P29A, V30I, S82T, F84L, G112H, and A142E; P29A, V30I, S82T, F84L, G115M, and A142E; P29A, V30I, S82T, F84L, R107C, G112H, G115M, and A142E; E27S, P29A, V30L, I49K, S82T, F84L, R107C, G112H, G115M, and A142E; V4K, and A114C; V4K, and D77G; F6Y, G100A, and H122R; V4T, I76R, and H122G; F6Y, and I76W; F6Y, and D119N; F6Y, and A114C; V4K, I76W, and H122T; F6G, I76R, and G100K; F6H, and H122N; F6Y, 176H, H122R, and T1661; R23Q, and I76R; I76H, H122R, and A158V; F6Y, and T111H; T111H, H122G, and A162C; F6Y, and I76R; T17W, I76H, H122G, and A158V; V4S, I76Y, A143E, and Q159S; N127I, A162Q; E27H, Y76I, F84M, and F149Y; E27H, I49K, Y76I, and F149Y; T17A, E27H, I49M, Y76I, M118L, and F149Y; T17A, A48G, S82T, A142E, and F149Y; E27G, and F149Y; E27G, I49N, and F149Y; E27H, Y76I, F84M, Y147D, F149Y, T1661, and D167N; E27H, I49K, Y76I, Y147D, F149Y, T1661, D167N; T17A, E27H, I49M, Y76I, M118L, Y147D, F149Y, T1661, and D167N; T17A, A48G, S82T, A142E, Y147D, F149Y, T166I, and D167N; E27G, Y147D, F149Y, T1661, and D167N; E27G, I49N, Y147D, F149Y, T1661, and D167N; F6Y, E27H, I49K, Y76W, S82T, R107C, G112H, G115M, and A142E; F6Y, E27H, I49K, D77G, S82T, R107C, G112H, G115M, and A142E; F6Y, E27H, I49K, S82T, R107C, G112H, A114C, G115M, and A142E; F6Y, E27H, I49K, S82T, R107C, G112H, G115M, D119N, and A142E; F6Y, E27H, I49K, S82T, R107C, G112H, G115M, H122G, and A142E; F6Y, E27H, I49K, S82T, R107C, G112H, G115M, N127P, and A142E; F6Y, E27H, I49K, S82T, R107C, G112H, G115M, A142E, and A143E; F6Y, E27H, I49K, S82T, R107C, G112H, G115M, and A143E; F6Y, E27H, I49K, Y76W, D77G, S82T, R107C, G112H, G115M, and A142E; F6Y, E27H, I49K, Y76W, S82T, R107C, G112H, A114C, G115M, and A142E; F6Y, E27H, I49K, Y76W, S82T, R107C, G112H, G115M, D119N, and A142E; F6Y, E27H, I49K, Y76W, S82T, R107C, G112H, G115M, H122G, and A142E; F6Y, E27H, I49K, Y76W, S82T, R107C, G112H, G115M, N127P, and A142E; F6Y, E27H, I49K, Y76W, S82T, R107C, G112H, G115M, A142E, and A143E; F6Y, E27H, I49K, Y76W, S82T, R107C, G112H, G115M, and A143E; F6Y, E27H, I49K, D77G, S82T, R107C, G112H, A114C, G115M, and A142E; F6Y, E27H, I49K, D77G, S82T, R107C, G112H, G115M, D119N, and A142E; F6Y, E27H, I49K, D77G, S82T, R107C, G112H, G115M, H122G, and A142E; F6Y, E27H, I49K, D77G, S82T, R107C, G112H, G115M, N127P, and A142E; F6Y, E27H, I49K, D77G, S82T, R107C, G112H, G115M, A142E, and A143E; F6Y, E27H, I49K, D77G, S82T, R107C, G112H, G115M, and A143E; F6Y, E27H, I49K, S82T, R107C, G112H, A114C, G115M, D119N, and A142E; F6Y, E27H, I49K, S82T, R107C, G112H, A114C, G115M, H122G, and A142E; F6Y, E27H, I49K, S82T, R107C, G112H, A114C, G115M, N127P, and A142E; F6Y, E27H, I49K, S82T, R107C, G112H, G115M, D119N, H122G, and A142E; F6Y, E27H, I49K, S82T, R107C, G112H, G115M, D119N, N127P, and A142E; F6Y, E27H, I49K, S82T, R107C, G112H, G115M, H122G, N127P, and A142E; F6Y, E27H, I49K, Y76W, D77G, S82T, R107C, G112H, A114C, G115M, and A142E; F6Y, E27H, I49K, Y76W, D77G, S82T, R107C, G112H, G115M, D119N, and A142E; F6Y, E27H, I49K, Y76W, D77G, S82T, R107C, G112H, G115M, H122G, and A142E; F6Y, E27H, I49K, Y76W, D77G, S82T, R107C, G112H, G115M, N127P, and A142E; F6Y, E27H, I49K, Y76W, D77G, S82T, R107C, G112H, G115M, A142E, and A143E; F6Y, E27H, I49K, Y76W, D77G, S82T, R107C, G112H, G115M, and A143E; F6Y, E27H, I49K, Y76W, S82T, R107C, G112H, A114C, G115M, D119N, and A142E; F6Y, E27H, I49K, Y76W, S82T, R107C, G112H, A114C, G115M, H122G, and A142E; F6Y, E27H, I49K, Y76W, S82T, R107C, G112H, A114C, G115M, N127P, and A142E; F6Y, E27H, I49K, Y76W, S82T, R107C, G112H, A114C, G115M, A142E, and A143E; F6Y, E27H, I49K, Y76W, S82T, R107C, G112H, A114C, G115M, and A143E; F6Y, E27H, I49K, Y76W, S82T, R107C, G112H, G115M, D119N, H122G, and A142E; F6Y, E27H, I49K, Y76W, S82T, R107C, G112H, G115M, D119N, N127P, and A142E; F6Y, E27H, I49K, Y76W, S82T, R107C, G112H, G115M, H122G, N127P, and A142E; F6Y, E27H, I49K, Y76W, S82T, R107C, G112H, A114C, G115M, D119N, H122G, and A142E; F6Y, E27H, I49K, Y76W, S82T, R107C, G112H, A114C, G115M, D119N, N127P, and A142E; F6Y, E27H, I49K, Y76W, S82T, R107C, G112H, A114C, G115M, H122G, N127P, and A142E; F6Y, E27H, I49K, Y76W, D77G, S82T, R107C, G112H, A114C, G115M, D119N, and A142E; F6Y, E27H, I49K, Y76W, D77G, S82T, R107C, G112H, A114C, G115M, H122G, and A142E; F6Y, E27H, I49K, Y76W, D77G, S82T, R107C, G112H, A114C, G115M, N127P, and A142E; F6Y, E27H, I49K, Y76W, D77G, S82T, R107C, G112H, A114C, G115M, A142E, and A143E; F6Y, E27H, I49K, Y76W, D77G, S82T, R107C, G112H, A114C, G115M, and A143E; F6Y, E27H, I49K, Y76W, D77G, S82T, R107C, G112H, A114C, G115M, D119N, H122G,and A142E; F6Y, E27H, I49K, Y76W, D77G, S82T, R107C, G112H, A114C, G115M, D119N, N127P, and A142E; F6Y, E27H, I49K, Y76W, D77G, S82T, R107C, G112H, A114C, G115M, H122G, N127P, and A142E; F6Y, E27H, I49K, Y76W, S82T, R107C, G112H, A114C, G115M, D119N, H122G, N127P, A142E, and A143E; F6Y, E27H, I49K, Y76W, S82T, R107C, G112H, A114C, G115M, D119N, H122G, N127P, and A143E; F6Y, E27H, I49K, Y76W, D77G, S82T, R107C, G112H, A114C, G115M, D119N, H122G, N127P, A142E, and A143E; and F6Y, E27H, I49K, Y76W, D77G, S82T, R107C, G112H, A114C, G115M, D119N, H122G, N127P, and A143E; of an amino acid sequence having at least about 70%, 75%, 80%, 85%, 90%, 95%, 98%, 99% or greater identity to SEQ ID NO: 1, or a corresponding combination of alterations in another deaminase.

In some embodiments, the adenosine deaminase variant is an adenosine deaminase comprising an amino acid alteration or combination of amino acid alterations selected from those listed in any of Tables 6.2A-6.2F.

The residue identity of exemplary adenosine deaminase variants that are capable of deaminating adenine and/or cytidine in a target polynucleotide (e.g., DNA) is provided in Tables 6.2A-6.2F below. Further examples of adenosine deaminse variants include the following variants of 1.17 (see Table 6.2A): 1.17+E27H; 1.17+E27K; 1.17+E27S; 1.17+E27S+I49K; 1.17+E27G; 1.17+149N; 1.17+E27G+I49N; and 1.17+E27Q. In some embodiments, any of the amino acid alterations provided herein are substituted with a conservative amino acid. Additional mutations known in the art can be further added to any of the adenosine deaminase variants provided herein.

In some embodiments, base editing is carried out to induce therapeutic changes in the genome of a cell of a subject (e.g., human). Cells are collected from a subject and contacted with one or more guide RNAs and a nucleobase editor polypeptide comprising a nucleic acid programmable DNA binding protein (napDNAbp) (e.g., Cas9) and an adenosine deaminase variant capable of deaminating both adenine and cytosine in a target polynucleotide (e.g., DNA). In some embodiments, cells are contacted with one or more guide RNAs and a fusion protein comprising a nucleic acid programmable DNA binding protein (napDNAbp) (e.g., Cas9) and an adenosine deaminase variant capable of deaminating both adenine and cytosine in a target polynucleotide (e.g., DNA). In some embodiments, the napDNAbp is a Cas9.

In some embodiments, cells are contacted with a multi-molecular complex. In some embodiments, cells are contacted with a base editor system as provided herein. In some embodiments, the base editor systems as provided herein comprise an adenosine base editor (ABE) variant (e.g., a CABE). In some embodiments, the CABE variant is an ABE8 variant. In some embodiments, the ABE8 variant is an ABE8.20 variant. In some embodiments, CABEs as provided herein have both A to G and C to T base editing activity. Therefore, multiple edits may be introduced into the genome of a subject (e.g., human). The ability to target both A to G and C to T base editing activity allows for diverse targeting of polynucleotides in the genome in a subject to treat a genetic disease or disorder.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6.2A
Adenosine Deaminase Variants (CABE-1; T_{AD}AC-1). Mutations are indicated with reference to TadA*8.20.

| | location in structure | | | | | | | | | | | | | |
|---|---|---|---|---|---|---|---|---|---|---|---|---|---|---|
| | | | | | near | near | near | near | | | | | | |
| | | surface | surface | surface | active | active | active | active | | | | | | |
| | N/A | h1 | h1 | h1 | site | site | site | site | | surface | | | | |
| | Amino Acid No. (*START Met is AA#1) | | | | | | | | | | | | | |
| | 2 | 8 | 13 | 17 | 27 | 47 | 48 | 49 | 67 | 76 | 77 | | | |
| | TadA*8.20 | | | | | | | | | | | | | |
| | S | H | R | T | E | R | A | I | G | Y | D | | | |
| | TadA*8.19 | | | | | | | | | | | Plasmid #s | | |
| | | | | | | | | | | I | | without ugi | with ugi | bacterial |
| 1.1 | | | | | H | | | | | I | | pDKL-11 | pDKL-23 | pDKL-35 |
| 1.2 | | | | | H | | | K | | I | | pDKL-12 | pDKL-24 | pDKL-36 |
| 1.3 | | | | | S | | | K | | I | | pDKL-13 | pDKL-25 | pDKL-37 |
| 1.4 | | | | | S | | | K | | I | | pDKL-14 | pDKL-26 | pDKL-38 |
| 1.5 | | | | | K | | | | | | | PDKL-15 | pDKL-27 | pDKL-39 |
| 1.6 | | | | | K | | | | | | | pDKL-16 | pDKL-28 | pDKL-40 |
| 1.7 | | | | | H | | | | | I | | pDKL-17 | pDKL-29 | pDKL-41 |
| 1.8 | | | | | S | | | K | W | | | pDKL-18 | pDKL-30 | pDKL-42 |
| 1.9 | | | | | | | | T | W | | | pDKL-19 | pDKL-31 | pDKL-43 |
| 1.10 | | | | | C | | | | | I | | pDKL-20 | pDKL-32 | pDKL-44 |
| 1.11 | | | G | | Q | | | | | | | pDKL-21 | pDKL-33 | pDKL-45 |
| 1.12 | | | | A | H | | | M | | I | | pDKL-22 | pDKL-34 | pDKL-46 |
| 1.13 | | | | | | | | Q | | I | | pDKL-47 | pDKL-55 | pDKL-63 |
| 1.14 | H | | | | | | | K | | I | | pDKL-48 | pDKL-56 | pDKL-64 |
| 1.15 | | | | | | S | | | | | | pDKL-49 | pDKL-57 | pDKL-65 |
| 1.16 | | Q | | | | | | Q | | I | | pDKL-50 | pDKL-58 | pDKL-66 |
| 1.17 | | | | A | | | G | | | | | pDKL-51 | pDKL-59 | pDKL-67 |
| 1.18 | | | | | G | | | | | | | pDKL-52 | pDKL-60 | pDKL-68 |
| 1.19 | | | | | G | | | N | | | | pDKL-53 | pDKL-61 | pDKL-69 |
| 1.20 | | | | | G | | | | | | G | pDKL-54 | pDKL-62 | pDKL-70 |

| | location in structure | | | | | | | | | | | | | | |
|---|---|---|---|---|---|---|---|---|---|---|---|---|---|---|---|
| | | near | | near | | | | | | | | | | | |
| | | active | | active | | | | | | | | | | | |
| | internal | site | | site | surface | | surface | surface | surface | | surface | | | | |
| | Amino Acid No. (*START Met is AA#1) | | | | | | | | | | | | | | |
| | 82 | 84 | 96 | 107 | 112 | 115 | 118 | 119 | 127 | 142 | 162 | 165 | | | |
| | TadA*8.20 | | | | | | | | | | | | | | |
| | S | F | H | R | G | G | M | D | N | A | A | S | Plasmid #s | | |
| | TadA*8.19 | | | | | | | | | | | | without ugi | with ugi | bacterial |
| 1.1 | | M | | | | | | | | | | | pDKL-11 | pDKL-23 | pDKL-35 |
| 1.2 | | | | | | | | | | | | | pDKL-12 | pDKL-24 | pDKL-36 |
| 1.3 | | | | | | | | | | | | | pDKL-13 | pDKL-25 | pDKL-37 |
| 1.4 | | | | | | | | | | | N | | pDKL-14 | pDKL-26 | pDKL-38 |
| 1.5 | | | | | | | | | | | | | pDKL-15 | pDKL-27 | pDKL-39 |
| 1.6 | | | | | | | | N | | | | | pDKL-16 | pDKL-28 | pDKL-40 |
| 1.7 | | | | | | | | | | | | | pDKL-17 | pDKL-29 | pDKL-41 |
| 1.8 | | | | | | | | | | | | | pDKL-18 | pDKL-30 | pDKL-42 |
| 1.9 | | | N | | | | | | | | | | pDKL-19 | pDKL-31 | pDKL-43 |
| 1.10 | | | | | | | | N | | | | | pDKL-20 | pDKL-32 | pDKL-44 |
| 1.11 | | | | | | | | | K | | | | pDKL-21 | pDKL-33 | pDKL-45 |
| 1.12 | | | | | | | L | | | | | | pDKL-22 | pDKL-34 | pDKL-46 |
| 1.13 | | | | | | M | | | | | | | pDKL-47 | pDKL-55 | pDKL-63 |
| 1.14 | | | | | H | | | | | | | | pDKL-48 | pDKL-56 | pDKL-64 |
| 1.15 | | | | C | | | | | | | | | pDKL-49 | pDKL-57 | pDKL-65 |
| 1.16 | | | | | | | | | | | | | pDKL-50 | pDKL-58 | pDKL-66 |
| 1.17 | T | | | | | | | | | E | | | pDKL-51 | pDKL-59 | pDKL-67 |
| 1.18 | | | | | | | | | | | | | pDKL-52 | pDKL-60 | pDKL-68 |
| 1.19 | | | | | | | | | | | | | pDKL-53 | pDKL-61 | pDKL-69 |
| 1.20 | | | | | | | | | | | | P | pDKL-54 | pDKL-62 | pDKL-70 |

TABLE 6.2B
Rationally Designed Candidate Editors (CABE-2s; T_{AD}AC-
2s). Mutations are indicated with reference to TadA*8.20.

| | Position No. | | | | | | | | | |
|---|---|---|---|---|---|---|---|---|---|---|
| | 27 | 29 | 30 | 49 | 82 | 84 | 107 | 112 | 115 | 142 |
| | TadA*8.20 | | | | | | | | | |
| | E | P | V | I | S | F | R | G | G | A |
| | Alterations Evaluated | | | | | | | | | |
| | G/S/H | G/A/K | I/L/F | K | T | L/A | C | H | M | E |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | C | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | E |
| S1.54 | S | | | K | T | L | | | M | E |
| S1.55 | S | | | K | T | L | C | H | M | E |
| S1.56 | S | | I | K | T | L | | | | |
| S1.57 | S | | I | K | T | L | C | | | |
| S1.58 | S | | I | K | T | L | | H | | |
| S1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | I | K | T | L | | H | | E |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | E |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | E |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | C | | | E |
| S1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | C | | M | |
| S1.173 | | A | | | T | | C | | | E |
| S1.174 | | A | | | T | | | H | | E |
| S1.175 | | A | | | T | | | | M | E |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| S1.180 | S | | I | | T | | | | M | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 6.2C
Candidate base editors (CABE-2e; T_{AD}AC-2e). Mutations are indicated with reference to variant 1.2 (Table 6.2A).

| Variant | Alternative Variant | Residue identity (START Met is amino acid #1) | | | | | | | | | | | | | | | | | |
|---|---|---|---|---|---|---|---|---|---|---|---|---|---|---|---|---|---|---|---|
| Name | Names | 4 | 6 | 17 | 23 | 76 | 77 | 100 | 111 | 114 | 119 | 122 | 127 | 143 | 147 | 158 | 159 | 162 | 166 |
| Reference | 1.2 (see Table 1A) | V | F | T | R | I | D | G | T | A | D | H | N | A | R | A | Q | A | T |
| TadAC2.1 | pDKL-135; 2.1 | K | | | | | | | | C | | | | | | | | | |
| TadAC2.2 | pDKL-136; 2.2 | K | | | | | G | | | | | | | | | | | | |
| TadAC2.3 | pDKL-137; 2.3 | | Y | | | | | A | | | | R | | | | | | | |
| TadAC2.4 | pDKL-138; 2.4 | T | | | | R | | | | | | G | | | | | | | |
| TadAC2.5 | pDKL-139; 2.5 | | Y | | | W | | | | | | | | | | | | | |
| TadAC2.6 | pDKL-140; 2.6 | | Y | | | | | | | | N | | | | | | | | |
| TadAC2.7 | pDKL-141; 2.7 | | Y | | | | | | | C | | | | | | | | | |
| TadAC2.8 | pDKL-142; 2.8 | | Y | | | | | | | | | | | | | | | | |
| TadAC2.9 | pDKL-143; 2.9 | K | | | | W | | | | | | T | | | | | | | |
| TadAC2.10 | pDKL-144; 2.10 | | G | | | R | | K | | | | | | | | | | | |
| TadAC2.11 | pDKL-145; 2.11 | | H | | | | | | | | | N | | | | | | | |
| TadAC2.12 | pDKL-146; 2.12 | | | | | | | | | C | | | | | | | | | |
| TadAC2.13 | pDKL-147; 2.13 | | Y | | | H | | | | | | R | | | | | | | I |
| TadAC2.14 | pDKL-148; 2.14 | | | | | | | | | | | | P | | | | | | |
| TadAC2.15 | pDKL-149; 2.15 | | | | Q | R | | | | | | | | | | | | | |
| TadAC2.16 | pDKL-150; 2.16 | | | | | H | | | | | | R | | | | V | | | |
| TadAC2.17 | pDKL-151; 2.17 | | Y | | | | | | H | | | | | | | | | | |
| TadAC2.18 | pDKL-152; 2.18 | | | | | W | | | | | | | | | | | | | |
| TadAC2.19 | pDKL-153; 2.19 | | | | | | | | H | | | G | | | | | | C | |
| TadAC2.20 | pDKL-154; 2.20 | | | | | | | | | | | | | E | | | | | |
| TadAC2.21 | pDKL-155; 2.21 | | Y | | | R | | | | | | | | | | | | | |
| TadAC2.22 | pDKL-156; 2.22 | | | W | | H | | | | | | G | | | | V | | | |
| TadAC2.23 | pDKL-157; 2.23 | S | | | | Y | | | | | | | | E | | | S | | |
| TadAC2.24 | pDKL-158; 2.24 | | | | | | | | | | | | I | | | | | Q | |

TABLE 6.2D
Rationally Designed Candidate Editors (CBE-T1; T_{AD}C-
1). Mutations are indicated with reference to ABE8.20m.

| | AA Positions | | | | | | | | | | | | | | |
|---|---|---|---|---|---|---|---|---|---|---|---|---|---|---|---|
| | 6 | 27 | 49 | 76 | 77 | 82 | 107 | 112 | 114 | 115 | 119 | 122 | 127 | 142 | 143 |
| | TadA8.20 | | | | | | | | | | | | | | |
| | F | E | I | Y | D | S | R | G | A | G | D | H | N | A | A |
| S1.154 | F | H | K | Y | D | T | C | H | | M | | | | E | |
| Alterations | Y | | | W | G | | | | C | | N | G | P | | E |
| S2.1 | Y | H | K | W | | T | C | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | G | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | G | T | C | H | | M | | | | A | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| S2.41 | Y | H | K | W | | T | C | H | | M | | G | P | E | |
| S2.42 | Y | H | K | W | | T | C | H | C | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | W | G | T | C | H | C | M | | | P | E | |
| S2.48 | Y | H | K | W | G | T | C | H | C | M | | | | E | E |
| S2.49 | Y | H | K | W | G | T | C | H | C | M | | | | A | E |
| S2.50 | Y | H | K | W | G | T | C | H | C | M | N | G | | E | |
| S2.51 | Y | H | K | W | G | T | C | H | C | M | N | | P | E | |
| S2.52 | Y | H | K | W | G | T | C | H | C | M | | G | P | E | |
| S2.53 | Y | H | K | W | | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6.2E
Hybrid constructs. Mutations are indicated with reference to ABE7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| ABE7.10 | I | V | A | T | D | H | Y | Y | F | Q | T | D |
| ABE8e | | | S | R | N | N | | D | Y | | I | N |
| ABE8.20 | Y | S | | | | | H | R | | R | | |
| ABE8.17 | | S | | | | | | | | R | | |
| pNMG- | Y | S | | | | | H | D | | R | | |
| B878 | | | | | | | | | | | | |
| pNMG- | Y | S | | | | | H | R | Y | R | | |
| B879 | | | | | | | | | | | | |
| pNMG- | Y | S | | | | | H | R | | R | I | |
| B880 | | | | | | | | | | | | |
| pNMG- | Y | S | | | | | H | R | | R | | N |
| B881 | | | | | | | | | | | | |
| pNMG- | Y | S | | | | | H | D | Y | R | I | N |
| B882 | | | | | | | | | | | | |
| pNMG- | Y | S | | R | N | | H | R | | R | | |
| B883 | | | | | | | | | | | | |
| pNMG- | Y | S | S | R | N | N | H | R | | R | | |
| B884 | | | | | | | | | | | | |
| pNMG- | Y | S | S | | | | H | R | | R | | |
| B885 | | | | | | | | | | | | |
| pNMG- | Y | S | | R | | | H | R | | R | | |
| B886 | | | | | | | | | | | | |
| pNMG- | Y | S | | | N | | H | R | | R | | |
| B887 | | | | | | | | | | | | |
| pNMG- | Y | S | | | | N | H | R | | R | | |
| B888 | | | | | | | | | | | | |
| pNMG- | Y | S | S | R | | | H | R | | R | | |
| B889 | | | | | | | | | | | | |
| ABE7.10 | I | V | A | T | D | H | Y | Y | F | Q | T | D |
| ABE8e | | | S | R | N | N | | D | Y | | I | N |
| ABE8.20 | Y | S | | | | | H | R | | R | | |
| ABE8.17 | | S | | | | | | | | R | | |
| pNMG- | Y | S | | | N | N | H | R | | R | | |
| B890 | | | | | | | | | | | | |
| pNMG- | Y | S | S | R | N | N | H | D | Y | R | I | N |
| B891 | | | | | | | | | | | | |

TABLE 6.2F
Base editor variants. Mutations are indicated with reference to ABE8.19m/8.20m.

| AA positions: | 17 | 27 | 48 | 49 | 76 | 82 | 84 | 118 | 142 | 147 | 149 | 166 | 167 |
|---|---|---|---|---|---|---|---|---|---|---|---|---|---|
| ABE8.19m/8.20m | T | E | A | I | Y/I | S | F | M | A | Y | F | T | D |
| 1.1 + 8e(B879) | | H | | | I | | M | | | | Y | | |
| 1.2 + 8e(B879) | | H | | K | I | | | | | | Y | | |
| 1.12 + 8e(B879) | A | H | | M | I | | | L | | | Y | | |
| 1.17 + 8e(B879) | A | | G | | | T | | | E | | Y | | |
| 1.18 + 8e(B879) | | G | | | | | | | | | Y | | |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, and then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., et al. Science 337:816-821(2012), the entire contents of which is hereby incorporated by reference. Cas9 recognizes a short motif in the CRISPR repeat sequences (the PAM or protospacer adjacent motif) to help distinguish self versus non-self. See e.g., “Complete genome sequence of an M1 strain of Streptococcus pyogenes.” Ferretti, J. J. et al., Natl. Acad. Sci. U.S.A. 98:4658-4663(2001); “CRISPR RNA maturation by trans-encoded small RNA and host factor RNase III.” Deltcheva E. et al., Nature 471:602-607(2011); and “Programmable dual-RNA-guided DNA endonuclease in adaptive bacterial immunity.” Jinek M. et al, Science 337:816-821(2012), the entire contents of each of which are incorporated herein by reference).

The PAM sequence can be any PAM sequence known in the art. Suitable PAM sequences include, but are not limited to, NGG, NGA, NGC, NGN, NGT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

In an embodiment, a guide polynucleotide described herein can be RNA or DNA. In one embodiment, the guide polynucleotide is a guide RNA (gRNA). Exemplary guide RNAs spacer sequences are provided in Tables 1A and 1B. An RNA/Cas complex can assist in “guiding” a Cas protein to a target DNA. Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

In some embodiments, the guide polynucleotide is at least one tracrRNA. In some embodiments, the guide polynucleotide does not require PAM sequence to guide the polynucleotide-programmable DNA-binding domain (e.g., Cas9 or Cpf1) to the target nucleotide sequence.

A guide polynucleotide may include natural or non-natural (or unnatural) nucleotides (e.g., peptide nucleic acid or nucleotide analogs). In some cases, the targeting region of a guide nucleic acid sequence can be at least 15, 16, 17, 18, 19, 20, 21, 22, 23, 24, 25, 26, 27, 28, 29, or 30 nucleotides in length. A targeting region of a guide nucleic acid can be between 10-30 nucleotides in length, or between 15-25 nucleotides in length, or between 15-20 nucleotides in length.

In some embodiments, the base editor provided herein utilizes one or more guide polynucleotide (e.g., multiple gRNA). In some embodiments, a single guide polynucleotide is utilized for different base editors described herein. For example, a single guide polynucleotide can be utilized for a cytidine base editor and an adenosine base editor.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327. Thus, a skilled artisan can change the genomic target of the Cas protein specificity is partially determined by how specific the gRNA targeting sequence is for the genomic target compared to the rest of the genome.

In other embodiments, a guide polynucleotide can comprise both the polynucleotide targeting portion of the nucleic acid and the scaffold portion of the nucleic acid in a single molecule (i.e., a single-molecule guide nucleic acid). For example, a single-molecule guide polynucleotide can be a single guide RNA (sgRNA or gRNA). Herein the term guide polynucleotide sequence contemplates any single, dual or multi-molecule nucleic acid capable of interacting with and directing a base editor to a target polynucleotide sequence.

Typically, a guide polynucleotide (e.g., crRNA/trRNA complex or a gRNA) comprises a “polynucleotide-targeting segment” that includes a sequence capable of recognizing and binding to a target polynucleotide sequence, and a “protein-binding segment” that stabilizes the guide polynucleotide within a polynucleotide programmable nucleotide binding domain component of a base editor. In some embodiments, the polynucleotide targeting segment of the guide polynucleotide recognizes and binds to a DNA polynucleotide, thereby facilitating the editing of a base in DNA. In other cases, the polynucleotide targeting segment of the guide polynucleotide recognizes and binds to an RNA polynucleotide, thereby facilitating the editing of a base in RNA. Herein a “segment” refers to a section or region of a molecule, e.g., a contiguous stretch of nucleotides in the guide polynucleotide. A segment can also refer to a region/section of a complex such that a segment can comprise regions of more than one molecule. For example, where a guide polynucleotide comprises multiple nucleic acid molecules, the protein-binding segment of can include all or a portion of multiple separate molecules that are for instance hybridized along a region of complementarity. In some embodiments, a protein-binding segment of a DNA-targeting RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and can include regions of RNA molecules that are of any total length and can include regions with complementarity to other molecules.

The guide polynucleotides can be synthesized chemically, synthesized enzymatically, or a combination thereof. For example, the gRNA can be synthesized using standard phosphoramidite-based solid-phase synthesis methods. Alternatively, the gRNA can be synthesized in vitro by operably linking DNA encoding the gRNA to a promoter control sequence that is recognized by a phage RNA polymerase. Examples of suitable phage promoter sequences include T7, T3, SP6 promoter sequences, or variations thereof. In embodiments in which the gRNA comprises two separate molecules (e.g., crRNA and tracrRNA), the crRNA can be chemically synthesized and the tracrRNA can be enzymatically synthesized.

A guide polynucleotide may be expressed, for example, by a DNA that encodes the gRNA, e.g., a DNA vector comprising a sequence encoding the gRNA. The gRNA may be encoded alone or together with an encoded base editor. Such DNA sequences may be introduced into an expression system, e.g., a cell, together or separately. For example, DNA sequences encoding a polynucleotide programmable nucleotide binding domain and a gRNA may be introduced into a cell, each DNA sequence can be part of a separate molecule (e.g., one vector containing the polynucleotide programmable nucleotide binding domain coding sequence and a second vector containing the gRNA coding sequence) or both can be part of a same molecule (e.g., one vector containing coding (and regulatory) sequence for both the polynucleotide programmable nucleotide binding domain and the gRNA). An RNA can be transcribed from a synthetic DNA molecule, e.g., a gBlocks® gene fragment. A gRNA molecule can be transcribed in vitro.

A gRNA or a guide polynucleotide can comprise three regions: a first region at the 5′ end that can be complementary to a target site in a chromosomal sequence, a second internal region that can form a stem loop structure, and a third 3′ region that can be single-stranded. A first region of each gRNA can also be different such that each gRNA guides a fusion protein to a specific target site. Further, second and third regions of each gRNA can be identical in all gRNAs.

A first region of a gRNA or a guide polynucleotide can be complementary to sequence at a target site in a chromosomal sequence such that the first region of the gRNA can base pair with the target site. In some cases, a first region of a gRNA can comprise from or from about 10 nucleotides to 25 nucleotides (i.e., from 10 nucleotides to nucleotides; or from about 10 nucleotides to about 25 nucleotides; or from 10 nucleotides to about 25 nucleotides; or from about 10 nucleotides to 25 nucleotides) or more. For example, a region of base pairing between a first region of a gRNA and a target site in a chromosomal sequence can be or can be about 10, 11, 12, 13, 14, 15, 16, 17, 18, 19, 20, 22, 23, 24, 25, or more nucleotides in length. Sometimes, a first region of a gRNA can be or can be about 19, 20, or 21 nucleotides in length.

A gRNA or a guide polynucleotide can also comprise a second region that forms a secondary structure. For example, a secondary structure formed by a gRNA can comprise a stem (or hairpin) and a loop. A length of a loop and a stem can vary. For example, a loop can range from or from about 3 to 10 nucleotides in length, and a stem can range from or from about 6 to 20 base pairs in length. A stem can comprise one or more bulges of 1 to 10 or about 10 nucleotides. The overall length of a second region can range from or from about 16 to 60 nucleotides in length. For example, a loop can be or can be about 4 nucleotides in length and a stem can be or can be about 12 base pairs.

A gRNA or a guide polynucleotide can also comprise a third region at the 3′ end that can be essentially single-stranded. For example, a third region is sometimes not complementarity to any chromosomal sequence in a cell of interest and is sometimes not complementarity to the rest of a gRNA. Further, the length of a third region can vary. A third region can be more than or more than about 4 nucleotides in length. For example, the length of a third region can range from or from about 5 to 60 nucleotides in length.

A gRNA or a guide polynucleotide can target any exon or intron of a gene target. In some cases, a guide can target exon 1 or 2 of a gene, in other cases; a guide can target exon 3 or 4 of a gene. In some embodiments, a composition comprises multiple gRNAs that all target the same exon or multiple gRNAs that target different exons. An exon and/or an intron of a gene can be targeted.

A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

Methods for selecting, designing, and validating guide polynucleotides, e.g., gRNAs and targeting sequences are described herein and known to those skilled in the art. For example, to minimize the impact of potential substrate promiscuity of a deaminase domain in the nucleobase editor system (e.g., an AID domain), the number of residues that could unintentionally be targeted for deamination (e.g., off-target C residues that could potentially reside on single strand DNA within the target nucleic acid locus) may be minimized. In addition, software tools can be used to optimize the gRNAs corresponding to a target nucleic acid sequence, e.g., to minimize total off-target activity across the genome. For example, for each possible targeting domain choice using S. pyogenes Cas9, all off-target sequences (preceding selected PAMs, e.g., NAG or NGG) may be identified across the genome that contain up to certain number (e.g., 1, 2, 3, 4, 5, 6, 7, 8, 9, or 10) of mismatched base-pairs. First regions of gRNAs complementary to a target site can be identified, and all first regions (e.g., crRNAs) can be ranked according to its total predicted off-target score; the top-ranked targeting domains represent those that are likely to have the greatest on-target and the least off-target activity. Candidate targeting gRNAs can be functionally evaluated by using methods known in the art and/or as set forth herein.

As a non-limiting example, target DNA hybridizing sequences in crRNAs of a gRNA for use with Cas9s may be identified using a DNA sequence searching algorithm. gRNA design is carried out using custom gRNA design software based on the public tool cas-OFFinder as described in Bae S., Park J., & Kim J.-S. Cas-OFFinder: A fast and versatile algorithm that searches for potential off-target sites of Cas9 RNA-guided endonucleases. Bioinformatics 30, 1473-1475 (2014). This software scores guides after calculating their genome-wide off-target propensity. Typically matches ranging from perfect matches to 7 mismatches are considered for guides ranging in length from 17 to 24. Once the off-target sites are computationally-determined, an aggregate score is calculated for each guide and summarized in a tabular output using a web-interface. In addition to identifying potential target sites adjacent to PAM sequences, the software also identifies all PAM adjacent sequences that differ by 1, 2, 3 or more than 3 nucleotides from the selected target sites. Genomic DNA sequences for a target nucleic acid sequence, e.g., a target gene may be obtained and repeat elements may be screened using publicly available tools, for example, the RepeatMasker program. RepeatMasker searches input DNA sequences for repeated elements and regions of low complexity. The output is a detailed annotation of the repeats present in a given query sequence.

Following identification, first regions of gRNAs, e.g., crRNAs, are ranked into tiers based on their distance to the target site, their orthogonality and presence of 5′ nucleotides for close matches with relevant PAM sequences (for example, a 5′ G based on identification of close matches in the human genome containing a relevant PAM e.g., NGG PAM for S. pyogenes, NNGRRT or NNGRRV PAM for S. aureus). As used herein, orthogonality refers to the number of sequences in the human genome that contain a minimum number of mismatches to the target sequence. A “high level of orthogonality” or “good orthogonality” may, for example, refer to 20-mer targeting domains that have no identical sequences in the human genome besides the intended target, nor any sequences that contain one or two mismatches in the target sequence. Targeting domains with good orthogonality may be selected to minimize off-target DNA cleavage.

A gRNA can then be introduced into a cell or embryo as an RNA molecule or a non-RNA nucleic acid molecule, e.g., DNA molecule. In one embodiment, a DNA encoding a gRNA is operably linked to promoter control sequence for expression of the gRNA in a cell or embryo of interest. A RNA coding sequence can be operably linked to a promoter sequence that is recognized by RNA polymerase III (Pol III). Plasmid vectors that can be used to express gRNA include, but are not limited to, px330 vectors and px333 vectors. In some cases, a plasmid vector (e.g., px333 vector) can comprise at least two gRNA-encoding DNA sequences. Further, a vector can comprise additional expression control sequences (e.g., enhancer sequences, Kozak sequences, polyadenylation sequences, transcriptional termination sequences, etc.), selectable marker sequences (e.g., GFP or antibiotic resistance genes such as puromycin), origins of replication, and the like. A DNA molecule encoding a gRNA can also be linear. A DNA molecule encoding a gRNA or a guide polynucleotide can also be circular.

In some embodiments, a reporter system is used for detecting base-editing activity and testing candidate guide polynucleotides. In some embodiments, a reporter system comprises a reporter gene based assay where base editing activity leads to expression of the reporter gene. For example, a reporter system may include a reporter gene comprising a deactivated start codon, e.g., a mutation on the template strand from 3′-TAC-5′ to 3′-CAC-5′. Upon successful deamination of the target C, the corresponding mRNA will be transcribed as 5′-AUG-3′ instead of 5′-GUG-3′, enabling the translation of the reporter gene. Suitable reporter genes will be apparent to those of skill in the art. Non-limiting examples of reporter genes include gene encoding green fluorescence protein (GFP), red fluorescence protein (RFP), luciferase, secreted alkaline phosphatase (SEAP), or any other gene whose expression are detectable and apparent to those skilled in the art. The reporter system can be used to test many different gRNAs, e.g., in order to determine which residue(s) with respect to the target DNA sequence the respective deaminase will target. sgRNAs that target non-template strand can also be tested in order to assess off-target effects of a specific base editing protein, e.g., a Cas9 deaminase fusion protein. In some embodiments, such gRNAs can be designed such that the mutated start codon will not be base-paired with the gRNA. The guide polynucleotides can comprise standard ribonucleotides, modified ribonucleotides (e.g., pseudouridine), ribonucleotide isomers, and/or ribonucleotide analogs. In some embodiments, the guide polynucleotide can comprise at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and are preferably separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In a particular embodiment, the chemical modifications are 2′-O-methyl (2′-OMe) modifications. The modified guide RNAs may improve saCas9 efficacy and also specificity. The effect of an individual modification varies based on the position and combination of chemical modifications used as well as the inter- and intramolecular interactions with other modified nucleotides. By way of example, S-cEt has been used to improve oligonucleotide intramolecular folding.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises four modified nucleosides at the 5′ end and four modified nucleosides at the 3′ end of the guide. In some embodiments, the modified nucleoside comprises a 2′ O-methyl or a phosphorothioate.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length protospacer. In some embodiments, the guide comprises a 20-40 nucleotide protospacer. In some embodiments, the guide comprises a protospacer comprising at least about 20-25 nucleotides or at least about 30-35 nucleotides. In some embodiments, the protospacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length protospacer; and
a protospacer comprising modified nucleotides.

In embodiments, the gRNA contains numerous modified nucleotides and/or chemical modifications (“heavy mods”). Such heavy mods can increase base editing ˜2 fold in vivo or in vitro. For such modifications, mN=2′-OMe; Ns=phosphorothioate (PS), where “N” represents the any nucleotide, as would be understood by one having skill in the art. In some cases, a nucleotide (N) may contain two modifications, for example, both a 2′-OMe and a PS modification. For example, a nucleotide with a phosphorothioate and 2′ OMe is denoted as “mNs;” when there are two modifications next to each other, the notation is “mNsmNs.

In some embodiments of the modified gRNA, the gRNA comprises one or more chemical modifications selected from the group consisting of 2′-O-methyl (2′-OMe), phosphorothioate (PS), 2′-O-methyl thioPACE (MSP), 2′-O-methyl-PACE (MP), 2′-O-methyl thioPACE (MSP), 2′-fluoro RNA (2′-F-RNA), and constrained ethyl (S-cEt). In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

In some cases, a gRNA or a guide polynucleotide can comprise modifications. A modification can be made at any location of a gRNA or a guide polynucleotide. More than one modification can be made to a single gRNA or a guide polynucleotide. A gRNA or a guide polynucleotide can undergo quality control after a modification. In some cases, quality control can include PAGE, HPLC, MS, or any combination thereof.

A modification of a gRNA or a guide polynucleotide can be a substitution, insertion, deletion, chemical modification, physical modification, stabilization, purification, or any combination thereof.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a modification is permanent. In other cases, a modification is transient. In some cases, multiple modifications are made to a gRNA or a guide polynucleotide. A gRNA or a guide polynucleotide modification can alter physiochemical properties of a nucleotide, such as their conformation, polarity, hydrophobicity, chemical reactivity, base-pairing interactions, or any combination thereof.

A guide polynucleotide can be transferred into a cell by transfecting the cell with an isolated gRNA or a plasmid DNA comprising a sequence coding for the guide RNA and a promoter. A gRNA or a guide polynucleotide can also be transferred into a cell in other way, such as using virus-mediated gene delivery. A gRNA or a guide polynucleotide can be isolated. For example, a gRNA can be transfected in the form of an isolated RNA into a cell or organism. A gRNA can be prepared by in vitro transcription using any in vitro transcription system known in the art. A gRNA can be transferred to a cell in the form of isolated RNA rather than in the form of plasmid comprising encoding sequence for a gRNA.

A modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a gRNA or a guide polynucleotide. A modification can also enhance biological activity. In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

In some embodiments, the guide RNA is designed to disrupt a splice site (i.e., a splice acceptor (SA) or a splice donor (SD). In some embodiments, the guide RNA is designed such that the base editing results in a premature STOP codon.

### Protospacer Adjacent Motif

The term “protospacer adjacent motif (PAM)” or PAM-like motif refers to a 2-6 base pair DNA sequence immediately following the DNA sequence targeted by the Cas9 nuclease in the CRISPR bacterial adaptive immune system. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence is essential for target binding, but the exact sequence depends on a type of Cas protein. The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence. A PAM site is a nucleotide sequence in proximity to a target polynucleotide sequence. Some aspects of the disclosure provide for base editors comprising all or a portion of CRISPR proteins that have different PAM specificities.

For example, typically Cas9 proteins, such as Cas9 from S. pyogenes (spCas9), require a canonical NGG PAM sequence to bind a particular nucleic acid region, where the “N” in “NGG” is adenine (A), thymine (T), guanine (G), or cytosine (C), and the G is guanine. A PAM can be CRISPR protein-specific and can be different between different base editors comprising different CRISPR protein-derived domains. A PAM can be 5′ or 3′ of a target sequence. A PAM can be upstream or downstream of a target sequence. A PAM can be 1, 2, 3, 4, 5, 6, 7, 8, 9, 10 or more nucleotides in length. Often, a PAM is between 2-6 nucleotides in length.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 7 below.

TABLE 7
Cas9 proteins and corresponding PAM sequences

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′ (TTTV) |
| SpyMac | 5′-NAA-3′ |

In some embodiments, the PAM is NGC. In some embodiments, the NGC PAM is recognized by a Cas9 variant. In some embodiments, the NGC PAM variant includes one or more amino acid substitutions selected from D1135M, S1136Q, G1218K, E1219F, A1322R, D1332A, R1335E, and T1337R (collectively termed “MQKFRAER”).

In some embodiments, the PAM is NGT. In some embodiments, the NGT PAM is recognized by a Cas9 variant. In some embodiments, the NGT PAM variant is generated through targeted mutations at one or more residues 1335, 1337, 1135, 1136, 1218, and/or 1219. In some embodiments, the NGT PAM variant is created through targeted mutations at one or more residues 1219, 1335, 1337, 1218. In some embodiments, the NGT PAM variant is created through targeted mutations at one or more residues 1135, 1136, 1218, 1219, and 1335. In some embodiments, the NGT PAM variant is selected from the set of targeted mutations provided in Tables 8A and 8B below.

TABLE 8A
NGT PAM Variant Mutations at residues 1219, 1335, 1337, 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |
| 9 | L | L | T | |
| 10 | L | L | R | |
| 11 | L | L | Q | |
| 12 | L | L | L | |
| 13 | F | I | T | |
| 14 | F | I | R | |
| 15 | F | I | Q | |
| 16 | F | I | L | |
| 17 | F | G | C | |
| 18 | H | L | N | |
| 19 | F | G | C | A |
| 20 | H | L | N | V |
| 21 | L | A | W | |
| 22 | L | A | F | |
| 23 | L | A | Y | |
| 24 | I | A | W | |
| 25 | I | A | F | |
| 26 | I | A | Y | |

TABLE 8B
NGT PAM Variant Mutations at residues
1135, 1136, 1218, 1219, and 1335

| Variant | D1135L | S1136R | G1218S | E1219V | R1335Q |
|---|---|---|---|---|---|
| 27 | G | | | | |
| 28 | V | | | | |
| 29 | I | | | | |
| 30 | | A | | | |
| 31 | | W | | | |
| 32 | | H | | | |
| 33 | | K | | | |
| 34 | | | K | | |
| 35 | | | R | | |
| 36 | | | Q | | |
| 37 | | | T | | |
| 38 | | | N | | |
| 39 | | | | I | |
| 40 | | | | A | |
| 41 | | | | N | |
| 42 | | | | Q | |
| 43 | | | | G | |
| 44 | | | | L | |
| 45 | | | | S | |
| 46 | | | | T | |
| 47 | | | | | L |
| 48 | | | | | I |
| 49 | | | | | V |
| 50 | | | | | N |
| 51 | | | | | S |
| 52 | | | | | T |
| 53 | | | | | F |
| 54 | | | | | Y |
| 55 | N1286Q | I1331F | | | |

In some embodiments, the NGT PAM variant is selected from variant 5, 7, 28, 31, or 36 in Table 8A and Table 8B. In some embodiments, the variants have improved NGT PAM recognition.

In some embodiments, the NGT PAM variants have mutations at residues 1219, 1335, 1337, and/or 1218. In some embodiments, the NGT PAM variant is selected with mutations for improved recognition from the variants provided in Table 9 below.

TABLE 9
NGT PAM Variant Mutations at residues
1219, 1335, 1337, and 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |

In some embodiments, the NGT PAM is selected from the variants provided in Table 10 below.

TABLE 10
NGT PAM variants

| | NGTN | | | | | | | |
|---|---|---|---|---|---|---|---|---|
| | variant | D1135 | S1136 | G1218 | E1219 | A1322R | R1335 | T1337 |
| Variant 1 | LRKIQK | L | R | K | I | — | Q | K |
| Variant 2 | LRSVQK | L | R | S | V | — | Q | K |
| Variant 3 | LRSVQL | L | R | S | V | — | Q | L |
| Variant 4 | LRKIRQK | L | R | K | I | R | Q | K |
| Variant 5 | LRSVRQK | L | R | S | V | R | Q | K |
| Variant 6 | LRSVRQL | L | R | S | V | R | Q | L |

In some embodiments the NGTN variant is variant 1. In some embodiments, the NGTN variant is variant 2. In some embodiments, the NGTN variant is variant 3. In some embodiments, the NGTN variant is variant 4. In some embodiments, the NGTN variant is variant 5. In some embodiments, the NGTN variant is variant 6.

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises a D9X mutation, or a corresponding mutation in any of the amino acid sequences provided herein, wherein X is any amino acid except for D. In some embodiments, the SpCas9 comprises a D9A mutation, or a corresponding mutation in any of the amino acid sequences provided herein. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having an NGG, a NGA, or a NGCG PAM sequence.

In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135E, R1335Q, and T1337R mutation, or a corresponding mutation in any of the amino acid sequences provided herein. In some embodiments, the SpCas9 domain comprises a D1135E, a R1335Q, and a T1337R mutation, or corresponding mutations in any of the amino acid sequences provided herein. In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135V, a R1335Q, and a T1337R mutation, or a corresponding mutation in any of the amino acid sequences provided herein. In some embodiments, the SpCas9 domain comprises a D1135V, a R1335Q, and a T1337R mutation, or corresponding mutations in any of the amino acid sequences provided herein. In some embodiments, the SpCas9 domain comprises one or more of a D1135X, a G1218X, a R1335X, and a T1337X mutation, or a corresponding mutation in any of the amino acid sequences provided herein, wherein X is any amino acid. In some embodiments, the SpCas9 domain comprises one or more of a D1135V, a G1218R, a R1335Q, and a T1337R mutation, or a corresponding mutation in any of the amino acid sequences provided herein. In some embodiments, the SpCas9 domain comprises a D1135V, a G1218R, a R1335Q, and a T1337R mutation, or corresponding mutations in any of the amino acid sequences provided herein.

In some examples, a PAM recognized by a CRISPR protein-derived domain of a base editor disclosed herein can be provided to a cell on a separate oligonucleotide to an insert (e.g., an AAV insert) encoding the base editor. In such embodiments, providing PAM on a separate oligonucleotide can allow cleavage of a target sequence that otherwise would not be able to be cleaved, because no adjacent PAM is present on the same polynucleotide as the target sequence.

In an embodiment, S. pyogenes Cas9 (SpCas9) can be used as a CRISPR endonuclease for genome engineering. However, others can be used. In some embodiments, a different endonuclease can be used to target certain genomic targets. In some embodiments, synthetic SpCas9-derived variants with non-NGG PAM sequences can be used. Additionally, other Cas9 orthologues from various species have been identified and these “non-SpCas9s” can bind a variety of PAM sequences that can also be useful for the present disclosure. For example, the relatively large size of SpCas9 (approximately 4kb coding sequence) can lead to plasmids carrying the SpCas9 cDNA that cannot be efficiently expressed in a cell. Conversely, the coding sequence for Staphylococcus aureus Cas9 (SaCas9) is approximately 1 kilobase shorter than SpCas9, possibly allowing it to be efficiently expressed in a cell. Similar to SpCas9, the SaCas9 endonuclease is capable of modifying target genes in mammalian cells in vitro and in mice in vivo. In some embodiments, a Cas protein can target a different PAM sequence. In some embodiments, a target gene can be adjacent to a Cas9 PAM, 5′-NGG, for example. In other embodiments, other Cas9 orthologs can have different PAM requirements. For example, other PAMs such as those of S. thermophilus (5′-NNAGAA for CRISPRI and 5′-NGGNG for CRISPR3) and Neisseria meningitidis (5′-NNNNGATT) can also be found adjacent to a target gene.

In some embodiments, for a S. pyogenes system, a target gene sequence can precede (i.e., be 5′ to) a 5′-NGG PAM, and a 20-nt guide RNA sequence can base pair with an opposite strand to mediate a Cas9 cleavage adjacent to a PAM. In some embodiments, an adjacent cut can be or can be about 3 base pairs upstream of a PAM. In some embodiments, an adjacent cut can be or can be about 10 base pairs upstream of a PAM. In some embodiments, an adjacent cut can be or can be about 0-20 base pairs upstream of a PAM. For example, an adjacent cut can be next to, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs upstream of a PAM. An adjacent cut can also be downstream of a PAM by 1 to 30 base pairs. The sequences of exemplary SpCas9 proteins capable of binding a PAM sequence follow:

In some embodiments, engineered SpCas9 variants are capable of recognizing protospacer adjacent motif (PAM) sequences flanked by a 3′ H (non-G PAM) (see Tables 3A-3D). In some embodiments, the SpCas9 variants recognize NRNH PAMs (where R is A or G and H is A, C or T). In some embodiments, the non-G PAM is NRRH, NRTH, or NRCH (see e.g., Miller, S. M., et al. Continuous evolution of SpCas9 variants compatible with non-G PAMs, Nat. Biotechnol. (2020), the contents of which is incorporated herein by reference in its entirety).

In some embodiments, the Cas9 domain is a recombinant Cas9 domain. In some embodiments, the recombinant Cas9 domain is a SpyMacCas9 domain. In some embodiments, the SpyMacCas9 domain is a nuclease active SpyMacCas9, a nuclease inactive SpyMacCas9 (SpyMacCas9d), or a SpyMacCas9 nickase (SpyMacCas9n). In some embodiments, the SaCas9 domain, the SaCas9d domain, or the SaCas9n domain can bind to a nucleic acid sequence having a non-canonical PAM. In some embodiments, the SpyMacCas9 domain, the SpCas9d domain, or the SpCas9n domain can bind to a nucleic acid sequence having a NAA PAM sequence.

The sequence of an exemplary Cas9 A homolog of Spy Cas9 in Streptococcus macacae with native 5′-NAAN-3′ PAM specificity is known in the art and described, for example, by Chatterjee, et al., “A Cas9 with PAM recognition for adenine dinucleotides”, Nature Communications, vol. 11, article no. 2474 (2020), and is in the Sequence Listing as SEQ ID NO: 237.

In some embodiments, a variant Cas9 protein harbors, H840A, P475A, W476A, N477A, D1125A, W1126A, and D1218A mutations such that the polypeptide has a reduced ability to cleave a target DNA or RNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors D10A, H840A, P475A, W476A, N477A, D1125A, Wi 126A, and D1218A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, when a variant Cas9 protein harbors W476A and Wi 126A mutations or when the variant Cas9 protein harbors P475A, W476A, N477A, D1125A, W1126A, and D1218A mutations, the variant Cas9 protein does not bind efficiently to a PAM sequence. Thus, in some such cases, when such a variant Cas9 protein is used in a method of binding, the method does not require a PAM sequence. In other words, in some embodiments, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a CRISPR protein-derived domain of a base editor can comprise all or a portion of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556(7699), 57-63; Miller et al., “Continuous evolution of SpCas9 variants compatible with non-G PAMs” Nat. Biotechnol., 2020 April; 38(4):471-481; the entire contents of each are hereby incorporated by reference.

Fusion Proteins Comprising a NapDNAbp and a Cytidine Deaminase and/or Adenosine Deaminase

Some aspects of the disclosure provide fusion proteins comprising a Cas9 domain or other nucleic acid programmable DNA binding protein (e.g., Cas12) and one or more cytidine deaminase or adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion protein comprises the following domains A-C, A-D, or A-E:
NH₂-[A-B-C]-COOH;
NH₂-[A-B-C-D]-COOH; or
NH₂-[A-B-C-D-E]-COOH;
   wherein A and C or A, C, and E, each comprises one or more of the following:
an adenosine deaminase domain or an active fragment thereof,
a cytidine deaminase domain or an active fragment thereof,
and
wherein B or B and D, each comprises one or more domains having nucleic acid sequence specific binding activity.

In some embodiments, the fusion protein comprises the following structure:
NH₂-[Aₙ-Bₒ-Cₙ]-COOH;
NH₂-[Aₙ-Bₒ-Cₙ-Dₒ]-COOH; or
NH₂-[Aₙ-Bₒ-Cₚ-Dₒ-E_{q}]-COOH;
   wherein A and C or A, C, and E, each comprises one or more of the following:
an adenosine deaminase domain or an active fragment thereof,
a cytidine deaminase domain or an active fragment thereof, and
   wherein n is an integer: 1, 2, 3, 4, or 5, wherein p is an integer: 0, 1, 2, 3, 4, or 5; wherein q is an integer 0, 1, 2, 3, 4, or 5; and wherein B or B and D each comprises a domain having nucleic acid sequence specific binding activity; and wherein o is an integer: 1, 2, 3, 4, or 5.

For example, and without limitation, in some embodiments, the fusion protein comprises the structure:
NH₂-[adenosine deaminase]-[Cas9 domain]-COOH;
NH₂-[Cas9 domain]-[adenosine deaminase]-COOH;
NH₂-[cytidine deaminase]-[Cas9 domain]-COOH;
NH₂-[Cas9 domain]-[cytidine deaminase]-COOH;
NH₂-[cytidine deaminase]-[Cas9 domain]-[adenosine deaminase]-COOH;
NH₂-[adenosine deaminase]-[Cas9 domain]-[cytidine deaminase]-COOH;
NH₂-[adenosine deaminase]-[cytidine deaminase]-[Cas9 domain]-COOH;
NH₂-[cytidine deaminase]-[adenosine deaminase]-[Cas9 domain]-COOH;
NH₂-[Cas9 domain]-[adenosine deaminase]-[cytidine deaminase]-COOH; or
NH₂-[Cas9 domain]-[cytidine deaminase]-[adenosine deaminase]-COOH.

In some embodiments, any of the Cas12 domains or Cas12 proteins provided herein may be fused with any of the cytidine or adenosine deaminases provided herein. For example, and without limitation, in some embodiments, the fusion protein comprises the structure:
NH2-[adenosine deaminase]-[Cas12 domain]-COOH;
NH2-[Cas12 domain]-[adenosine deaminase]-COOH;
NH2-[cytidine deaminase]-[Cas12 domain]-COOH;
NH2-[Cas12 domain]-[cytidine deaminase]-COOH;
NH2-[cytidine deaminase]-[Cas12 domain]-[adenosine deaminase]-COOH;
NH2-[adenosine deaminase]-[Cas12 domain]-[cytidine deaminase]-COOH;
NH2-[adenosine deaminase]-[cytidine deaminase]-[Cas12 domain]-COOH;
NH2-[cytidine deaminase]-[adenosine deaminase]-[Cas12 domain]-COOH;
NH2-[Cas12 domain]-[adenosine deaminase]-[cytidine deaminase]-COOH; or
NH2-[Cas12 domain]-[cytidine deaminase]-[adenosine deaminase]-COOH.

In some embodiments, the adenosine deaminase is a TadA*8. Exemplary fusion protein structures include the following:
NH2-[TadA*8]-[Cas9 domain]-COOH;
NH2-[Cas9 domain]-[TadA*8]-COOH;
NH2-[TadA*8]-[Cas12 domain]-COOH; or
NH2-[Cas12 domain]-[TadA*8]-COOH.

In some embodiments, the adenosine deaminase of the fusion protein comprises a TadA*8 and a cytidine deaminase and/or an adenosine deaminase. In some embodiments, the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

Exemplary fusion protein structures include the following:
NH2-[TadA*8]-[Cas9/Cas12]-[adenosine deaminase]-COOH;
NH2-[adenosine deaminase]-[Cas9/Cas12]-[TadA*8]-COOH;
NH2-[TadA*8]-[Cas9/Cas12]-[cytidine deaminase]-COOH; or
NH2-[cytidine deaminase]-[Cas9/Cas12]-[TadA*8]-COOH.

In some embodiments, the adenosine deaminase of the fusion protein comprises a TadA*9 and a cytidine deaminase and/or an adenosine deaminase. Exemplary fusion protein structures include the following:
NH2-[TadA*9]-[Cas9/Cas12]-[adenosine deaminase]-COOH;
NH2-[adenosine deaminase]-[Cas9/Cas12]-[TadA*9]-COOH;
NH2-[TadA*9]-[Cas9/Cas12]-[cytidine deaminase]-COOH; or
NH2-[cytidine deaminase]-[Cas9/Cas12]-[TadA*9]-COOH.

In some embodiments, the fusion protein can comprise a deaminase flanked by an N-terminal fragment and a C-terminal fragment of a Cas9 or Cas12 polypeptide. In some embodiments, the fusion protein comprises a cytidine deaminase flanked by an N-terminal fragment and a C-terminal fragment of a Cas9 or Cas12 polypeptide. In some embodiments, the fusion protein comprises an adenosine deaminase flanked by an N-terminal fragment and a C-terminal fragment of a Cas9 or Cas 12 polypeptide.

In some embodiments, the fusion proteins comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, the “-” used in the general architecture above indicates the presence of an optional linker. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins of the present disclosure may comprise one or more additional features. For example, in some embodiments, the fusion protein may comprise inhibitors, cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

Exemplary, yet nonlimiting, fusion proteins are described in International PCT Application Nos. PCT/2017/044935, PCT/US2019/044935, and PCT/US2020/016288, each of which is incorporated herein by reference for its entirety.

### Fusion Proteins Comprising a Nuclear Localization Sequence (NLS)

In some embodiments, the fusion proteins provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, an NLS comprises the amino acid sequence PKKKRKVEGADKRTADGSEFESPKKKRKV (SEQ ID NO: 328), KRTADGSEFESPKKKRKV (SEQ ID NO: 190), KRPAATKKAGQAKKKK (SEQ ID NO: 191), KKTELQTTNAENKTKKL (SEQ ID NO: 192), KRGINDRNFWRGENGRKTR (SEQ ID NO: 193), RKSGKIAAIVVKRPRKPKKKRKV (SEQ ID NO: 329), or MDSLLMNRRKFLYQFKNVRWAKGRRETYLC (SEQ ID NO: 196).

In some embodiments, the fusion proteins comprising a cytidine or adenosine deaminase, a Cas9 domain, and an NLS do not comprise a linker sequence. In some embodiments, linker sequences between one or more of the domains or proteins (e.g., cytidine or adenosine deaminase, Cas9 domain or NLS) are present. In some embodiments, a linker is present between the cytidine deaminase and adenosine deaminase domains and the napDNAbp. In some embodiments, the “-” used in the general architecture below indicates the presence of an optional linker. In some embodiments, the cytidine deaminase and adenosine deaminase and the napDNAbp are fused via any of the linkers provided herein. For example, in some embodiments the cytidine deaminase and adenosine deaminase and the napDNAbp are fused via any of the linkers provided herein.

In some embodiments, the general architecture of exemplary napDNAbp (e.g., Cas9 or Cas12) fusion proteins with a cytidine or adenosine deaminase and a napDNAbp (e.g., Cas9 or Cas12) domain comprises any one of the following structures, where NLS is a nuclear localization sequence (e.g., any NLS provided herein), NH₂ is the N-terminus of the fusion protein, and COOH is the C-terminus of the fusion protein:
NH₂-NLS-[cytidine deaminase]-[napDNAbp domain]-COOH;
NH₂-NLS [napDNAbp domain]-[cytidine deaminase]-COOH;
NH₂-[cytidine deaminase]-[napDNAbp domain]-NLS-COOH;
NH₂-[napDNAbp domain]-[cytidine deaminase]-NLS—COOH;
NH₂-NLS-[adenosine deaminase]-[napDNAbp domain]-COOH;
NH₂-NLS [napDNAbp domain]-[adenosine deaminase]-COOH;
NH₂-[adenosine deaminase]-[napDNAbp domain]-NLS—COOH;
NH₂-[napDNAbp domain]-[adenosine deaminase]-NLS—COOH;
NH₂-NLS-[cytidine deaminase]-[napDNAbp domain]-[adenosine deaminase]-COOH;
NH₂-NLS-[adenosine deaminase]-[napDNAbp domain]-[cytidine deaminase]-COOH;
NH₂-NLS-[adenosine deaminase] [cytidine deaminase]-[napDNAbp domain]-COOH;
NH₂-NLS-[cytidine deaminase]-[adenosine deaminase]-[napDNAbp domain]-COOH;
NH₂-NLS-[napDNAbp domain]-[adenosine deaminase]-[cytidine deaminase]-COOH;
NH₂-NLS-[napDNAbp domain]-[cytidine deaminase]-[adenosine deaminase]-COOH;
NH₂-[cytidine deaminase]-[napDNAbp domain]-[adenosine deaminase]-NLS—COOH;
NH₂-[adenosine deaminase]-[napDNAbp domain]-[cytidine deaminase]-NLS—COOH;
NH₂-[adenosine deaminase] [cytidine deaminase]-[napDNAbp domain]-NLS—COOH;
NH₂-[cytidine deaminase]-[adenosine deaminase]-[napDNAbp domain]-NLS—COOH;
NH₂-[napDNAbp domain]-[adenosine deaminase]-[cytidine deaminase]-NLS—COOH; or

NH₂-[napDNAbp domain]-[cytidine deaminase]-[adenosine deaminase]-NLS—COOH. In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR[PAATKKAGQA]KKKK (SEQ ID NO: 191), is the prototype of the ubiquitous bipartite signal: two clusters of basic amino acids, separated by a spacer of about 10 amino acids. The sequence of an exemplary bipartite NLS follows: PKKKRKVEGADKRTADGSEFESPKKKRKV (SEQ ID NO: 328)

A vector that encodes a CRISPR enzyme comprising one or more nuclear localization sequences (NLSs) can be used. For example, there can be or be about 1, 2, 3, 4, 5, 6, 7, 8, 9, 10 NLSs used. A CRISPR enzyme can comprise the NLSs at or near the amino-terminus, about or more than about 1, 2, 3, 4, 5, 6, 7, 8, 9, 10 NLSs at or near the carboxy-terminus, or any combination thereof (e.g., one or more NLS at the amino-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise about 6 NLSs. Aₙ NLS is considered near the N- or C-terminus when the nearest amino acid to the NLS is within about 50 amino acids along a polypeptide chain from the N- or C-terminus, e.g., within 1, 2, 3, 4, 5, 10, 15, 20, 25, 30, 40, or 50 amino acids.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor can comprise a nuclease, a nickase, a recombinase, a deaminase, a methyltransferase, a methylase, an acetylase, an acetyltransferase, a transcriptional activator, or a transcriptional repressor domain.

In some embodiments, a base editor can comprise an uracil glycosylase inhibitor (UGI) domain. In some embodiments, cellular DNA repair response to the presence of U: G heteroduplex DNA can be responsible for a decrease in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein comprising a UGI domain.

In some embodiments, a base editor comprises as a domain all or a portion of a double-strand break (DSB) binding protein. For example, a DSB binding protein can include a Gam protein of bacteriophage Mu that can bind to the ends of DSBs and can protect them from degradation. See Komor, A. C., et al., “Improved base excision repair inhibition and bacteriophage Mu Gam protein yields C:G-to-T:A base editors with higher efficiency and product purity” Science Advances 3:eaao4774 (2017), the entire content of which is hereby incorporated by reference.

Additionally, in some embodiments, a Gam protein can be fused to an N terminus of a base editor. In some embodiments, a Gam protein can be fused to a C terminus of a base editor. The Gam protein of bacteriophage Mu can bind to the ends of double strand breaks (DSBs) and protect them from degradation. In some embodiments, using Gam to bind the free ends of DSB can reduce indel formation during the process of base editing. In some embodiments, 174-residue Gam protein is fused to the N terminus of the base editors. See Komor, A. C., et al., “Improved base excision repair inhibition and bacteriophage Mu Gam protein yields C:G-to-T:A base editors with higher efficiency and product purity” Science Advances 3:eaao4774 (2017). In some embodiments, a mutation or mutations can change the length of a base editor domain relative to a wild type domain. For example, a deletion of at least one amino acid in at least one domain can reduce the length of the base editor. In another case, a mutation or mutations do not change the length of a domain relative to a wild type domain. For example, substitutions in any domain does not change the length of the base editor.

Non-limiting examples of such base editors, where the length of all the domains is the same as the wild type domains, can include:
NH2-[nucleobase editing domain]-Linker1-[APOBEC1]-Linker2-[nucleobase editing domain]-COOH;
NH2-[nucleobase editing domain]-Linker1-[APOBEC1]-[nucleobase editing domain]-COOH;
NH2-[nucleobase editing domain]-[APOBEC1]-Linker2-[nucleobase editing domain]-COOH;
NH2-[nucleobase editing domain]-[APOBEC1]-[nucleobase editing domain]-COOH;
NH2-[nucleobase editing domain]-Linker1-[APOBEC1]-Linker2-[nucleobase editing domain]-[UGI]-COOH;
NH2-[nucleobase editing domain]-Linker1-[APOBEC1]-[nucleobase editing domain]-[UGI]-COOH;
NH2-[nucleobase editing domain]-[APOBEC1]-Linker2-[nucleobase editing domain]-[UGI]-COOH;
NH2-[nucleobase editing domain]-[APOBEC1]-[nucleobase editing domain]-[UGI]-COOH;
NH2-[UGI]-[nucleobase editing domain]-Linker1-[APOBEC1]-Linker2-[nucleobase editing domain]-COOH;
NH2-[UGI]-[nucleobase editing domain]-Linker1-[APOBEC1]-[nucleobase editing domain]-COOH;
NH2-[UGI]-[nucleobase editing domain]-[APOBEC1]-Linker2-[nucleobase editing domain]-COOH; or
NH2-[UGI]-[nucleobase editing domain]-[APOBEC1]-[nucleobase editing domain]-COOH.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

In some embodiments, a base editing system as provided herein provides a new approach to genome editing that uses a fusion protein containing a catalytically defective Streptococcus pyogenes Cas9, a deaminase (e.g., cytidine or adenosine deaminase), and an inhibitor of base excision repair to induce programmable, single nucleotide (C→T or A→G) changes in DNA without generating double-strand DNA breaks, without requiring a donor DNA template, and without inducing an excess of stochastic insertions and deletions.

Details of nucleobase editing proteins are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleic acid (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the first base is adenine, and the second base is not a G, C, A, or T. In some embodiments, the second base is inosine.

In some embodiments, a single guide polynucleotide may be utilized to target a deaminase to a target nucleic acid sequence. In some embodiments, a single pair of guide polynucleotides may be utilized to target different deaminases to a target nucleic acid sequence.

The nucleobase components and the polynucleotide programmable nucleotide binding component of a base editor system may be associated with each other covalently or non-covalently. For example, in some embodiments, the deaminase domain can be targeted to a target nucleotide sequence by a polynucleotide programmable nucleotide binding domain. In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component, e.g., the deaminase component can comprise an additional heterologous portion or domain that is capable of interacting with, associating with, or capable of forming a complex with an additional heterologous portion or domain that is part of a polynucleotide programmable nucleotide binding domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a steril alpha motif, a telomerase Ku binding motif and Ku protein, a telomerase Sm7 binding motif and Sm7 protein, or an RNA recognition motif.

A base editor system may further comprise a guide polynucleotide component. It should be appreciated that components of the base editor system may be associated with each other via covalent bonds, noncovalent interactions, or any combination of associations and interactions thereof. In some embodiments, a deaminase domain can be targeted to a target nucleotide sequence by a guide polynucleotide. For example, in some embodiments, the nucleobase editing component of the base editor system, e.g., the deaminase component, can comprise an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain (e.g., polynucleotide binding domain such as an RNA or DNA binding protein) can be fused or linked to the deaminase domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or an RNA recognition motif.

In some embodiments, a base editor system can further comprise an inhibitor of base excision repair (BER) component. It should be appreciated that components of the base editor system may be associated with each other via covalent bonds, noncovalent interactions, or any combination of associations and interactions thereof. The inhibitor of BER component may comprise a base excision repair inhibitor. In some embodiments, the inhibitor of base excision repair can be a uracil DNA glycosylase inhibitor (UGI). In some embodiments, the inhibitor of base excision repair can be an inosine base excision repair inhibitor. In some embodiments, the inhibitor of base excision repair can be targeted to the target nucleotide sequence by the polynucleotide programmable nucleotide binding domain. In some embodiments, a polynucleotide programmable nucleotide binding domain can be fused or linked to an inhibitor of base excision repair. In some embodiments, a polynucleotide programmable nucleotide binding domain can be fused or linked to a deaminase domain and an inhibitor of base excision repair. In some embodiments, a polynucleotide programmable nucleotide binding domain can target an inhibitor of base excision repair to a target nucleotide sequence by non-covalently interacting with or associating with the inhibitor of base excision repair. For example, in some embodiments, the inhibitor of base excision repair component can comprise an additional heterologous portion or domain that is capable of interacting with, associating with, or capable of forming a complex with an additional heterologous portion or domain that is part of a polynucleotide programmable nucleotide binding domain. In some embodiments, the inhibitor of base excision repair can be targeted to the target nucleotide sequence by the guide polynucleotide. For example, in some embodiments, the inhibitor of base excision repair can comprise an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain of the guide polynucleotide (e.g., polynucleotide binding domain such as an RNA or DNA binding protein) can be fused or linked to the inhibitor of base excision repair. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or an RNA recognition motif.

In some embodiments, the base editor inhibits base excision repair (BER) of the edited strand. In some embodiments, the base editor protects or binds the non-edited strand. In some embodiments, the base editor comprises UGI activity. In some embodiments, the base editor comprises a catalytically inactive inosine-specific nuclease. In some embodiments, the base editor comprises nickase activity. In some embodiments, the intended edit of base pair is upstream of a PAM site. In some embodiments, the intended edit of base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edit of base-pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site.

In some embodiments, the method does not require a canonical (e.g., NGG) PAM site. In some embodiments, the nucleobase editor comprises a linker or a spacer. In some embodiments, the linker or spacer is 1-25 amino acids in length. In some embodiments, the linker or spacer is 5-20 amino acids in length. In some embodiments, the linker or spacer is 10, 11, 12, 13, 14, 15, 16, 17, 18, 19, or 20 amino acids in length.

In some embodiments, the base editing fusion proteins provided herein need to be positioned at a precise location, for example, where a target base is placed within a defined region (e.g., a “deamination window”). In some embodiments, a target can be within a 4 base region. In some embodiments, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

In some embodiments, the target region comprises a target window, wherein the target window comprises the target nucleobase pair. In some embodiments, the target window comprises 1-10 nucleotides. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edit of base pair is within the target window. In some embodiments, the target window comprises the intended edit of base pair. In some embodiments, the method is performed using any of the base editors provided herein. In some embodiments, a target window is a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Other exemplary features that can be present in a base editor as disclosed herein are localization sequences, such as cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, non-limiting exemplary cytidine base editors (CBE) include BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, or saB4E-Gam. BE4 extends the APOBEC1-Cas9n(D10A) linker to 32 amino acids and the Cas9n-UGI linker to 9 amino acids, and appends a second copy of UGI to the C-terminus of the construct with another 9-amino acid linker into a single base editor construct. The base editors saBE3 and saBE4 have the S. pyogenes Cas9n(D10A) replaced with the smaller S. aureus Cas9n(D10A). BE3-Gam, saBE3-Gam, BE4-Gam, and saBE4-Gam have 174 residues of Gam protein fused to the N-terminus of BE3, saBE3, BE4, and saBE4 via the 16 amino acid XTEN linker.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises evolved TadA variant. In some embodiments, the ABE is ABE 1.2 (TadA*-XTEN-nCas9-NLS). In some embodiments, TadA* comprises A106V and D108N mutations.

In some embodiments, the ABE is a second-generation ABE. In some embodiments, the ABE is ABE2.1, which comprises additional mutations D147Y and E155V in TadA* (TadA*2.1). In some embodiments, the ABE is ABE2.2, ABE2.1 fused to catalytically inactivated version of human alkyl adenine DNA glycosylase (AAG with E125Q mutation). In some embodiments, the ABE is ABE2.3, ABE2.1 fused to catalytically inactivated version of E. coli Endo V (inactivated with D35A mutation). In some embodiments, the ABE is ABE2.6 which has a linker twice as long (32 amino acids, (SGGS)₂ (SEQ ID NO: 330)-XTEN-(SGGS)₂ (SEQ ID NO: 330)) as the linker in ABE2.1. In some embodiments, the ABE is ABE2.7, which is ABE2.1 tethered with an additional wild-type TadA monomer. In some embodiments, the ABE is ABE2.8, which is ABE2.1 tethered with an additional TadA*2.1 monomer. In some embodiments, the ABE is ABE2.9, which is a direct fusion of evolved TadA (TadA*2.1) to the N-terminus of ABE2.1. In some embodiments, the ABE is ABE2.10, which is a direct fusion of wild-type TadA to the N-terminus of ABE2.1. In some embodiments, the ABE is ABE2.11, which is ABE2.9 with an inactivating E59A mutation at the N-terminus of TadA* monomer. In some embodiments, the ABE is ABE2.12, which is ABE2.9 with an inactivating E59A mutation in the internal TadA* monomer.

In some embodiments, the ABE is a third generation ABE. In some embodiments, the ABE is ABE3.1, which is ABE2.3 with three additional TadA mutations (L84F, H123Y, and I156F).

In some embodiments, the ABE is a fourth generation ABE. In some embodiments, the ABE is ABE4.3, which is ABE3.1 with an additional TadA mutation A142N (TadA*4.3).

In some embodiments, the ABE is a fifth generation ABE. In some embodiments, the ABE is ABE5.1, which is generated by importing a consensus set of mutations from surviving clones (H36L, R51L, S146C, and K157N) into ABE3.1. In some embodiments, the ABE is ABE5.3, which has a heterodimeric construct containing wild-type E. coli TadA fused to an internal evolved TadA*. In some embodiments, the ABE is ABE5.2, ABE5.4, ABE5.5, ABE5.6, ABE5.7, ABE5.8, ABE5.9, ABE5.10, ABE5.11, ABE5.12, ABE5.13, or ABE5.14, as shown in Table 11 below. In some embodiments, the ABE is a sixth generation ABE. In some embodiments, the ABE is ABE6.1, ABE6.2, ABE6.3, ABE6.4, ABE6.5, or ABE6.6, as shown in Table 11 below. In some embodiments, the ABE is a seventh generation ABE. In some embodiments, the ABE is ABE7.1, ABE7.2, ABE7.3, ABE7.4, ABE7.5, ABE7.6, ABE7.7, ABE7.8, ABE 7.9, or ABE7.10, as shown in Table 11 below.

TABLE 11
Genotypes of ABEs

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| ABE0.1 | W | R | H | N | P | | R | N | L | S | A | D | H | G | A | S | D | R | E | I | K | K |
| ABE0.2 | W | R | H | N | P | | R | N | L | S | A | D | H | G | A | S | D | R | E | I | K | K |
| ABE1.1 | W | R | H | N | P | | R | N | L | S | A | N | H | G | A | S | D | R | E | I | K | K |
| ABE1.2 | W | R | H | N | P | | R | N | L | S | V | N | H | G | A | S | D | R | E | I | K | K |
| ABE2.1 | W | R | H | N | P | | R | N | L | S | V | N | H | G | A | S | Y | R | V | I | K | K |
| ABE2.2 | W | R | H | N | P | | R | N | L | S | V | N | H | G | A | S | Y | R | V | I | K | K |
| ABE2.3 | W | R | H | N | P | | R | N | L | S | V | N | H | G | A | S | Y | R | V | I | K | K |
| ABE2.4 | W | R | H | N | P | | R | N | L | S | V | N | H | G | A | S | Y | R | V | I | K | K |
| ABE2.5 | W | R | H | N | P | | R | N | L | S | V | N | H | G | A | S | Y | R | V | I | K | K |
| ABE2.6 | W | R | H | N | P | | R | N | L | S | V | N | H | G | A | S | Y | R | V | I | K | K |
| ABE2.7 | W | R | H | N | P | | R | N | L | S | V | N | H | G | A | S | Y | R | V | I | K | K |
| ABE2.8 | W | R | H | N | P | | R | N | L | S | V | N | H | G | A | S | Y | R | V | I | K | K |
| ABE2.9 | W | R | H | N | P | | R | N | L | S | V | N | H | G | A | S | Y | R | V | I | K | K |
| ABE2.10 | W | R | H | N | P | | R | N | L | S | V | N | H | G | A | S | Y | R | V | I | K | K |
| ABE2.11 | W | R | H | N | P | | R | N | L | S | V | N | H | G | A | S | Y | R | V | I | K | K |
| ABE2.12 | W | R | H | N | P | | R | N | L | S | V | N | Y | G | A | S | Y | R | V | I | K | K |
| ABE3.1 | W | R | H | N | P | | R | N | F | S | V | N | Y | G | A | S | Y | R | V | F | K | K |
| ABE3.2 | W | R | H | N | P | | R | N | F | S | V | N | Y | G | A | S | Y | R | V | F | K | K |
| ABE3.3 | W | R | H | N | P | | R | N | F | S | V | N | Y | G | A | S | Y | R | V | F | K | K |
| ABE3.4 | W | R | H | N | P | | R | N | F | S | V | N | Y | G | A | S | Y | R | V | F | K | K |
| ABE3.5 | W | R | H | N | P | | R | N | F | S | V | N | Y | G | A | S | Y | R | V | F | K | K |
| ABE3.6 | W | R | H | N | P | | R | N | F | S | V | N | Y | G | A | S | Y | R | V | F | K | K |
| ABE3.7 | W | R | H | N | P | | R | N | F | S | V | N | Y | G | A | S | Y | R | V | F | K | K |
| ABE3.8 | W | R | H | N | P | | R | N | F | S | V | N | Y | G | A | S | Y | R | V | F | K | K |
| ABE4.1 | W | R | H | N | P | | R | N | L | S | V | N | H | G | N | S | Y | R | V | I | K | K |
| ABE4.2 | W | G | H | N | P | | R | N | L | S | V | N | H | G | N | S | Y | R | V | I | K | K |
| ABE4.3 | W | R | H | N | P | | R | N | F | S | V | N | Y | G | N | S | Y | R | V | F | K | K |
| ABE5.1 | W | R | L | N | P | | L | N | F | S | V | N | Y | G | A | C | Y | R | V | F | N | K |
| ABE5.2 | W | R | H | S | P | | R | N | F | S | V | N | Y | G | A | S | Y | R | V | F | K | T |
| ABE5.3 | W | R | L | N | P | | L | N | I | S | V | N | Y | G | A | C | Y | R | V | F | N | K |
| ABE5.4 | W | R | H | S | P | | R | N | F | S | V | N | Y | G | A | S | Y | R | V | F | K | T |
| ABE5.5 | W | R | L | N | P | | L | N | F | S | V | N | Y | G | A | C | Y | R | V | F | N | K |
| ABE5.6 | W | R | L | N | P | | L | N | F | S | V | N | Y | G | A | C | Y | R | V | F | N | K |
| ABE5.7 | W | R | L | N | P | | L | N | F | S | V | N | Y | G | A | C | Y | R | V | F | N | K |
| ABE5.8 | W | R | L | N | P | | L | N | F | S | V | N | Y | G | A | C | Y | R | V | F | N | K |
| ABE5.9 | W | R | L | N | P | | L | N | F | S | V | N | Y | G | A | C | Y | R | V | F | N | K |
| ABE5.10 | W | R | L | N | P | | L | N | F | S | V | N | Y | G | A | C | Y | R | V | F | N | K |
| ABE5.11 | W | R | L | N | P | | L | N | F | S | V | N | Y | G | A | C | Y | R | V | F | N | K |
| ABE5.12 | W | R | L | N | P | | L | N | F | S | V | N | Y | G | A | C | Y | R | V | F | N | K |
| ABE5.13 | W | R | H | N | P | | L | D | F | S | V | N | Y | A | A | S | Y | R | V | F | K | K |
| ABE5.14 | W | R | H | N | S | | L | N | F | C | V | N | Y | G | A | S | Y | R | V | F | K | K |
| ABE6.1 | W | R | H | N | S | | L | N | F | S | V | N | Y | G | N | S | Y | R | V | F | K | K |
| ABE6.2 | W | R | H | N | T | V | L | N | F | S | V | N | Y | G | N | S | Y | R | V | F | N | K |
| ABE6.3 | W | R | L | N | S | | L | N | F | S | V | N | Y | G | A | C | Y | R | V | F | N | K |
| ABE6.4 | W | R | L | N | S | | L | N | F | S | V | N | Y | G | N | C | Y | R | V | F | N | K |
| ABE6.5 | W | R | L | N | T | V | L | N | F | S | V | N | Y | G | A | C | Y | R | V | F | N | K |
| ABE6.6 | W | R | L | N | T | V | L | N | F | S | V | N | Y | G | N | C | Y | R | V | F | N | K |
| ABE7.1 | W | R | L | N | A | | L | N | F | S | V | N | Y | G | A | C | Y | R | V | F | N | K |
| ABE7.2 | W | R | L | N | A | | L | N | F | S | V | N | Y | G | N | C | Y | R | V | F | N | K |
| ABE7.3 | L | R | L | N | A | | L | N | F | S | V | N | Y | G | A | C | Y | R | V | F | N | K |
| ABE7.4 | R | R | L | N | A | | L | N | F | S | V | N | Y | G | A | C | Y | R | V | F | N | K |
| ABE7.5 | W | R | L | N | A | | L | N | F | S | V | N | Y | G | A | C | Y | H | V | F | N | K |
| ABE7.6 | W | R | L | N | A | | L | N | I | S | V | N | Y | G | A | C | Y | P | V | F | N | K |
| ABE7.7 | L | R | L | N | A | | L | N | F | S | V | N | Y | G | A | C | Y | P | V | F | N | K |
| ABE7.8 | L | R | L | N | A | | L | N | F | S | V | N | Y | G | N | C | Y | R | V | F | N | K |
| ABE7.9 | L | R | L | N | A | | L | N | F | S | V | N | Y | G | N | C | Y | P | V | F | N | K |
| ABE7.10 | R | R | L | N | A | | L | N | F | S | V | N | Y | G | A | C | Y | P | V | F | N | K |

In some embodiments, the base editor is an eighth generation ABE (ABE8). In some embodiments, the ABE8 contains a TadA*8 variant. In some embodiments, the ABE8 has a monomeric construct containing a TadA*8 variant (“ABE8.x-m”). In some embodiments, the ABE8 is ABE8.1-m, which has a monomeric construct containing TadA*7.10 with a Y147T mutation (TadA*8.1). In some embodiments, the ABE8 is ABE8.2-m, which has a monomeric construct containing TadA*7.10 with a Y147R mutation (TadA*8.2). In some embodiments, the ABE8 is ABE8.3-m, which has a monomeric construct containing TadA*7.10 with a Q154S mutation (TadA*8.3). In some embodiments, the ABE8 is ABE8.4-m, which has a monomeric construct containing TadA*7.10 with a Y123H mutation (TadA*8.4). In some embodiments, the ABE8 is ABE8.5-m, which has a monomeric construct containing TadA*7.10 with a V82S mutation (TadA*8.5). In some embodiments, the ABE8 is ABE8.6-m, which has a monomeric construct containing TadA*7.10 with a T166R mutation (TadA*8.6). In some embodiments, the ABE8 is ABE8.7-m, which has a monomeric construct containing TadA*7.10 with a Q154R mutation (TadA*8.7). In some embodiments, the ABE8 is ABE8.8-m, which has a monomeric construct containing TadA*7.10 with Y147R, Q154R, and Y123H mutations (TadA*8.8). In some embodiments, the ABE8 is ABE8.9-m, which has a monomeric construct containing TadA*7.10 with Y147R, Q154R and I76Y mutations (TadA*8.9). In some embodiments, the ABE8 is ABE8.10-m, which has a monomeric construct containing TadA*7.10 with Y147R, Q154R, and T166R mutations (TadA*8.10). In some embodiments, the ABE8 is ABE8.11-m, which has a monomeric construct containing TadA*7.10 with Y147T and Q154R mutations (TadA*8.11). In some embodiments, the ABE8 is ABE8.12-m, which has a monomeric construct containing TadA*7.10 with Y147T and Q154S mutations (TadA*8.12).

In some embodiments, the ABE8 is ABE8.13-m, which has a monomeric construct containing TadA*7.10 with Y123H (Y123H reverted from H123Y), Y147R, Q154R and I76Y mutations (TadA*8.13). In some embodiments, the ABE8 is ABE8.14-m, which has a monomeric construct containing TadA*7.10 with I76Y and V82S mutations (TadA*8.14). In some embodiments, the ABE8 is ABE8.15-m, which has a monomeric construct containing TadA*7.10 with V82S and Y147R mutations (TadA*8.15). In some embodiments, the ABE8 is ABE8.16-m, which has a monomeric construct containing TadA*7.10 with V82S, Y123H (Y123H reverted from H123Y) and Y147R mutations (TadA*8.16). In some embodiments, the ABE8 is ABE8.17-m, which has a monomeric construct containing TadA*7.10 with V82S and Q154R mutations (TadA*8.17). In some embodiments, the ABE8 is ABE8.18-m, which has a monomeric construct containing TadA*7.10 with V82S, Y123H (Y123H reverted from H123Y) and Q154R mutations (TadA*8.18). In some embodiments, the ABE8 is ABE8.19-m, which has a monomeric construct containing TadA*7.10 with V82S, Y123H (Y123H reverted from H123Y), Y147R and Q154R mutations (TadA*8.19). In some embodiments, the ABE8 is ABE8.20-m, which has a monomeric construct containing TadA*7.10 with I76Y, V82S, Y123H (Y123H reverted from H123Y), Y147R and Q154R mutations (TadA*8.20). In some embodiments, the ABE8 is ABE8.21-m, which has a monomeric construct containing TadA*7.10 with Y147R and Q154S mutations (TadA*8.21). In some embodiments, the ABE8 is ABE8.22-m, which has a monomeric construct containing TadA*7.10 with V82S and Q154S mutations (TadA*8.22). In some embodiments, the ABE8 is ABE8.23-m, which has a monomeric construct containing TadA*7.10 with V82S and Y123H (Y123H reverted from H123Y) mutations (TadA*8.23). In some embodiments, the ABE8 is ABE8.24-m, which has a monomeric construct containing TadA*7.10 with V82S, Y123H (Y123H reverted from H123Y), and Y147T mutations (TadA*8.24).

In some embodiments, the ABE8 has a heterodimeric construct containing wild-type E. coli TadA fused to a TadA*8 variant (“ABE8.x-d”). In some embodiments, the ABE8 is ABE8.1-d, which has a heterodimeric construct containing wild-type E. coli TadA fused to TadA*7.10 with a Y147T mutation (TadA*8.1). In some embodiments, the ABE8 is ABE8.2-d, which has a heterodimeric construct containing wild-type E. coli TadA fused to TadA*7.10 with a Y147R mutation (TadA*8.2). In some embodiments, the ABE8 is ABE8.3-d, which has a heterodimeric construct containing wild-type E. coli TadA fused to TadA*7.10 with a Q154S mutation (TadA*8.3). In some embodiments, the ABE8 is ABE8.4-d, which has a heterodimeric construct containing wild-type E. coli TadA fused to TadA*7.10 with a Y123H mutation (TadA*8.4). In some embodiments, the ABE8 is ABE8.5-d, which has a heterodimeric construct containing wild-type E. coli TadA fused to TadA*7.10 with a V82S mutation (TadA*8.5). In some embodiments, the ABE8 is ABE8.6-d, which has a heterodimeric construct containing wild-type E. coli TadA fused to TadA*7.10 with a T166R mutation (TadA*8.6). In some embodiments, the ABE8 is ABE8.7-d, which has a heterodimeric construct containing wild-type E. coli TadA fused to TadA*7.10 with a Q154R mutation (TadA*8.7). In some embodiments, the ABE8 is ABE8.8-d, which has a heterodimeric construct containing wild-type E. coli TadA fused to TadA*7.10 with Y147R, Q154R, and Y123H mutations (TadA*8.8). In some embodiments, the ABE8 is ABE8.9-d, which has a heterodimeric construct containing wild-type E. coli TadA fused to TadA*7.10 with Y147R, Q154R and I76Y mutations (TadA*8.9). In some embodiments, the ABE8 is ABE8.10-d, which has a heterodimeric construct containing wild-type E. coli TadA fused to TadA*7.10 with Y147R, Q154R, and T166R mutations (TadA*8.10). In some embodiments, the ABE8 is ABE8.11-d, which has a heterodimeric construct containing wild-type E. coli TadA fused to TadA*7.10 with Y147T and Q154R mutations (TadA*8.11). In some embodiments, the ABE8 is ABE8.12-d, which has heterodimeric construct containing wild-type E. coli TadA fused to TadA*7.10 with Y147T and Q154S mutations (TadA*8.12). In some embodiments, the ABE8 is ABE8.13-d, which has a heterodimeric construct containing wild-type E. coli TadA fused to TadA*7.10 with Y123H (Y123H reverted from H123Y), Y147R, Q154R and I76Y mutations (TadA*8.13). In some embodiments, the ABE8 is ABE8.14-d, which has a heterodimeric construct containing wild-type E. coli TadA fused to TadA*7.10 with I76Y and V82S mutations (TadA*8.14). In some embodiments, the ABE8 is ABE8.15-d, which has a heterodimeric construct containing wild-type E. coli TadA fused to TadA*7.10 with V82S and Y147R mutations (TadA*8.15). In some embodiments, the ABE8 is ABE8.16-d, which has a heterodimeric construct containing wild-type E. coli TadA fused to TadA*7.10 with V82S, Y123H (Y123H reverted from H123Y) and Y147R mutations (TadA*8.16). In some embodiments, the ABE8 is ABE8.17-d, which has a heterodimeric construct containing wild-type E. coli TadA fused to TadA*7.10 with V82S and Q154R mutations (TadA*8.17). In some embodiments, the ABE8 is ABE8.18-d, which has a heterodimeric construct containing wild-type E. coli TadA fused to TadA*7.10 with V82S, Y123H (Y123H reverted from H123Y) and Q154R mutations (TadA*8.18). In some embodiments, the ABE8 is ABE8.19-d, which has a heterodimeric construct containing wild-type E. coli TadA fused to TadA*7.10 with V82S, Y123H (Y123H reverted from H123Y), Y147R and Q154R mutations (TadA*8.19). In some embodiments, the ABE8 is ABE8.20-d, which has a heterodimeric construct containing wild-type E. coli TadA fused to TadA*7.10 with I76Y, V82S, Y123H (Y123H reverted from H123Y), Y147R and Q154R mutations (TadA*8.20). In some embodiments, the ABE8 is ABE8.21-d, which has a heterodimeric construct containing wild-type E. coli TadA fused to TadA*7.10 with Y147R and Q154S mutations (TadA*8.21). In some embodiments, the ABE8 is ABE8.22-d, which has a heterodimeric construct containing wild-type E. coli TadA fused to TadA*7.10 with V82S and Q154S mutations (TadA*8.22). In some embodiments, the ABE8 is ABE8.23-d, which has a heterodimeric construct containing wild-type E. coli TadA fused to TadA*7.10 with V82S and Y123H (Y123H reverted from H123Y) mutations (TadA*8.23). In some embodiments, the ABE8 is ABE8.24-d, which has a heterodimeric construct containing wild-type E. coli TadA fused to TadA*7.10 with V82S, Y123H (Y123H reverted from H123Y), and Y147T mutations (TadA*8.24).

In some embodiments, the ABE8 has a heterodimeric construct containing TadA*7.10 fused to a TadA*8 variant (“ABE8.x-7”). In some embodiments, the ABE8 is ABE8.1-7, which has a heterodimeric construct containing TadA*7.10 fused to TadA*7.10 with a Y147T mutation (TadA*8.1). In some embodiments, the ABE8 is ABE8.2-7, which has a heterodimeric construct containing TadA*7.10 fused to TadA*7.10 with a Y147R mutation (TadA*8.2). In some embodiments, the ABE8 is ABE8.3-7, which has a heterodimeric construct containing TadA*7.10 fused to TadA*7.10 with a Q154S mutation (TadA*8.3). In some embodiments, the ABE8 is ABE8.4-7, which has a heterodimeric construct containing TadA*7.10 fused to TadA*7.10 with a Y123H mutation (TadA*8.4). In some embodiments, the ABE8 is ABE8.5-7, which has a heterodimeric construct containing TadA*7.10 fused to TadA*7.10 with a V82S mutation (TadA*8.5). In some embodiments, the ABE8 is ABE8.6-7, which has a heterodimeric construct containing TadA*7.10 fused to TadA*7.10 with a T166R mutation (TadA*8.6). In some embodiments, the ABE8 is ABE8.7-7, which has a heterodimeric construct containing TadA*7.10 fused to TadA*7.10 with a Q154R mutation (TadA*8.7). In some embodiments, the ABE8 is ABE8.8-7, which has a heterodimeric construct containing TadA*7.10 fused to TadA*7.10 with Y147R, Q154R, and Y123H mutations (TadA*8.8). In some embodiments, the ABE8 is ABE8.9-7, which has a heterodimeric construct containing TadA*7.10 fused to TadA*7.10 with Y147R, Q154R and I76Y mutations (TadA*8.9). In some embodiments, the ABE8 is ABE8.10-7, which has a heterodimeric construct containing TadA*7.10 fused to TadA*7.10 with Y147R, Q154R, and T166R mutations (TadA*8.10). In some embodiments, the ABE8 is ABE8.11-7, which has a heterodimeric construct containing TadA*7.10 fused to TadA*7.10 with Y147T and Q154R mutations (TadA*8.11). In some embodiments, the ABE8 is ABE8.12-7, which has a heterodimeric construct containing TadA*7.10 fused to TadA*7.10 with Y147T and Q154S mutations (TadA*8.12). In some embodiments, the ABE8 is ABE8.13-7, which has a heterodimeric construct containing TadA*7.10 fused to TadA*7.10 with Y123H (Y123H reverted from H123Y), Y147R, Q154R and I76Y mutations (TadA*8.13). In some embodiments, the ABE8 is ABE8.14-7, which has a heterodimeric construct containing TadA*7.10 fused to TadA*7.10 with I76Y and V82S mutations (TadA*8.14). In some embodiments, the ABE8 is ABE8.15-7, which has a heterodimeric construct containing TadA*7.10 fused to TadA*7.10 with V82S and Y147R mutations (TadA*8.15). In some embodiments, the ABE8 is ABE8.16-7, which has a heterodimeric construct containing TadA*7.10 fused to TadA*7.10 with V82S, Y123H (Y123H reverted from H123Y) and Y147R mutations (TadA*8.16). In some embodiments, the ABE8 is ABE8.17-7, which has a heterodimeric construct containing TadA*7.10 fused to TadA*7.10 with V82S and Q154R mutations (TadA*8.17). In some embodiments, the ABE8 is ABE8.18-7, which has a heterodimeric construct containing TadA*7.10 fused to TadA*7.10 with V82S, Y123H (Y123H reverted from H123Y) and Q154R mutations (TadA*8.18). In some embodiments, the ABE8 is ABE8.19-7, which has a heterodimeric construct containing TadA*7.10 fused to TadA*7.10 with V82S, Y123H (Y123H reverted from H123Y), Y147R and Q154R mutations (TadA*8.19). In some embodiments, the ABE8 is ABE8.20-7, which has a heterodimeric construct containing TadA*7.10 fused to TadA*7.10 with I76Y, V82S, Y123H (Y123H reverted from H123Y), Y147R and Q154R mutations (TadA*8.20). In some embodiments, the ABE8 is ABE8.21-7, which has a heterodimeric construct containing TadA*7.10 fused to TadA*7.10 with Y147R and Q154S mutations (TadA*8.21). In some embodiments, the ABE8 is ABE8.22-7, which has a heterodimeric construct containing TadA*7.10 fused to TadA*7.10 with V82S and Q154S mutations (TadA*8.22). In some embodiments, the ABE8 is ABE8.23-7, which has a heterodimeric construct containing TadA*7.10 fused to TadA*7.10 with V82S and Y123H (Y123H reverted from H123Y) mutations (TadA*8.23). In some embodiments, the ABE8 is ABE8.24-7, which has a heterodimeric construct containing TadA*7.10 fused to TadA*7.10 with V82S, Y123H (Y123H reverted from H123Y), and Y147T mutations (TadA*8.24 In some embodiments, the ABE is ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, or ABE8.24-d as shown in Table 12 below.

TABLE 12
Adenosine Base Editor 8 (ABE8) Variants

| ABE8 | Adenosine Deaminase | Adenosine Deaminase Description |
|---|---|---|
| ABE8.1-m | TadA*8.1 | Monomer_TadA*7.10 + Y147T |
| ABE8.2-m | TadA*8.2 | Monomer_TadA*7.10 + Y147R |
| ABE8.3-m | TadA*8.3 | Monomer_TadA*7.10 + Q154S |
| ABE8.4-m | TadA*8.4 | Monomer_TadA*7.10 + Y123H |
| ABE8.5-m | TadA*8.5 | Monomer_TadA*7.10 + V82S |
| ABE8.6-m | TadA*8.6 | Monomer_TadA*7.10 + T166R |
| ABE8.7-m | TadA*8.7 | Monomer_TadA*7.10 + Q154R |
| ABE8.8-m | TadA*8.8 | Monomer_TadA*7.10 + Y147R_Q154R_Y123H |
| ABE8.9-m | TadA*8.9 | Monomer_TadA*7.10 + Y147R_Q154R_I76Y |
| ABE8.10-m | TadA*8.10 | Monomer_TadA*7.10 + Y147R_Q154R_T166R |
| ABE8.11-m | TadA*8.11 | Monomer_TadA*7.10 + Y147T_Q154R |
| ABE8.12-m | TadA*8.12 | Monomer_TadA*7.10 + Y147T_Q154S |
| ABE8.13-m | TadA*8.13 | Monomer_TadA*7.10 + |
| | | Y123H_Y147R_Q154R_I76Y |
| ABE8.14-m | TadA*8.14 | Monomer_TadA*7.10 + I76Y_V82S |
| ABE8.15-m | TadA*8.15 | Monomer_TadA*7.10 + V82S_Y147R |
| ABE8.16-m | TadA*8.16 | Monomer_TadA*7.10 + V82S_Y123H_Y147R |
| ABE8.17-m | TadA*8.17 | Monomer_TadA*7.10 + V82S_Q154R |
| ABE8.18-m | TadA*8.18 | Monomer_TadA*7.10 + V82S_Y123H_Q154R |
| ABE8.19-m | TadA*8.19 | Monomer_TadA*7.10 + |
| | | V82S_Y123H_Y147R_Q154R |
| ABE8.20-m | TadA*8.20 | Monomer_TadA*7.10 + |
| | | I76Y_V82S_Y123H_Y147R_Q154R |
| ABE8.21-m | TadA*8.21 | Monomer_TadA*7.10 + Y147R_Q154S |
| ABE8.22-m | TadA*8.22 | Monomer_TadA*7.10 + V82S_Q154S |
| ABE8.23-m | TadA*8.23 | Monomer_TadA*7.10 + V82S_Y123H |
| ABE8.24-m | TadA*8.24 | Monomer_TadA*7.10 + V82S_Y123H_Y147T |
| ABE8.1-d | TadA*8.1 | Heterodimer_(WT) + (TadA*7.10 + Y147T) |
| ABE8.2-d | TadA*8.2 | Heterodimer_(WT) + (TadA*7.10 + Y147R) |
| ABE8.3-d | TadA*8.3 | Heterodimer_(WT) + (TadA*7.10 + Q154S) |
| ABE8.4-d | TadA*8.4 | Heterodimer_(WT) + (TadA*7.10 + Y123H) |
| ABE8.5-d | TadA*8.5 | Heterodimer_(WT) + (TadA*7.10 + V82S) |
| ABE8.6-d | TadA*8.6 | Heterodimer_(WT) + (TadA*7.10 + T166R) |
| ABE8.7-d | TadA*8.7 | Heterodimer_(WT) + (TadA*7.10 + Q154R) |
| ABE8.8-d | TadA*8.8 | Heterodimer_(WT) + (TadA*7.10 + |
| | | Y147R_Q154R_Y123H) |
| ABE8.9-d | TadA*8.9 | Heterodimer_(WT) + (TadA*7.10 + |
| | | Y147R_Q154R_I76Y) |
| ABE8.10-d | TadA*8.10 | Heterodimer_(WT) + (TadA*7.10 + |
| | | Y147R_Q154R_T166R) |
| ABE8.11-d | TadA*8.11 | Heterodimer_(WT) + (TadA*7.10 + Y147T_Q154R) |
| ABE8.12-d | TadA*8.12 | Heterodimer_(WT) + (TadA*7.10 + Y147T_Q154S) |
| ABE8.13-d | TadA*8.13 | Heterodimer_(WT) + (TadA*7.10 + |
| | | Y123H_Y147T_Q154R_I76Y) |
| ABE8.14-d | TadA*8.14 | Heterodimer_(WT) + (TadA*7.10 + I76Y_V82S) |
| ABE8.15-d | TadA*8.15 | Heterodimer_(WT) + (TadA*7.10 + V82S_Y147R) |
| ABE8.16-d | TadA*8.16 | Heterodimer_(WT) + (TadA*7.10 + |
| | | V82S_Y123H_Y147R) |
| ABE8.17-d | TadA*8.17 | Heterodimer_(WT) + (TadA*7.10 + V82S_Q154R) |
| ABE8.18-d | TadA*8.18 | Heterodimer_(WT) + (TadA*7.10 + |
| | | V82S_Y123H_Q154R) |
| ABE8.19-d | TadA*8.19 | Heterodimer_(WT) + (TadA*7.10 + |
| | | V82S_Y123H_Y147R_Q154R) |
| ABE8.20-d | TadA*8.20 | Heterodimer_(WT) + (TadA*7.10 + |
| | | I76Y_V82S_Y123H_Y147R_Q154R) |
| ABE8.21-d | TadA*8.21 | Heterodimer_(WT) + (TadA*7.10 + Y147R_Q154S) |
| ABE8.22-d | TadA*8.22 | Heterodimer_(WT) + (TadA*7.10 + V82S_Q154S) |
| ABE8.23-d | TadA*8.23 | Heterodimer_(WT) + (TadA*7.10 + V82S_Y123H) |
| ABE8.24-d | TadA*8.24 | Heterodimer_(WT) + (TadA*7.10 + |
| | | V82S_Y123H_Y147T) |

In some embodiments, the ABE8 is ABE8a-m, which has a monomeric construct containing TadA*7.10 with R26C, A109S, T111R, D119N, H122N, Y147D, F149Y, T1661, and D167N mutations (TadA*8a). In some embodiments, the ABE8 is ABE8b-m, which has a monomeric construct containing TadA*7.10 with V88A, A109S, T111R, D119N, H122N, F149Y, T166I, and D167N mutations (TadA*8b). In some embodiments, the ABE8 is ABE8c-m, which has a monomeric construct containing TadA*7.10 with R26C, A109S, T111R, D119N, H122N, F149Y, T166I, and D167N mutations (TadA*8c). In some embodiments, the ABE8 is ABE8d-m, which has a monomeric construct containing TadA*7.10 with V88A, T111R, D119N, and F149Y mutations (TadA*8d). In some embodiments, the ABE8 is ABE8e-m, which has a monomeric construct containing TadA*7.10 with A109S, T111R, D119N, H122N, Y147D, F149Y, T166I, and D167N mutations (TadA*8e).

In some embodiments, the ABE8 is ABE8a-d, which has a heterodimeric construct containing wild-type E. coli TadA fused to TadA*7.10 with R26C, A109S, T111R, D119, H122N, Y147D, F149Y, T166I, and D167N mutations (TadA*8a). In some embodiments, the ABE8 is ABE8b-d, which has a heterodimeric construct containing wild-type E. coli TadA fused to TadA*7.10 with V88A, A109S, T111R, D119N, H122N, F149Y, T166I, and D167N mutations (TadA*8b). In some embodiments, the ABE8 is ABE8c-d, which has a heterodimeric construct containing wild-type E. coli TadA fused to TadA*7.10 with R26C, A109S, T111R, D119N, H122N, F149Y, T166I, and D167N mutations (TadA*8c). In some embodiments, the ABE8 is ABE8d-d, which has a heterodimeric construct containing wild-type E. coli TadA fused to TadA*7.10 with V88A, T111R, D119N, and F149Y mutations (TadA*8d). In some embodiments, the ABE8 is ABE8e-d, which has a heterodimeric construct containing wild-type E. coli TadA fused to TadA*7.10 with A109S, T111R, Di 19N, H122N, Y147D, F149Y, T166I, and D167N mutations (TadA*8e).

In some embodiments, the ABE8 is ABE8a-7, which has a heterodimeric construct containing TadA*7.10 fused to TadA*7.10 with R26C, A109S, T111R, D119, H122N, Y147D, F149Y, T166I, and D167N mutations (TadA*8a). In some embodiments, the ABE8 is ABE8b-7, which has a heterodimeric construct containing TadA*7.10 fused to TadA*7.10 with V88A, A109S, T111R, D119N, H122N, F149Y, T166I, and D167N mutations (TadA*8b). In some embodiments, the ABE8 is ABE8c-7, which has a heterodimeric construct containing TadA*7.10 fused to TadA*7.10 with R26C, A109S, T111R, Di 19N, H122N, F149Y, T1661, and D167N mutations (TadA*8c). In some embodiments, the ABE8 is ABE8d-7, which has a heterodimeric construct containing TadA*7.10 fused to TadA*7.10 with V88A, T111R, Di 19N, and F149Y mutations (TadA*8d). In some embodiments, the ABE8 is ABE8e-7, which has a heterodimeric construct containing TadA*7.10 fused to TadA*7.10 with A109S, T111R, D119N, H122N, Y147D, F149Y, T166I, and D167N mutations (TadA*8e).

In some embodiments, the ABE is ABE8a-m, ABE8b-m, ABE8c-m, ABE8d-m, ABE8e-m, ABE8a-d, ABE8b-d, ABE8c-d, ABE8d-d, or ABE8e-d, as shown in Table 13 below. In some embodiments, the ABE is ABE8e-m or ABE8e-d. ABE8e shows efficient adenine base editing activity and low indel formation when used with Cas homologues other than SpCas9, for example, SaCas9, SaCas9-KKH, Cas12a homologues, e.g., LbCas12a, enAs-Cas12a, SpCas9-NG and circularly permuted CP1028-SpCas9 and CP1041-SpCas9. In addition to the mutations shown for ABE8e in Table 13, off-target RNA and DNA editing were reduced by introducing a V106W substitution into the TadA domain (as described in M. Richter et al., 2020, Nature Biotechnology, doi.org/10.1038/s41587-020-0453-z, the entire contents of which are incorporated by reference herein).

TABLE 13
Additional Adenosine Base Editor 8 Variants. In the table, “monomer”
indicates an ABE comprising a single TadA*7.10 comprising the indicated alterations
and “heterodimer” indicates an ABE comprising a TadA*7.10 comprising
the indicated alterations fused to an E. coli TadA adenosine deaminase.

| ABE8 Base | Adenosine | |
|---|---|---|
| Editor | Deaminase | Adenosine Deaminase Description |
| ABE8a-m | TadA*8a | Monomer_TadA*7.10 + R26C + A109S + T111R + D119N + |
| | | H122N + Y147D + F149Y + T166I + D167N |
| ABE8b-m | TadA*8b | Monomer_TadA*7.10 + V88A + A109S + T111R + D119N + |
| | | H122N + F149Y + T166I + D167N |
| ABE8c-m | TadA*8c | Monomer_TadA*7.10 + R26C + A109S + T111R + D119N + |
| | | H122N + F149Y + T166I + D167N |
| ABE8d-m | TadA*8d | Monomer_TadA*7.10 + V88A + T111R + D119N + F149Y |
| ABE8e-m | TadA*8e | Monomer_TadA*7.10 + A109S + T111R + D119N + H122N + |
| | | Y147D + F149Y + T166I + D167N |
| ABE8a-d | TadA*8a | Heterodimer_(WT) + (TadA*7.10 + R26C + A109S + T111R + |
| | | D119N + H122N + Y147D + F149Y + T166I + D167N) |
| ABE8b-d | TadA*8b | Heterodimer_(WT) + (TadA*7.10 + V88A + A109S + T111R + |
| | | D119N + H122N + F149Y + T166I + D167N) |
| ABE8c-d | TadA*8c | Heterodimer_(WT) + (TadA*7.10 + R26C + A109S + T111R + |
| | | D119N + H122N + F149Y + T166I + D167N) |
| ABE8d-d | TadA*8d | Heterodimer_(WT) + (TadA*7.10 + V88A + T111R + D119N + |
| | | F149Y) |
| ABE8e-d | TadA*8e | Heterodimer_(WT) + (TadA*7.10 + A109S + T111R + |
| | | D119N + H122N + Y147D + F149Y + T166I + D167N) |

In some embodiments, base editors (e.g., ABE8) are generated by cloning an adenosine deaminase variant (e.g., TadA*8) into a scaffold that includes a circular permutant Cas9 (e.g., CP5 or CP6) and a bipartite nuclear localization sequence. In some embodiments, the base editor (e.g., ABE7.9, ABE7.10, or ABE8) is an NGC PAM CP5 variant (S. pyogenes Cas9 or spVRQR Cas9). In some embodiments, the base editor (e.g., ABE7.9, ABE7.10, or ABE8) is an AGA PAM CP5 variant (S. pyogenes Cas9 or spVRQR Cas9). In some embodiments, the base editor (e.g., ABE7.9, ABE7.10, or ABE8) is an NGC PAM CP6 variant (S. pyogenes Cas9 or spVRQR Cas9). In some embodiments, the base editor (e.g. ABE7.9, ABE7.10, or ABE8) is an AGA PAM CP6 variant (S. pyogenes Cas9 or spVRQR Cas9).

In some embodiments, the ABE has a genotype as shown in Table 14 below.

TABLE 14
Genotypes of ABEs

| | 23 | 26 | 36 | 37 | 48 | 49 | 51 | 72 | 84 | 87 | 105 | 108 | 123 | 125 | 142 | 145 | 147 | 152 | 155 | 156 | 157 | 161 |
|---|---|---|---|---|---|---|---|---|---|---|---|---|---|---|---|---|---|---|---|---|---|---|
| ABE7.9 | L | R | L | N | A | | L | N | F | S | V | N | Y | G | N | C | Y | P | V | F | N | K |
| ABE7.10 | R | R | L | N | A | | L | N | F | S | V | N | Y | G | A | C | Y | P | V | F | N | K |

As shown in Table 15 below, genotypes of 40 ABE8s are described. Residue positions in the evolved E. coli TadA portion of ABE are indicated. Mutational changes in ABE8 are shown when distinct from ABE7.10 mutations. In some embodiments, the ABE has a genotype of one of the ABEs as shown in Table 15 below.

TABLE 15
Residue Identity in Evolved TadA

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| ABE7.10 | R | L | A | L | I | V | F | V | N | Y | C | Y | P | Q | V | F | N | T |
| ABE8.1-m | | | | | | | | | | | | T | | | | | | |
| ABE8.2-m | | | | | | | | | | | | R | | | | | | |
| ABE8.3-m | | | | | | | | | | | | | | S | | | | |
| ABE8.4-m | | | | | | | | | | H | | | | | | | | |
| ABE8.5-m | | | | | | S | | | | | | | | | | | | |
| ABE8.6-m | | | | | | | | | | | | | | | | | | R |
| ABE8.7-m | | | | | | | | | | | | | | R | | | | |
| ABE8.8-m | | | | | | | | | | H | | R | | R | | | | |
| ABE8.9-m | | | | | Y | | | | | | | R | | R | | | | |
| ABE8.10-m | | | | | | | | | | | | R | | R | | | | R |
| ABE8.11-m | | | | | | | | | | | | T | | R | | | | |
| ABE8.12-m | | | | | | | | | | | | T | | S | | | | |
| ABE8.13-m | | | | | Y | | | | | H | | R | | R | | | | |
| ABE8.14-m | | | | | Y | S | | | | | | | | | | | | |
| ABE8.15-m | | | | | | S | | | | | | R | | | | | | |
| ABE8.16-m | | | | | | S | | | | H | | R | | | | | | |
| ABE8.17-m | | | | | | S | | | | | | | | R | | | | |
| ABE8.18-m | | | | | | S | | | | H | | | | R | | | | |
| ABE8.19-m | | | | | | S | | | | H | | R | | R | | | | |
| ABE8.20-m | | | | | Y | S | | | | H | | R | | R | | | | |
| ABE8.21-m | | | | | | | | | | | | R | | S | | | | |
| ABE8.22-m | | | | | | S | | | | | | | | S | | | | |
| ABE8.23-m | | | | | | S | | | | H | | | | | | | | |
| ABE8.24-m | | | | | | S | | | | H | | T | | | | | | |
| ABE8.1-d | | | | | | | | | | | | T | | | | | | |
| ABE8.2-d | | | | | | | | | | | | R | | | | | | |
| ABE8.3-d | | | | | | | | | | | | | | S | | | | |
| ABE8.4-d | | | | | | | | | | H | | | | | | | | |
| ABE8.5-d | | | | | | S | | | | | | | | | | | | |
| ABE8.6-d | | | | | | | | | | | | | | | | | | R |
| ABE8.7-d | | | | | | | | | | | | | | R | | | | |
| ABE8.8-d | | | | | | | | | | H | | R | | R | | | | |
| ABE8.9-d | | | | | Y | | | | | | | R | | R | | | | |
| ABE8.10-d | | | | | | | | | | | | R | | R | | | | R |
| ABE8.11-d | | | | | | | | | | | | T | | R | | | | |
| ABE8.12-d | | | | | | | | | | | | T | | S | | | | |
| ABE8.13-d | | | | | Y | | | | | H | | R | | R | | | | |
| ABE8.14-d | | | | | Y | S | | | | | | | | | | | | |
| ABE8.15-d | | | | | | S | | | | | | R | | | | | | |
| ABE7.10 | R | L | A | L | I | V | F | V | N | Y | C | Y | P | Q | V | F | N | T |
| ABE8.16-d | | | | | | S | | | | H | | R | | | | | | |
| ABE8.17-d | | | | | | S | | | | | | | | R | | | | |
| ABE8.18-d | | | | | | S | | | | H | | | | R | | | | |
| ABE8.19-d | | | | | | S | | | | H | | R | | R | | | | |
| ABE8.20-d | | | | | Y | S | | | | H | | R | | R | | | | |
| ABE8.21-d | | | | | | | | | | | | R | | S | | | | |
| ABE8.22-d | | | | | | S | | | | | | | | S | | | | |
| ABE8.23-d | | | | | | S | | | | H | | | | | | | | |
| ABE8.24-d | | | | | | S | | | | H | | T | | | | | | |

In some embodiments, the base editor is ABE8.1, which comprises or consists essentially of the following sequence or a fragment thereof having adenosine deaminase activity:
ABE8.1_Y147T_CP5_NGC PAM_monomer

(SEQ ID NO: 331)
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHYP
GMNHRVEITEGILADECAALLCTFFRMPRQVFNAQKKAQSSTDSGGSSGGSSGSETPGTSES
ATPESSGGSSGGSEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKYGGFMQPT
VAYSVLVVAKVEKGKSKKLKSVKELLGITIMERSSFEKNPIDFLEAKGYKEVKKDLIIKLPK
YSLFELENGRKRMLASAKFLQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPRAF
KYFDTTIARKEYRSTKEVLDATLIHQSITGLYETRIDLSQLGGDGGSGGSGGSGGSGGSGGS
GGMDKKYSIGLAIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQE
EFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDFLDNEENEDILED
IVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRSDKNRGK
SDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEGADKRTADGSEFESPKKKRKV

In the above sequence, the plain text denotes an adenosine deaminase sequence, bold sequence indicates sequence derived from Cas9, the italicized sequence denotes a linker sequence, and the underlined sequence denotes a bipartite nuclear localization sequence. Other ABE8 sequences are provided in the attached sequence listing (SEQ TD NOs: 332-354).

In some embodiments, the base editor is a ninth generation ABE (ABE9). In some embodiments, the ABE9 contains a TadA*9 variant. ABE9 base editors include an adenosine deaminase variant comprising an amino acid sequence, which contains alterations relative to an ABE 7*10 reference sequence, as described herein. Exemplary ABE9 variants are listed in Table 16. Details ofABE9 base editors are described in International PCT Application No. PCT/2020/049975, which is incorporated herein by reference for its entirety.

TABLE 16
Adenosine Base Editor 9 (ABE9) Variants. In the table, “monomer”
indicates an ABE comprising a single TadA*7.10 comprising
the indicated alterations and “heterodimer” indicates
an ABE comprising a TadA*7.10 comprising the indicated alterations
fused to an E. coli TadA adenosine deaminase.

| ABE9 Description | Alterations |
|---|---|
| ABE9.1_monomer | E25F, V82S, Y123H, T133K, Y147R, Q154R |
| ABE9.2_monomer | E25F, V82S, Y123H, Y147R, Q154R |
| ABE9.3_monomer | V82S, Y123H, P124W, Y147R, Q154R |
| ABE9.4_monomer | L51W, V82S, Y123H, C146R, Y147R, Q154R |
| ABE9.5_monomer | P54C, V82S, Y123H, Y147R, Q154R |
| ABE9.6_monomer | Y73S, V82S, Y123H, Y147R, Q154R |
| ABE9.7_monomer | N38G, V82T, Y123H, Y147R, Q154R |
| ABE9.8_monomer | R23H, V82S, Y123H, Y147R, Q154R |
| ABE9.9_monomer | R21N, V82S, Y123H, Y147R, Q154R |
| ABE9.10_monomer | V82S, Y123H, Y147R, Q154R, A158K |
| ABE9.11_monomer | N72K, V82S, Y123H, D139L, Y147R, Q154R, |
| ABE9.12_monomer | E25F, V82S, Y123H, D139M, Y147R, Q154R |
| ABE9.13_monomer | M70V, V82S, M94V, Y123H, Y147R, Q154R |
| ABE9.14_monomer | Q71M, V82S, Y123H, Y147R, Q154R |
| ABE9.15_heterodimer | E25F, V82S, Y123H, T133K, Y147R, Q154R |
| ABE9.16_heterodimer | E25F, V82S, Y123H, Y147R, Q154R |
| ABE9.17_heterodimer | V82S, Y123H, P124W, Y147R, Q154R |
| ABE9.18_heterodimer | L51W, V82S, Y123H, C146R, Y147R, Q154R |
| ABE9.19_heterodimer | P54C, V82S, Y123H, Y147R, Q154R |
| ABE9.2_heterodimer | Y73S, V82S, Y123H, Y147R, Q154R |
| ABE9.21_heterodimer | N38G, V82T, Y123H, Y147R, Q154R |
| ABE9.22_heterodimer | R23H, V82S, Y123H, Y147R, Q154R |
| ABE9.23_heterodimer | R21N, V82S, Y123H, Y147R, Q154R |
| ABE9.24_heterodimer | V82S, Y123H, Y147R, Q154R, A158K |
| ABE9.25_heterodimer | N72K, V82S, Y123H, D139L, Y147R, Q154R, |
| ABE9.26_heterodimer | E25F, V82S, Y123H, D139M, Y147R, Q154R |
| ABE9.27_heterodimer | M70V, V82S, M94V, Y123H, Y147R, Q154R |
| ABE9.28_heterodimer | Q71M, V82S, Y123H, Y147R, Q154R |
| ABE9.29_monomer | E25F_I76Y_V82S_Y123H_Y147R_Q154R |
| ABE9.30_monomer | I76Y_V82T_Y123H_Y147R_Q154R |
| ABE9.31_monomer | N38G_I76Y_V82S_Y123H_Y147R_Q154R |
| ABE9.32_monomer | N38G_I76Y_V82T_Y123H_Y147R_Q154R |
| ABE9.33_monomer | R23H_I76Y_V82S_Y123H_Y147R_Q154R |
| ABE9.34_monomer | P54C_I76Y_V82S_Y123H_Y147R_Q154R |
| ABE9.35_monomer | R21N_I76Y_V82S_Y123H_Y147R_Q154R |
| ABE9.36_monomer | I76Y_V82S_Y123H_D138M_Y147R_Q154R |
| ABE9.37_monomer | Y72S_I76Y_V82S_Y123H_Y147R_Q154R |
| ABE9.38_heterodimer | E25F_I76Y_V82S_Y123H_Y147R_Q154R |
| ABE9.39_heterodimer | I76Y_V82T_Y123H_Y147R_Q154R |
| ABE9.40_heterodimer | N38G_I76Y_V82S_Y123H_Y147R_Q154R |
| ABE9.41_heterodimer | N38G_I76Y_V82T_Y123H_Y147R_Q154R |
| ABE9.42_heterodimer | R23H_I76Y_V82S_Y123H_Y147R_Q154R |
| ABE9.43_heterodimer | P54C_I76Y_V82S_Y123H_Y147R_Q154R |
| ABE9.44_heterodimer | R21N_I76Y_V82S_Y123H_Y147R_Q154R |
| ABE9.45_heterodimer | I76Y_V82S_Y123H_D138M_Y147R_Q154R |
| ABE9.46_heterodimer | Y72S_I76Y_V82S_Y123H_Y147R_Q154R |
| ABE9.47_monomer | N72K_V82S, Y123H, Y147R, Q154R |
| ABE9.48_monomer | Q71M_V82S, Y123H, Y147R, Q154R |
| ABE9.49_monomer | M70V, V82S, M94V, Y123H, Y147R, Q154R |
| ABE9.50_monomer | V82S, Y123H, T133K, Y147R, Q154R |
| ABE9.51_monomer | V82S, Y123H, T133K, Y147R, Q154R, |
| | A158K |
| ABE9.52_monomer | M70V, Q71M, N72K, V82S, Y123H, Y147R, |
| | Q154R |
| ABE9.53_heterodimer | N72K_V82S, Y123H, Y147R, Q154R |
| ABE9.54_heterodimer | Q71M_V82S, Y123H, Y147R, Q154R |
| ABE9.55_heterodimer | M70V, V82S, M94V, Y123H, Y147R, Q154R |
| ABE9.56_heterodimer | V82S, Y123H, T133K, Y147R, Q154R |
| ABE9.57_heterodimer | V82S, Y123H, T133K, Y147R, Q154R, |
| | A158K |
| ABE9.58_heterodimer | M70V, Q71M, N72K, V82S, Y123H, Y147R, |
| | Q154R |

In some embodiments, the base editor includes an adenosine deaminase variant comprising an amino acid sequence, which contains alterations relative to an ABE 7*10 reference sequence, as described herein. The term “monomer” as used in Table 16.1 refers to a monomeric form of TadA*7.10 comprising the alterations described. The term “heterodimer” as used in Table 16.1 refers to the specified wild-type E. coli TadA adenosine deaminase fused to a TadA*7.10 comprising the alterations as described.

TABLE 16.1
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + I76Y + V82G + Y147T + |
| | | Q154S + N157K |
| ABE-828m | MSP828 | monomer_TadA*7.10 + I76Y + V82G + Y147D + F149Y + |
| | | Q154S + D167N |
| ABE-829m | MSP829 | monomer_TadA*7.10 + L36H + I76Y + V82G + Y147D + |
| | | F149Y + Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + 176Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + |
| | | Y147T + Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + |
| | | Y147D + F149Y + Q154S + N157K + D167N) |

In some embodiments, the base editor comprises a domain comprising all or a portion of a uracil glycosylase inhibitor (UGI). In some embodiments, the base editor comprises a domain comprising all or a portion of a nucleic acid polymerase. In some embodiments, a base editor can comprise as a domain all or a portion of a nucleic acid polymerase (NAP). For example, a base editor can comprise all or a portion of a eukaryotic NAP. In some embodiments, a NAP or portion thereof incorporated into a base editor is a DNA polymerase. In some embodiments, a NAP or portion thereof incorporated into a base editor has translesion polymerase activity. In some embodiments, a NAP or portion thereof incorporated into a base editor is a translesion DNA polymerase. In some embodiments, a NAP or portion thereof incorporated into a base editor is a Rev7, Rev1 complex, polymerase iota, polymerase kappa, or polymerase eta. In some embodiments, a NAP or portion thereof incorporated into a base editor is a eukaryotic polymerase alpha, beta, gamma, delta, epsilon, gamma, eta, iota, kappa, lambda, mu, or nu component. In some embodiments, a NAP or portion thereof incorporated into a base editor comprises an amino acid sequence that is at least 75%, 80%, 85%, 90%, 95%, 96%, 97%, 98%, 99%, or 99.5% identical to a nucleic acid polymerase (e.g., a translesion DNA polymerase). In some embodiments, a nucleic acid polymerase or portion thereof incorporated into a base editor is a translesion DNA polymerase.

In some embodiments, a domain of the base editor can comprise multiple domains. For example, the base editor comprising a polynucleotide programmable nucleotide binding domain derived from Cas9 can comprise a REC lobe and an NUC lobe corresponding to the REC lobe and NUC lobe of a wild-type or natural Cas9. In another example, the base editor can comprise one or more of a RuvCI domain, BH domain, REC1 domain, REC2 domain, RuvCII domain, L1 domain, HNH domain, L2 domain, RuvCIII domain, WED domain, TOPO domain or CTD domain. In some embodiments, one or more domains of the base editor comprise a mutation (e.g., substitution, insertion, deletion) relative to a wild-type version of a polypeptide comprising the domain. For example, an HNH domain of a polynucleotide programmable DNA binding domain can comprise an H840A substitution. In another example, a RuvCI domain of a polynucleotide programmable DNA binding domain can comprise a D10A substitution.

Different domains (e.g., adjacent domains) of the base editor disclosed herein can be connected to each other with or without the use of one or more linker domains (e.g., an XTEN linker domain). In some embodiments, a linker domain can be a bond (e.g., covalent bond), chemical group, or a molecule linking two molecules or moieties, e.g., two domains of a fusion protein, such as, for example, a first domain (e.g., Cas9-derived domain) and a second domain (e.g., an adenosine deaminase domain or a cytidine deaminase domain). In some embodiments, a linker is a covalent bond (e.g., a carbon-carbon bond, disulfide bond, carbon-hetero atom bond, etc.). In certain embodiments, a linker is a carbon nitrogen bond of an amide linkage. In certain embodiments, a linker is a cyclic or acyclic, substituted or unsubstituted, branched or unbranched aliphatic or heteroaliphatic linker. In certain embodiments, a linker is polymeric (e.g., polyethylene, polyethylene glycol, polyamide, polyester, etc.). In certain embodiments, a linker comprises a monomer, dimer, or polymer of aminoalkanoic acid. In some embodiments, a linker comprises an aminoalkanoic acid (e.g., glycine, ethanoic acid, alanine, beta-alanine, 3-aminopropanoic acid, 4-aminobutanoic acid, 5-pentanoic acid, etc.). In some embodiments, a linker comprises a monomer, dimer, or polymer of aminohexanoic acid (Ahx). In certain embodiments, a linker is based on a carbocyclic moiety (e.g., cyclopentane, cyclohexane). In other embodiments, a linker comprises a polyethylene glycol moiety (PEG). In certain embodiments, a linker comprises an aryl or heteroaryl moiety. In certain embodiments, the linker is based on a phenyl ring. A linker can include functionalized moieties to facilitate attachment of a nucleophile (e.g., thiol, amino) from the peptide to the linker. Any electrophile can be used as part of the linker. Exemplary electrophiles include, but are not limited to, activated esters, activated amides, Michael acceptors, alkyl halides, aryl halides, acyl halides, and isothiocyanates. In some embodiments, a linker joins a gRNA binding domain of an RNA-programmable nuclease, including a Cas9 nuclease domain, and the catalytic domain of a nucleic acid editing protein. In some embodiments, a linker joins a dCas9 and a second domain (e.g., UGI, etc.).

### Linkers

In certain embodiments, linkers may be used to link any of the peptides or peptide domains of the invention. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Typically, a linker is positioned between, or flanked by, two groups, molecules, or other moieties and connected to each one via a covalent bond, thus connecting the two. In some embodiments, a linker is an amino acid or a plurality of amino acids (e.g., a peptide or protein). In some embodiments, a linker is an organic molecule, group, polymer, or chemical moiety. In some embodiments, a linker is 2-100 amino acids in length, for example, 2, 3, 4, 5,6,7, 8,9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the linker is about 3 to about 104 (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100) amino acids in length. Longer or shorter linkers are also contemplated.

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS)n (SEQ ID NO: 246), (GGGGS)n (SEQ ID NO: 247), and (G)n to more rigid linkers of the form (EAAAK)n (SEQ ID NO: 248), (SGGS)n (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGS
PTSTEEGTSTEPSEGSAPGTSTEPSEGSAPGTSESATPESGPGSEPATS
GGSGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS. In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence:

(SEQ ID NO: 360)
SGGSSGGSSGSETPGTSESATPESSGGSSGGSSGGSSGGS.

In some embodiments, the linker is 64 amino acids in length. In some embodiments, the linker comprises the amino acid sequence.

(SEQ ID NO: 361)
SGGSSGGSSGSETPGTSESATPESSGGSSGGSSGGSSGGSSGSETPGTS
ESATPESSGGSSGGS.

In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEE
GTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.

In another embodiment, the base editor system comprises a component (protein) that interacts non-covalently with a deaminase (DNA deaminase), e.g., an adenosine or a cytidine deaminase, and transiently attracts the adenosine or cytidine deaminase to the target nucleobase in a target polynucleotide sequence for specific editing, with minimal or reduced bystander or target-adjacent effects. Such a non-covalent system and method involving deaminase-interacting proteins serves to attract a DNA deaminase to a particular genomic target nucleobase and decouples the events of on-target and target-adjacent editing, thus enhancing the achievement of more precise single base substitution mutations. In an embodiment, the deaminase-interacting protein binds to the deaminase (e.g., adenosine deaminase or cytidine deaminase) without blocking or interfering with the active (catalytic) site of the deaminase from engaging the target nucleobase (e.g., adenosine or cytidine, respectively). Such as system, termed “MagnEdit,” involves interacting proteins tethered to a Cas9 and gRNA complex and can attract a co-expressed adenosine or cytidine deaminase (either exogenous or endogenous) to edit a specific genomic target site, and is described in McCann, J. et al., 2020, “MagnEdit—interacting factors that recruit DNA-editing enzymes to single base targets,” Life-Science-Alliance, Vol. 3, No. 4 (e201900606), (doi 10.26508/Isa.201900606), the contents of which are incorporated by reference herein in their entirety. In an embodiment, the DNA deaminase is an adenosine deaminase variant (e.g., TadA*8) as described herein.

In another embodiment, a system called “Suntag,” involves non-covalently interacting components used for recruiting protein (e.g., adenosine deaminase or cytidine deaminase) components, or multiple copies thereof, of base editors to polynucleotide target sites to achieve base editing at the site with reduced adjacent target editing, for example, as described in Tanenbaum, M. E. et al., “A protein tagging system for signal amplification in gene expression and fluorescence imaging,” Cell. 2014 Oct. 23; 159(3): 635-646. doi:10.1016/j.cell.2014.09.039; and in Huang, Y.-H. et al., 2017, “DNA epigenome editing using CRISPR-Cas SunTag-directed DNMT3A,” Genome Biol 18: 176. doi:10.1186/s13059-017-1306-z, the contents of each of which are incorporated by reference herein in their entirety. In an embodiment, the DNA deaminase is an adenosine deaminase variant (e.g., TadA*8) as described herein.

Nucleic Acid Programmable DNA Binding Proteins with Guide RNAs

Provided herein are compositions and methods for base editing in cells (e.g., immune cells (e.g., T- or NK-cells)). Further provided herein are compositions comprising a guide polynucleic acid sequence, e.g. a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g. a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in an immune cell (e.g., T- or NK-cell) through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide complexes comprising any of the fusion proteins provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In some embodiments, the target sequence is a DNA sequence. In some embodiments, the target sequence is an RNA sequence. In some embodiments, the target sequence is a sequence in the genome of a mammal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 7 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins provided herein, and with at least one guide RNA, wherein the guide RNA is about 15-100 nucleotides long and comprises a sequence of at least 10 contiguous nucleotides that is complementary to a target sequence. In some embodiments, the 3′ end of the target sequence is immediately adjacent to an AGC, GAG, TTT, GTG, or CAA sequence. In some embodiments, the 3′ end of the target sequence is immediately adjacent to an NGA, NGCG, NGN, NNGRRT, NNNRRT, NGCG, NGCN, NGTN, NGTN, NGTN, or 5′ (TTTV) sequence. In some embodiments, the 3′ end of the target sequence is immediately adjacent to an e.g., TTN, DTTN, GTTN, ATTN, ATTC, DTTNT, WTTN, HATY, TTTN, TTTV, TTTC, TG, RTR,orYTN PAM site.

It will be understood that the numbering of the specific positions or residues in the respective sequences depends on the particular protein and numbering scheme used. Numbering might differ, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins disclosed herein, to a target site, e.g., a site comprising a mutation to be edited, it is typically necessary to co-express the fusion protein together with a guide RNA. As explained in more detail elsewhere herein, a guide RNA typically comprises a tracrRNA framework allowing for napDNAbp (e.g., Cas9 or Cas12) binding, and a guide sequence, which confers sequence specificity to the napDNAbp:nucleic acid editing enzyme/domain fusion protein. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting napDNAbp:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein.

Distinct portions of sgRNA are predicted to form various features that interact with Cas9 (e.g., SpyCas9) and/or the DNA target. Six conserved modules have been identified within native crRNA:tracrRNA duplexes and single guide RNAs (sgRNAs) that direct Cas9 endonuclease activity (see Briner et al., Guide RNA Functional Modules Direct Cas9 Activity and Orthogonality Mol Cell. 2014 Oct. 23; 56(2):333-339). The six modules include the spacer responsible for DNA targeting, the upper stem, bulge, lower stem formed by the CRISPR repeat:tracrRNA duplex, the nexus, and hairpins from the 3′ end of the tracrRNA. The upper and lower stems interact with Cas9 mainly through sequence-independent interactions with the phosphate backbone. In some embodiments, the upper stem is dispensable. In some embodiments, the conserved uracil nucleotide sequence at the base of the lower stem is dispensable. The bulge participates in specific side-chain interactions with the Rec domain of Cas9. The nucleobase of U44 interacts with the side chains of Tyr 325 and His 328, while G43 interacts with Tyr 329. The nexus forms the core of the sgRNA:Cas9 interactions and lies at the intersection between the sgRNA and both Cas9 and the target DNA. The nucleobases of A51 and A52 interact with the side chain of Phe 1105; U56 interacts with Arg 457 and Asn 459; the nucleobase of U59 inserts into a hydrophobic pocket defined by side chains of Arg 74, Asn 77, Pro 475, Leu 455, Phe 446, and Ile 448; C60 interacts with Leu 455, Ala 456, and Asn 459, and C61 interacts with the side chain of Arg 70, which in turn interacts with C15. In some embodiments, one or more of these mutations are made in the bulge and/or the nexus of a sgRNA for a Cas9 (e.g., spyCas9) to optimize sgRNA:Cas9 interactions.

Moreover, the tracrRNA nexus and hairpins are critical for Cas9 pairing and can be swapped to cross orthogonality barriers separating disparate Cas9 proteins, which is instrumental for further harnessing of orthogonal Cas9 proteins. In some embodiments, the nexus and hairpins are swapped to target orthogonal Cas9 proteins. In some embodiments, a sgRNA is dispensed of the upper stem, hairpin 1, and/or the sequence flexibility of the lower stem to design a guide RNA that is more compact and conformationally stable. In some embodiments, the modules are modified to optimize multiplex editing using a single Cas9 with various chimeric guides or by concurrently using orthogonal systems with different combinations of chimeric sgRNAs. Details regarding guide functional modules and methods thereof are described, for example, in Briner et al., Guide RNA Functional Modules Direct Cas9 Activity and Orthogonality Mol Cell. 2014 Oct. 23; 56(2):333-339, the contents of which is incorporated by reference herein in its entirety.

The domains of the base editor disclosed herein can be arranged in any order. Non-limiting examples of a base editor comprising a fusion protein comprising e.g., a polynucleotide-programmable nucleotide-binding domain (e.g., Cas9 or Cas12) and a deaminase domain (e.g., cytidine or adenosine deaminase) can be arranged as follows:
NH2-[nucleobase editing domain]-Linker1-[nucleobase editing domain]-COOH;
NH2-[deaminase]-Linker1-[nucleobase editing domain]-COOH;
NH2-[deaminase]-Linker1-[nucleobase editing domain]-Linker2-[UGI]-COOH;
NH2-[deaminase]-Linker1-[nucleobase editing domain]-COOH;
NH2-[adenosine deaminase]-Linker1-[nucleobase editing domain]-COOH;
NH2-[nucleobase editing domain]-[deaminase]-COOH;
NH2-[deaminase]-[nucleobase editing domain]-[inosine BER inhibitor]-COOH;
NH2-[deaminase]-[inosine BER inhibitor]-[nucleobase editing domain]-COOH;
NH2-[inosine BER inhibitor]-[deaminase]-[nucleobase editing domain]-COOH;
NH2-[nucleobase editing domain]-[deaminase]-[inosine BER inhibitor]-COOH;
NH2-[nucleobase editing domain]-[inosine BER inhibitor]-[deaminase]-COOH;
NH2-[inosine BER inhibitor]-[nucleobase editing domain]-[deaminase]-COOH;
NH2-[nucleobase editing domain]-Linker1-[deaminase]-Linker2-[nucleobase editing domain]-COOH;
NH2-[nucleobase editing domain]-Linker1-[deaminase]-[nucleobase editing domain]-COOH;
NH2-[nucleobase editing domain]-[deaminase]-Linker2-[nucleobase editing domain]-COOH;
NH2-[nucleobase editing domain]-[deaminase]-[nucleobase editing domain]-COOH;
NH2-[nucleobase editing domain]-Linker1-[deaminase]-Linker2-[nucleobase editing domain]-[inosine BER inhibitor]-COOH;
NH2-[nucleobase editing domain]-Linker1-[deaminase]-[nucleobase editing domain]-[inosine BER inhibitor]-COOH;
NH2-[nucleobase editing domain]-[deaminase]-Linker2-[nucleobase editing domain]-[inosine BER inhibitor]-COOH;
NH2-[nucleobase editing domain]-[deaminase]-[nucleobase editing domain]-[inosine BER inhibitor]-COOH;
NH2-[inosine BER inhibitor]-[nucleobase editing domain]-Linker1-[deaminase]-Linker2-[nucleobase editing domain]-COOH;
NH2-[inosine BER inhibitor]-[nucleobase editing domain]-Linker1-[deaminase]-[nucleobase editing domain]-COOH;
NH2-[inosine BER inhibitor]-[nucleobase editing domain]-[deaminase]-Linker2-[nucleobase editing domain]-COOH; or
NH2-[inosine BER inhibitor]NH2-[nucleobase editing domain]-[deaminase]-[nucleobase editing domain]-COOH.

In some embodiments, the base editing fusion proteins provided herein need to be positioned at a precise location, for example, where a target base is placed within a defined region (e.g., a “deamination window”). In some embodiments, a target can be within a 4-base region. In some embodiments, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a napDNAbp domain. In some embodiments, an NLS of the base editor is localized C-terminal to a napDNAbp domain.

Non-limiting examples of protein domains which can be included in the fusion protein include a deaminase domain (e.g., adenosine deaminase or cytidine deaminase), a uracil glycosylase inhibitor (UGI) domain, epitope tags, reporter gene sequences, and/or protein domains having one or more of the activities described herein.

A domain may be detected or labeled with an epitope tag, a reporter protein, other binding domains. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). Additional protein sequences can include amino acid sequences that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions.

### Methods of Using Fusion Proteins Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins provided herein, and with at least one guide RNA described herein.

In some embodiments, a fusion protein of the invention is used for editing a target gene or polynucleotide sequence of interest (e.g., an A2AR, A2BR, HIF1α, HIF1α.I3 target gene). In particular, a cytidine deaminase or adenosine deaminase nucleobase editor described herein is capable of making multiple mutations within a target sequence. These mutations may affect the function or expression of the target. For example, when a cytidine deaminase or adenosine deaminase nucleobase editor is used to target a regulatory region the function of the regulatory region is altered and the expression of the downstream protein is reduced or eliminated.

It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins comprising a Cas9 domain and a cytidine or adenosine deaminase, as disclosed herein, to a target site, e.g., a site comprising a mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein.

### Base Editor Efficiency

In some embodiments, the purpose of the methods provided herein is to alter a gene (e.g., A2AR, A2BR, HIF1α, HIF1α.I3 polynucleotides) via base editing. The nucleobase editing proteins provided herein can be used for gene editing-based human therapeutics in vitro or in vivo. It will be understood by the skilled artisan that the nucleobase editing proteins provided herein, e.g., the fusion proteins comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations. In some embodiments, an intended mutation is a mutation that is generated by a specific base editor (e.g., adenosine base editor or cytidine base editor) bound to a guide polynucleotide (e.g., gRNA), specifically designed to generate the intended mutation. In some embodiments, the intended mutation is in a gene associated with a target antigen associated with a disease or disorder, e.g., cancer, neoplasia, solid tumor. In some embodiments, the intended mutation is an adenine (A) to guanine (G) point mutation (e.g., SNP) in a gene associated with a target antigen associated with a disease or disorder, e.g. cancer, neoplasia, solid tumor. In some embodiments, the intended mutation is an adenine (A) to guanine (G) point mutation within the coding region or non-coding region of a gene (e.g., regulatory region or element). In some embodiments, the intended mutation is a cytosine (C) to thymine (T) point mutation (e.g., SNP) in a gene associated with a target antigen associated with a disease or disorder, e.g., cancer, neoplasia, solid tumor. In some embodiments, the intended mutation is a cytosine (C) to thymine (T) point mutation within the coding region or non-coding region of a gene (e.g., regulatory region or element). In some embodiments, the intended mutation is a point mutation that generates a STOP codon, for example, a premature STOP codon within the coding region of a gene. In some embodiments, the intended mutation is a mutation that eliminates a stop codon.

The base editors of the invention advantageously modify a specific nucleotide base encoding a protein without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate) a specific nucleotide within a nucleic acid, without generating a large number of insertions or deletions (i.e., indels) in the nucleic acid. In some embodiments, it is desirable to generate base editors that efficiently modify (e.g. mutate or methylate) a specific nucleotide within a nucleic acid, without generating a large number of insertions or deletions (i.e., indels) in the nucleic acid. In certain embodiments, any of the base editors provided herein can generate a greater proportion of intended modifications (e.g., methylations) versus indels. In certain embodiments, any of the base editors provided herein can generate a greater proportion of intended modifications (e.g., mutations) versus indels.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, a number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors provided herein are capable of efficiently generating an intended mutation in a nucleic acid (e.g. a nucleic acid within a genome of a subject) without generating a considerable number of unintended mutations (e.g., spurious off-target editing or bystander editing). In some embodiments, an intended mutation is a mutation that is generated by a specific base editor bound to a gRNA, specifically designed to generate the intended mutation. In some embodiments, the intended mutation is a mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g., intended mutations:unintended mutations) that is greater than 1:1. In some embodiments, any of the base editors provided herein are capable of generating a ratio of intended mutations to unintended mutations that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described herein may be applied to any of the fusion proteins, or methods of using the fusion proteins provided herein.

Base editing is often referred to as a “modification” or “alteration”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence, and may affect the gene product. In essence therefore, the gene editing modification described herein may result in a modification of the gene, structurally and/or functionally, wherein the expression of the gene product may be modified, for example, the expression of the gene is knocked out; or conversely, enhanced, or, in some circumstances, the gene function or activity may be modified. Using the methods disclosed herein, a base editing efficiency may be determined as the knockdown efficiency of the gene in which the base editing is performed, wherein the base editing is intended to knockdown the expression of the gene. A knockdown level may be validated quantitatively by determining the expression level by any detection assay, such as assay for protein expression level, for example, by flow cytometry; assay for detecting RNA expression such as quantitative RT-PCR, northern blot analysis, or any other suitable assay such as pyrosequencing; and may be validated qualitatively by nucleotide sequencing reactions.

In some embodiments, the modification, e.g., single base edit results in at least 10% reduction of the gene targeted expression. In some embodiments, the base editing efficiency may result in at least 10% reduction of the gene targeted expression. In some embodiments, the base editing efficiency may result in at least 15% reduction of the gene targeted expression. In some embodiments, the base editing efficiency may result in at least 20% reduction of the gene targeted expression. In some embodiments, the base editing efficiency may result in at least 25% reduction of the gene targeted expression. In some embodiments, the base editing efficiency may result in at least 30% reduction of the gene targeted expression. In some embodiments, the base editing efficiency may result in at least 40% reduction of the gene targeted expression. In some embodiments, the base editing efficiency may result in at least 50% reduction of the gene targeted expression. In some embodiments, the base editing efficiency may result in at least 60% reduction of the targeted gene expression. In some embodiments, the base editing efficiency may result in at least 70% reduction of the targeted gene expression. In some embodiments, the expression of a target gene (e.g., A2AR, A2BR, HIF1α, HIF1α.I3) is eliminated or rendered virtually undetectable. In some embodiments, the base editing efficiency may result in at least 80% reduction of the targeted gene expression. In some embodiments, the base editing efficiency may result in at least 90% reduction of the targeted gene expression. In some embodiments, the base editing efficiency may result in at least 91% reduction of the targeted gene expression. In some embodiments, the base editing efficiency may result in at least 92% reduction of the targeted gene expression. In some embodiments, the base editing efficiency may result in at least 93% reduction of the targeted gene expression. In some embodiments, the base editing efficiency may result in at least 94% reduction of the targeted gene expression. In some embodiments, the base editing efficiency may result in at least 95% reduction of the targeted gene expression. In some embodiments, the base editing efficiency may result in at least 96% reduction of the targeted gene expression. In some embodiments, the base editing efficiency may result in at least 97% reduction of the targeted gene expression. In some embodiments, the base editing efficiency may result in at least 98% reduction of the targeted gene expression. In some embodiments, the base editing efficiency may result in at least 99% reduction of the targeted gene expression. In some embodiments, the base editing efficiency may result in knockout (100% knockdown of the gene expression) of the gene that is targeted.

In some embodiments, any of the base editor systems provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

In some embodiments, targeted modifications, e.g., single base editing, are used simultaneously to target at least 4, 5, 6, 7, 8, 9, 10, 11, 12 13, 14, 15, 16, 17,18, 19, 20, 21, 22,23,24,25,26,27,28,29,30,31,32,33,34,35,36,37,38,39,40,41,42,43,44,45,46, 47, 48, 49 or 50 different endogenous sequences for base editing with different guide RNAs. In some embodiments, targeted modifications, e.g. single base editing, are used to sequentially target at least 4, 5, 6, 7, 8, 9, 10, 11, 12 13, 14, 15, 16, 17,18, 19, 20, 21, 22, 23, 24,25,26,27,28,29,30,31,32,33,34,35,36,37,38,39,40,41,42,43,44,45,46,47,48, 49 50, or more different endogenous gene sequences for base editing with different guide RNAs.

Some aspects of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations (i.e., mutation of bystanders). In some embodiments, any of the base editors provided herein are capable of generating at least 0.01% of intended mutations (i.e., at least 0.01% base editing efficiency). In some embodiments, any of the base editors provided herein are capable of generating at least 0.01%, 1%, 2%, 3%, 4%, 5%10%, 15%, 20%, 25%, 30%, 40%, 45%, 50%, 60%, 70%, 80%, 90%, 95%, or 99% of intended mutations.

In some embodiments, any of the base editor systems comprising one of the ABE8 and/or ABE9 base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence. In some embodiments, any of the base editor systems comprising one of the ABE8 and/or ABE9 base editor variants described herein result in less than 0.8% indel formation in the target polynucleotide sequence. In some embodiments, any of the base editor systems comprising one of the ABE8 and/or ABE9 base editor variants described herein result in at most 0.8% indel formation in the target polynucleotide sequence. In some embodiments, any of the base editor systems comprising one of the ABE8 and/or ABE9 base editor variants described herein result in less than 0.3% indel formation in the target polynucleotide sequence. In some embodiments, any of the base editor systems comprising one of the ABE8 base editor variants described results in lower indel formation in the target polynucleotide sequence compared to a base editor system comprising one of ABE7 base editors. In some embodiments, any of the base editor systems comprising one of the ABE8 and/or ABE9 base editor variants described herein results in lower indel formation in the target polynucleotide sequence compared to a base editor system comprising an ABE7.10.

In some embodiments, any of the base editor systems comprising one of the ABE8 and/or ABE9 base editor variants described herein has reduction in indel frequency compared to a base editor system comprising one of the ABE7 base editors. In some embodiments, any of the base editor systems comprising one of the ABE8 and/or ABE9 base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising one of the ABE7 base editors. In some embodiments, a base editor system comprising one of the ABE8 and/or ABE9 base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising an ABE7.10.

The invention provides adenosine deaminase variants (e.g., ABE8 and/or ABE9 variants) that have increased efficiency and specificity. In particular, the adenosine deaminase variants described herein are more likely to edit a desired base within a polynucleotide, and are less likely to edit bases that are not intended to be altered (e.g., “bystanders”).

In some embodiments, any of the base editing system comprising one of the ABE8 and/or ABE9 base editor variants described herein has reduced bystander editing or mutations. In some embodiments, an unintended editing or mutation is a bystander mutation or bystander editing, for example, base editing of a target base (e.g., A or C) in an unintended or non-target position in a target window of a target nucleotide sequence. In some embodiments, any of the base editing system comprising one of the ABE8 and/or ABE9 base editor variants described herein has reduced bystander editing or mutations compared to a base editor system comprising an ABE7 base editor, e.g., ABE7.10. In some embodiments, any of the base editing system comprising one of the ABE8 and/or ABE9 base editor variants described herein has reduced bystander editing or mutations by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE8 and/or ABE9 base editor variants described herein has reduced bystander editing or mutations by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the base editing system comprising one of the ABE8 and/or ABE9 base editor variants described herein has reduced spurious editing. In some embodiments, an unintended editing or mutation is a spurious mutation or spurious editing, for example, non-specific editing or guide independent editing of a target base (e.g., A or C) in an unintended or non-target region of the genome. In some embodiments, any of the base editing system comprising one of the ABE8 and/or ABE9 base editor variants described herein has reduced spurious editing compared to a base editor system comprising an ABE7 base editor, e.g., ABE7.10. In some embodiments, any of the base editing system comprising one of the ABE8 and/or ABE9 base editor variants described herein has reduced spurious editing by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE8 and/or ABE9 base editor variants described herein has reduced spurious editing by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the ABE8 base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% base editing efficiency. In some embodiments, the base editing efficiency may be measured by calculating the percentage of edited nucleobases in a population of cells. In some embodiments, any of the ABE8 and/or ABE9 base editor variants described herein have base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases in a population of cells.

In some embodiments, any of the ABE8 and/or ABE9 base editor variants described herein has higher base editing efficiency compared to the ABE7 base editors. In some embodiments, any of the ABE8 base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher base editing efficiency compared to an ABE7 base editor, e.g., ABE7.10.

In some embodiments, any of the ABE8 and/or ABE9 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher base editing efficiency compared to an ABE7 base editor, e.g., ABE7.10.

In some embodiments, any of the ABE8 and/or ABE9 base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% on-target base editing efficiency. In some embodiments, any of the ABE8 and/or ABE9 base editor variants described herein have on-target base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited target nucleobases in a population of cells.

In some embodiments, any of the ABE8 and/or ABE9 base editor variants described herein has higher on-target base editing efficiency compared to the ABE7 base editors. In some embodiments, any of the ABE8 base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher on-target base editing efficiency compared to an ABE7 base editor, e.g., ABE7.10.

In some embodiments, any of the ABE8 or ABE9 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target base editing efficiency compared to an ABE7 base editor, e.g., ABE7.10.

The ABE8 and/or ABE9 base editor variants described herein may be delivered to a host cell via a plasmid, a vector, a LNP complex, or an mRNA. In some embodiments, any of the ABE8 or ABE9 base editor variants described herein is delivered to a host cell as an mRNA. In some embodiments, an ABE8 or ABE9 base editor delivered via a nucleic acid based delivery system, e.g., an mRNA, has on-target editing efficiency of at least at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases. In some embodiments, an ABE8 or ABE9 base editor delivered by an mRNA system has higher base editing efficiency compared to an ABE8 or ABE9 base editor delivered by a plasmid or vector system. In some embodiments, any of the ABE8 or ABE9 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300% higher, at least 310%, at least 320%, at least 330%, at least 340%, at least 350%, at least 360%, at least 370%, at least 380%, at least 390%, at least 400%, at least 450%, or at least 500% on-target editing efficiency when delivered by an mRNA system compared to when delivered by a plasmid or vector system. In some embodiments, any of the ABE8 or ABE9 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target editing efficiency when delivered by an mRNA system compared to when delivered by a plasmid or vector system.

In some embodiments, any of the base editor systems comprising one of the ABE8 or ABE9 base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in the target polynucleotide sequence.

In some embodiments, any of the ABE8 or ABE9 base editor variants described herein has lower guided off-target editing efficiency when delivered by an mRNA system compared to when delivered by a plasmid or vector system. In some embodiments, any of the ABE8 or ABE9 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guided off-target editing efficiency when delivered by an mRNA system compared to when delivered by a plasmid or vector system. In some embodiments, any of the ABE8 or ABE9 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold lower guided off-target editing efficiency when delivered by an mRNA system compared to when delivered by a plasmid or vector system. In some embodiments, any of the ABE8 or ABE9 base editor variants described herein has at least about 2.2 fold decrease in guided off-target editing efficiency when delivered by an mRNA system compared to when delivered by a plasmid or vector system.

In some embodiments, any of the ABE8 or ABE9 base editor variants described herein has lower guide-independent off-target editing efficiency when delivered by an mRNA system compared to when delivered by a plasmid or vector system. In some embodiments, any of the ABE8 or ABE9 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guide-independent off-target editing efficiency when delivered by an mRNA system compared to when delivered by a plasmid or vector system. In some embodiments, any of the ABE8 or ABE9 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 5.0 fold, at least 10.0 fold, at least 20.0 fold, at least 50.0 fold, at least 70.0 fold, at least 100.0 fold, at least 120.0 fold, at least 130.0 fold, or at least 150.0 fold lower guide-independent off-target editing efficiency when delivered by an mRNA system compared to when delivered by a plasmid or vector system. In some embodiments, ABE8 or ABE9 base editor variants described herein has 134.0 fold decrease in guide-independent off-target editing efficiency (e.g., spurious RNA deamination) when delivered by an mRNA system compared to when delivered by a plasmid or vector system. In some embodiments, ABE8 or ABE9 base editor variants described herein does not increase guide-independent mutation rates across the genome.

In some embodiments, a single gene delivery event (e.g., by transduction, transfection, electroporation or any other method) can be used to target base editing of 5 sequences within a cell's genome. In some embodiments, a single gene delivery event can be used to target base editing of 6 sequences within a cell's genome. In some embodiments, a single gene delivery event can be used to target base editing of 7 sequences within a cell's genome. In some embodiments, a single electroporation event can be used to target base editing of 8 sequences within a cell's genome. In some embodiments, a single gene delivery event can be used to target base editing of 9 sequences within a cell's genome. In some embodiments, a single gene delivery event can be used to target base editing of 10 sequences within a cell's genome. In some embodiments, a single gene delivery event can be used to target base editing of 20 sequences within a cell's genome. In some embodiments, a single gene delivery event can be used to target base editing of 30 sequences within a cell's genome. In some embodiments, a single gene delivery event can be used to target base editing of 40 sequences within a cell's genome. In some embodiments, a single gene delivery event can be used to target base editing of 50 sequences within a cell's genome.

In some embodiments, the method described herein, for example, the base editing methods has minimum to no off-target effects.

In some embodiments, the base editing method described herein results in at least 50% of a cell population that have been successfully edited (i.e., cells that have been successfully engineered). In some embodiments, the base editing method described herein results in at least 55% of a cell population that have been successfully edited. In some embodiments, the base editing method described herein results in at least 60% of a cell population that have been successfully edited. In some embodiments, the base editing method described herein results in at least 65% of a cell population that have been successfully edited. In some embodiments, the base editing method described herein results in at least 70% of a cell population that have been successfully edited. In some embodiments, the base editing method described herein results in at least 75% of a cell population that have been successfully edited. In some embodiments, the base editing method described herein results in at least 80% of a cell population that have been successfully edited. In some embodiments, the base editing method described herein results in at least 85% of a cell population that have been successfully edited. In some embodiments, the base editing method described herein results in at least 90% of a cell population that have been successfully edited. In some embodiments, the base editing method described herein results in at least 95% of a cell population that have been successfully edited. In some embodiments, the base editing method described herein results in about 91%, 92%, 93%, 94%, 95%, 96%, 97%, 98%, 99% or 100% of a cell population that have been successfully edited.

In some embodiments, the live cell recovery following a base editing intervention is greater than at least 60%, 70%, 80%, 90% of the starting cell population at the time of the base editing event. In some embodiments, the live cell recovery as described above is about 70%. In some embodiments, the live cell recovery as described above is about 75%. In some embodiments, the live cell recovery as described above is about 80%. In some embodiments, the live cell recovery as described above is about 85%. In some embodiments, the live cell recovery as described above is about 90%, or about 91%, 92%, 93%, 94% 95%, 96%, 97%, 98%, or 99%, or 100% of the cells in the population at the time of the base editing event.

In some embodiments the engineered cell population can be further expanded in vitro by about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The number of intended mutations and indels can be determined using any suitable method, for example, as described in International PCT Application Nos. PCT/2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

The number of indels formed at a target nucleotide region can depend on the amount of time a nucleic acid (e.g., a nucleic acid within the genome of a cell) is exposed to a base editor. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a base editor. It should be appreciated that the characteristics of the base editors as described herein can be applied to any of the fusion proteins, or methods of using the fusion proteins provided herein.

Details of base editor efficiency are described in International PCT Application Nos. PCT/2017/045381 (WO 2018/027078) and PCT/US2016/058344 (WO 2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference. In some embodiments, editing of a plurality of nucleobase pairs in one or more genes using the methods provided herein results in formation of at least one intended mutation. In some embodiments, said formation of said at least one intended mutation results in the disruption the normal function of a gene. In some embodiments, said formation of said at least one intended mutation results decreases or eliminates the expression of a protein encoded by a gene. It should be appreciated that multiplex editing can be accomplished using any method or combination of methods provided herein.

### Multiplex Editing

In some embodiments, the base editor system provided herein is capable of multiplex editing of a plurality of nucleobase pairs in one or more genes. In some embodiments, the plurality of nucleobase pairs is located in the same gene or in one or more genes or polynucleotide sequences, wherein at least one gene is located in a different locus. In some embodiments, the base editor system provided herein is capable of multiplex editing of a plurality of nucleobase pairs in one or more genes involved in hypoxic and/or adenosinergic pathways or regulatory components thereof. In some embodiments, the base editor system provided herein is capable of multiplex editing of a plurality of nucleobase pairs in one or more genes selected from A2AR, A2BR, HIF1α, and/or HIF1α.I3.

In some embodiments, the multiplex editing can comprise one or more guide polynucleotides. In some embodiments, the multiplex editing can comprise one or more base editor systems. In some embodiments, the multiplex editing can comprise one or more base editor systems with a single guide polynucleotide or a plurality of guide polynucleotides. In some embodiments, the multiplex editing can comprise one or more guide polynucleotides with a single base editor system. In some embodiments, the multiplex editing can comprise at least one guide polynucleotide that does or does not require a PAM sequence to target binding to a target polynucleotide sequence. In some embodiments, the multiplex editing can comprise a mix of at least one guide polynucleotide that does not require a PAM sequence to target binding to a target polynucleotide sequence and at least one guide polynucleotide that require a PAM sequence to target binding to a target polynucleotide sequence. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the plurality of nucleobase pairs are in one more genes or polynucleotide sequences. In some embodiments, the plurality of nucleobase pairs is in the same gene or polynucleotide sequence. In some embodiments, at least one gene in the one more genes is located in a different locus.

In some embodiments, the editing is editing of the plurality of nucleobase pairs in at least one protein coding region, in at least one protein non-coding region, or in at least one protein coding region and at least one protein non-coding region.

In some embodiments, the editing is in conjunction with one or more guide polynucleotides. In some embodiments, the base editor system can comprise one or more base editor systems. In some embodiments, the base editor system can comprise one or more base editor systems in conjunction with a single guide polynucleotide or a plurality of guide polynucleotides. In some embodiments, the editing is in conjunction with one or more guide polynucleotide with a single base editor system. In some embodiments, the editing is in conjunction with at least one guide polynucleotide that does not require a PAM sequence to target binding to a target polynucleotide sequence or with at least one guide polynucleotide that requires a PAM sequence to target binding to a target polynucleotide sequence, or with a mix of at least one guide polynucleotide that does not require a PAM sequence to target binding to a target polynucleotide sequence and at least one guide polynucleotide that does require a PAM sequence to target binding to a target polynucleotide sequence. It should be appreciated that the characteristics of the multiplex editing using any of the base editors as described herein can be applied to any of combination of the methods of using any of the base editors provided herein. It should also be appreciated that the editing can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors. In some embodiments, the base editor system capable of multiplex editing comprising one of the ABE8 base editor variants described herein has higher multiplex editing efficiency compared to the base editor system capable of multiplex editing comprising one of ABE7 base editors. In some embodiments, the base editor system capable of multiplex editing comprising one of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300% higher, at least 310%, at least 320%, at least 330%, at least 340%, at least 350%, at least 360%, at least 370%, at least 380%, at least 390%, at least 400%, at least 450%, or at least 500% higher multiplex editing efficiency compared the base editor system capable of multiplex editing comprising one of ABE7 base editors. In some embodiments, the base editor system capable of multiplex editing comprising one of the ABE8 and/or ABE9 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 4.0 fold, at least 4.5 fold, at least 5.0 fold, at least 5.5 fold, or at least 6.0 fold higher multiplex editing efficiency compared the base editor system capable of multiplex editing comprising one of ABE7 base editors.

### Expression of Polypeptides in a Host Cell

Polypeptides of the invention may be expressed in virtually any host cell of interest, including mammalian cells (e.g., human cells), using routine methods known to the skilled artisan. In some embodiments, the host cell of interest is a human cell. In some embodiments, the host cell is an immune cell (e.g., T- or NK-cell). In some embodiments, the host cell is a CAR-T cell.

For example, a DNA encoding a polypeptide of the invention can be cloned by designing suitable primers for the upstream and downstream of CDS based on the cDNA sequence The cloned DNA may be directly, or after digestion with a restriction enzyme when desired, or after addition of a suitable linker and/or a nuclear localization signal, ligated with a DNA encoding one or more additional components of a base editing system. The base editing system is translated in a host cell to form a complex.

A DNA encoding a protein domain described herein can be obtained by chemically synthesizing the DNA, or by connecting synthesized partly overlapping oligoDNA short chains by utilizing the PCR method and the Gibson Assembly method to construct a DNA encoding the full length thereof. The advantage of constructing a full-length DNA by chemical synthesis or a combination of PCR method or Gibson Assembly method is that the codon to be used can be designed in CDS full-length according to the host into which the DNA is introduced. In the expression of a heterologous DNA, the protein expression level is expected to increase by converting the DNA sequence thereof to a codon highly frequently used in the host organism. As the data of codon use frequency in host to be used, for example, the genetic code use frequency database (kazusa.or.jp/codon/index.html) disclosed in the home page of Kazusa DNA Research Institute can be used, or documents showing the codon use frequency in each host may be referred to. By reference to the obtained data and the DNA sequence to be introduced, codons showing low use frequency in the host from among those used for the DNA sequence may be converted to a codon coding the same amino acid and showing high use frequency.

An expression vector containing a DNA encoding a nucleic acid sequence-recognizing module and/or a nucleic acid base converting enzyme can be produced, for example, by linking the DNA to the downstream of a promoter in a suitable expression vector.

As the expression vector, animal cell expression plasmids and animal virus vectors such as retrovirus, vaccinia virus, adenovirus and the like, and the like are used.

Regarding the promoter to be used, any promoter appropriate for a host to be used for gene expression can be used. In a conventional method using double-stranded breaks, since the survival rate of the host cell sometimes decreases markedly due to the toxicity, it is desirable to increase the number of cells by the start of the induction by using an inductive promoter. However, since sufficient cell proliferation can also be afforded by expressing the nucleic acid-modifying enzyme complex of the present invention, a constitutive promoter can be used without limitation.

For example, when the host is an animal cell, an SR.alpha. promoter, SV40 promoter, LTR promoter, cytomegalovirus (CMV) promoter, MND promoter, Rous sarcoma virus (RSV) promoter, Moloney mouse leukemia virus (MoMuLV), LTR, herpes simplex virus thymidine kinase (HSV-TK) promoter, and the like can be used. Of these, CMV promoter, SR.alpha. promoter and the like are preferable.

Expression vectors for use in the present invention, besides those mentioned above, can comprise an enhancer, a splicing signal, a terminator, a polyA addition signal, a selection marker, such as drug resistance gene, an auxotrophic complementary gene and the like, a replication origin, and the like can be used.

An RNA encoding a protein domain described herein can be prepared by, for example, in vitro transcription of a nucleic acid sequence encoding any of the polypeptides disclosed herein.

A polypeptide of the invention can be intracellularly expressed by introducing into the cell an expression vector comprising a nucleic acid sequence encoding the polypeptide.

Animal cells contemplated in the present invention include, but are not limited to, cell lines such as monkey COS-7 cells, monkey Vero cells, Chinese hamster ovary (CHO) cells, dhfr gene-deficient CHO cells, mouse L cells, mouse AtT-20 cells, mouse myeloma cells, rat GH3 cells, human FL cells and the like, pluripotent stem cells such as iPS cells, ES cells derived humans and other mammals, and primary cultured cells prepared from various tissues. Furthermore, zebrafish embryo, Xenopus oocyte, and the like can also be used.

All the above-mentioned host cells may be haploid (monoploid), or polyploid (e.g., diploid, triploid, tetraploid, etc.). Using conventional methods, mutations, in principle, introduced into only one homologous chromosome produce a heterogenous cell. Therefore, the desired phenotype is not expressed unless the mutation is dominant. For recessive mutations, acquiring a homozygous cell can be inconvenient due to labor and time requirements. In contrast, according to the present invention, since a mutation can be introduced into any allele on the homologous chromosome in the genome, the desired phenotype can be expressed in a single generation even in the case of recessive mutation, thereby solving the problem associated with conventional mutagenesis methods.

An expression vector can be introduced by a known method (e.g., the lysozyme method, the competent method, the PEG method, the CaCl₂) coprecipitation method, electroporation, microinjection, particle gun method, lipofection, Agrobacterium-mediated delivery, etc.) according to the kind of the host.

A vector can be introduced into an animal cell according to the methods described in, for example, Cell Engineering additional volume 8, New Cell Engineering Experiment Protocol, 263-267 (1995) (published by Shujunsha), and Virology, 52, 456 (1973).

As a medium for culturing an animal cell, for example, minimum essential medium (MEM) containing about 5 to about 20% of fetal bovine serum (Science, 122, 501 (1952)), Dulbecco's modified Eagle medium (DMEM) (Virology, 8, 396 (1959)], RPMI 1640 medium (The Journal of the American Medical Association, 199, 519 (1967)], 199 medium (Proceeding of the Society for the Biological Medicine, 73, 1 (1950)] and the like are used. The pH of the medium is preferably about 6 to about 8. The culture is performed at generally about 30° C. to about 40° C. Where necessary, aeration and stirring may be performed.

When a higher eukaryotic cell, such as animal cell is used as a host cell, a DNA encoding a base editing system of the present invention is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Alternatively, the above-mentioned inductive promoter can also be utilized as a vector removal mechanism when higher eukaryotic cells, such as animal cells, and the like are used as a host cell. That is, a vector is mounted with a replication origin that functions in a host cell, and a nucleic acid encoding a protein necessary for replication (e.g., SV40 on and large T antigen, oriP and EBNA-1 etc. for animal cells), of the expression of the nucleic acid encoding the protein is regulated by the above-mentioned inducible promoter. As a result, while the vector is autonomously replicable in the presence of an induction substance, when the induction substance is removed, autonomous replication is not available, and the vector naturally falls off along with cell division (autonomous replication is not possible by the addition of tetracycline and doxycycline in Tet-OFF system vector).

### Delivery System

The suitability of nucleobase editors to target one or more nucleotides in a polynucleotide sequence (e.g., aA2AR, A2BR, HIF1α, HIF1α.I3 gene) is evaluated as described herein. In one embodiment, a single cell of interest is transfected, transduced, or otherwise modified with a nucleic acid molecule or molecules encoding a base editing system described herein together with a small amount of a vector encoding a reporter (e.g., GFP). These cells can be any cell line known in the art, including immune cells (e.g., T- or NK-cells), or immortalized human cell lines, such as 293T, K562 or U20S. Alternatively, primary cells (e.g., human immune cells) may be used. Cells may also be obtained from a subject or individual, such as from tissue biopsy, surgery, blood, plasma, serum, or other biological fluid. Such cells may be relevant to the eventual cell target.

Delivery may be performed using a viral vector. In one embodiment, transfection may be performed using lipid transfection (such as Lipofectamine or Fugene) or by electroporation. Following transfection, expression of a reporter (e.g., GFP) can be determined either by fluorescence microscopy or by flow cytometry to confirm consistent and high levels of transfection. These preliminary transfections can comprise different nucleobase editors to determine which combinations of editors give the greatest activity. The system can comprise one or more different vectors. In one embodiment, the base editor is codon optimized for expression of the desired cell type, preferentially a eukaryotic cell, preferably a mammalian cell or a human cell.

The activity of the nucleobase editor is assessed as described herein, i.e., by sequencing the genome of the cells to detect alterations in a target sequence. For Sanger sequencing, purified PCR amplicons are cloned into a plasmid backbone, transformed, miniprepped and sequenced with a single primer. Sequencing may also be performed using next generation sequencing (NGS) techniques. When using next generation sequencing, amplicons may be 300-500 bp with the intended cut site placed asymmetrically. Following PCR, next generation sequencing adapters and barcodes (for example Illumina multiplex adapters and indexes) may be added to the ends of the amplicon, e.g., for use in high throughput sequencing (for example on an Illumina MiSeq). The fusion proteins that induce the greatest levels of target specific alterations in initial tests can be selected for further evaluation.

In particular embodiments, the nucleobase editors are used to target polynucleotides of interest. In one embodiment, a nucleobase editor of the invention is delivered to cells (e.g., immune cells (e.g., T- or NK-cells)) in conjunction with one or more guide RNAs that are used to target one or more nucleic acid sequences of interest within the genome of a cell, thereby altering the target gene(s) (e.g., A2AR, A2BR, HIF1α, HIF1α.I3). In some embodiments, a base editor is targeted by one or more guide RNAs to introduce one or more edits to the sequence of one or more genes of interest (e.g., A2AR, A2BR, HIF1α, HIF1α.I3).

In some embodiments, the one or more edits to the sequence of one or more genes of interest decrease or eliminate expression of the protein encoded by the gene in the host cell (e.g., immune cells (e.g., T- or NK-cells)). In some embodiments, expression of one or more proteins encoded by one or more genes of interest (e.g., A2AR, A2BR, HIF1α, HIF1α.I3) is completely knocked out or eliminated in the host cell (e.g., immune cells (e.g., T- or NK-cells)). In some embodiments, the one or more genes of interest are selected from A2AR, A2BR, HIF1α, and/or HIF1α.I3.

In some embodiments, the host cell is selected from a bacterial cell, plant cell, insect cell, human cell, or mammalian cell. In some embodiments, the host cell is a mammalian cell. In some embodiments, the host cell is a human cell. In some embodiments, the cell is in vitro. In some embodiments, the cell is in vivo.

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions.

Nucleic acids encoding cytidine or adenosine base editors can be delivered directly to cells (e.g., immune cells) as naked DNA or RNA, for instance by means of transfection or electroporation, or can be conjugated to molecules (e.g., N-acetylgalactosamine) promoting uptake by the target cells. Nucleic acid vectors, such as the vectors described herein can also be used. In particular embodiments, a polynucleotide, e.g. a mRNA encoding a base editor or a functional component thereof may be co-electroporated with a combination of multiple guide RNAs as described herein.

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g. lipid and/or polymer) nanoparticles are suitable for use as delivery vehicles in certain embodiments of this disclosure. Exemplary lipids for use in nanoparticle formulations, and/or gene transfer are shown in Table 17 (below).

TABLE 17
Lipids used for gene transfer.

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 18 lists exemplary polymers for use in gene transfer and/or nanoparticle formulations.

TABLE 18
Polymers used for gene transfer.

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 19 summarizes delivery methods for a polynucleotide encoding a fusion protein described herein.

TABLE 19
Delivery methods.

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In another aspect, the delivery of base editor system components or nucleic acids encoding such components, for example, a polynucleotide programmable nucleotide binding domain (e.g., Cas9) such as, for example, Cas9 or variants thereof, and a gRNA targeting a nucleic acid sequence of interest, may be accomplished by delivering the ribonucleoprotein (RNP) to cells. The RNP comprises a polynucleotide programmable nucleotide binding domain (e.g., Cas9), in complex with the targeting gRNA. RNPs or polynucleotides described herein may be delivered to cells using known methods, such as electroporation, nucleofection, or cationic lipid-mediated methods, for example, as reported by Zuris, J. A. et al., 2015, Nat. Biotechnology, 33(1):73-80, which is incorporated by reference in its entirety. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants) and to direct homology directed repair (HDR).

Nucleic acid molecules encoding a base editor system can be delivered directly to cells (e.g., T- or NK-cells) as naked DNA or RNA by means of transfection or electroporation, for example, or can be conjugated to molecules (e.g., N-acetylgalactosamine) promoting uptake by the target cells. Vectors encoding base editor systems and/or their components can also be used. In particular embodiments, a polynucleotide, e.g. a mRNA encoding a base editor system or a functional component thereof, may be co-electroporated with one or more guide RNAs as described herein.

Nucleic acid vectors can comprise one or more sequences encoding a domain of a fusion protein described herein. A vector can also encode a protein component of a base editor system operably linked to a nuclear localization signal, nucleolar localization signal, or mitochondrial localization signal. As one example, a vector can include a Cas9 coding sequence that includes one or more nuclear localization sequences (e.g., a nuclear localization sequence from SV40), and one or more deaminases.

The vector can also include any suitable number of regulatory/control elements, e.g., promoters, enhancers, introns, polyadenylation signals, Kozak consensus sequences, or internal ribosome entry sites (IRES). These elements are well known in the art.

Vectors according to this disclosure include recombinant viral vectors. Exemplary viral vectors are set forth herein above. Other viral vectors known in the art can also be used. In addition, viral particles can be used to deliver base editor system components in nucleic acid and/or protein form. For example, “empty” viral particles can be assembled to contain a base editor system or component as cargo. Viral vectors and viral particles can also be engineered to incorporate targeting ligands to alter target tissue specificity.

Vectors described herein may comprise regulatory elements to drive expression of a base editor system or component thereof. Such vectors include adeno-associated viruses with inverted long terminal repeats (AAV ITR). The use of AAV-ITR can be advantageous for eliminating the need for an additional promoter element, which can take up space in the vector. The additional space freed up can be used to drive the expression of additional elements, such as a guide nucleic acid or a selectable marker. ITR activity can be used to reduce potential toxicity due to over expression.

Any suitable promoter can be used to drive expression of a base editor system or component thereof and, where appropriate, the guide nucleic acid. For ubiquitous expression, promoters include CMV, CAG, CBh, PGK, SV40, Ferritin heavy or light chains. For brain or other CNS cell expression, suitable promoters include: SynapsinI for all neurons, CaMKIIalpha for excitatory neurons, GAD67 or GAD65 or VGAT for GABAergic neurons. For liver cell expression, suitable promoters include the Albumin promoter. For lung cell expression, suitable promoters include SP-B. For endothelial cells, suitable promoters include ICAM. For hematopoietic cell expression suitable promoters include IFNbeta or CD45. For osteoblast expression suitable promoters can include OG-2.

In some embodiments, a base editor system of the present disclosure is of small enough size to allow separate promoters to drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters, such as U6 or H1 Use of Pol II promoter and intronic cassettes to express gRNA Adeno Associated Virus (AAV).

In particular embodiments, a fusion protein of the invention is encoded by a polynucleotide present in a viral vector (e.g., adeno-associated virus (AAV), AAV3, AAV3b, AAV4, AAV5, AAV6, AAV7, AAV8, AAV9, AAVrh8, AAV10, and variants thereof), or a suitable capsid protein of any viral vector. Thus, in some aspects, the disclosure relates to the viral delivery of a fusion protein. Examples of viral vectors include retroviral vectors (e.g. Maloney murine leukemia virus, MML-V), adenoviral vectors (e.g. AD100), lentiviral vectors (HIV and FIV-based vectors), herpesvirus vectors (e.g. HSV-2).

In some aspects, the methods described herein for editing specific genes in a cell can be used to genetically modify an immune cell (e.g., T- or NK-cell). In some embodiments, the methods described herein for editing specific genes in an immune cell can be used to genetically modify a CAR-T cell. Such CAR-T cells, and methods to produce such CAR-T cells are described in International Application Nos. PCT/US2016/060736, PCT/US2016/060734, PCT/US2016/034873, PCT/US2015/040660, PCT/EP2016/055332, PCT/IB2015/058650, PCT/EP2015/067441, PCT/EP2014/078876, PCT/EP2014/059662, PCT/IB2014/061409, PCT/US2016/019192, PCT/US2015/059106, PCT/US2016/052260, PCT/US2015/020606, PCT/US2015/055764, PCT/CN2014/094393, PCT/US2017/059989, PCT/US2017/027606, and PCT/US2015/064269, the contents of each is hereby incorporated in its entirety.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors. For example, a base editor and guide nucleic acid can be encoded on a single viral vector. In other embodiments, the base editor and guide nucleic acid are encoded on different viral vectors. In either case, the base editor and guide nucleic acid can each be operably linked to a promoter and terminator. The combination of components encoded on a viral vector can be determined by the cargo size constraints of the chosen viral vector.

The use of RNA or DNA viral based systems for the delivery of a base editor takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (See, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some aspects, a base editor of the present disclosure is of sufficient size so as to enable efficient packaging and delivery into a target cell via a retroviral vector. In some embodiments, a base editor is of a size so as to allow efficient packing and delivery even when expressed together with a guide nucleic acid and/or other components of a targetable nuclease system.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, Adeno-associated virus (“AAV”) vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. AAV vectors can also be used to transduce cells with target nucleic acids, e.g., in the in vitro production of nucleic acids and peptides, and for in vivo and ex vivo gene therapy procedures (See, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

In some embodiments, AAV vectors are used to transduce a cell of interest with a polynucleotide encoding a base editor or base editor system as provided herein. AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (ITRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vpl.

Similar to wt AAV, recombinant AAV (rAAV) utilizes the cis-acting 145-bp ITRs to flank vector transgene cassettes, providing up to 4.5 kb for packaging of foreign DNA. Subsequent to infection, rAAV can express a fusion protein of the invention and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

In some embodiments, lentiviral vectors are used to transduce an immune cell of interest (e.g., T- or NK-cell) with a polynucleotide encoding a base editor or base editor system as provided herein. In some embodiments, lentiviral vectors are used to transduce an modified immune cell (e.g., T- or NK-cell) with a chimeric antigen receptor (CAR). Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 μl Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the base editor-coding sequence and/or the guide nucleic acid can be modified to include one or more modified nucleoside e.g. using pseudo-U or 5-Methyl-C.

The small packaging capacity of AAV vectors makes the delivery of a number of genes that exceed this size and/or the use of large physiological regulatory elements challenging. These challenges can be addressed, for example, by dividing the protein(s) to be delivered into two or more fragments, wherein the N-terminal fragment is fused to a split intein-N and the C-terminal fragment is fused to a split intein-C. These fragments are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

A fragment of a fusion protein of the invention can vary in length. In some embodiments, a protein fragment ranges from 2 amino acids to about 1000 amino acids in length. In some embodiments, a protein fragment ranges from about 5 amino acids to about 500 amino acids in length. In some embodiments, a protein fragment ranges from about 20 amino acids to about 200 amino acids in length. In some embodiments, a protein fragment ranges from about 10 amino acids to about 100 amino acids in length. Suitable protein fragments of other lengths will be apparent to a person of skill in the art.

In one embodiment, dual AAV vectors are generated by splitting a large transgene expression cassette in two separate halves (5′ and 3′ ends, or head and tail), where each half of the cassette is packaged in a single AAV vector (of <5 kb). The re-assembly of the full-length transgene expression cassette is then achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

### Inteins

Inteins (intervening protein) are auto-processing domains found in a variety of diverse organisms, which carry out a process known as protein splicing. Protein splicing is a multi-step biochemical reaction comprised of both the cleavage and formation of peptide bonds. While the endogenous substrates of protein splicing are proteins found in intein-containing organisms, inteins can also be used to chemically manipulate virtually any polypeptide backbone.

In protein splicing, the intein excises itself out of a precursor polypeptide by cleaving two peptide bonds, thereby ligating the flanking extein (external protein) sequences via the formation of a new peptide bond. This rearrangement occurs post-translationally (or possibly co-translationally). Intein-mediated protein splicing occurs spontaneously, requiring only the folding of the intein domain.

About 5% of inteins are split inteins, which are transcribed and translated as two separate polypeptides, the N-intein and C-intein, each fused to one extein. Upon translation, the intein fragments spontaneously and non-covalently assemble into the canonical intein structure to carry out protein splicing in trans. The mechanism of protein splicing entails a series of acyl-transfer reactions that result in the cleavage of two peptide bonds at the intein-extein junctions and the formation of a new peptide bond between the N- and C-exteins. This process is initiated by activation of the peptide bond joining the N-extein and the N-terminus of the intein. Virtually all inteins have a cysteine or serine at their N-terminus that attacks the carbonyl carbon of the C-terminal N-extein residue. This N to O/S acyl-shift is facilitated by a conserved threonine and histidine (referred to as the TXXH motif), along with a commonly found aspartate, which results in the formation of a linear (thio)ester intermediate. Next, this intermediate is subject to trans-(thio)esterification by nucleophilic attack of the first C-extein residue (+1), which is a cysteine, serine, or threonine. The resulting branched (thio)ester intermediate is resolved through a unique transformation: cyclization of the highly conserved C-terminal asparagine of the intein. This process is facilitated by the histidine (found in a highly conserved HNF motif) and the penultimate histidine and may also involve the aspartate. This succinimide formation reaction excises the intein from the reactive complex and leaves behind the exteins attached through a non-peptidic linkage. This structure rapidly rearranges into a stable peptide bond in an intein-independent fashion.

Non-limiting examples of inteins include any intein or intein-pair known in the art, which include a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138(7):2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 459-466.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]—C. In some embodiments, an intein-C is fused to the N-terminus of the C-terminal portion of the split Cas9, i.e., to form a structure of N-[intein-C]--[C-terminal portion of the split Cas9]-C. The mechanism of intein-mediated protein splicing for joining the proteins the inteins are fused to (e.g., split Cas9) is known in the art, e.g., as described in Shah et al., Chem Sci. 2014; 5(1):446-461, incorporated herein by reference. Methods for designing and using inteins are known in the art and described, for example by WO2014004336, WO2017132580, US20150344549, and US20180127780, each of which is incorporated herein by reference in their entirety.

In some embodiments, a portion or fragment of a nuclease (e.g., Cas9) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, an N-terminal fragment of a base editor (e.g., ABE, CBE) is fused to a split intein-N and a C-terminal fragment is fused to a split intein-C. These fragments are then packaged into two or more AAV vectors. In some embodiments, the N-terminus of an intein is fused to the C-terminus of a fusion protein and the C-terminus of the intein is fused to the N-terminus of an AAV capsid protein.

In one embodiment, inteins are utilized to join fragments or portions of a cytidine or adenosine base editor protein that is grafted onto an AAV capsid protein. The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, which are indicated in capital letters in the sequence below (called the “Cas9 reference sequence”).

| (SEQ ID NO: 197) | |
|---|---|
| 1 | mdkkysigld igtnsvgwav itdeykvpsk kfkvlgntdr hsikknliga llfdsgetae |
| 61 | atrlkrtarr rytrrknric ylqeifsnem akvddsffhr leesflveed kkherhpifg |
| 121 | nivdevayhe kyptiyhlrk klvdstdkad lrliylalah mikfrghfli egdlnpdnsd |
| 181 | vdklfiqlvq tynqlfeenp inasgvdaka ilsarlsksr rlenliaqlp gekknglfgn |
| 241 | lialslgltp nfksnfdlae daklqlskdt ydddldnlla qigdqyadlf laaknlsdai |
| 301 | llSdilrvnT eiTkaplsas mikrydehhq dltllkalvr qqlpekykei ffdqSkngya |
| 361 | gyidggasqe efykfikpil ekmdgteell vklnredllr kqrtfdngsi phqihlgelh |
| 421 | ailrrqedfy pflkdnreki ekiltfripy yvgplArgnS rfAwmTrkSe eTiTpwnfee |
| 481 | vvdkgasaqs fiermtnfdk nlpnekvlpk hsllyeyftv yneltkvkyv tegmrkpafl |
| 541 | sgeqkkaivd llfktnrkvt vkqlkedyfk kieCfdSvei sgvedrfnAS lgtyhdllki |
| 601 | ikdkdfldne enedilediv ltltlfedre mieerlktya hlfddkvmkq lkrrrytgwg |
| 661 | rlsrklingi rdkqsgktil dflksdgfan rnfmqlihdd sltfkediqk aqvsgqgdsl |
| 721 | hehianlags paikkgilqt vkvvdelvkv mgrhkpeniv iemarenqtt qkgqknsrer |
| 781 | mkrieegike lgsqilkehp ventqlqnek lylyylqngr dmyvdqeldi nrlsdydvdh |
| 841 | ivpqsflkdd sidnkvltrs dknrgksdnv pseevvkkmk nywrqllnak litqrkfdnl |
| 901 | tkaergglse ldkagfikrq lvetrqitkh vaqildsrmn tkydendkli revkvitlks |
| 961 | klvsdfrkdf qfykvreinn yhhahdayln avvgtalikk ypklesefvy gdykvydvrk |
| 1021 | miakseqeig katakyffys nimnffktei tlangeirkr plietngetg eivwdkgrdf |
| 1081 | atvrkvlsmp qvnivkktev qtggfskesi lpkrnsdkli arkkdwdpkk yggfdsptva |
| 1141 | ysvlvvakve kgkskklksv kellgitime rssfeknpid fleakgykev kkdliiklpk |
| 1201 | yslfelengr krmlasagel qkgnelalps kyvnflylas hyeklkgspe dneqkqlfve |
| 1261 | qhkhyldeii eqisefskrv iladanldkv lsaynkhrdk pireqaenii hlftltnlga |
| 1321 | paafkyfdtt idrkrytstk evldatlihq sitglyetri dlsqlggd |

### Pharmaceutical Compositions

In some aspects, the present invention provides a pharmaceutical composition comprising any of the genetically modified immune cells, polynucleotides, vectors, base editors, base editor systems, guide polynucleotides, fusion proteins, or the fusion protein-guide polynucleotide complexes described herein. More specifically, provided herein are pharmaceutical compositions comprising a genetically modified immune cell (e.g., T- or NK-cell), or a population of such immune cells, wherein said modified immune cell has at least one edited gene involved in hypoxic and adenosinergic pathways (e.g., A2AR, A2BR, HIF1α, HIF1α.I3), or regulatory elements thereof, to provide increased resistance to hypoxic-adenosinergic immunosuppression and/or increased cytokine production. In some embodiments, the at least one edited gene is A2AR, A2BR, HIF1α, and/or HIF1α.I3. In embodiments, the pharmaceutical compositions comprise or are administered in combination with an A2AR, A2BR, HIF1α, and/or HIF1α.I3 antagonist (e.g., AZD4635).

The pharmaceutical compositions of the present invention can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g., tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In addition to a modified immune cell, or population thereof, and a carrier, the pharmaceutical compositions of the present invention can include at least one additional therapeutic agent useful in the treatment of disease. For example, some embodiments of the pharmaceutical composition described herein further comprises a chemotherapeutic agent. In some embodiments, the pharmaceutical composition further comprises a cytokine peptide or a nucleic acid sequence encoding a cytokine peptide. In some embodiments, the pharmaceutical compositions comprising the modified immune cell or population thereof can be administered separately from an additional therapeutic agent.

One consideration concerning the therapeutic use of genetically modified cells of the invention is the quantity of cells necessary to achieve an optimal or satisfactory effect. The quantity of cells to be administered may vary for the subject being treated. In one embodiment, between 10⁴ to 10¹⁰, between 10⁵ to 10⁹, or between 10⁶ and 10⁸ genetically modified immune cells of the invention are administered to a human subject. In some embodiments, at least about 1×10⁸, 2×10⁸, 3×10⁸, 4×10⁸, and 5×10⁸ genetically modified cells of the invention are administered to a human subject. Determining the precise effective dose may be based on factors for each individual subject, including their size, age, sex, weight, and condition. Dosages can be readily ascertained by those skilled in the art from this disclosure and the knowledge in the art.

The skilled artisan can readily determine the number of cells and amount of optional additives, vehicles, and/or carriers in compositions and to be administered in methods of the invention. Typically, additives (in addition to the cell(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD50 in a suitable animal model (e.g., a rodent such as a mouse); and, the dosage of the composition(s), concentration of components therein, and the timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., solid tumor microenvironment). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (see, e.g., Langer, 1990, Science 249: 1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228: 190; During et al., 1989, Ann. Neurol. 25:351; Howard et al., 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, any of the fusion proteins, gRNAs, and/or complexes described herein are provided as part of a pharmaceutical composition. In some embodiments, the pharmaceutical composition comprises any of the fusion proteins provided herein. In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. Pharmaceutical compositions can optionally comprise one or more additional therapeutically active substances.

In some embodiments, compositions provided herein are administered to a subject, for example, to a human subject, in order to effect a targeted genomic modification within the subject. In some embodiments, cells are obtained from the subject and contacted with any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts, for example, for veterinary use.

Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated in its entirety herein by reference) discloses various excipients used in formulating pharmaceutical compositions and known techniques for the preparation thereof. See also PCT application PCT/US2010/055131 (Publication number WO2011/053982 A8, filed Nov. 2, 2010), incorporated in its entirety herein by reference, for additional suitable methods, reagents, excipients and solvents for producing pharmaceutical compositions comprising a nuclease.

Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions. In some embodiments, the pharmaceutical compositions of the present invention can be used to treat any disease or condition (e.g., cancer, neoplasias, solid tumors) that is susceptible to hypoxia-adenosinergic immunosuppression. In some embodiments, the pharmaceutical compositions of the present invention are used to treat solid tumors.

### Methods of Treatment

Some aspects of the present invention provide methods of treating a subject in need, the method comprising administering to a subject in need an effective therapeutic amount of a pharmaceutical composition as described herein. More specifically, the methods of treatment include administering to a subject in need thereof one or more pharmaceutical compositions comprising one or more immune cells having at least one edited gene. In other embodiments, the methods of the invention comprise expressing or introducing into an immune cell a base editor polypeptide and one or more guide RNAs capable of targeting a nucleic acid molecule encoding at least one polypeptide. In some embodiments, the invention provides methods of treating a subject in need with a solid tumor.

In some embodiments, the methods of treatment comprise administering to a subject in need thereof a pharmaceutical composition comprising a population of modified immune cells having at least one edited gene (e.g., A2AR, A2BR, HIF1α, HIF1α.I3), wherein the at least one edited gene increases the immune cell's resistance to hypoxia-adenosinergic immunosuppression. In embodiments, the pharmaceutical compositions comprise or are administered in combination with an A2AR, A2BR, HIF1α, and/or HIF1α.I3 antagonist (e.g., AZD4635). In some embodiments, the methods of treatment comprise administering to a subject an effective amount of a modified immune effector cell (e.g., CAR-T cell) or a population thereof that has at least one single target nucleobase modification in one or more of genes in a hypoxic and/or adenosinergic pathway component or a regulatory element thereof (e.g., A2AR, A2BR, HIF1α, and/or HIF1α.I3) and has increased resistance to hypoxia-adenosinergic immunosuppression. In some embodiments, the methods of treatment comprise administering to a subject an effective amount of a modified immune effector cell (e.g., CAR-T cell) or a population thereof that has at least one single target nucleobase modification in one or more of genes in a hypoxic and/or adenosinergic pathway component or a regulatory element thereof (e.g., A2AR, A2BR, HIF1α, and/or HIF1α.I3) and has increased cytokine production. In some embodiments, the one or more of genes involved in a hypoxic and/or adenosinergic pathway component or a regulatory element thereof are selected from one or more of A2AR, A2BR, HIF1α, and/or HIF1α.I3. In some embodiments, the modified immune cell is a T cell. In some embodiments, the modified immune cell is a CAR-T cell.

In one embodiment, a subject is administered at least 0.1×10⁵ cells, at least 0.5×10⁵ cells, at least 1×10⁵ cells, at least 5×10⁵ cells, at least 1×10⁶ cells, at least 0.5×10⁷ cells, at least 1×10⁷ cells, at least 0.5×10⁸ cells, at least 1×10⁸ cells, at least 0.5×10⁹ cells, at least 1×10⁹ cells, at least 2×10⁹ cells, at least 3×10⁹ cells, at least 4×10⁹ cells, at least 5×10⁹ cells, or at least 1×10¹⁰ cells. In particular embodiments, about 1×10⁷ cells to about 1×10⁹ cells, about 2×10⁷ cells to about 0.9×10⁹ cells, about 3×10⁷ cells to about 0.8×10⁹ cells, about 4×10⁷ cells to about 0.7×10⁹ cells, about 5×10⁷ cells to about 0.6×10⁹ cells, or about 5×10⁷ cells to about 0.5×10⁹ cells are administered to the subject.

In one embodiment, a subject is administered at least 0.1×10⁴ cells/kg of bodyweight, at least 0.5×10⁴ cells/kg of bodyweight, at least 1×10⁴ cells/kg of bodyweight, at least 5×10⁴ cells/kg of bodyweight, at least 1×10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10′ cells/kg of bodyweight. In particular embodiments, about 1×10⁶ cells/kg of bodyweight to about 1×10⁸ cells/kg of bodyweight, about 2×10⁶ cells/kg of bodyweight to about 0.9×10⁸ cells/kg of bodyweight, about 3×10⁶ cells/kg of bodyweight to about 0.8×10⁸ cells/kg of bodyweight, about 4×10⁶ cells/kg of bodyweight to about 0.7×10⁸ cells/kg of bodyweight, about 5×10⁶ cells/kg of bodyweight to about 0.6×10⁸ cells/kg of bodyweight, or about 5×10⁶ cells/kg of bodyweight to about 0.5×10⁸ cells/kg of bodyweight are administered to the subject.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more. In any of such methods, the methods may comprise administering to the subject an effective amount of an modified immune cell or a base editor system or polynucleotide encoding such system. In any of such methods, the methods may comprise administering one or more doses of an effective amount of the modified immune cells per day. In any of such methods, the methods may comprise administering two or more doses of an effective amount of the modified immune cells per day. In any of such methods, the methods may comprise administering three or more doses of an effective amount of the modified immune cells per day. In any of such methods, the methods may comprise administering one or more doses of an effective amount of the modified immune cells per week. In any of such methods, the methods may comprise administering two or more doses of an effective amount of the modified immune cells per week. In any of such methods, the methods may comprise administering three or more doses of an effective amount of the modified immune cells per week. In any of such methods, the methods may comprise administering one or more doses of an effective amount of the modified immune cells per month. In any of such methods, the methods may comprise administering two or more doses of an effective amount of the modified immune cells per month. In any of such methods, the methods may comprise administering three or more doses of an effective amount of the modified immune cells per month.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

In some embodiments, a composition described herein (e.g., modified immune cell, base editor system) is administered in a dosage that is about 0.5-30 mg per kilogram body weight of the human subject. In another embodiment, the amount of the composition administered is about 0.5-20 mg per kilogram body weight of the human subject. In another embodiment, the amount of the composition administered is about 0.5-10 mg per kilogram body weight of the human subject. In another embodiment, the amount of the composition administered is about 0.04 mg, about 0.08 mg, about 0.16 mg, about 0.32 mg, about 0.64 mg, about 1.25 mg, about 1.28 mg, about 1.92 mg, about 2.5 mg, about 3.56 mg, about 3.75 mg, about 5.0 mg, about 7.12 mg, about 7.5 mg, about 10 mg, about 14.24 mg, about 15 mg, about 20 mg, or about 30 mg per kilogram body weight of the human subject. In another embodiment, the amount of the compo composition und administered is about 1.92 mg, about 3.75 mg, about 7.5 mg, about 15.0 mg, or about 30.0 mg per kilogram body weight of the human subject and the composition is administered two times a week. In another embodiment, the amount of the composition administered is about 1.28 mg, about 2.56 mg, about 5.0 mg, about 10 mg, or about 20 mg per kilogram body weight of the human subject and the composition is administered two times a week. In another embodiment, the amount of the composition administered is about 1.92 mg, about 3.75 mg, about 7.5 mg, about 15.0 mg, or about 30.0 mg per kilogram body weight of the human subject and the composition is administered once a week. In another embodiment, the amount of the composition administered is about 1.28 mg, about 2.56 mg, about 5.0 mg, about 10 mg, or about 20 mg per kilogram body weight of the human subject and the composition is administered once a week. In another embodiment, the amount of the composition administered is about 1.92 mg, about 3.75 mg, about 7.5 mg, about 15.0 mg, or about 30.0 mg per kilogram body weight of the human subject and the composition is administered once a day three, five or seven times in a seven day period. In another embodiment, the composition is administered intravenously once a day, seven times in a seven day period. In another embodiment, the amount of the composition administered is about 1.28 mg, about 2.56 mg, about 5.0 mg, about 10 mg, or about 20 mg per kilogram body weight of the human subject and the composition is administered once a day three, five or seven times in a seven day period. In another embodiment, the composition is administered intravenously once a day, seven times in a seven day period.

In some embodiments, the composition is administered over a period of 0.25 h, 0.5 h, 1 h, 2 h, 3 h, 4 h, 5 h, 6 h, 7 h, 8 h, 9 h, 10 h, 11 h, or 12 h. In another embodiment, the composition is administered over a period of 0.25-2 h. In another embodiment, the composition is gradually administered over a period of 1 h. In another embodiment, the composition is gradually administered over a period of 2 h.

### Kits

The invention provides kits for the treatment of cancers, neoplasias, solid tumors in a subject. The invention further provides kits featuring a modified immune cell (e.g., T- or NK-cell) as provided herein. In some embodiments, the kit includes a modified CAR-T cell as provided herein. In some embodiments, the kit further includes a base editor, base editor system or a polynucleotide encoding a base editor or base editor system as provided herein. In some embodiments, the kit further includes one or more guide polynucleotides (e.g., a guide polynucleotide targeting a genomic sequence) as provided herein. In some embodiments, the base editor system comprises a nucleic acid programmable DNA binding protein (napDNAbp), a deaminase (e.g., cytidine deaminase or adenosine deaminase), and a guide RNA for modifying an immune cell (e.g., T- or NK-cell). In some embodiments, the deaminase is a cytidine deaminase or an adenosine deaminase. In some embodiments, the base editor is an ABE, CBE, or ABE/CBE. In some embodiments, the guide RNA targets a site selected from those listed in Table 1A and/or a site corresponding to a spacer listed in Table 1A or Table 1B.

The kits may further comprise written instructions for using the base editor, base editor system and/or modified immune cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Knocking Out HIF1α, A2AR, or A2BR Had Minimal or No Impact on CAR Expression in CAR T Cells

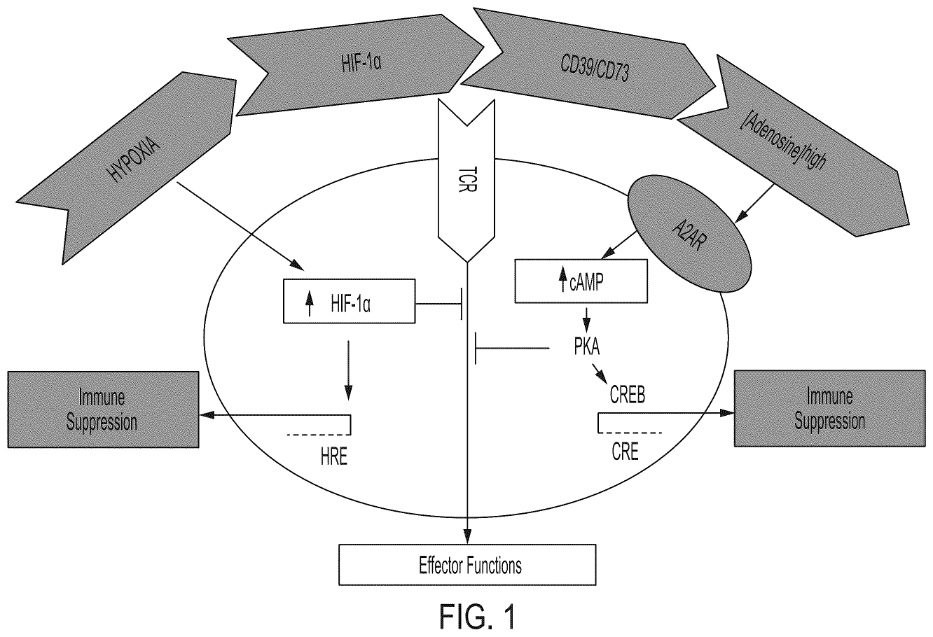

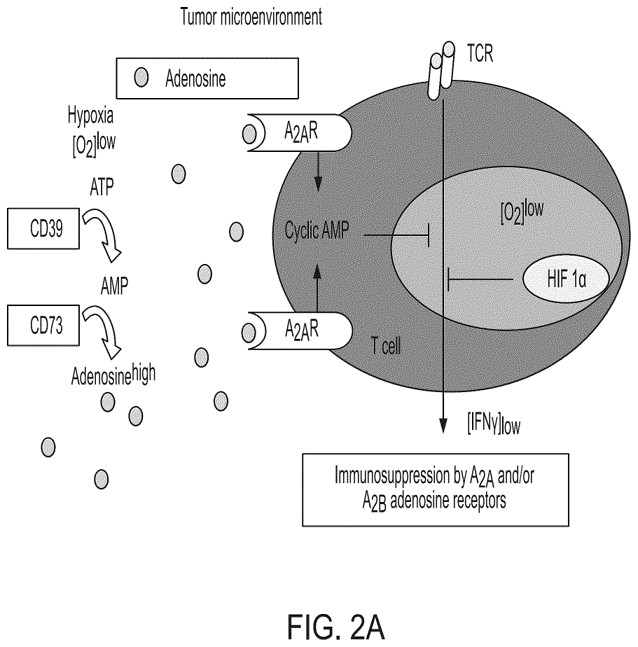

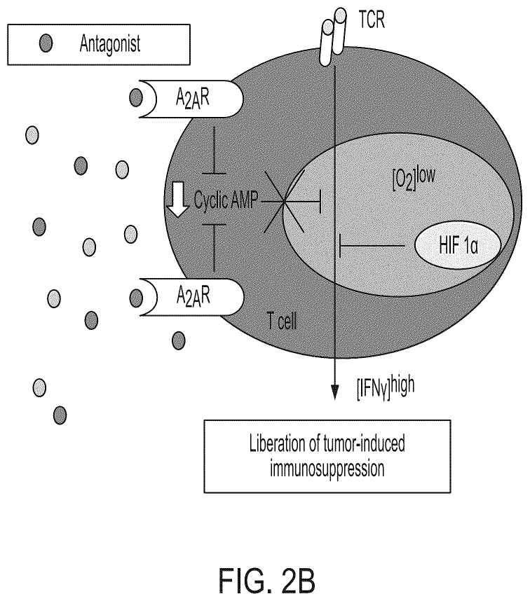

As a first step in developing chimeric antigen receptor (CAR) T cells genetically resistant to hypoxia-adenosinergic immunosuppression and, thus, having improved capacity to be used in the clinical treatment of solid tumors, the impact on CAR expression of knocking out the target genes HIF1a, A2AR, or A2BR using base editors was evaluated. HIF1α, A2AR, or A2BR were chosen as targets for knocking out using base editing because, as shown in FIGS. 1, 2A, and 2B, these genes encode proteins playing a role in the hypoxic and adenosinergic pathways that play synergistic roles in suppressing T cells in the tumor microenvironment. Knocking out one or more of these target genes is associated with a reduction in suppression of T cells in the tumor microenvironment (e.g., a hypoxic environment and/or an environment comprising elevated levels of adenosine). Indeed, as shown in FIGS. 2A and 2B, adenosine receptor antagonists alleviate T cell immunosuppression in the tumor microenvironment.

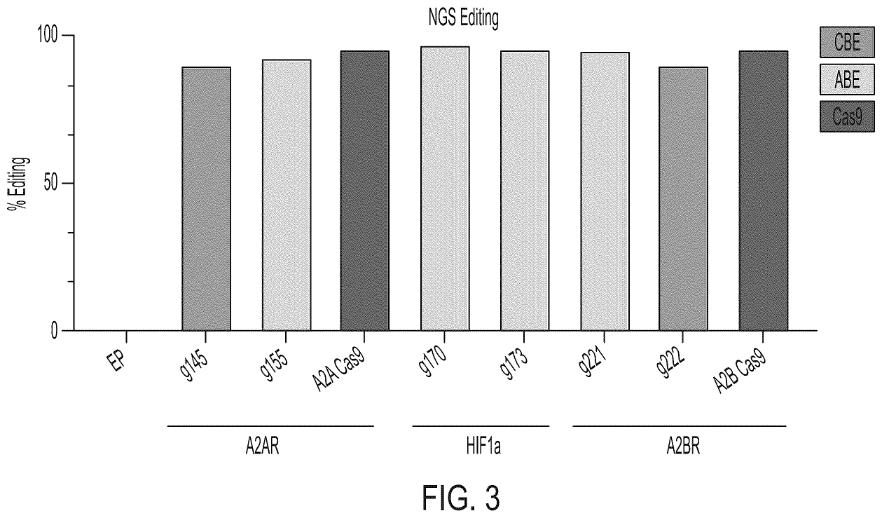

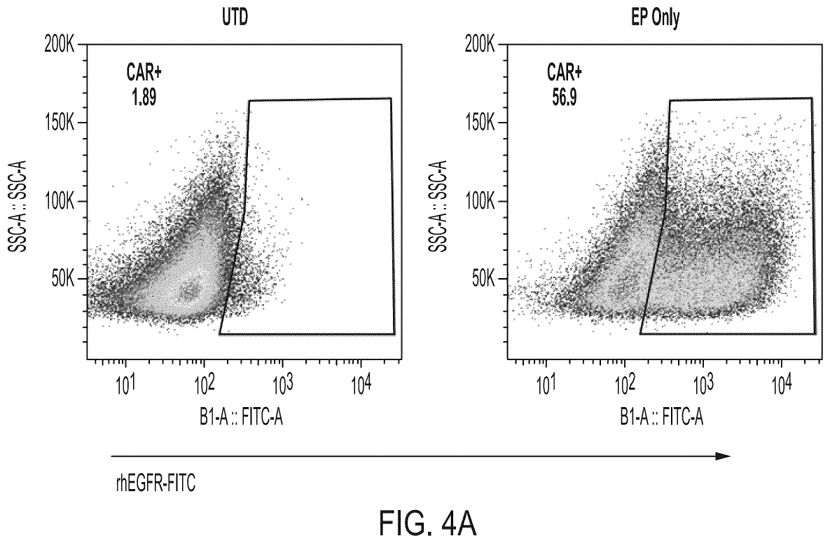

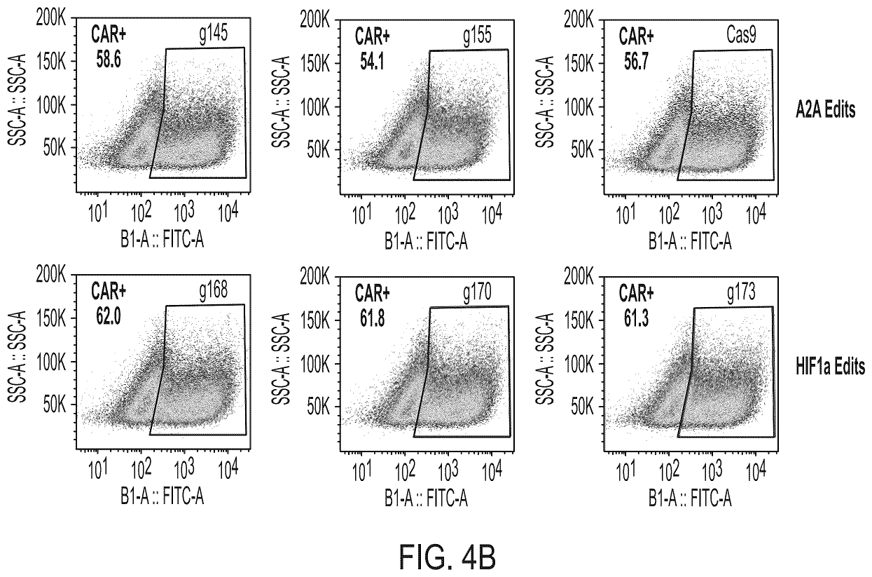

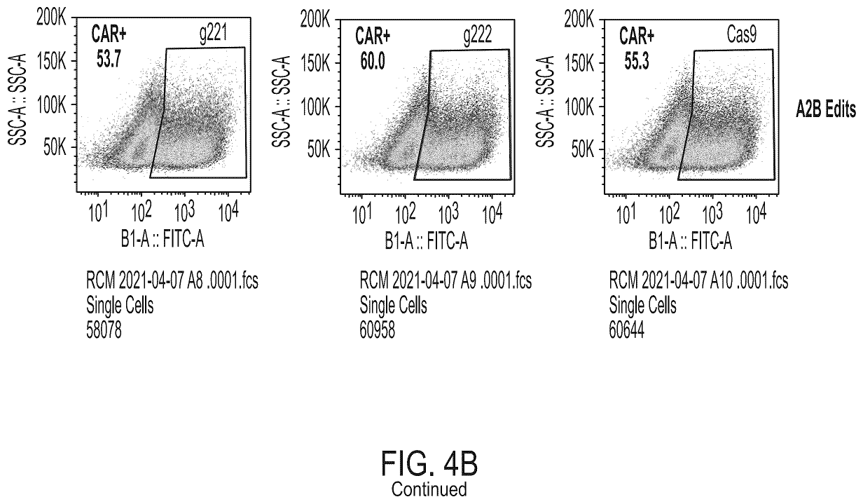

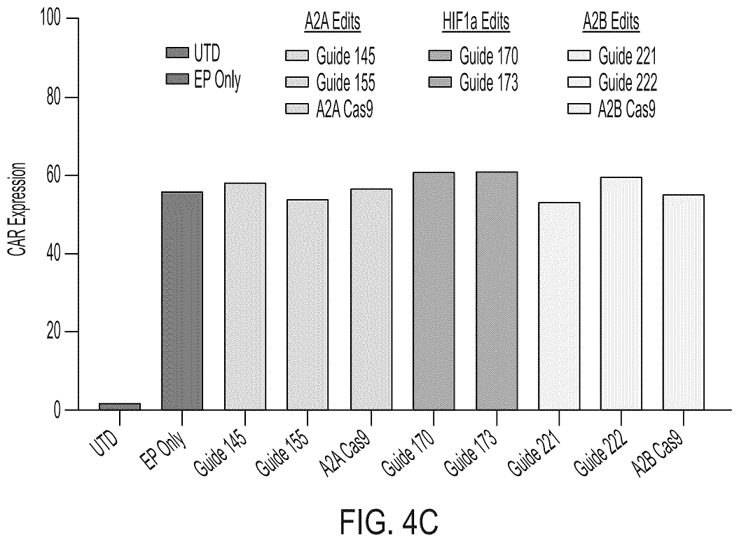

As shown in FIG. 3, it was determined using next generation sequencing of edited cells that a cytosine base editor (CBE) (rBE4) used in combination with gRNA 145 to edit A2AR and with gRNA 222 to edit A2BR, an adenosine base editor ABE (ABE8.20) used in combination with gRNA 155 to edit A2AR, with gRNA 170 or 172 to edit HIF1a, or with gRNA 221 to edit A2BR, or Cas9 used in combination with an A2A Cas9 guide RNA or an A2B Cas9 guide RNA yielded high molecular editing (i.e., base editing efficiencies>90%) at the targeted sites in CAR-T cells. The guide RNA sequences target a site provided in Table 1A or corresponding to a spacer listed in Table 1A and/or Table 1B. Further, as shown in FIGS. 4A-4C, 60% chimeric antigen receptor (CAR) expression was seen across all guides and editing targets. No impact was observed on CAR expression. The CAR-T cells expressed an anti-human EGFR CAR polypeptide.

### Example 2: Knocking Out HIF1α Increased Cytokine Production Under Hypoxic Stress Relative to Unedited CAR T Cells

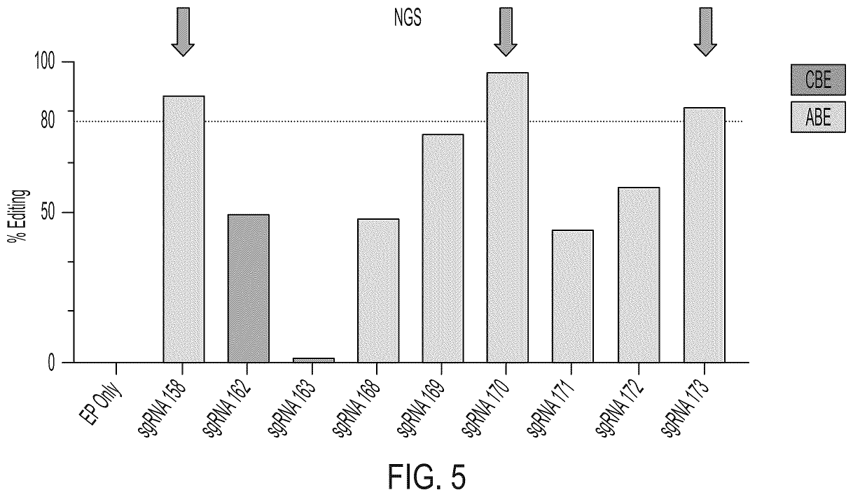

Next, experiments were completed to identify top-performing guide RNAs for use in knocking out the HIF1a gene using base editing. As shown in FIG. 5, it was determined through next generation sequencing (NGS) that guides 158, 170, and 173 in conjunction with an ABE (ABE8.20) resulted in HIF1α gene editing.

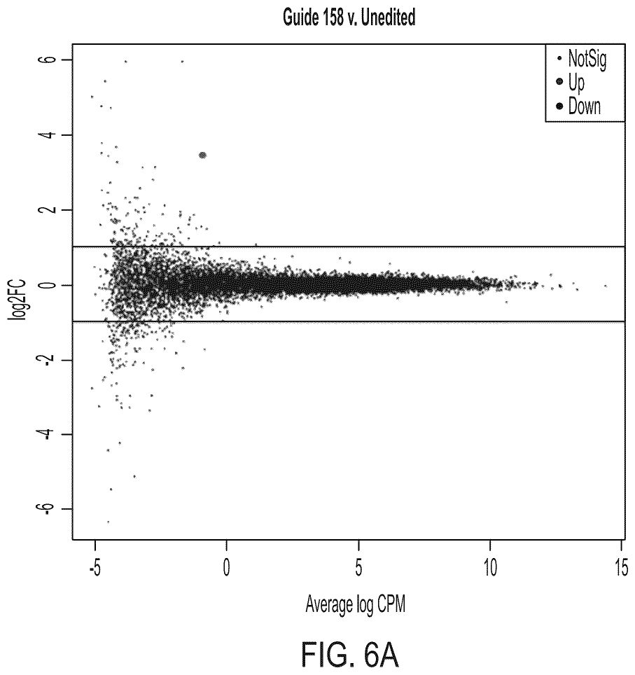

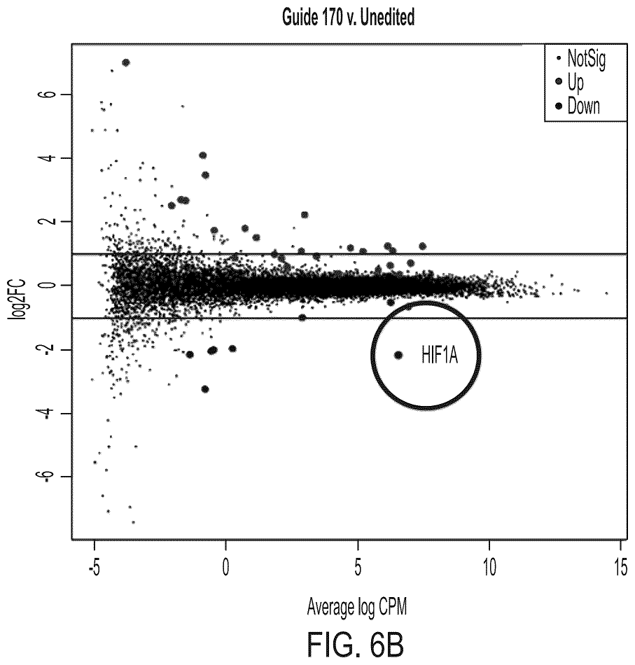

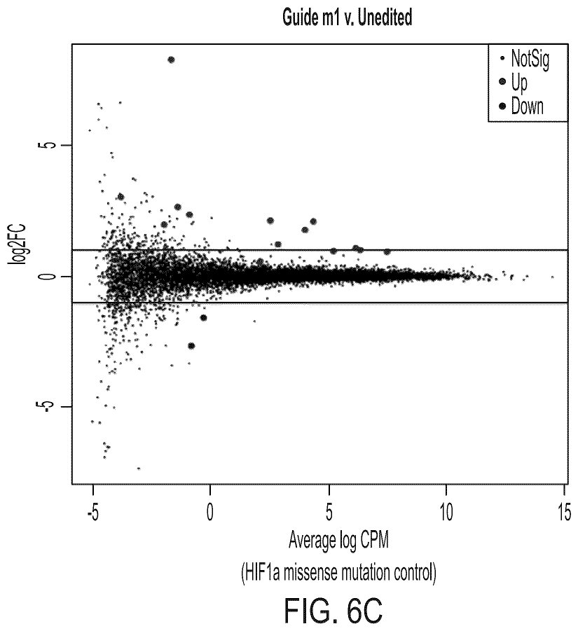

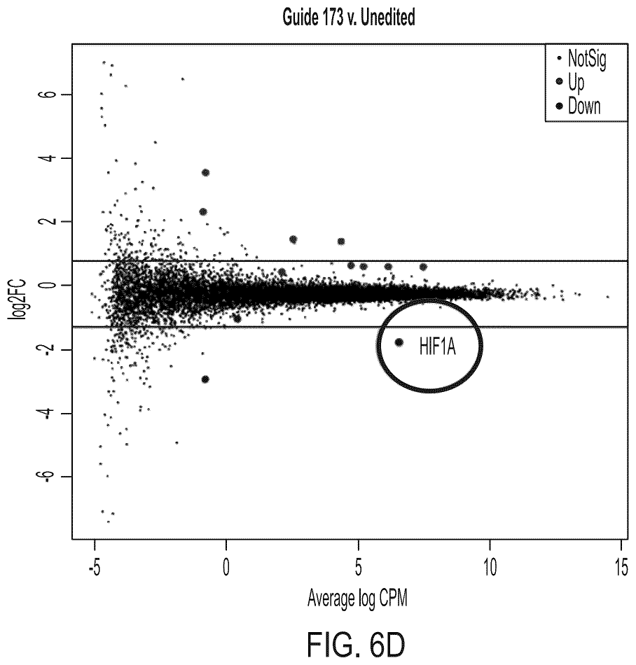

Transcripts produced from the edited HIF1α genes were analyzed using RNAseq to determine the impact of the edits on transcription and splicing. As shown in FIGS. 6A-6D, it was determined that guides 170 and 173 were associated with edits that decreased transcription of the HIF1α gene.

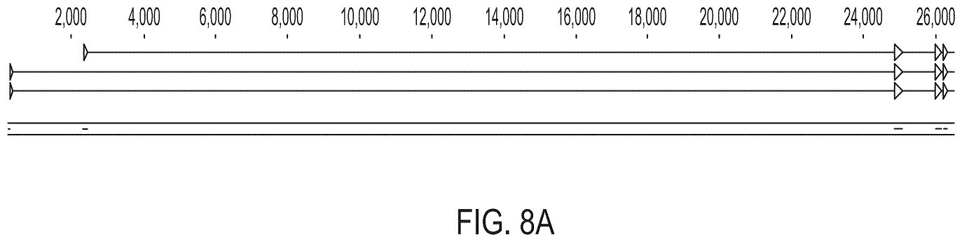

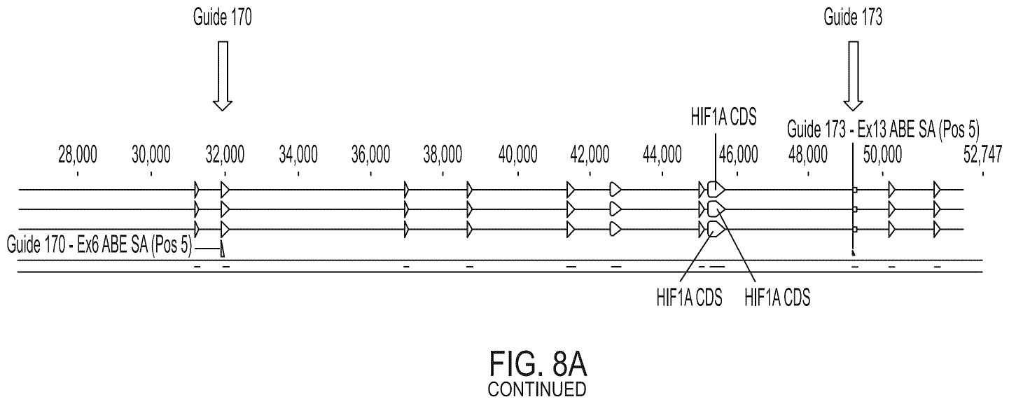

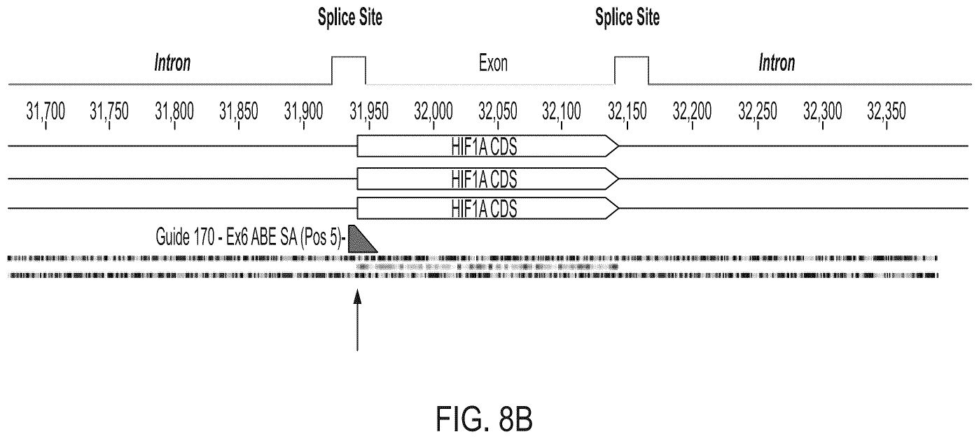

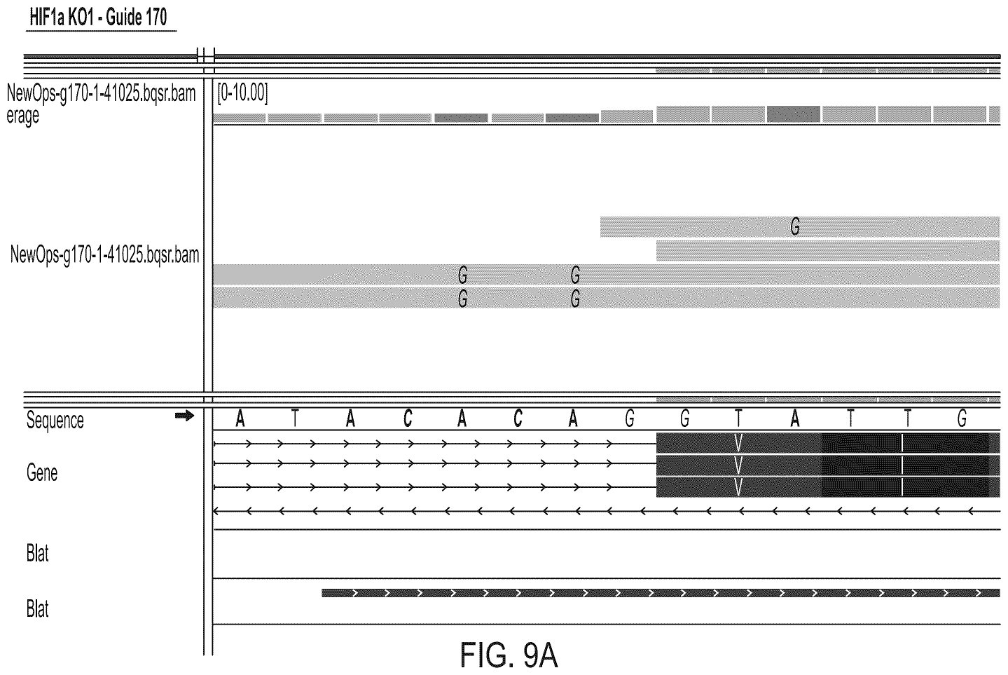

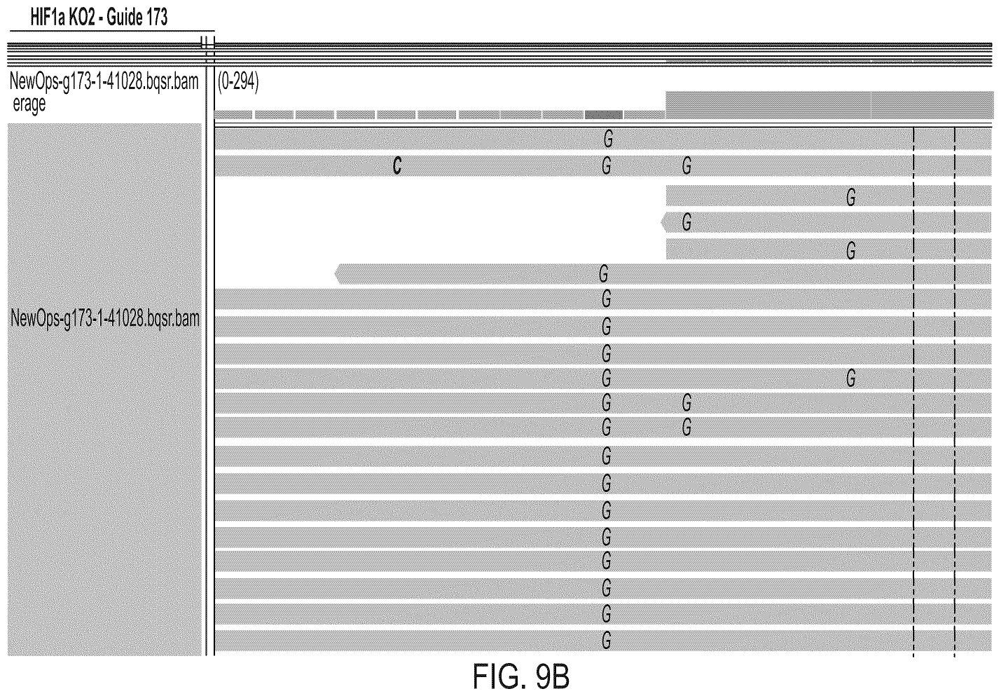

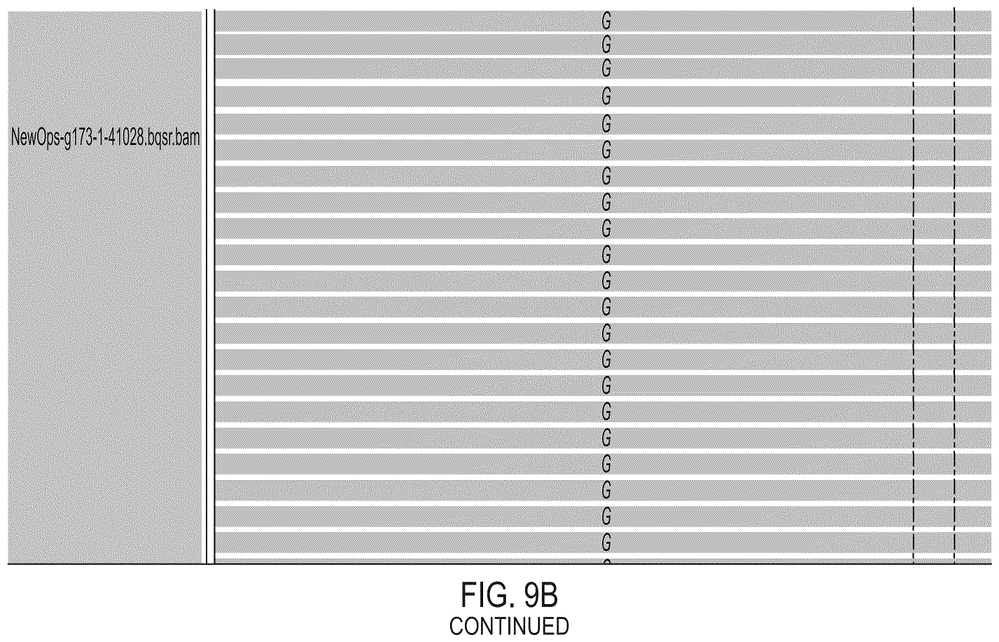

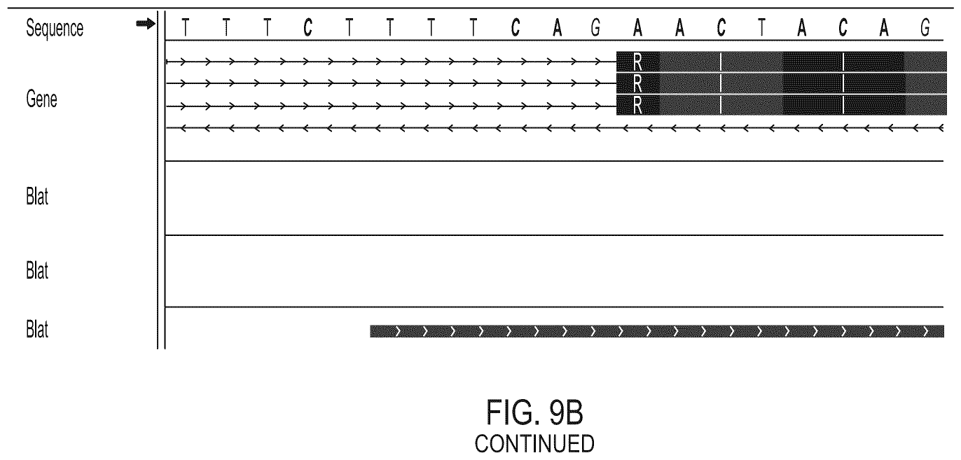

Editing of the HIF1α gene using guides 170 and 173 altered the splicing of mRNA transcribed from the edited genes (see FIGS. 9A and 9B), as determined using RNAseq. Without intending to be bound by theory, such alterations in splicing were associated with edits made proximal to the interface of introns and exons in the HIFIla gene (see, e.g., the sites targeted by Guide 170 and 173, as indicated in FIGS. 8A and 8B). In other words, without intending to be bound by theory, it is likely that guides 170 and 173 targeted different intron/exon splice sites across the HIF1α gene (see FIGS. 8A and 8B) and the edits associated with the use of these guides resulted in improper splicing, leading to effective protein knockout. As shown in FIGS. 9A and 9B, more robust editing and intron retention was associated with guide 173 than with guide 170. The HIF1α gene sequence is provided in the sequence listing as SEQ ID NO: 377.

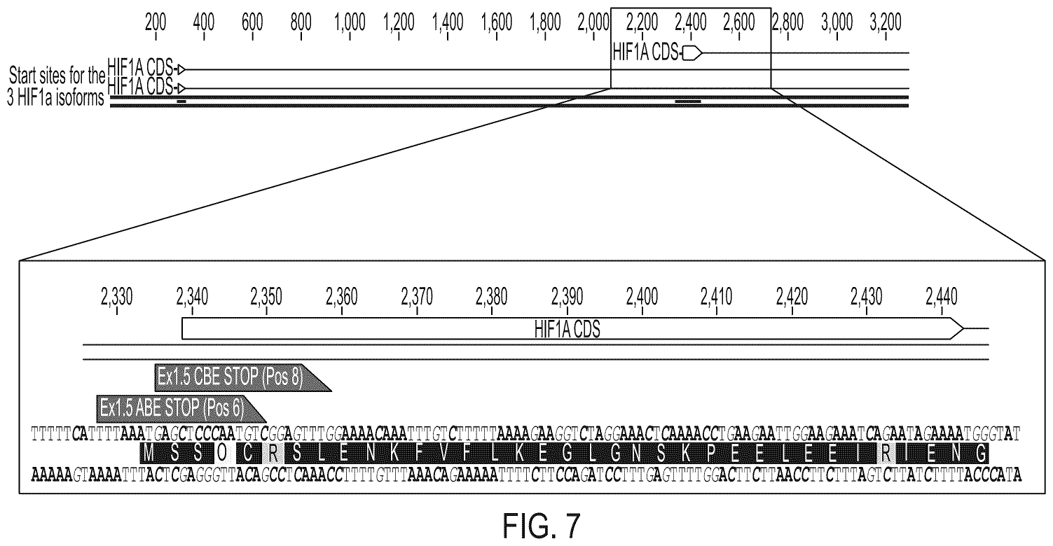

In some embodiments editing of a target site in the HIF1α gene is associated with knocking out of a particular isoform of HIF1α (see FIG. 7). For example, while not being bound by theory, knocking out expression of the HIF1cαI3 isoform while leaving ubiquitous HIF1α intact is associated in some instances with increased cytokine production under hypoxic stress.

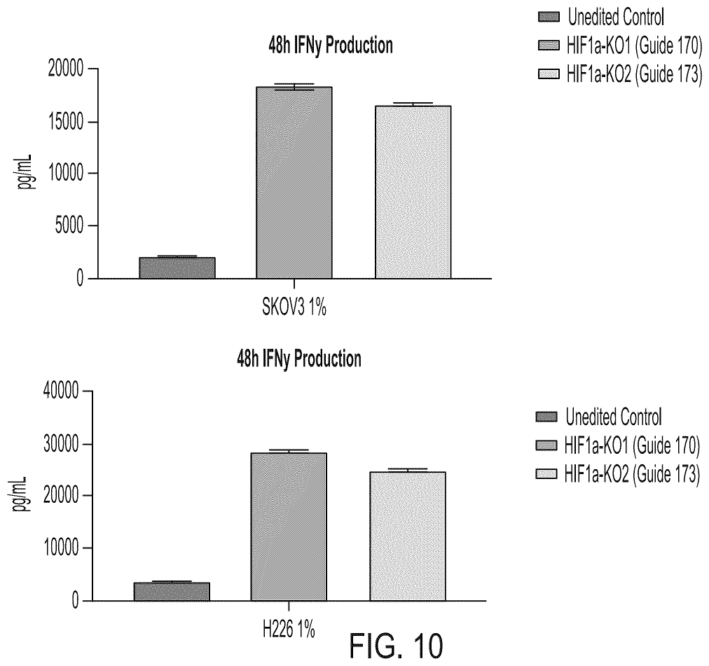

The impact of knocking out the HIF1a gene using guides 170 and 173 on cytokine production by anti-EGFR CAR-T cells under hypoxic stress was evaluated. The EGFR CAR-T cells were co-cultured at an E:T ratio of 1:1 with either SKOV3 or H226 cells in 1% O₂ for 48 hours. The HIF1α gene knockout edits resulted in increased cytokine production under hypoxic stress compared to unedited CAR-T cells, as shown in FIG. 10.

### Example 3: Knocking Out A2AR Protected CAR-T Cells from Adenosine-Mediated Cytokine Suppression

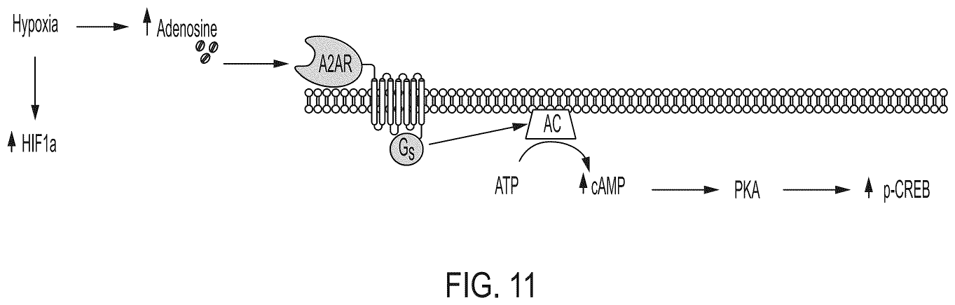

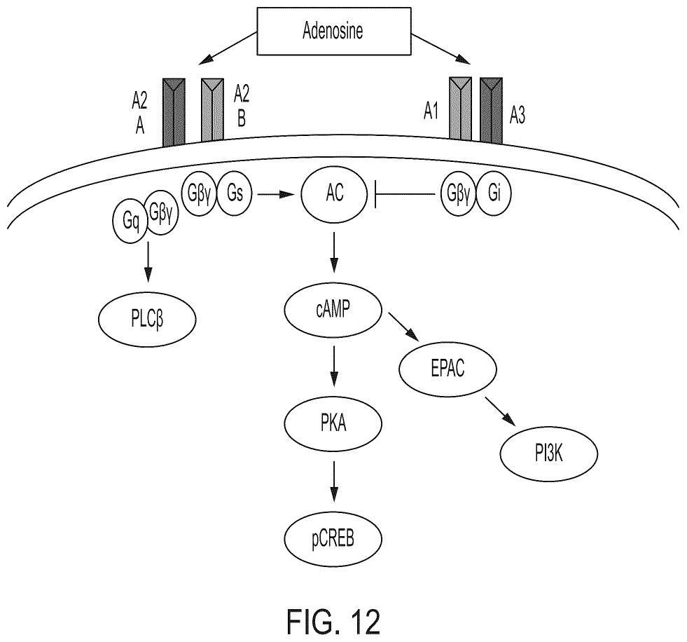

Next, gRNAs were screened for use in knocking out the A2AR gene. As shown in FIGS. 11 and 12, the A2AR gene is a component of the hypoxia-adenosinergic axis involved in suppressing cytotoxic T cell function in response to hypoxia. Thus, knocking out the A2AR gene is associated with a reduction in hypoxia/adenosine-mediated suppression of cytotoxic T cell function. Further, as shown in FIG. 12, knocking out of the A2BR gene has a similar effect. Without intending to be bound by theory, activation of A2AR (A2A in FIG. 12) and/or A2BR (A2B in FIG. 12) in response to hypoxia/adenosine result in an increase in intracellular levels of cAMP and pCREB.

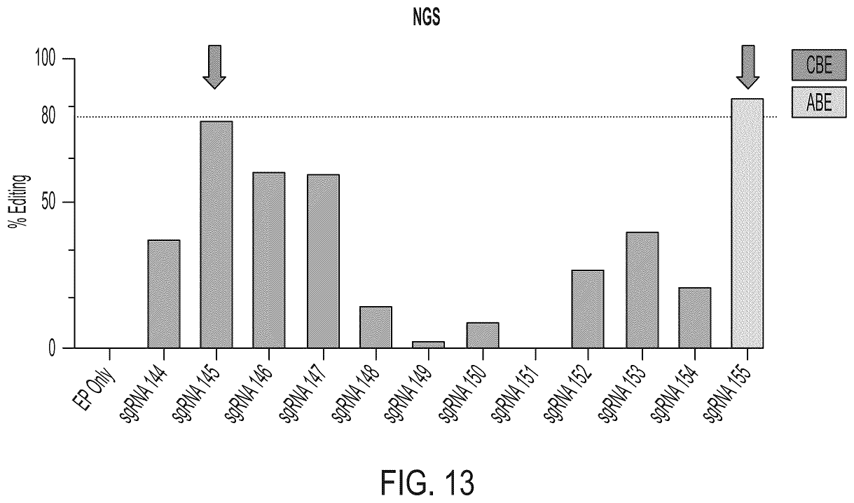

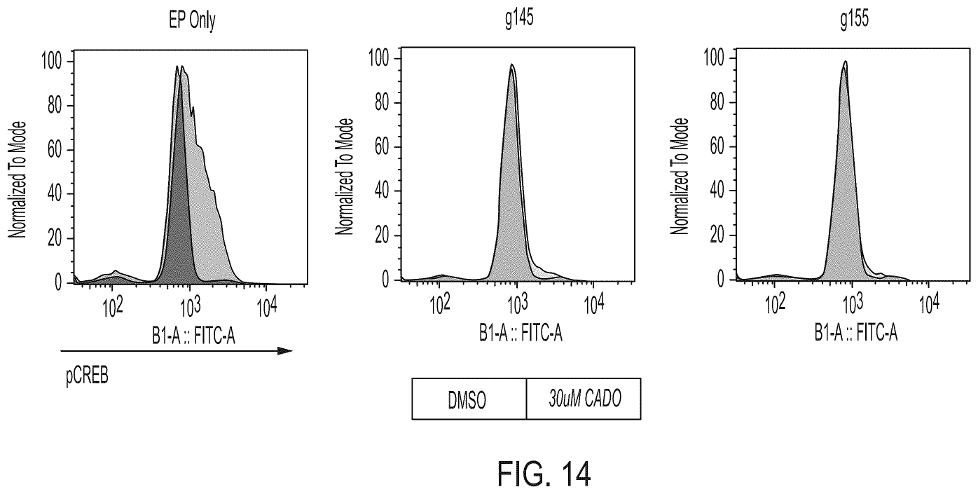

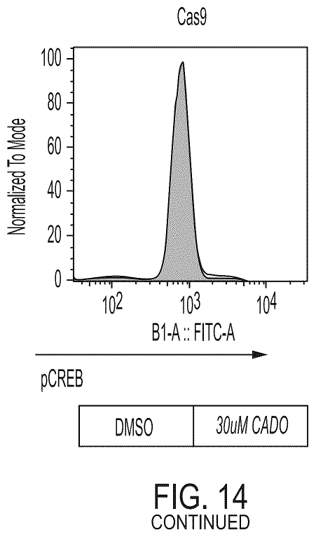

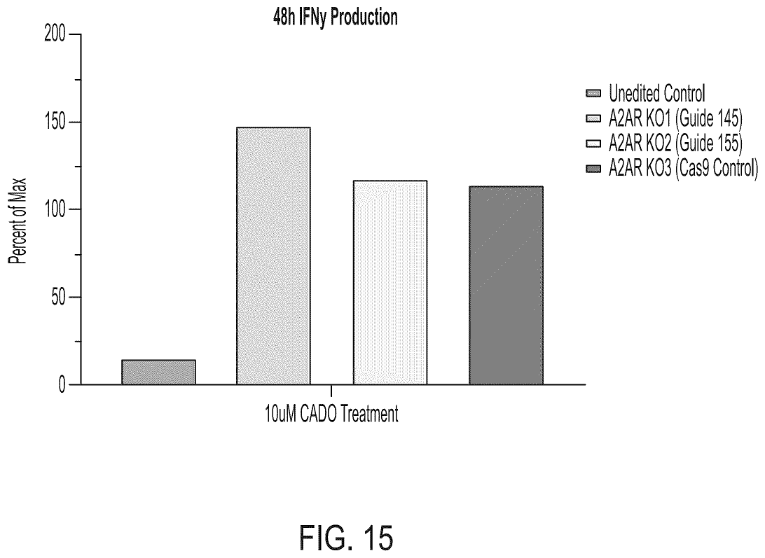

As shown in FIG. 13, it was determined through next generation sequencing (NGS) that guides 145 and 155 effectively edited the A2AR gene when using either a CBE (rBE4) or ABE (ABE8.20). Knocking out of the A2AR gene abrogated adenosine signaling and resulted in lack of upregulation in edited CAR-T cells of pCREB in the presence of 2-chloroadenosine (see FIG. 14). Further, it was determined that knocking out of the A2AR gene was associated with protection of the CAR-T cells from adenosine-mediated cytokine production (see FIG. 15).

### Example 4: Identifying gRNAs for Knocking Out A2BR

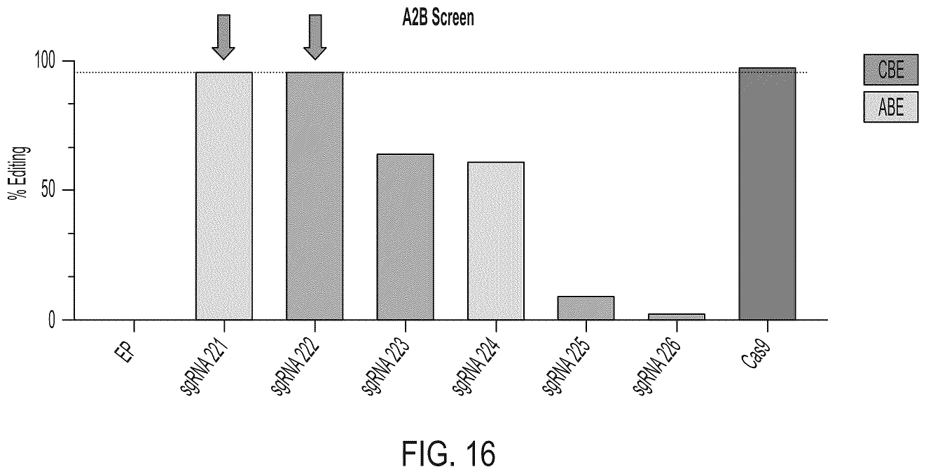

As noted above in Example 3, knocking out the A2BR gene reduced suppression of CAR T-cell cytokine production associated with hypoxic microenvironments and/or in microenvironments containing adenosine (FIG. 16). Thus, guide RNAs were screened for use in knocking out the A2BR gene using the cytosine base editor (CBE) rBE4 or the adenosine base editor (ABE) ABE8.20. Using next generation sequencing, it was determined that gRNA 221 in conjunction with an ABE (ABE8.20), and gRNA 222 in conjunction with a CBE (rBE4) achieved editing efficiencies of greater than about 95%.

### Example 5: Multiplex Base Editing Protected Allogeneic EGFR-Targeting CAR-T Cells from Inhibition by Extracellular Adenosine in the Tumor Microenvironment

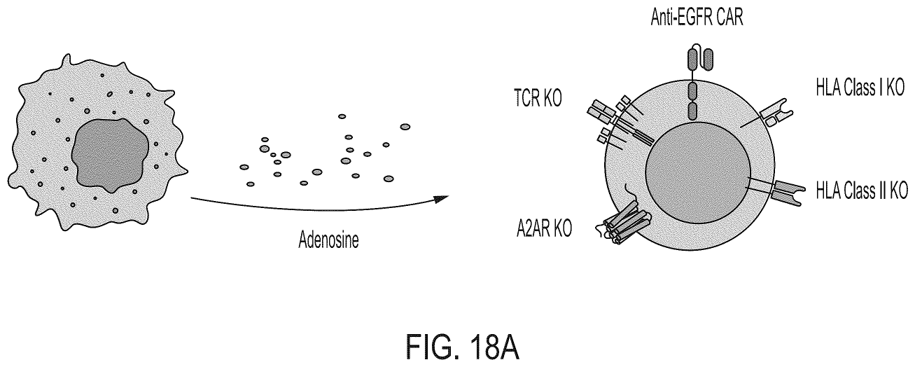

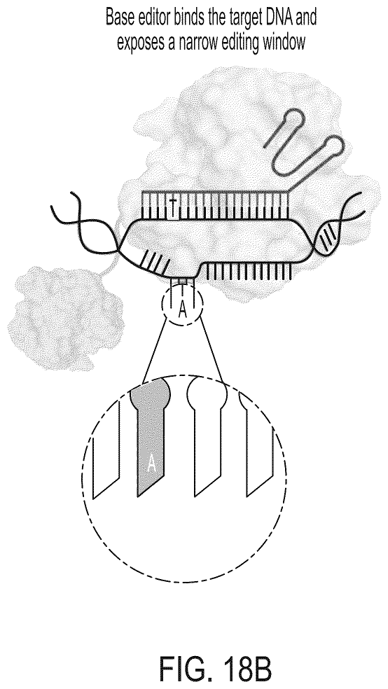

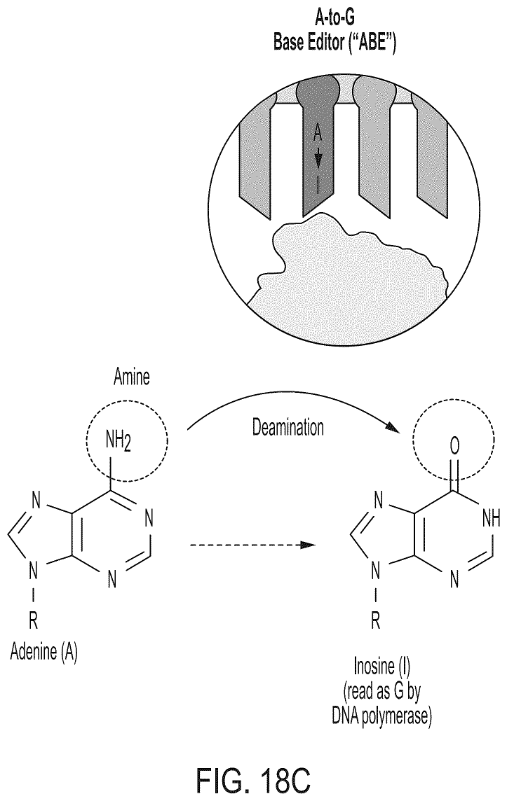

Due to the powerful immunosuppression generated the tumor microenvironment (TME), clinical CAR-T efficacy against solid tumors to-date has been minimal. CAR-T cells encounter both biochemical and immunological barriers in the TME, including the hypoxia-driven accumulation of extracellular adenosine (Ado). Signaling by Ado through A2A adenosine receptors (A2AR) on T cells significantly inhibits many effector functions including cytokine section and anti-tumor cytotoxicity (FIG. 18A). Generating CAR-T cells that are genetically-resistant to adenosinergic signaling can prevent their suppression within the TME. As one approach to increasing the quality of the cell product and expanding patient access to these treatments, these therapies can be derived from healthy donor-derived allogeneic cells. Base editing can target gene splice sites to simultaneously knock out multiple genes without introducing double strand DNA breaks (FIGS. 18B and 18C). Leveraging the base editing platform provided herein, experiments were undertaken to produce and evaluate T cells base-edited to knock-out expression of endogenous TCR, HLA Class I, HLA Class II, and A2AR to produce allogeneic Adenosine-Resistant CAR-T cells (ARC T cells).

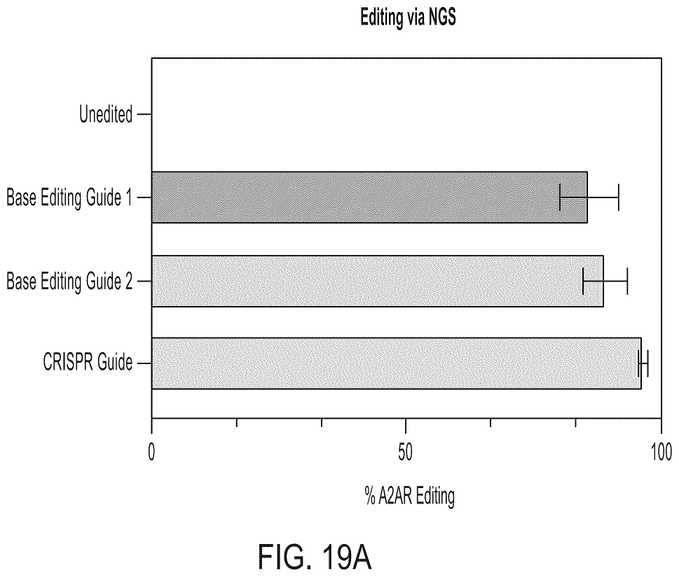

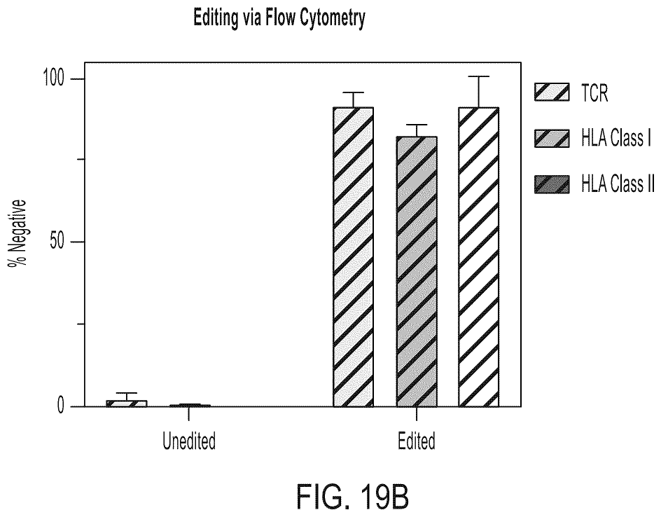

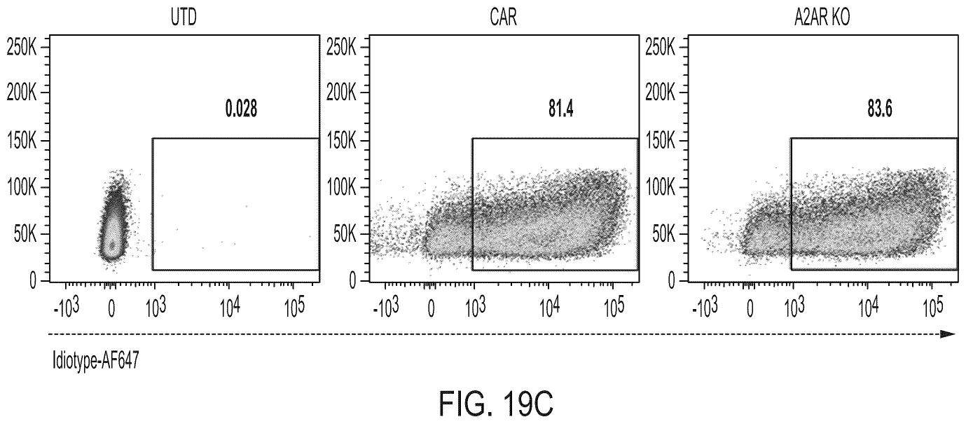

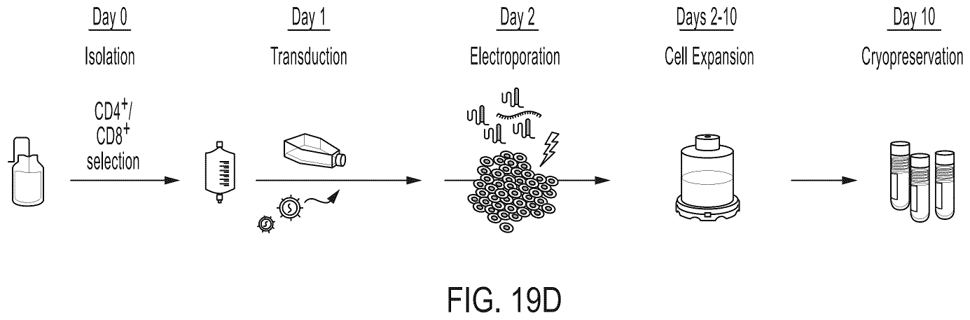

First, experiments were undertaken to produce ARC T cells. The cells were produced according to the scheme shown in FIG. 19D first transducing the cells with polynucleotides encoding an anti-EGFR chimeric antigen receptor (CAR) and then using electroporation to introduce to the cells base editor systems for knocking out expression of TCR, HLA Class I, HLA Class II, and A2AR using one of the two A2AR-targeting base-editing systems listed in Table 20 and each of the base editing systems targeting CD3ε, B2M, and CIITA listed in Table 20 (see Tables 1A and 1B for spacer sequences). The cells were base edited to knock-out expression of A2AR using either sgRNA 155 in combination with ABE8.20 or sgRNA 145 in combination with a CBE. Base editing using the guide sgRNA 155 in combination with ABE8.20 or the guide sgRNA 145 in combination with a CBE was effective in editing the A2AR gene to knock-out expression of A2AR in anti-EGFR T cells (FIG. 19A). The guides TSBTx4073, TSBTx760, and TSBTx763 used in combination with ABE8.20 were effective in base editing genes encoding CD3F, B2M, and CIITA to knock-out the expression thereof in the anti-EGFR T cells (FIG. 19B). The base edits to knock out A2AR expression did not adversely impact chimeric antigen receptor expression in the edited cells (FIG. 19C).

TABLE 20
Base editing systems used to prepare ARC T cells.

| Target | target site name | Guide ID | Editor |
|---|---|---|---|
| A2AR | RCM sgRNA 155 | TSBTx2043 | ABE8.20 |
| A2AR | RCM sgRNA 145 | TSBTx2042 | CBE (rBE4) |
| CD3ε | AE_gRNA_566 | TSBTx4073 | ABE8.20 |
| B2M | B2M | TSBTx760 | ABE8.20 |
| CIITA | CIITA exon 16 SD | TSBTx763 | ABE8.20 |

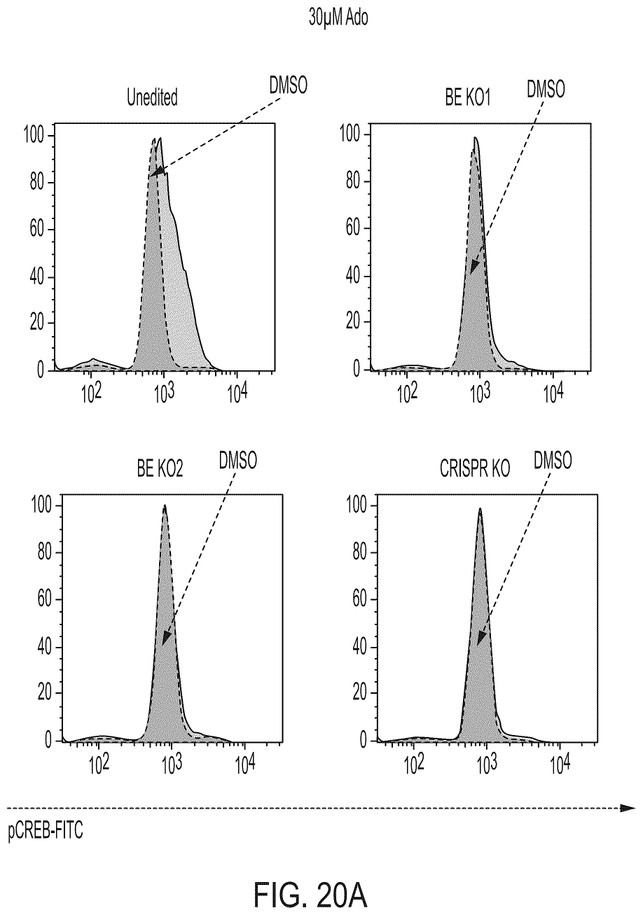

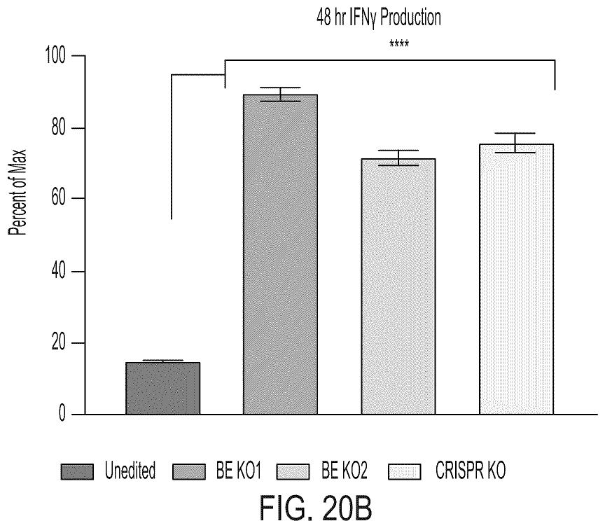

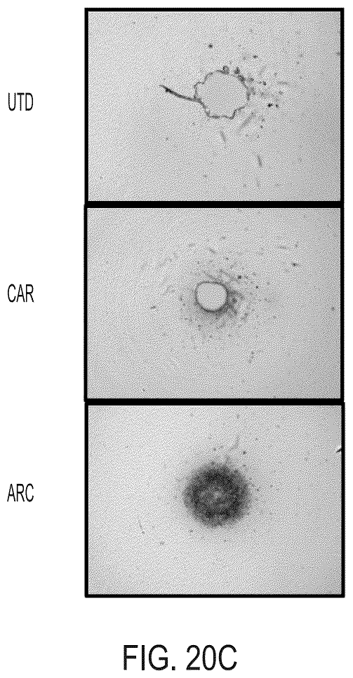

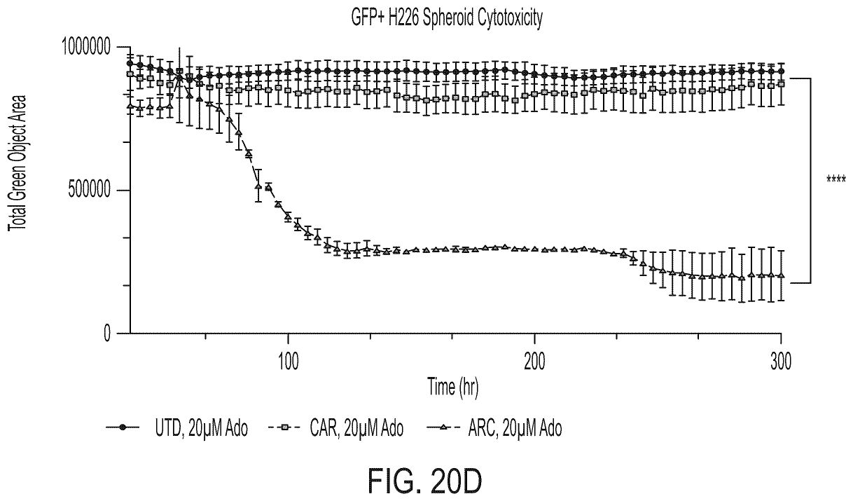

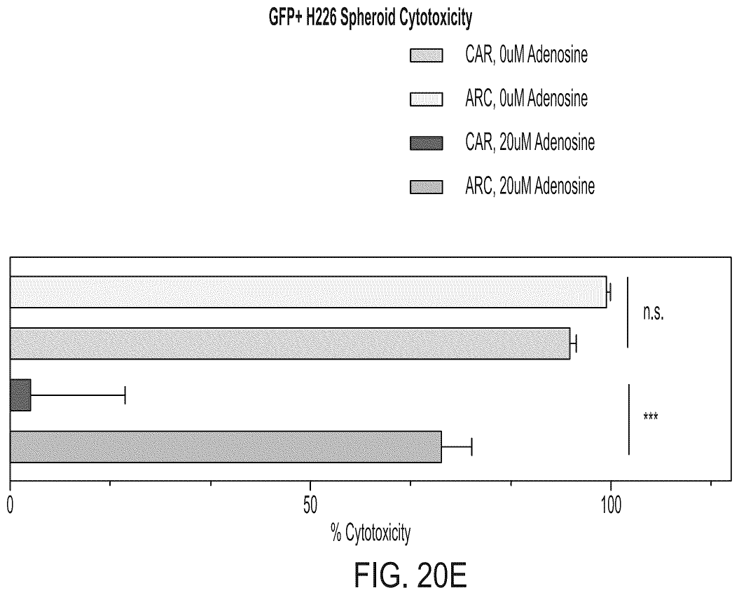

Experiments were next undertaken to determine whether the adenosine-resistant CAR-T cells were protected from adenosine-mediated suppression in vitro. First, flow cytometry was used to confirm that downstream signaling of A2AR was prevented in ARC T cells, as indicated by a reduction in phosphorylated CREB staining (FIG. 20A). Flow cytometry measurements also confirmed that the ARC T cells maintained the capacity to produce IFNγ when exposed to extracellular adenosine for 48 hours, while unedited cells did not. It was determined in vitro that the anti-EGFR ARC T cells were effective at killing H266 spheroid cells when contacted with H266 spheroids for a period of 9 days (FIGS. 20C and 20D). The anti-EGFR ARC T cells maintained cytotoxicity against the H266 spheroids even in the presence of 20 μM adenosine (FIGS. 20D and 20E). Unedited CAR T cells and untransduced T cells (i.e., unedited T cells not expressing any chimeric antigen receptor (CAR)) did not have cytotoxicity against the H266 spheroid cells in the presence of adenosine.

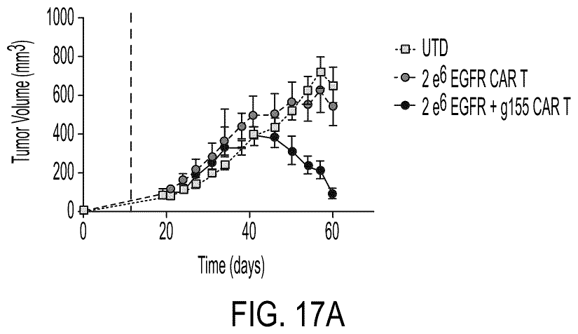

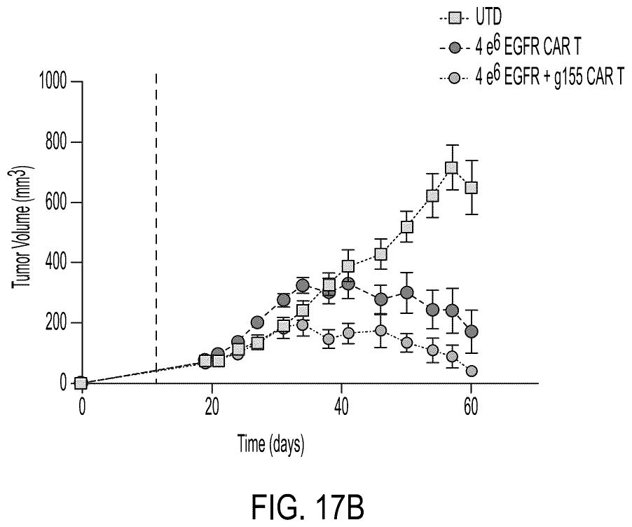

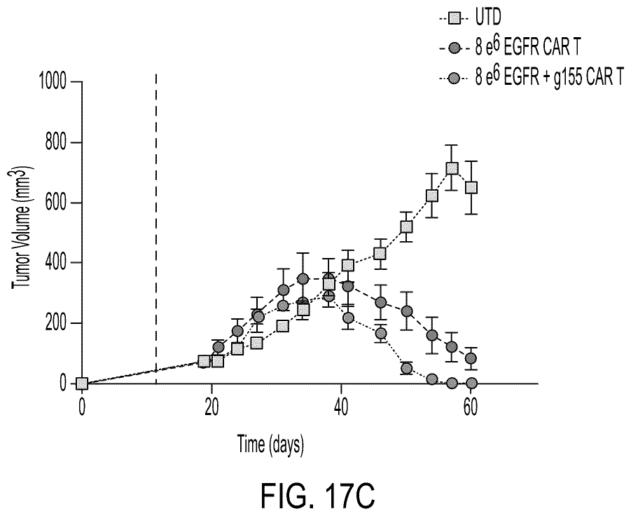

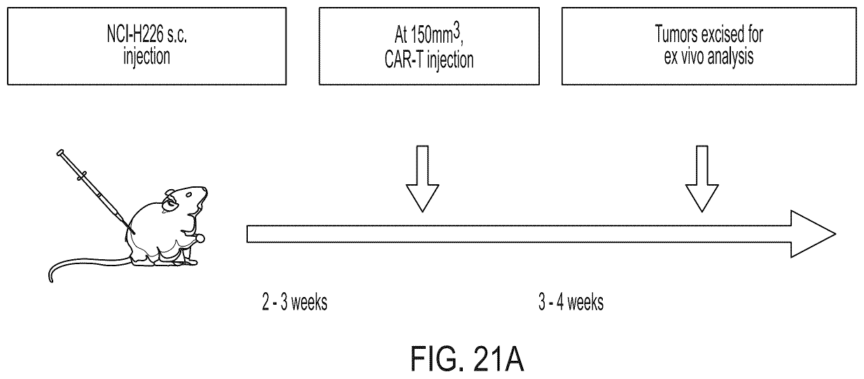

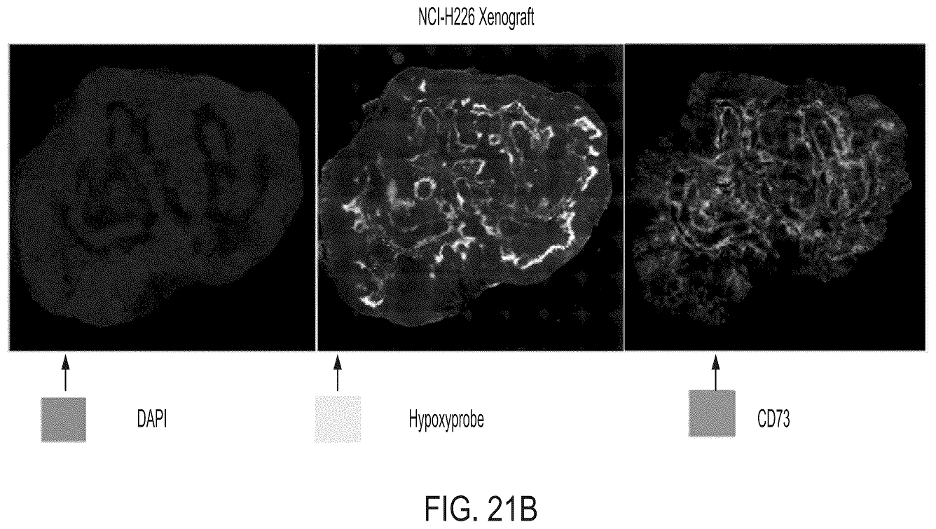

The in vivo efficacy of the ARC T cells was analyzed in a xenograft model of non-small cell lung cancer characterized as a squamous cell carcinoma. This was done by injecting NCI-H226 sells subcutaneously in an immunodeficient mouse, the NCG mouse. The NCG mouse lacks functional/mature T, B, and NK cells, and has reduced macrophage and dendritic cell function (FIG. 21A). NCG mice having an H226 xenograft were injected 2-3 weeks following administration of the H226 cells with 150 μL ARC T cells, unedited CAR t cells, or untransduced T cells (i.e., unedited T cells not expressing any chimeric antigen receptor) at three doses: 2×10⁶, 4×10⁶, or 8×10⁶ cells, or with control cells: anti-EGFR CAR T cells expressing the adenosine receptor and untransduced (UTD) cells. Alterations in tumor volume were monitored over 3-4 weeks. Measurements demonstrated that the tumor microenvironment (TMV) in the xenograft model included hypoxic conditions and cells expressing the adenosine-producing ectoenzyme CD73 (FIG. 21). Calipers were used to measure the tumors over time, and the measurements were used to calculate tumor volume. Once tumor volume reached 140 mm³, measurements of tumor size were taken every 2-3 days. Dramatic decreases in tumor volume were observed for tumors treated with the edited A2AR knockout anti-EGFR CAR-T cells (FIGS. 17A-17C). In fact, at every dose tested better tumor control was observed in mice treated with the edited A2AR knockout anti-EGFR CAR-T cells relative to mice treated with the unedited A2AR-expressing anti-EGFR CAR-T cells. Interestingly, while virtually no reduction in tumor volume was observed in mice that received the lowest dose tested, i.e., 2×10⁶ unedited A2AR-expressing anti-EGFR CAR-T cells, mice that received the same number of edited A2AR knockout anti-EGFR CAR-T cells displayed significantly lower tumor volume.

As demonstrated above, base editing enabled generation of multiplex-edited allogeneic CAR-T cells (i.e., ARC T cells) that exhibited enhanced potency against solid tumors in vivo and in vitro. In particular, elimination of adenosine-mediated immunosuppression in ARC T cells enhanced anti-tumor function. ARC T cells demonstrated reduction of A2AR signaling via lack of downstream phosphorylated CREB after stimulation with exogenous adenosine. ARC T cells produced greater levels of cytokine and exhibited superior anti-tumor cytotoxicity in the presence of exogenous adenosine in vitro. Also, ARC T cells showed increased in vivo anti-tumor potency in a mouse xenograft solid tumor model as compared to unedited CAR-T cells, most notably at a sub-optimal cell dosage.

### Example 6: Preparation of Multiplex-Edited Cells with TGFbR Expression Knocked-Out

Experiments were undertaken to prepare chimeric antigen receptor-expressing T cells (CAR T cells) base edited to knock out expression of TGFbR with or without expression of one or more of expression of A2AR, PD1, and/or TGFbR knocked out.

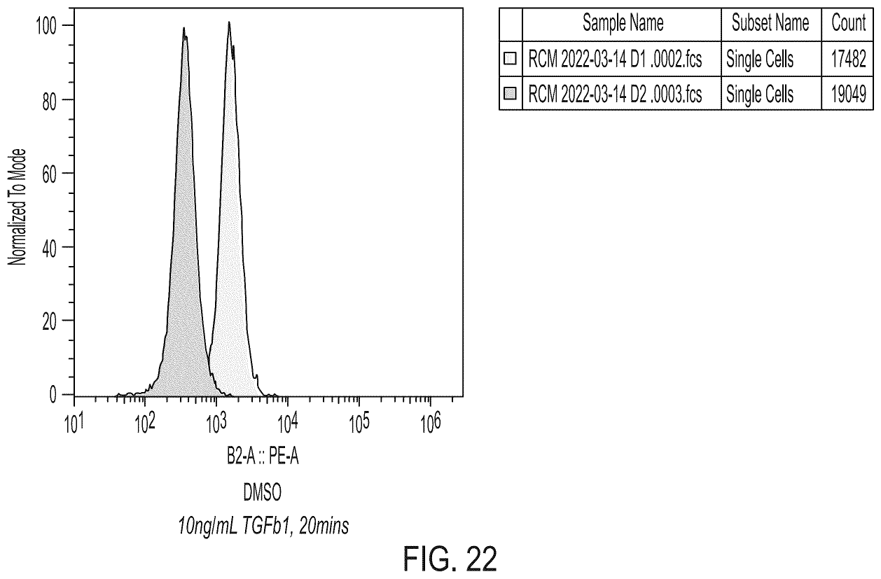

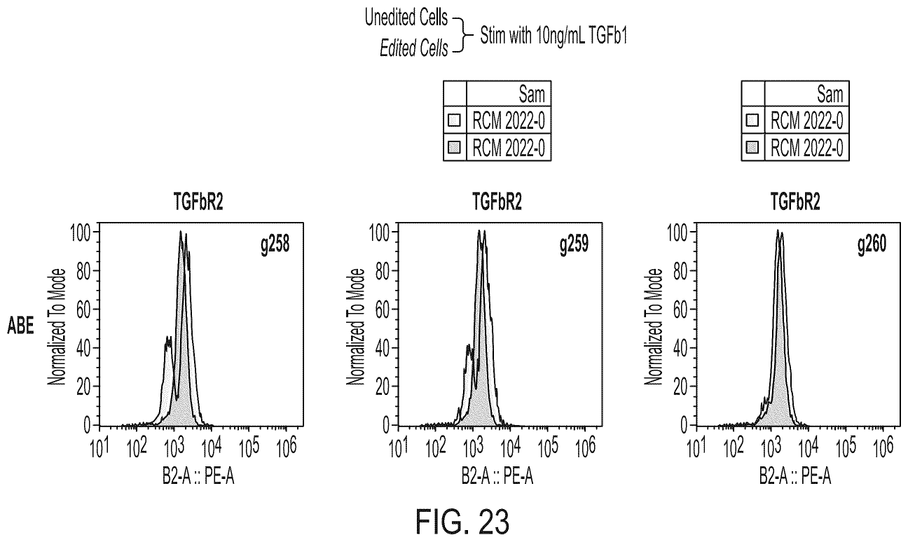

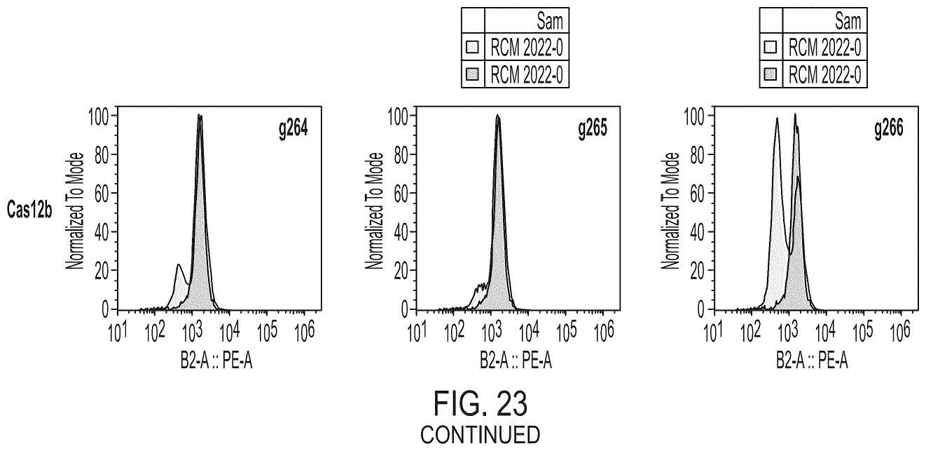

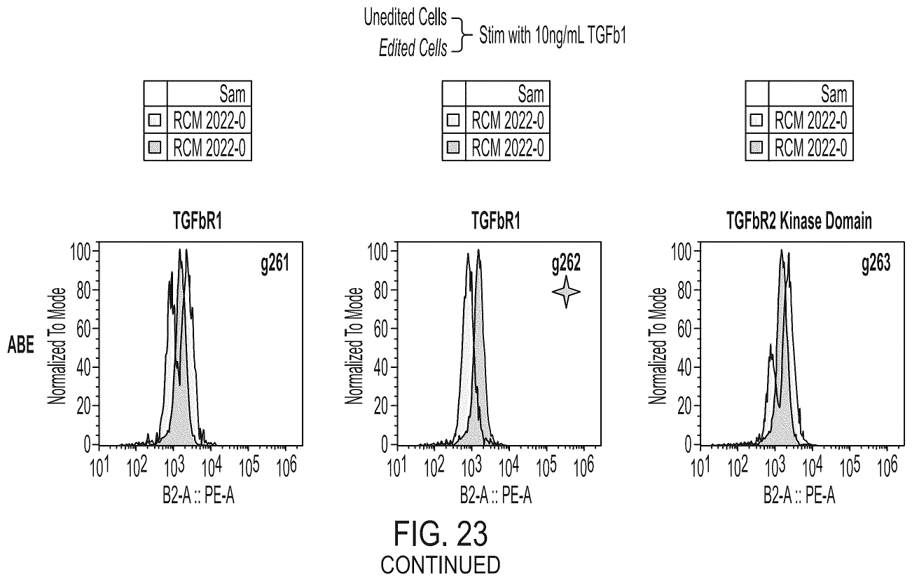

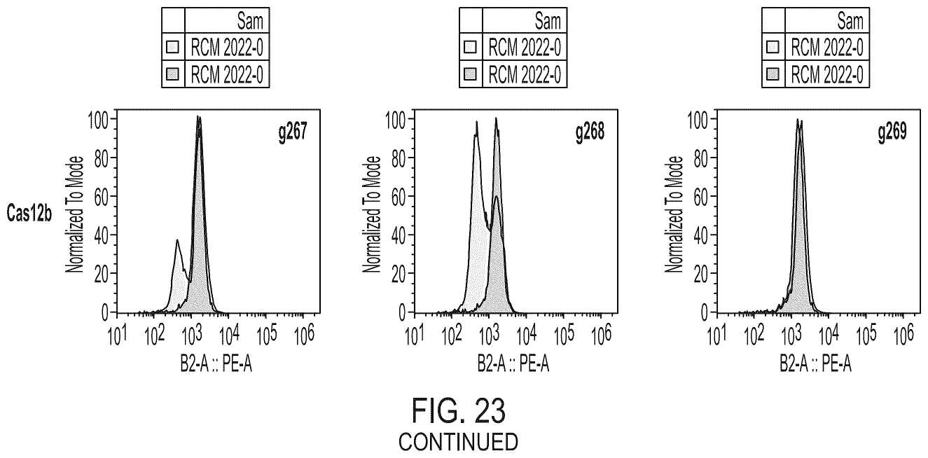

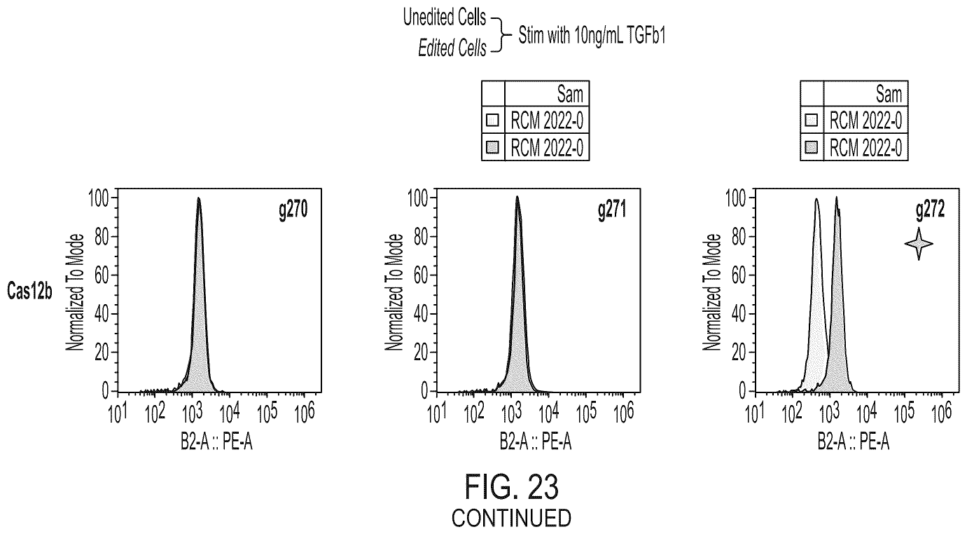

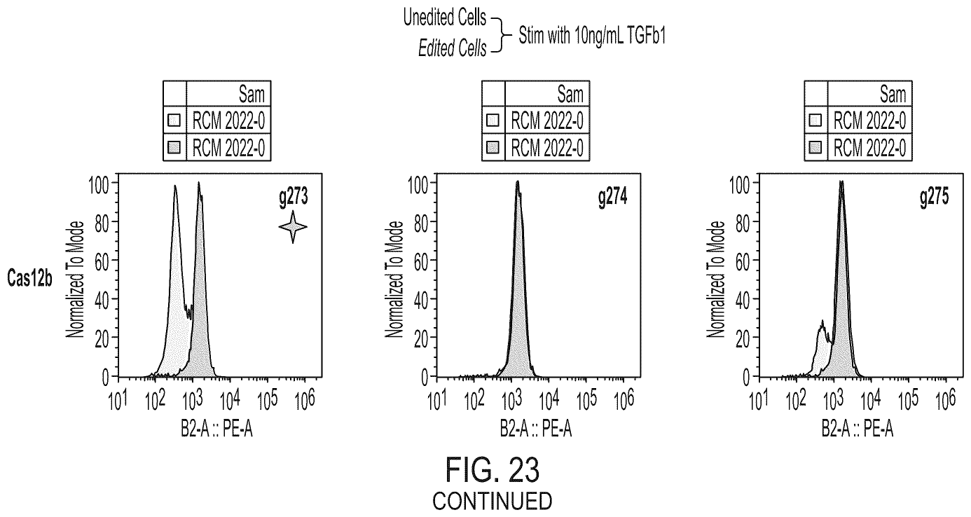

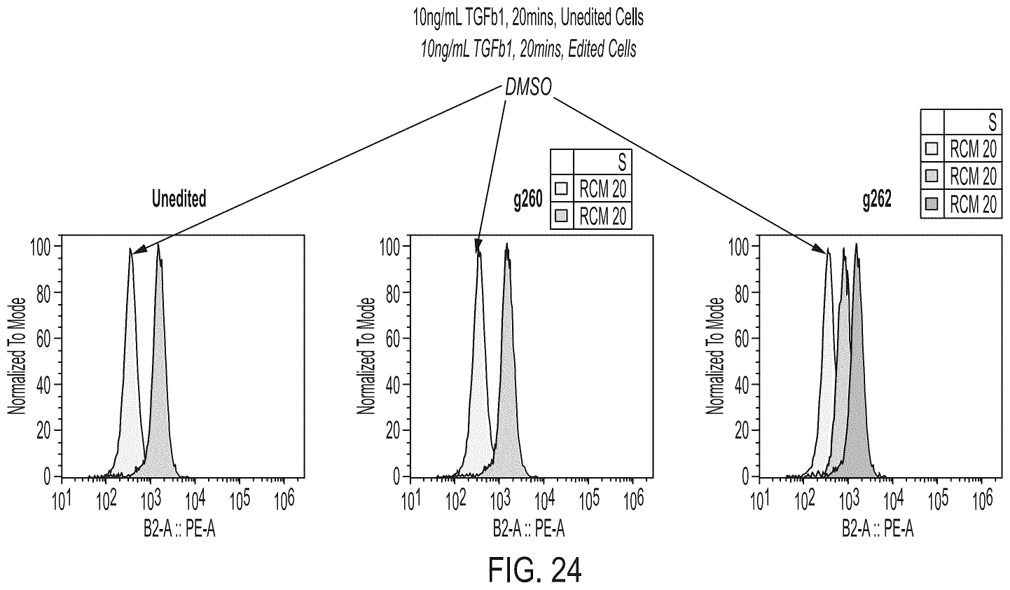

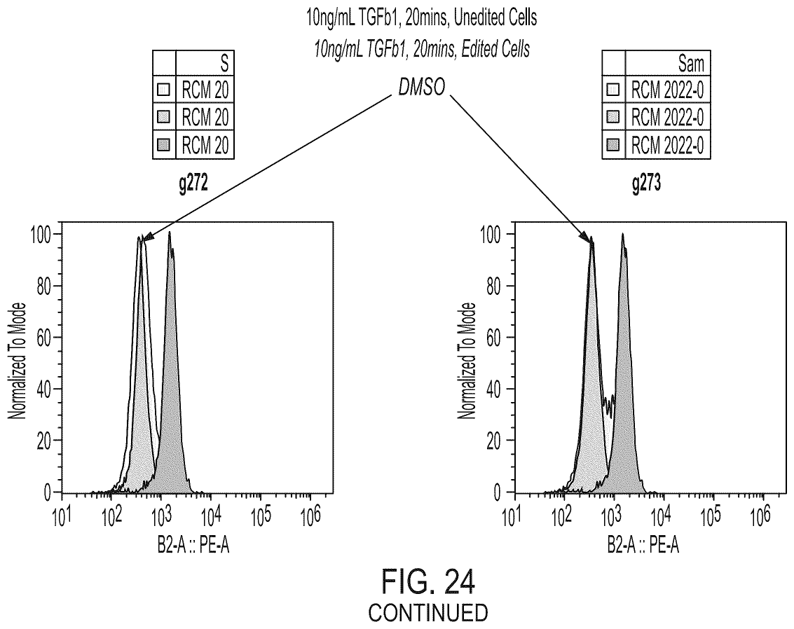

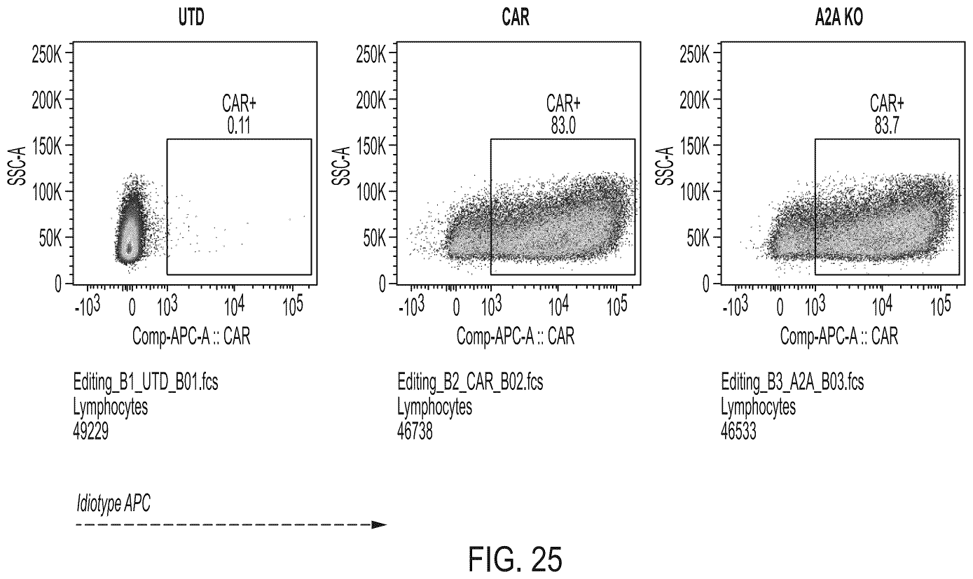

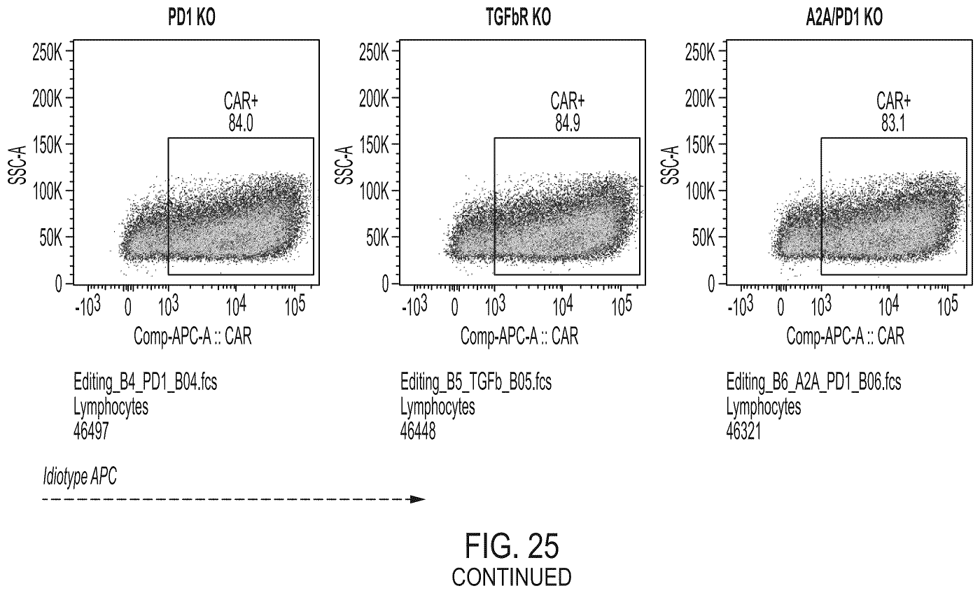

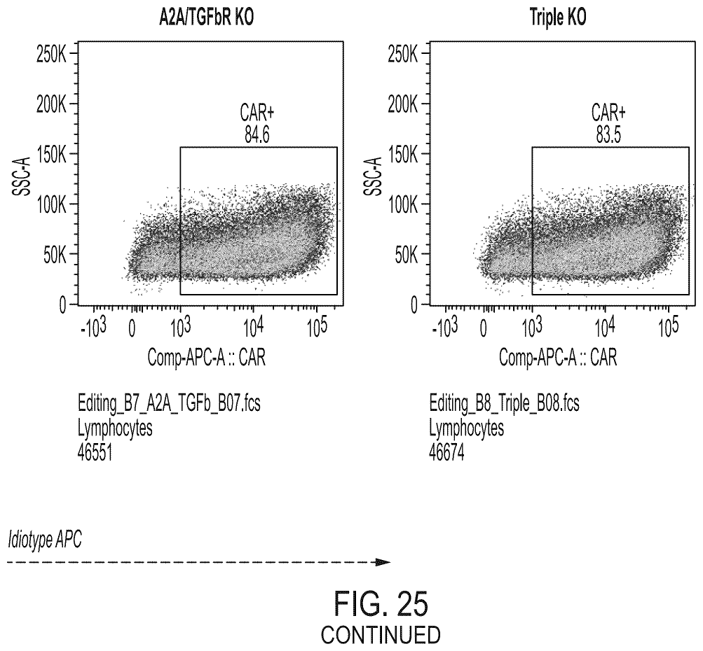

First, an experiment was undertaken to identify guides capable of facilitating knock-out of TGFbR2 or TGFbR1 in EGFR-targeting CAR T cells when used in combination with ABE8.20 or Cas12b (FIGS. 23 and 24 and Table 1B). The following guides were evaluated g258, g259, g260, g261, g262, g263, g264, g265, g266, g267, g268, g269, g270, g271, g272, g273, g274, g275 (see Table 1B). The base editor used in combination with the guides was ABE8.20. Knock-out efficiency was measured by measuring the levels of phospho-SMAD in the edited cells using a phosphor-SMAD assay (FIG. 22), which involved stimulating the CAR T cells with 10 ng/mL rhTGFb1 or DMSO for 20 minutes at 37° C., followed by permeabilization and staining of the cells. The cells were stained using a phospho-SMAD2/3 antibody and measurements of staining were made using flow cytometry (FIGS. 23 and 24). The guides g262, g272, and g273 were effective at facilitating editing to knock-out expression of TGFbR (FIGS. 23 and 24). Knocking out of TGFbR expression in the CAR T cells did not adversely impact expression levels of the chimeric antigen receptor in the CAR T cells (FIG. 25).

Next, EGFR-targeting CAR T cells were edited to knock-out expression one or more of A2AR, PD1, and TGFbR and CAR expression was then measured in the cells using flow cytometry (FIG. 25). A2AR expression was knocked out using the guide TSBTx2043, PD1 expression was knocked out using the guide TSBTx025, and TGFbR expression was knocked out using the guide TSBTx5277. Knocking out one or more of A2AR, PD1, and/or TGFbR in the CAR T cells did not adversely impact chimeric antigen receptor expression in the cells.

### Example 7: Base Editing of Chimeric Antigen Receptor (CAR) T Cells to Knock-Out Expression of HIF-1a Isoform 3 Improved Cytokine Secretion

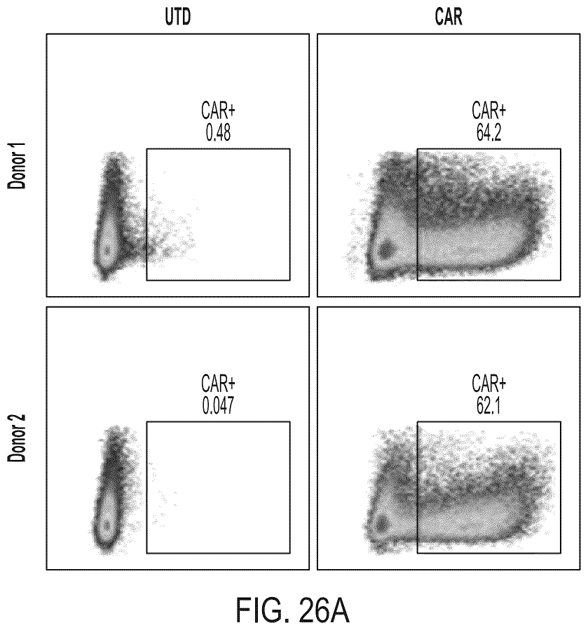

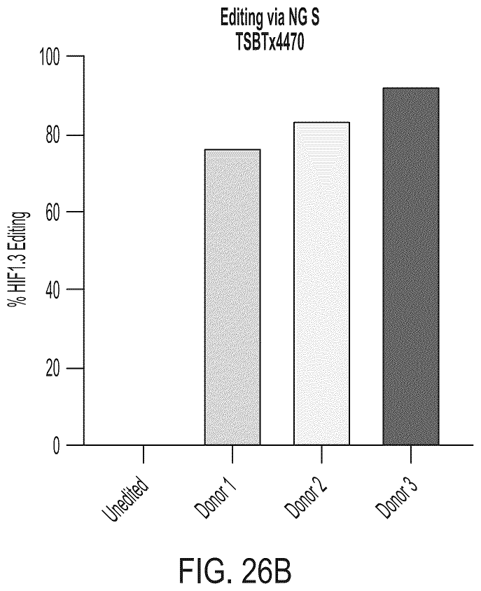

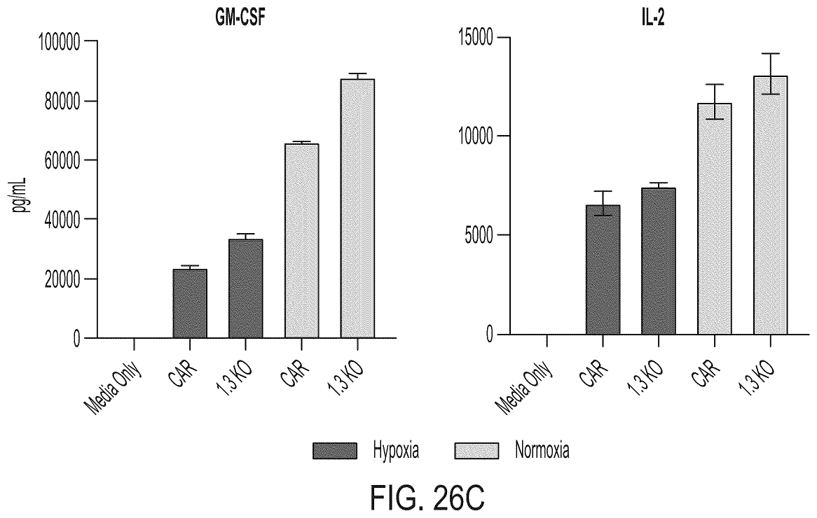

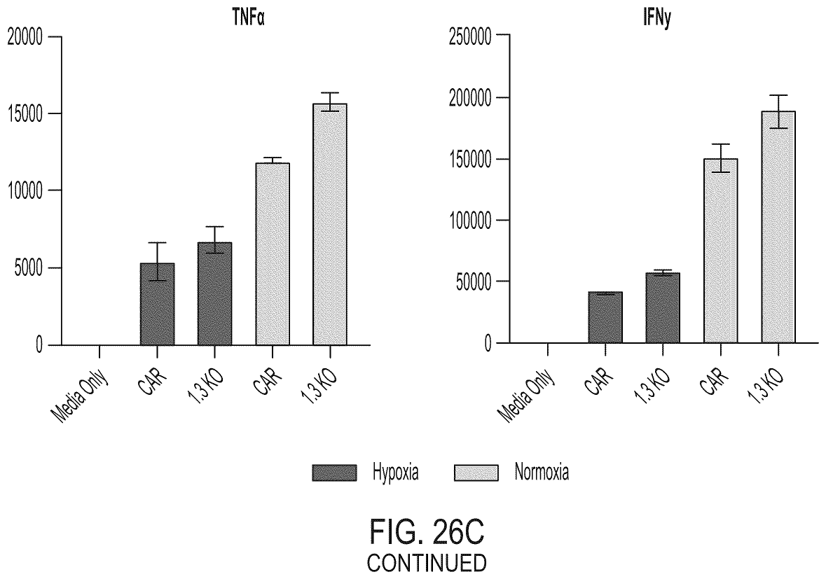

Experiments were undertaken to determine the impact of knocking out HIF-1a isoform 3 on cytokine production by anti-EGFR CAR T cells. First, anti-EGFR CAR T cells were produced by transducing T cells from donors with polynucleotides encoding an anti-EGFR CAR. The transduced CAR T cells surface-expressed the anti-EGFR CAR (FIG. 26A). Then, the guide polynucleotide TSBTx4470 (sgRNA 230) was designed to target an adenosine base editor (ABE) to deaminate a nucleobase in a start codon of a HIF-1a isoform 3 polynucleotide to knock out expression of HIF-1a isoform 3 in a cell. High base editing efficiencies were achieved in anti-EGFR CAR T cells contacted with a base editor system containing the guide polynucleotide TSBTx4470 (sgRNA 230) and the ABE (FIG. 26B). The base edited anti-EGFR CAR T cells showed superior cytokine secretion relative to anti-EGFR CAR T cells that were not base edited to knock out expression of the HIF-1a isoform 3 polypeptide when co-cultured in the presence of H226 tumor cells (1:2 effector to target cell ratio) under both normoxia (20% oxygen) and hypoxia (1% oxygen) conditions (FIG. 26C). Therefore, use of base editing to knock out expression of HIF-1a isoform 3 was an effective strategy for improving cytokine secretion of the CAR T cells under both hypoxic and normoxic conditions.

### Example 8: Improved Tumor Clearance by Base Edited Anti-EGFR Chimeric Antigen Receptor (CAR) T Cells In Vivo

Experiments were Undertaken to Evaluate In Vivo Tumor Clearance in Mice by Anti-EGFR CAR T cells base edited to knock out A2AR, B2M, CD3ε, CIITA, PD1, and/or TGFbR2. The mice were administered H226 mesothelioma cells subcutaneously and subsequently administered a dose of 2e6 or 4e6 of base edited anti-EGFR CAR T cells.

Three different base edited anti-EGFR CAR T cells were evaluated: CAR (base edited to knock-out expression of CD3ε, B2M, and CIITA), A2AR (base edited to knock-out expression of CD3ε, B2M, CIITA, and A2AR), and TKO (base edited to knock-out expression of CD3ε, B2M, CIITA, A2AR, and PD1, and edited using a nuclease to knock-out expression of TGFbR2). TGFbR2 was knocked-out using the guide polynucleotide TSBTx5277 in combination with Cas12b. Knock-out of all other targets (A2AR, CD3ε, B2M, CIITA, and PD1) was carried out using base editing. A2AR was base edited using ABE8.20 in combination with the guide polynucleotide TSBTx2043. CD3F was base edited using ABE8.20 in combination with the guide polynucleotide TSBTx4073. B2M was base edited using ABE8.20 in combination with the guide polynucleotide TSBTx760. CIITA was base edited using ABE8.20 in combination with the guide polynucleotide TSBTx763. PD1 was base edited using ABE8.20 in combination with the guide polynucleotide TSBTx025.

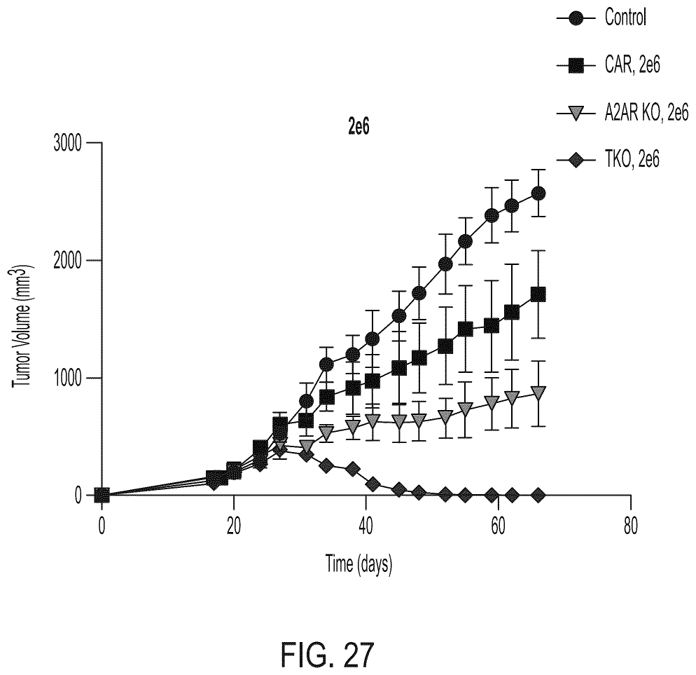

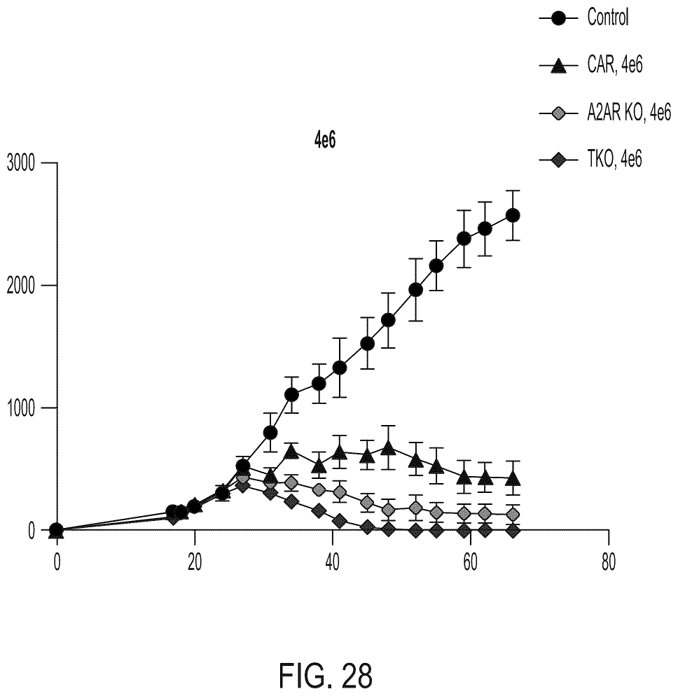

All of the base-edited CAR T cells were capable of reducing tumor volume in the mice at both doses evaluated (FIGS. 27 and 28). The TKO CAR T cells were capable of eliminating all or nearly all tumor cells from the mice within 50 days at both doses evaluated. Tumor clearance increased with dose.

The following materials and methods were employed in the above examples.

### Hypoxia Suppression Assay Protocol

The purpose of this protocol was, among other things, was to analyze cytokine release from CAR-T cells in response to tumor targets in the presence of 1% oxygen.

The reagents used in this protocol included CAR-T cells, SKOV3 cells, NCI-H226 Tumor cells, 96-well flat bottom plates (untreated), T cell growth media (TCGM) without IL2, dimethyl sulfoxide (DMSO), and phosphate-buffered saline (PBS).

The protocol involved the following steps:
1. Harvesting and counting T cells and tumor targets.
2. Washing T cells and tumor cells with 1×PBS.
3. Spinning down and resuspending both at 1e6/mL in TCGM without IL2.
4. Plating 100 μL T cells and 100 μL tumor targets per well into 96-well flat bottom plate.
   a. For T cell alone conditions, adding 100 μL TCGM without IL2.
   b. The result was a 1:1 E:T ratio: 100k T cells, and 100k tumor cells per well.
5. Placing at 37° C. in an incubator with oxygen levels set to 1% for 48 hours.
6. After 48h spinning plate down at 500g for 5 mins.
7. Removing ˜150 μL supernatant and transferring to new 96-well plate.
8. Running on Ella™ (a device for next generation enzyme-linked immunosorbent assays).
   a. Typical Dilutions for CAR-T cytokine production were 10-50x depending on CAR and tumor target.

### Adenosine Suppression Assay Protocol

The purpose of this protocol was, among other things, to analyze cytokine release from CAR-T cells in response to tumor targets in the presence of extracellular adenosine.

The reagents used in this protocol included CAR-T cells, SKOV3 cells, NCI-H226 Tumor cells, 96-well flat bottom plates (untreated), T cell growth media (TCGM) without IL2, dimethyl sulfoxide (DMSO), 2-chloro-adenosine (CADO), and phosphate-buffere saline (PBS).

The protocol involved the following steps:
1. Harvesting and counting T cells and tumor targets.
2. Washing T cells and tumor cells with PBS 1×.
3. Spinning down and resuspending both at 2e6/mL in TCGM without IL2.
4. Making up 2X stimulation solutions:
   a. Making a 20 μM CADO solution in TCGM without IL2.
   b. Using the same volume of DMSO to make up a control solution for unstimulated conditions.
5. Plating 50 μL T cells and 50 μL tumor targets per well into 96-well flat bottom plate.
   a. For the T cell alone conditions, adding 100 μL 2×DMSO solution.
   b. For adenosine stimulated conditions, adding 100 μL 2×CADO solution.
      Final concentration was 10 μM CADO in the assay.
   c. Result was 1:1 E:T ratio corresponding to 50k T cells and 50k tumor cells per well.
6. Placing at 37° C. for 48 hours.
7. After 48h spinning the plate down at 500g for 5 mins.
8. Removing about 150 μL supernatant and transferring to a new 96-well plate.
9. Running on Ella™ (a device for next generation enzyme-linked immunosorbent assays).
   a. Typical Dilutions for CAR-T cytokine production were 10-50x, depending on the CAR and the tumor target.
      pCREB Protocol

The purpose of this protocol, among other things, was to determine intracellular levels of phosphorylated cAMP Response Element Binding (CREB) protein in human T cells. The reagents used in this protocol included Primary human T cells, phosphate-buffered saline (PBS), fluorescence-activated cell sorting (FACS) buffer, XVIVO-15 medium, dimethyl sulfoxide (DMSO), 2-chloroadenosine (CADO), pCREB-FITC antibody (Cell Signaling), BD Cytofix™ Buffer, BD Perm III Buffer.

The protocol involved the following steps:

### Day 0 (Serum Starvation)

1. Harvesting the appropriate number of T cells for the assay from culture and transfer them into a 50 mL conical tube.
2. Rinsing cells with PBS.
3. Centrifuging cells at 500×g for 5 mins.
4. Resuspending the T cells in XVIVO-15 medium at a concentration of 1e6 cells/mL and placing in a 37° C. incubator with 1% 02 levels.
5. Resting the cells in 1% 02 for 48 hours.

### Day 2 (Stimulation)

1. Pre-warming BD Cytofix in a 37° C. water bath.
2. Pre-incubating the BD Perm Buffer III at −20° C.
3. Rinsing serum-starved T cells with PBS.
4. Resuspending the T cells at 2e6/mL in XVIVO-15
5. Plating 50 μL of the T cell suspension into a 96-well U-bottom plate.
6. Preparing a 2× concentration of XVIVO 15+CADO at 60 μM.
   a. The final assay stimulation concentration was 30 uM.
7. Preparing a 2× concentration of XVIVO-15+DMSO, where the volume of DMSO used was equal in volume to the volume of CADO used to prepare the 2× concentrated XVIVO 15+CADO mixture.
8. Stimulating the cells by adding 50 μL of either 2×DMSO or 2×CADO solution to each well.
9. Immediately after addition of cell stimulation solutions, transferring the plate into a 37° C. incubator and incubating for 1 hour.
10. After 1 hour, using a multichannel pipet, adding 100 μL pre-warmed Cytofix Buffer to each well, and pipetting up and down to mix.
11. Transferring the plate back into a 37° C. incubator and incubating for 15 mins.
12. Centrifuging the plate at 500×g for 5 minutes.
13. Decanting the supernatant.
14. Using a multichannel pipet to slowly add 100 μL of pre-chilled Perm Buffer III.
15. Incubating at 4° C. for 30 minutes.
16. Centrifuging the plate at 500×g for 5 minutes.
17. Decanting the supernatant.
18. Resuspending the cells in 200 μL fluorescence-activated cell sorting (FACS) buffer to wash.
19. Spinning the cells down at 500g for 5 mins.
20. Preparing the pFlow antibody master mix for staining panel.
   a. 5 μL/test for pCREB-FITC.
21. Resuspending cell pellets in 100 μL antibody cocktail.
22. Incubating for 30 mins at room temperature (RT) in the dark.
23. Washing the cells with 100 μL FACS buffer.
24. Spinning down the cells at 500g for 5 mins.
25. Decanting the supernatant and resuspending in 200 μL FACS Buffer.
26. Running the samples on a MACSQuantl6 analyzer.

### Electroporation (EP) Protocol

The purpose of this protocol, among other things, was to electroporate (EP) T cells using a Lonza Amaxa instrument at 100Lp scale.

The reagents used in this protocol included primary human cells (PBMC, TCT, T cells), phosphate-buffered saline (PBS), P3 electroporation (EP) Buffer, XVIVO-15 medium (no serum, no buffer, no IL2), T cell growth medium (TCGM)+IL2, rBE4 mRNA (2 mg/mL), Guide RNA (Biospring 2 mg/mL).

The protocol involved the following steps:
1. Thawing an aliquot of mRNA/gRNA on ice.
   a. Also thawing an aliquot of P3 EP buffer on ice.
2. Harvesting cells and spinning down at 500g for 5 mins.
3. Resuspending the cell pellet in XVIVO-15 and counting the cells using a NucleoCounter® NC-200.
4. Aliquoting the number of cells needed for EP into a 50 mL conical tube(s).
5. Spinning down at 500g for 5 mins and aspirating.
6. Using P3 buffer to resuspend cells at 100e6/mL.
7. Preparing 2× mRNA/gRNA master mix (MM) containing 5 μL mRNA, 2.5p L of each gRNA needed, and 35 μL P3 buffer per 5e6 cells.
8. Combining a 100e6/mL cell suspension 1:1 v/v with 2× mRNA/gRNA MM for n+1 electroporations (Eps).
   i. 50 μL cells+50 μL 2× MM=100 μL per electroporation (EP).
9. Aliquoting 100 μL per EP cuvette.
   a. 5e6 cells in 100 μL per zap.
10. Tapping EP cuvette on bench to ensure entire cell suspension is in the bottom of cuvette, touching the metal plates.
11. Placing the cuvette in the Lonza Amaxa electroporator and electroporating (EP).
12. After EP, adding 100 μL prewarmed TCGM+IL2 to each cuvette to quench the reaction.
   a. Pipetting up and down to ensure entire 200 μL contents is removed from cuvette.
13. Transferring the entire 200 μL to 5 mL prewarmed TXGM+IL2 in 6-well GRex®.
14. Placing at 37° C. to allow for cell expansion.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for reducing the expression of an Adenosine A2A Receptor Adenosine (A2AR) or A_{2B} Receptor (A2BR) or HIF1α.I3 polypeptide and/or polynucleotide in a cell, the method comprising contacting a cell comprising an A2AR, A2BR, HIF1α, or HIF1α.I3 gene with (i) a base editor or a polynucleotide encoding the base editor and (ii) one or more guide polynucleotides or a polynucleotide encoding the guide polynucleotides, wherein the base editor comprises a programmable DNA binding domain and a deaminase domain, and wherein each of the guide polynucleotides directs the base editor to effect a nucleobase alteration in a A2AR, A2BR, HIF1α, or HIF1α.I3 gene that alters a splice acceptor or splice donor site, introduces a stop codon, or otherwise disrupts expression of the gene, thereby reducing expression of an A2AR, A2BR, HIF1α.I3 polypeptide and/or polynucleotide in the cell.

2. A method for producing a modified immune cell comprising an alteration in a hypoxic and/or adenosinergic pathway, the method comprising contacting the cell with (i) a base editor or a polynucleotide encoding the base editor and (ii) one or more guide polynucleotides or a polynucleotide encoding the guide polynucleotides, wherein the base editor comprises a programmable DNA binding domain and a deaminase domain, and wherein each of the guide polynucleotides directs the base editor to effect a nucleobase alteration in a gene encoding a polypeptide component of the hypoxic and/or adenosinergic pathway or a regulatory element thereof, thereby producing a modified immune cell.

3. The method of claim 2, wherein the polypeptide component of the hypoxic and/or adenosinergic pathway is selected from the group consisting of A2AR, A2BR, HIF1α, and HIF1α.I3.

4. The method of any one of claim 2, wherein the method increases resistance to hypoxic-adenosinergic immunosuppression of the modified immune cell and/or increases cytokine production of the modified immune cell relative to an unmodified reference immune cell.

5. The method of claim 2, wherein the one or more guide polynucleotides target a site selected from those listed in Table 1A and/or contain a spacer listed in Table 1A and/or Table 1B.

6. The method of claim 1, wherein the deaminase domain is an adenosine deaminase domain and guides 158, 170, and 173 are used to edit an HIF1α target site, guides 221 and 224 are used to edit an A2BR target site, or guide 155 is used to edit an A2BR target site.

7. The method of claim 1, wherein the deaminase domain is a cytidine deaminase domain and guides 145 and 155, or guides 222, 223, 225, and 226 are used to are used to edit an A2AR target site.

8. The method claim 2, wherein the method results in a reduction in hypoxia/adenosine-mediated suppression of cytotoxic T cell function.

9. The method of claim 2, wherein the guide polynucleotide directs the base editor to effect a nucleobase alteration that results in a premature stop codon in the gene, or wherein the nucleobase alteration is at a splice acceptor site of the gene.

10. The method of claim 1, wherein the deaminase domain comprises an adenosine deaminase domain and the adenosine deaminase domain comprises one or more of the following mutations: V82S, Y147T, Y147R, Q154S, Y123H, Q154R, and T166R as numbered in the amino acid sequence MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1) or a corresponding mutation thereof, or
wherein the adenosine deaminase domain comprises a combination of mutations selected from the group consisting of: Y147T Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R as numbered in SEQ ID NO: 2, or corresponding mutations thereof.

11. The method of claim 1, further comprising altering the cell to:
a) reduce or eliminate expression of one or more polypeptides selected from the group consisting of B2M, CD3ε, PD1, CIITA, CTLA4, LAG3, TIM3, TGFbR1, and TGFbR2;
b) reduce or eliminate expression of each of HLA Class I polypeptides, HLA Class II polypeptides, and A2AR;
c) reduce or eliminate expression of the following polypeptides: CD3F, B2M, and CIITA;
d) reduce or eliminate expression of the following polypeptides: A2AR and HIF1α; and/or
e) reduce or eliminate expression of one or more polypeptides selected from the group consisting of CD3F, CD36, CD37, B2M, CIITA, TRAC, and TRBC.

12. The method of claim 1, further comprising over-expressing Human Leukocyte Antigen-E (HLA-E) or Human Leukocyte Antigen-G (HLA-G) in the cell.

13. A modified immune cell produced according to the method of claim 1.

14. The modified immune cell of claim 13, wherein the cell comprises or further comprises a combination of alterations to polypeptides, wherein the combination of polypeptides is selected from the group consisting of:
a) β2M, TAP1, TAP2, and Tapasin;
b) TRAC, CD52, CIITA, HLA-E, HLA-G, PD-L1, PD1, and CD47;
c) TRAC, CD52, and CIITA;
d) HLA-E, HLA-G, PD-L1, PD1, and CD47;
e) one or more of β2M, TAP1, TAP2, and Tapasin, and one or more of HLA-E, HLA-G, PD-L1, PD1, and CD47;
f) B2M, CD3ε, and CIITA;
g) A2AR, B2M, CD3ε, and CIITA; and
h) A2AR, B2M, CD3ε, CIITA, PD1, and TGFbR2.

15. A base editor system that comprises (i) a base editor, or a nucleic acid sequence encoding the same and (ii) a guide polynucleotide or a nucleic acid sequence encoding the guide polynucleotide, wherein the base editor comprises a programmable DNA binding domain and a deaminase domain, wherein the guide polynucleotide comprises a sequence selected from the group consisting of:
(SEQ ID NO: 387)
UCACCGGAGCGGGAUGCGGA;
(SEQ ID NO: 388)
CUGCUCACCGGAGCGGGAUG;
(SEQ ID NO: 389)
CACUCCCAGGGCUGCGGGGA;
(SEQ ID NO: 390)
CCACUCCCAGGGCUGCGGGG;
(SEQ ID NO: 391)
GCGACGACAGCUGAAGCAGA;
(SEQ ID NO: 392)
UGGAGAGCCAGCCUCUGCCG;
(SEQ ID NO: 393)
GGAGAGCCAGCCUCUGCCGG;
(SEQ ID NO: 394)
ACAUGAGCCAGAGAGGGGCG;
(SEQ ID NO: 395)
GAGGCAGCAAGAACCUUUCA;
(SEQ ID NO: 396)
UGGCCCACACUCCUGGCGGG;
(SEQ ID NO: 397)
CGUUGGCCCACACUCCUGGC;
(SEQ ID NO: 398)
UCUCCCCAGGUACAAUGGCU;
(SEQ ID NO: 399)
CAGUUGUUCCAACCUAGCAU;
(SEQ ID NO: 400)
GGCCAUGCUGCUGGAGACAC;
(SEQ ID NO: 401)
UCACCUGAGCGGGACACAGA;
(SEQ ID NO: 402)
UUACUGUUCCACCCCAGGAA;
(SEQ ID NO: 403)
UUUAAACAGGUAUAAAAGUU;
(SEQ ID NO: 404)
GCUUCAGCGCACUGAGCUGA;
(SEQ ID NO: 405)
UGCCAAGCAGAUGUCAAGAG;
(SEQ ID NO: 406)
CUUACUAUCAUGAUGAGUUU;
(SEQ ID NO: 407)
CAUAUACCUGAGUAGAAAAU;
(SEQ ID NO: 408)
UCAUAUACCUGAGUAGAAAA;
(SEQ ID NO: 409)
UGUUUACAGUUUGAACUAAC;
(SEQ ID NO: 410)
UCAUUAGGCCUUGUGAAAAA;
(SEQ ID NO: 411)
ACACAGGUAUUGCACUGCAC;
(SEQ ID NO: 412)
UAACAGAAUUACCGAAUUGA;
(SEQ ID NO: 413)
AACAGAAUUACCGAAUUGAU;
(SEQ ID NO: 414)
UUUCAGAACUACAGUUCCUG;
(SEQ ID NO: 415)
AGCUCCCAAUGUCGGAGUUU;
(SEQ ID NO: 416)
GAGCUCCCAAUGUCGGAGUU;
(SEQ ID NO: 417)
UUAAAUGAGCUCCCAAUGUC;
(SEQ ID NO: 418)
UUUAAAUGAGCUCCCAAUGU;
and
(SEQ ID NO: 419)
ACCAUACCCAUUUUCUAUUC.

16. The base editor system of claim 15, wherein the deaminase domain is an adenosine deaminase domain comprising one or more of the following mutations: V28S, Y147T, Y147R, Q154S, Y123H, Q154R, and T166R mutation as numbered in the amino acid sequence MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1) or a corresponding mutation thereof, or
wherein the adenosine deaminase domain comprises a combination of mutations selected from the group consisting of: Y147T Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+176Y; V82S+Y123H+Y147R+Q154R; and I76Y+V82S+Y123H+Y147R+Q154R as numbered in SEQ ID NO: 2, or corresponding mutations thereof.

17. A cell comprising the base editor system of claim 15.

18. A pharmaceutical composition comprising an effective amount a modified immune cell produced according to the method of claim 1 and a pharmaceutically acceptable excipient.

19. A composition comprising a guide polynucleotide and a polynucleotide encoding a fusion protein comprising a polynucleotide programmable DNA binding domain and a deaminase domain, wherein the guide polynucleotide comprises a nucleic acid sequence that is complementary to a gene selected from the group consisting of A2AR, A2BR, HIF1α, and HIF1α.I3 genes.

20. The composition of claim 19, wherein the guide polynucleotide targets a site selected from those listed in Table 1A and/or contains a spacer sequence listed in Table 1A or Table 1B.

21. A kit comprising a modified immune cell produced according to the method of claim 1 and written instructions for using the modified immune cell.

22. A method of treating cancer in a subject, the method comprising administering to the subject an effective amount of a modified immune cell produced according to the method of claim 1.

23. A modified immune effector cell, wherein the modified immune effector cell expresses a chimeric antigen receptor targeting an antigen associated with a disease or disorder, and wherein the modified immune effector cell comprises reduced or undetectable expression of the following polypeptides: A2AR, CD3ε, B2M, and CIITA; or A2AR, B2M, CD3ε, CIITA, PD1, and TGFbR2.
